(19) United States
(12) Patent Application Publication
(10) Pub. No.: US 2026/0190608 A1
(43) Pub. Date: Jul. 2, 2026

(54) ORGANIC COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Tiantian MA, Xi'an (CN); Lei YANG, Xi'an (CN); Zhen FENG, Xi'an (CN)

(21) Appl. No.: 18/858,846

(22) PCT Filed: Sep. 19, 2023
(86) PCT No.: PCT/CN2023/119860

§ 371 Date: Oct. 22, 2024

(30) Foreign Application Priority Data
Feb. 7, 2023 (CN) 202310086510.4

(51) Int. Cl. H10K 50/15; C07B 59/00; C07D 307/87; C09K 11/06; H10K 85/30; H10K 85/60

(52) U.S. Cl. CPC H10K 50/15; C07B 59/002; C07D 307/87; C09K 11/06; H10K 85/342; H10K 85/626; H10K 85/633; H10K 85/636; H10K 85/6572; H10K 85/6574; H10K 85/6576; C07B 2200/05; C09K 2211/1088

(57) ABSTRACT

The present application relates to an organic compound, an organic electroluminescent device, and an electronic apparatus. The organic compound of the present application has a structure shown in a formula 1, and when the organic compound is applied in an organic electroluminescent device, the performance of the device can be significantly improved.

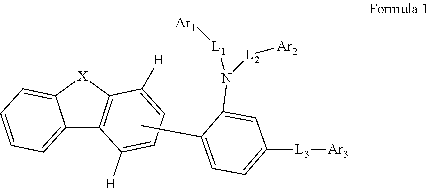

### CROSS REFERENCE TO RELATED APPLICATIONS

This application claims the priority of Chinese patent application No. CN202310086510.4, filed on Feb. 7, 2023, the contents of which are incorporated here in their entirety as part of this application.

### TECHNICAL FIELD

The present application belongs to the technical field of organic materials, and particularly relates to an organic compound, and an organic electroluminescent device and an electronic apparatus including the same.

### BACKGROUND

With the development of electronic technology and the advancement of material science, the research scope of electroluminescent or photoelectric conversion electronic components is becoming more and more extensive. Organic electroluminescent devices, also referred to as organic light-emitting diodes, refer to a phenomenon in which an organic light-emitting material emits light after being excited by an electric current under the action of an electric field. The electronic component generally includes a cathode and an anode which are oppositely disposed, and a functional layer disposed between the cathode and the anode. The functional layer is composed of a plurality of organic or inorganic film layers and generally includes an energy conversion layer, a hole transport layer located between the energy conversion layer and the anode, and an electron transport layer located between the energy conversion layer and the cathode. Taking an organic electroluminescent device as an example, the organic electroluminescent device generally includes an anode, a hole transport layer, an organic light-emitting layer as an energy conversion layer, an electron transport layer and a cathode which are sequentially stacked. When a voltage is applied to the cathode and the anode, an electric field is generated between the two electrodes, electrons on the cathode side move towards the organic light-emitting layer and holes on the anode side also move towards the organic light-emitting layer under the action of the electric field. The electrons and the holes are combined in the electroluminescent layer to form excitons, the excitons are in an excited state and release energy outwards, and then the electroluminescent layer emits light outwards.

At present, triarylamine materials in hole transport materials have excellent performance, and are one of the research hotspots. Although the prior art discloses that hole transport materials can be prepared in organic electroluminescent devices, existing triarylamine hole transport materials do not perform well in terms of voltage, luminous efficiency, power, and service life in the devices. Thus, it is still necessary to continue to develop new materials to further improve the performance of the electronic components.

### SUMMARY

In order to solve the above problems, the present application aims to provide an organic compound, and an organic electroluminescent device and an electronic apparatus including the same. The organic compound can improve the performance of the organic electroluminescent device and the electronic apparatus, such as reducing the driving voltage of the device, and improving the efficiency and service life of the device.

According to a first aspect of the present application, provided is an organic compound, having a structure as shown in a formula 1:

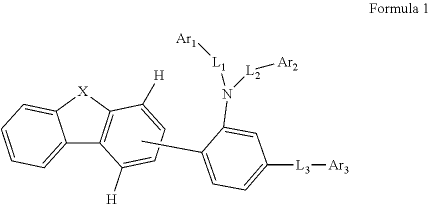

wherein X is O or S;
L₁ and L₂ are the same or different, and are respectively and independently selected from a single bond, a substituted or unsubstituted arylene with 6 to 30 carbon atoms, or a substituted or unsubstituted heteroarylene with 3 to 30 carbon atoms;
Ar₁ and Ar₂ are the same or different, and are respectively and independently selected from a substituted or unsubstituted aryl with 6 to 40 carbon atoms, or a substituted or unsubstituted heteroaryl with 3 to 40 carbon atoms;
L₃ is selected from a single bond, or substituted or unsubstituted arylene with 6 to 30 carbon atoms;
Ar₃ is selected from a substituted or unsubstituted aryl with 6 to 30 carbon atoms;
substituent(s) in L₃ and Ar₃ are the same or different, and are respectively and independently selected from deuterium, a halogen group, a cyano, an alkyl with 1 to 10 carbon atoms, a cycloalkyl with 3 to 20 carbon atoms, an aryl with 6 to 20 carbon atoms, or a deuterated aryl with 6 to 20 carbon atoms;
substituent(s) in L₁, L₂, Ar₁ and Ar₂ are the same or different, and are respectively and independently selected from deuterium, a halogen group, a cyano, an alkyl with 1 to 10 carbon atoms, a cycloalkyl with 3 to 20 carbon atoms, a heteroaryl with 12 to 20 carbon atoms, an aryl with 6 to 20 carbon atoms, a deuterated aryl with 6 to 20 carbon atoms, a haloaryl with 6 to 20 carbon atoms, a trialkylsilyl with 3 to 12 carbon atoms, a triarylsilyl with 18 to 24 carbon atoms, a haloalkyl with 1 to 10 carbon atoms, or a deuterated alkyl with 1 to 10 carbon atoms; and
optionally, in Ar₁ and Ar₂, any two adjacent substituents form a ring.

According to a second aspect of the present application, provided is an organic electroluminescent device, including an anode and a cathode which are oppositely disposed, and a functional layer disposed between the anode and the cathode, where the functional layer includes the above organic compound.

According to a third aspect of the present application, provided is an electronic apparatus, including the organic electroluminescent device in the second aspect.

The present application provides a compound with 2,5-disubstituted aniline as a core structure. An ortho position (2-position) of an amine group is linked to dibenzofuran/dibenzothiophene, and dibenzofuran/dibenzothiophene is specifically linked to aniline via a 2-position or a 3-position, which makes this moiety a partially electronic rigid planar group, so that the molecule can have good ortho-position spatial conjugation effects and photoelectric stability. Further, aryl is linked at a 5-position of aniline, so that the compound maintains molecular energy level characteristics while enhancing spatial effects, enabling the molecule to have a stable amorphous state and enhanced film-forming characteristics. When the compound of the present application is used as a material of a hole auxiliary layer in an organic electroluminescent device, the device can have improved luminous efficiency and service life performance while maintaining a low driving voltage.

Other features and advantages of the present application will be described in detail in the subsequent specific embodiments.

### BRIEF DESCRIPTION OF THE DRAWINGS

The accompanying drawings are used to provide a further understanding of the present application and constitute a part of the description, and are used to explain the present application together with the following specific embodiments, but do not constitute limitations on the present application.

FIG. 1 is a structural schematic diagram of an organic electroluminescent device according to the present application.

FIG. 2 is a structural schematic diagram of an electronic apparatus according to the present application.

### REFERENCE SIGNS

100, anode; 200, cathode; 300, functional layer; 310, hole injection layer; 320, hole transport layer; 330, hole auxiliary layer; 340, organic light-emitting layer; 350, electron transport layer; 360, electron injection layer; 400, electronic apparatus.

### DETAILED DESCRIPTION

Examples will now be described more fully with reference to the accompanying drawings. However, the examples can be implemented in a variety of forms, and should not be understood as a limitation to the instances set forth here; and on the contrary, these examples are provided such that the present application will be more comprehensive and complete, and the concepts of the examples are comprehensively conveyed to those skilled in the art. The described features, structures, or characteristics may be incorporated in one or more embodiments in any suitable manner. In the following description, many specific details are provided to give a sufficient understanding of the examples of the present application.

In a first aspect, the present application provides an organic compound, having a structure as shown in a formula 1:

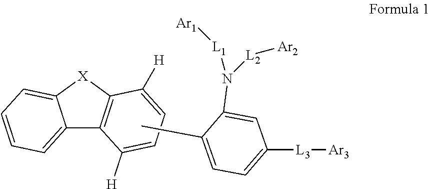

wherein X is O or S;
L₁ and L₂ are the same or different, and are respectively and independently selected from a single bond, a substituted or unsubstituted arylene with 6 to 30 carbon atoms, or a substituted or unsubstituted heteroarylene with 3 to 30 carbon atoms;
Ar₁ and Ar₂ are the same or different, and are respectively and independently selected from a substituted or unsubstituted aryl with 6 to 40 carbon atoms, or a substituted or unsubstituted heteroaryl with 3 to 40 carbon atoms;
L₃ is selected from a single bond, or a substituted or unsubstituted arylene with 6 to 30 carbon atoms;
Ar₃ is selected from a substituted or unsubstituted aryl with 6 to 30 carbon atoms;
substituent(s) in L₃ and Ar₃ are the same or different, and are respectively and independently selected from deuterium, a halogen group, a cyano, an alkyl with 1 to 10 carbon atoms, a cycloalkyl with 3 to 20 carbon atoms, an aryl with 6 to 20 carbon atoms, or a deuterated aryl with 6 to 20 carbon atoms;
substituent(s) in L₁, L₂, Ar₁ and Ar₂ are the same or different, and are respectively and independently selected from deuterium, a halogen group, a cyano, an alkyl with 1 to 10 carbon atoms, a cycloalkyl with 3 to 20 carbon atoms, a heteroaryl with 12 to 20 carbon atoms, an aryl with 6 to 20 carbon atoms, a deuterated aryl with 6 to 20 carbon atoms, a haloaryl with 6 to 20 carbon atoms, a trialkylsilyl with 3 to 12 carbon atoms, a triarylsilyl with 18 to 24 carbon atoms, a haloalkyl with 1 to 10 carbon atoms, or a deuterated alkyl with 1 to 10 carbon atoms; and
optionally, in Ar₁ and Ar₂, any two adjacent substituents form a ring.

In the present application, in the formula 1, a linking site for a group

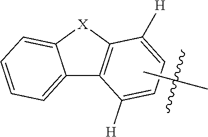

is as shown in a formula

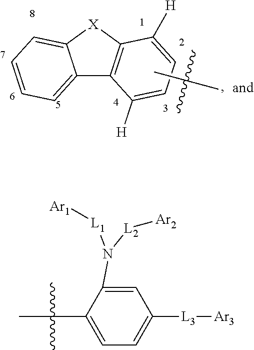

can only be linked at a 2-position or a 3-position of

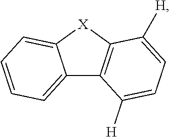

and none of the other positions have a substituent; that is,

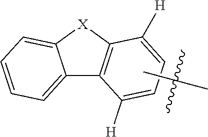

is only

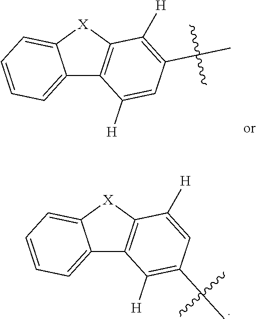

In the present application, the terms “optional” and “optionally” mean that the subsequently described event or circumstance may but need not occur, and that the description includes instances where the event or circumstance occurs and instances where the event or circumstance does not occur. For example, “optionally, any two adjacent substituents form a ring”, which means that the two substituents can form a ring but do not necessarily form a ring, including the scenario where two adjacent substituents form a ring and the scenario where two adjacent substituents do not form a ring.

In the present application, in the condition that “any two adjacent substituents form a ring”, “any two adjacent substituents” can include that the same atom have two substituents and can also include that two adjacent atoms each have one substituent; when the same atom have two substituents, the two substituents may form a saturated or unsaturated ring with the atom to which they are commonly connected; and when two adjacent atoms each have one substituent, the two substituents may be fused to form a ring. For example, when Ar₁ has 2 or more substituents and any adjacent substituents form a ring, a saturated or unsaturated cyclic group is formed, such as a benzene ring, a naphthalene ring, a phenanthrene ring, an anthracene ring, a fluorene ring, cyclopentane, cyclohexane, adamantane, and the like.

In the present application, fluorenyl may be substituted by 1 or 2 substituent(s), where in the case that the above fluorenyl is substituted, the substituted fluorenyl may be

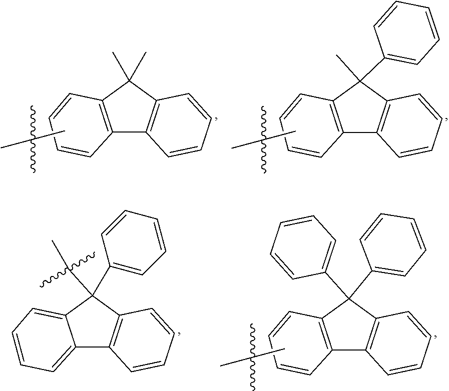

or the like, but is not limited to this.

In the present application, the adopted description modes “each . . . is independently”, “ . . . is respectively and independently” and “ . . . is each independently selected from” can be interchanged, and should be understood in a broad sense, which means that in different groups, specific options expressed between the same symbols do not influence each other, or in a same group, specific options expressed between the same symbols do not influence each other. For example, the meaning of

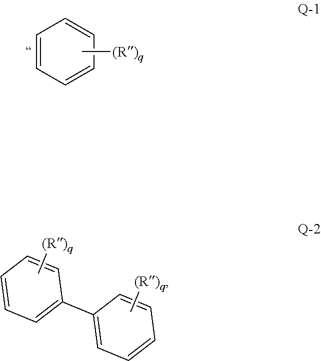

where each q is independently 0, 1, 2 or 3, and each R″ is independently selected from hydrogen, deuterium, fluorine and chlorine” is as follows: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present application, the term such as “substituted or unsubstituted” means that a functional group described behind the term may have or not have a substituent (in the below, the substituent is collectively referred to as Rc in order to facilitate description). For example, the “substituted or unsubstituted aryl” refers to aryl having the substituent Rc or unsubstituted aryl. Where the above substituent, i.e., Rc, for example, can be deuterium, a halogen group, cyano, alkyl, cycloalkyl, aryl, heteroaryl, deuterated aryl, haloaryl, trialkylsilyl, triarylsilyl, haloalkyl, deuterated alkyl, or the like.

In the present application, the number of carbon atoms in a substituted or unsubstituted functional group refers to the number of all carbon atoms. For example, if L₁ is substituted arylene with 12 carbon atoms, the number of all carbon atoms of the arylene and substituents on the arylene is 12.

In the present application, aryl refers to an optional functional group or substituent derived from an aromatic carbocyclic ring. The aryl may be monocyclic aryl (e.g., phenyl) or polycyclic aryl, in other words, the aryl may be monocyclic aryl, fused aryl, two or more monocyclic aryl linked by carbon-carbon bonds, monocyclic aryl and fused aryl which are linked by a carbon-carbon bond, or two or more fused aryl conjugatedly linked by carbon-carbon bonds. That is, unless otherwise indicated, two or more aromatic groups linked by carbon-carbon bonds may also be considered as the aryl in the present application. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. Examples of the aryl can include, but are not limited to, phenyl, naphthyl, fluorenyl, anthryl, phenanthryl, biphenyl, terphenyl, benzo[9,10]phenanthryl, pyrenyl, benzofluoranthenyl, triphenylene, chrysenyl, spirobifluorenyl, and the like. In the present application, the arylene involved refers to a divalent group formed by further loss of one hydrogen atom from aryl.

In the present application, terphenyl includes

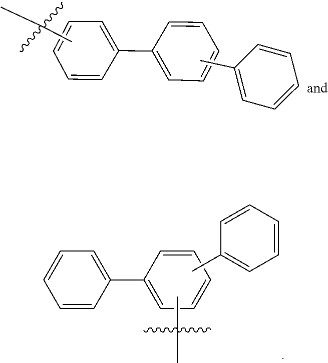

In the present application, the substituted aryl may be that one or two or more hydrogen atoms in the aryl are substituted by groups such as a deuterium atom, a halogen group, cyano, aryl, heteroaryl, alkyl, cycloalkyl, and the like. It should be understood that the number of carbon atoms of the substituted aryl refers to the total number of carbon atoms of the aryl and substituents on the aryl, for example, substituted aryl with 18 carbon atoms means that the total number of carbon atoms of the aryl and substituents is 18.

In the present application, heteroaryl refers to a monovalent aromatic ring containing 1, 2, 3, 4, 5, 6 or 7 heteroatoms in the ring or its derivative, and the heteroatom may be at least one of B, O, N, P, Si, Se, and S. The heteroaryl may be monocyclic heteroaryl or polycyclic heteroaryl, in other words, the heteroaryl may be a single aromatic ring system or a plurality of aromatic ring systems linked by carbon-carbon bonds, and any one aromatic ring system is a monocyclic aromatic ring or a fused aromatic ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, silafluorenyl, dibenzofuranyl, as well as N-phenylcarbazolyl, N-pyridylcarbazolyl, N-methylcarbazolyl and the like, but is not limited to this. Where thienyl, furyl, phenanthrolinyl, etc. are heteroaryl of the single aromatic ring system, and N-phenylcarbazolyl and N-pyridylcarbazolyl are heteroaryl of the plurality of aromatic ring systems linked by carbon-carbon bonds. In the present application, the heteroarylene involved refers to a divalent group formed by further loss of one hydrogen atom from heteroaryl.

In the present application, the substituted heteroaryl may be that one or two or more hydrogen atoms in the heteroaryl are substituted by groups such as a deuterium atom, a halogen group, cyano, aryl, heteroaryl, alkyl, cycloalkyl, and the like. It should be understood that the number of carbon atoms of the substituted heteroaryl refers to the total number of carbon atoms of heteroaryl and substituents on the heteroaryl.

In the present application, the number of carbon atoms of the substituted or unsubstituted aryl may be 6 to 25, for example, the number of carbon atoms may be 6, 7, 8, 9, 10, 11, 12, 13, 14, 15, 16, 17, 18, 19, 20, 21, 22, 23, 24, or 25.

In the present application, specific examples of aryl as a substituent include, but are not limited to, phenyl, biphenyl, naphthyl, fluorenyl, phenanthryl, anthryl, and chrysenyl.

In the present application, the number of carbon atoms of the substituted or unsubstituted heteroaryl may be 12 to 20, for example, the number of carbon atoms may be 12, 13, 14, 15, 16, 17, 18, 19, or 20.

In the present application, specific examples of heteroaryl as a substituent include, but are not limited to, carbazolyl, dibenzofuranyl, and dibenzothienyl.

In the present application, an unpositioned connecting bond refers to a single bond

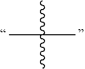

extending from a ring system, which means that one end of the connecting bond can be connected with any position in the ring system through which the bond penetrates, and the other end of the connecting bond is connected with the remaining part of a compound molecule.

For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1) to (f-10).

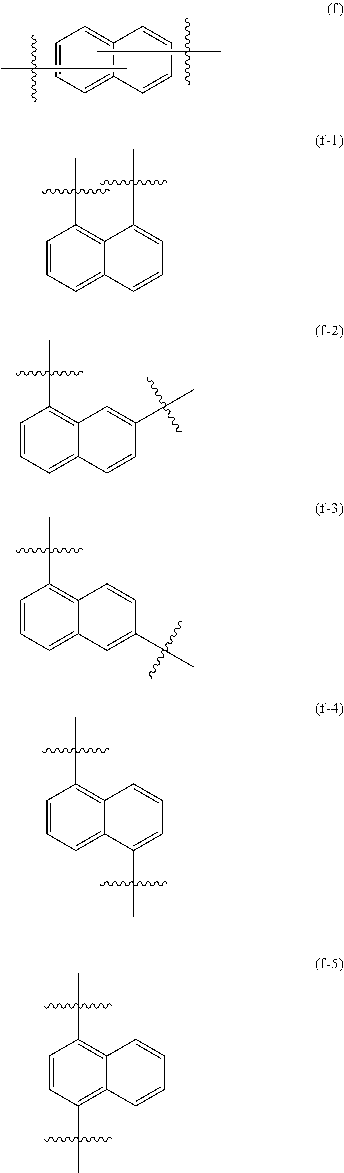

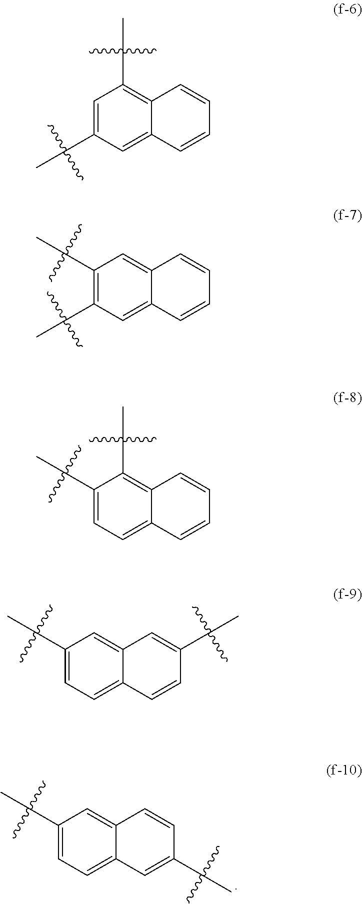 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

For another example, as shown in the following formula (X′), dibenzofuranyl represented by the formula (X′) is connected with other positions of a molecule through one unpositioned connecting bond extending from the middle of a benzene ring on one side, and its meaning includes any one possible connecting mode represented by formulae (X′-1) to (X′-4).

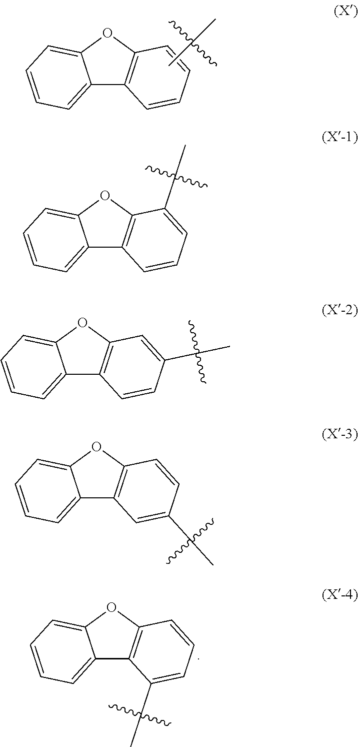

In the present application, the alkyl with 1 to 10 carbon atoms may include linear alkyl with 1 to 10 carbon atoms and branched alkyl with 3 to 10 carbon atoms. The number of carbon atoms of the alkyl may be, for example, 1, 2, 3, 4, 5, 6, 7, 8, 9, or 10, and specific examples of the alkyl include, but are not limited to, methyl, ethyl, n-propyl, isopropyl, n-butyl, isobutyl, tert-butyl, n-pentyl, isopentyl, neopentyl, n-hexyl, n-octyl, 2-ethylhexyl, nonyl, decyl, 3,7-dimethyloctyl, and the like.

In the present application, the halogen group may be, for example, fluorine, chlorine, bromine or iodine.

In the present application, the number of carbon atoms of the cycloalkyl with 3 to 20 carbon atoms may be, for example, 3, 4, 5, 6, 7, 8, 10, 11, 12, 13, 14, 15, 16, 17, 18, 19, or 20. Specific examples of the cycloalkyl include, but are not limited to, cyclopentyl and cyclohexyl.

In the present application, specific examples of the deuterated aryl group include, but are not limited to, pentadeuterophenyl.

In some embodiments of the present application, the organic compound is selected from compounds represented by a formula 1-1 or a formula 1-2:

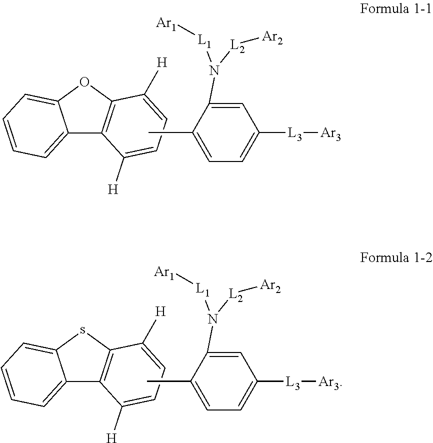

In some specific embodiments of the present application, the organic compound is selected from compounds represented by a formula A, a formula B, a formula C or a formula D:

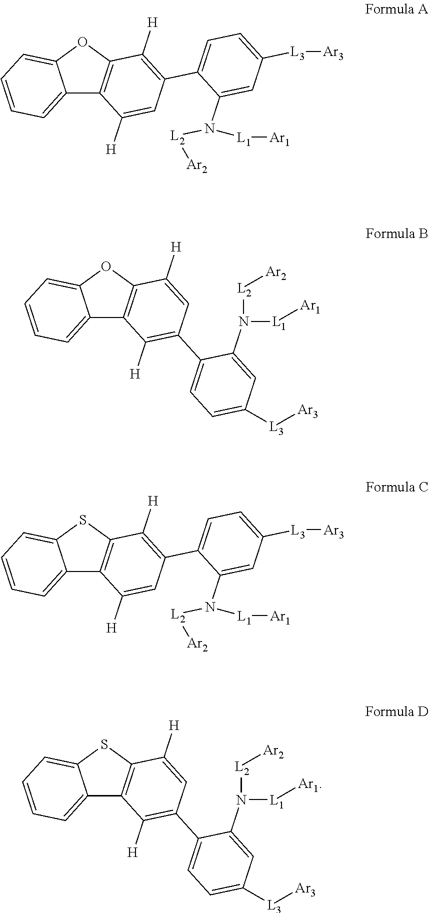

In some embodiments of the present application, L₁ and L₂ are the same or different, and are respectively and independently selected from a single bond, a substituted or unsubstituted arylene with 6 to 15 carbon atoms, or a substituted or unsubstituted heteroarylene with 12 to 20 carbon atoms.

Optionally, substituent(s) in L₁ and the L₂ are the same or different, and are respectively and independently selected from deuterium, a halogen group, a cyano, an alkyl with 1 to 5 carbon atoms or a phenyl.

In other embodiments of the present application, L₁ and L₂ are the same or different, and are respectively and independently selected from a single bond, a substituted or unsubstituted phenylene, a substituted or unsubstituted naphthylene, a substituted or unsubstituted biphenylene, a substituted or unsubstituted carbazolylene, a substituted or unsubstituted dibenzofurylene, or a substituted or unsubstituted dibenzothenylene.

Optionally, the substituent(s) in L₁ and the L₂ are the same or different, and are respectively and independently selected from deuterium, fluorine, cyano, methyl, ethyl, n-propyl, isopropyl, tert-butyl or phenyl.

In some embodiments of the present application, L₁ and L₂ are the same or different, and are respectively and independently selected from a single bond, and a substituted or unsubstituted group V, where the unsubstituted group V is selected from the group consisting of:

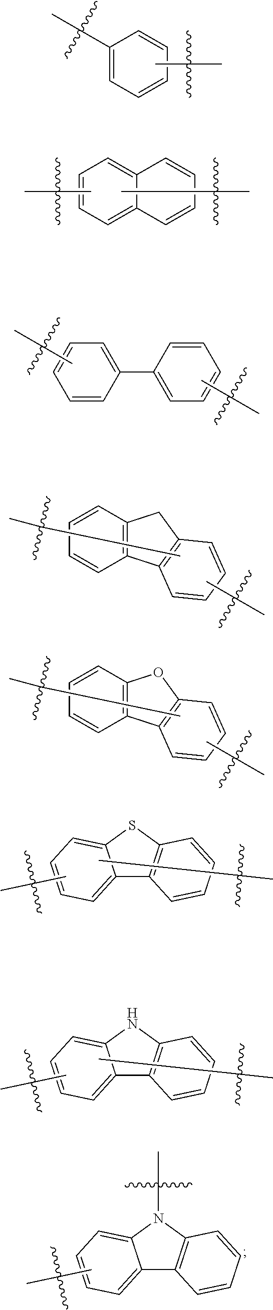

and
the substituted group V has one or two or more substituents independently selected from deuterium, fluorine, cyano, methyl, ethyl, isopropyl, tert-butyl or phenyl, and when the number of the substituents is greater than 1, the substituents are the same or different.

Optionally, L₁ and L₂ are the same or different, and are respectively and independently selected from a single bond or the group consisting of:

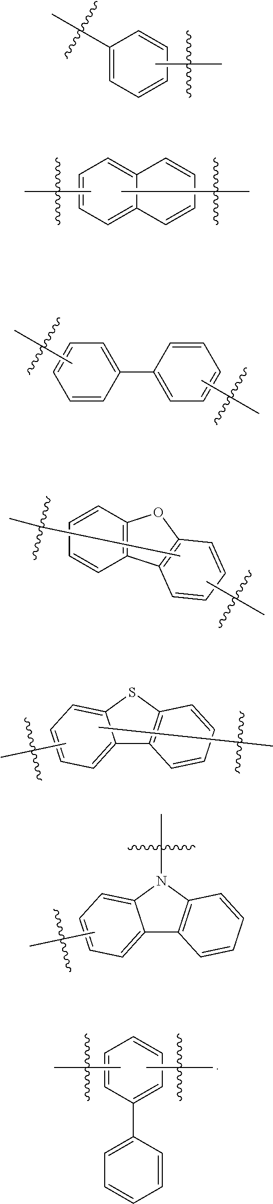

In particular, L₁ and L₂ are the same or different, and are respectively and independently selected from a single bond or the group consisting of:

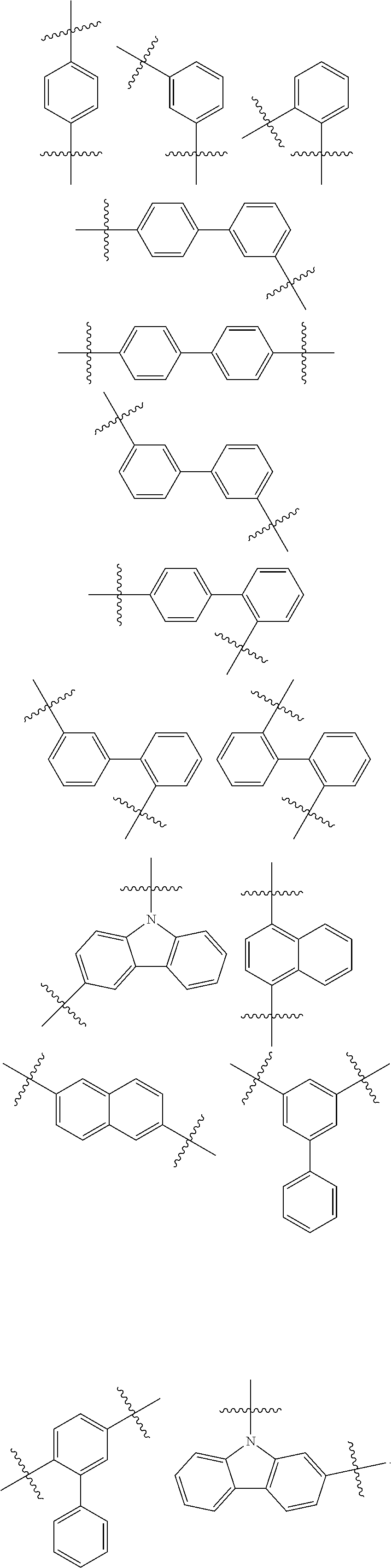

In some embodiments of the present application, Ar₁ and Ar₂ are the same or different, and are respectively and independently selected from substituted or unsubstituted aryl with 6 to 25 carbon atoms, and substituted or unsubstituted heteroaryl with 12 to 20 carbon atoms;
optionally, substituent(s) in Ar₁ and the Ar₂ are the same or different, and are respectively and independently selected from deuterium, a halogen group, a cyano, an alkyl with 1 to 5 carbon atoms, a phenyl, or a pentadeuterophenyl; and
optionally, in Ar₁ and Ar₂, any two adjacent substituents form a fluorene ring

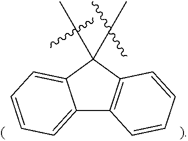 [SMILES: *C1(*)c2ccccc2-c2ccccc21]

In other embodiments of the present application, Ar₁ and Ar₂ are the same or different, and are respectively and independently selected from a substituted or unsubstituted terphenyl, a substituted or unsubstituted phenyl, a substituted or unsubstituted naphthyl, a substituted or unsubstituted biphenyl, a substituted or unsubstituted phenanthryl, a substituted or unsubstituted fluorenyl, a substituted or unsubstituted triphenylene, a substituted or unsubstituted carbazolyl, a substituted or unsubstituted dibenzofuranyl, a substituted or unsubstituted dibenzothienyl, or a substituted or unsubstituted spirobifluorenyl; and
optionally, the substituent(s) in Ar₁ and the Ar₂ are the same or different, and are respectively and independently selected from deuterium, fluorine, cyano, methyl, ethyl, n-propyl, isopropyl, tert-butyl, phenyl or pentadeuterophenyl.

In some embodiments of the present application, Ar₁ and Ar₂ are the same or different, and are respectively and independently selected from a substituted or unsubstituted group G, where the unsubstituted group G is selected from the group consisting of:

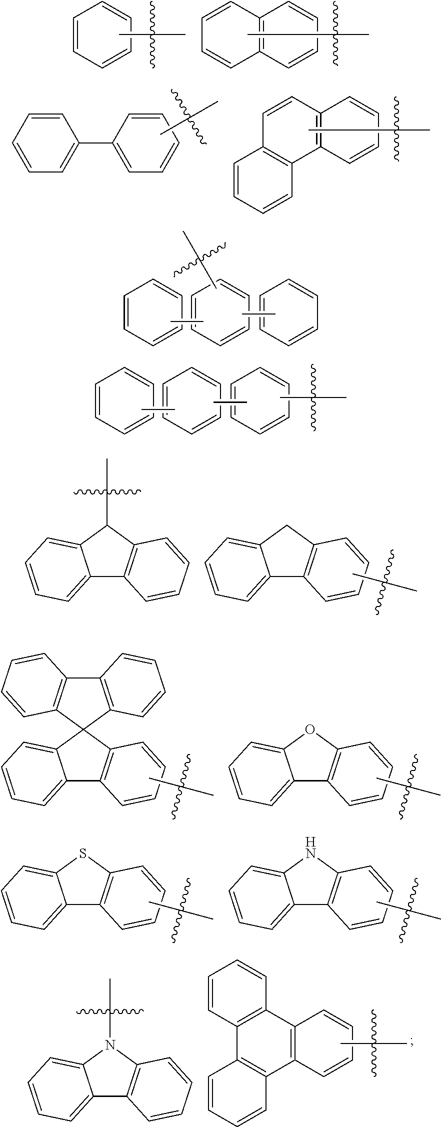

and

the substituted group G has one or two or more substituents, the substituents in the substituted group G are respectively and independently selected from the group consisting of deuterium, fluorine, cyano, phenyl, methyl, ethyl, n-propyl, isopropyl, tert-butyl, phenyl, and pentadeuterophenyl, and when the number of the substituents of the group G is greater than 1, the substituents are the same or different.

Optionally, Ar₁ and Ar₂ are the same or different, and are respectively and independently selected from the group consisting of:

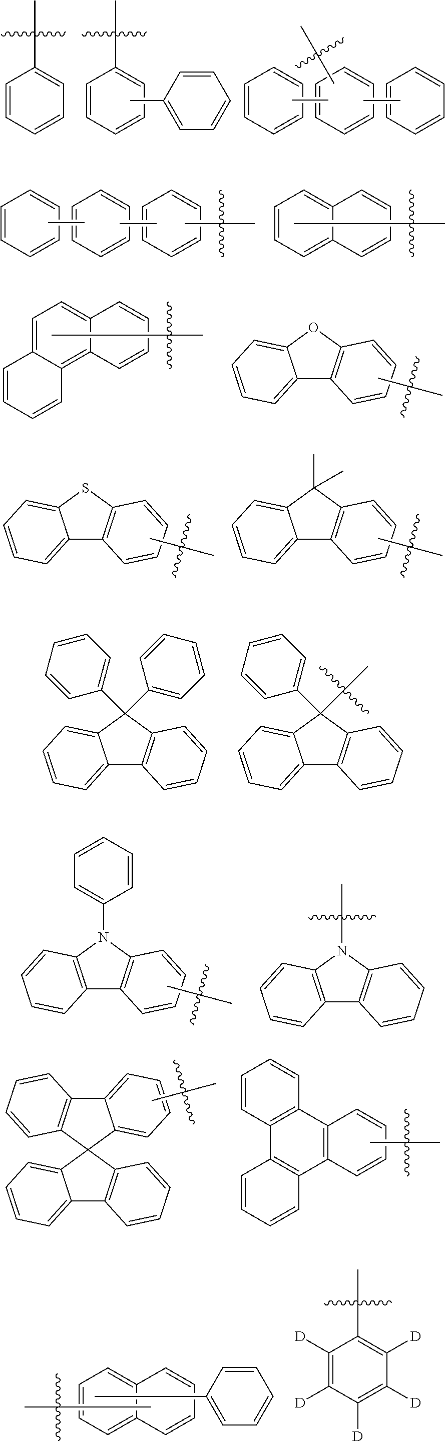

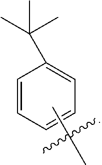

In particular, Ar₁ and Ar₂ are the same or different, and are respectively and independently selected from the group consisting of:

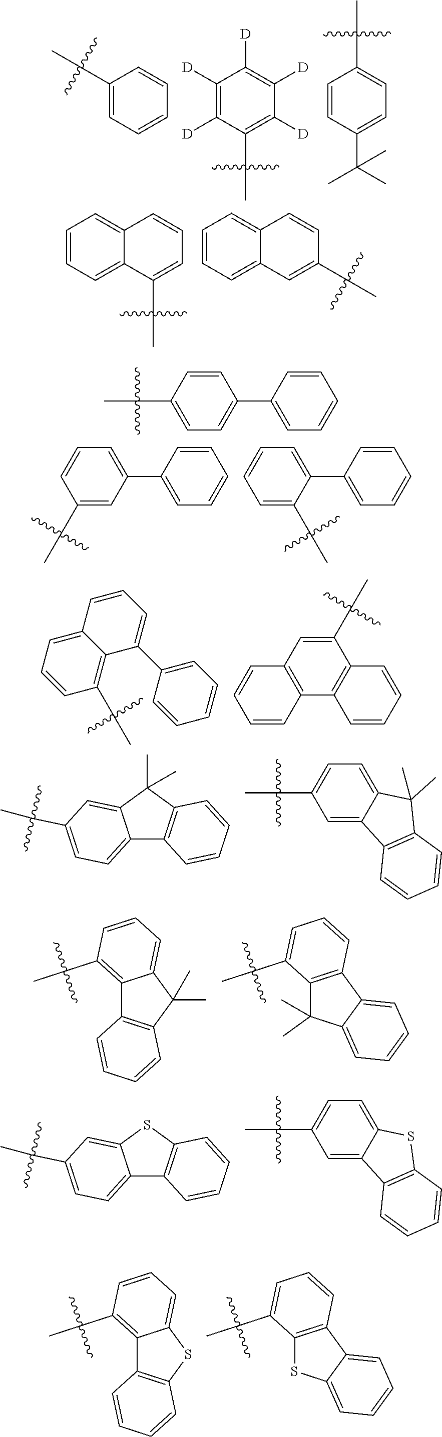

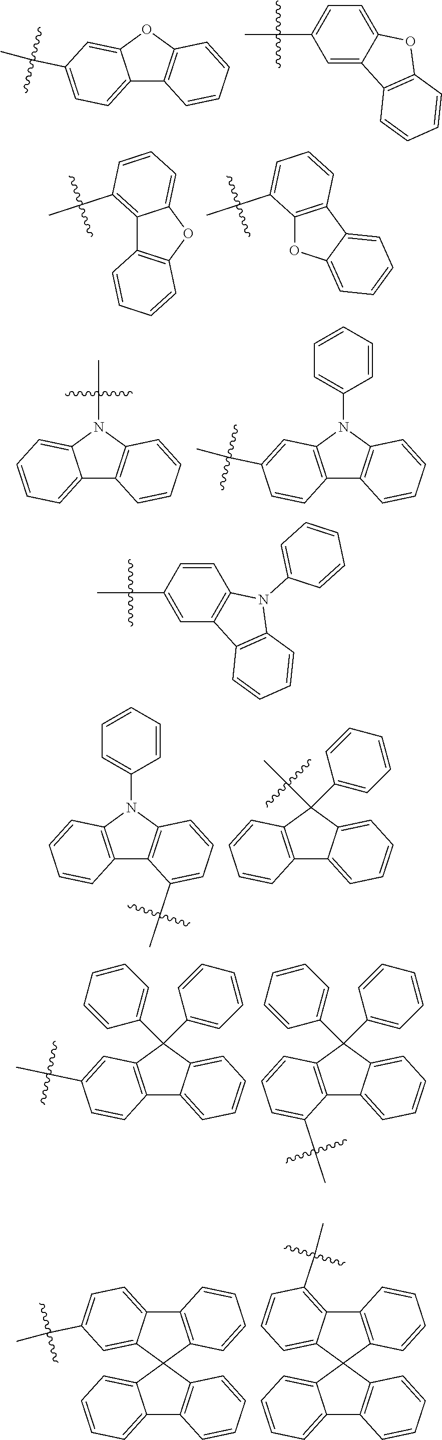

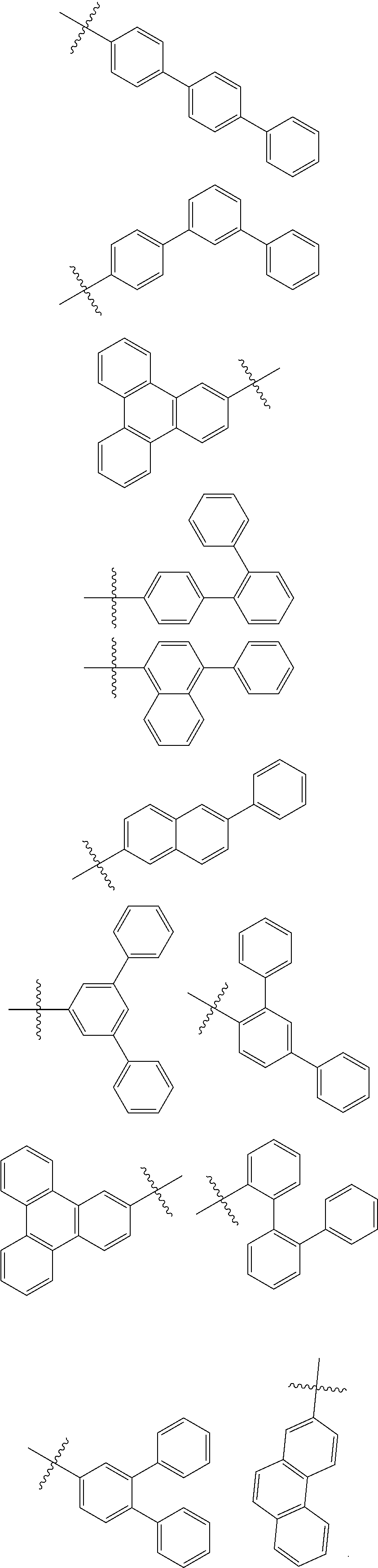

In some embodiments of the present application,

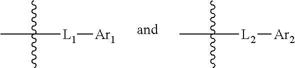

are the same or different, and are respectively and independently selected from the group consisting of:

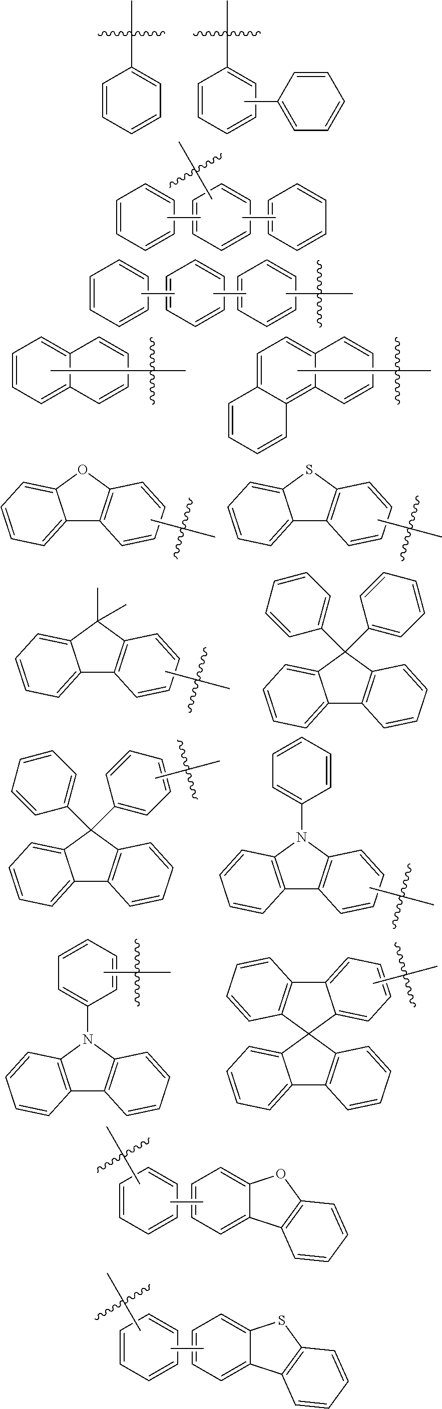

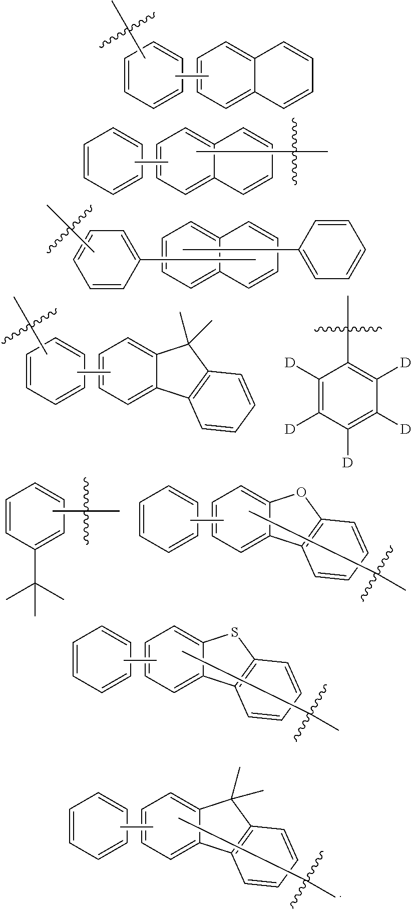

In particular,

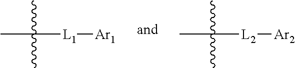

are the same or different, and are respectively and independently selected from the group consisting of:

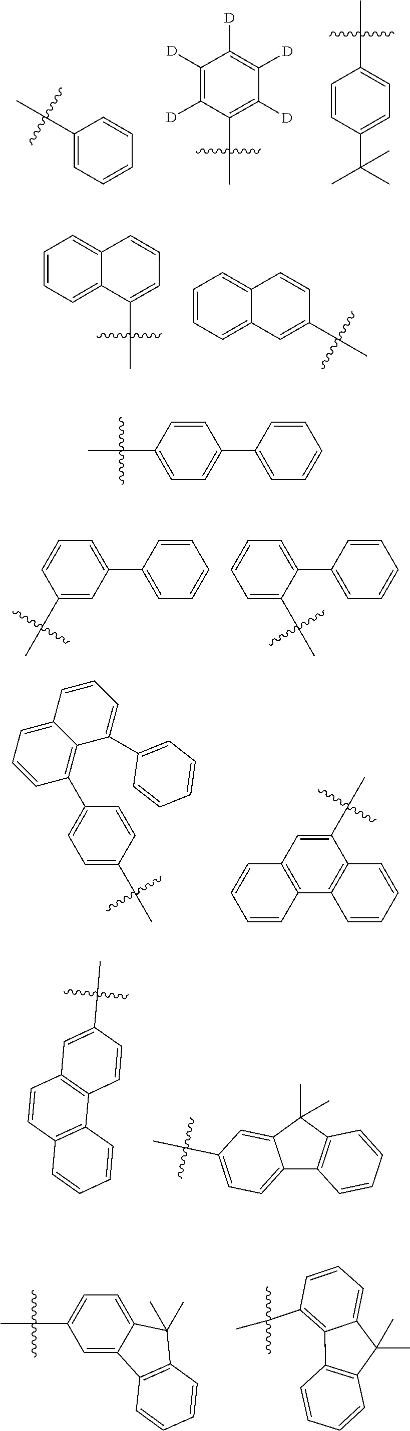

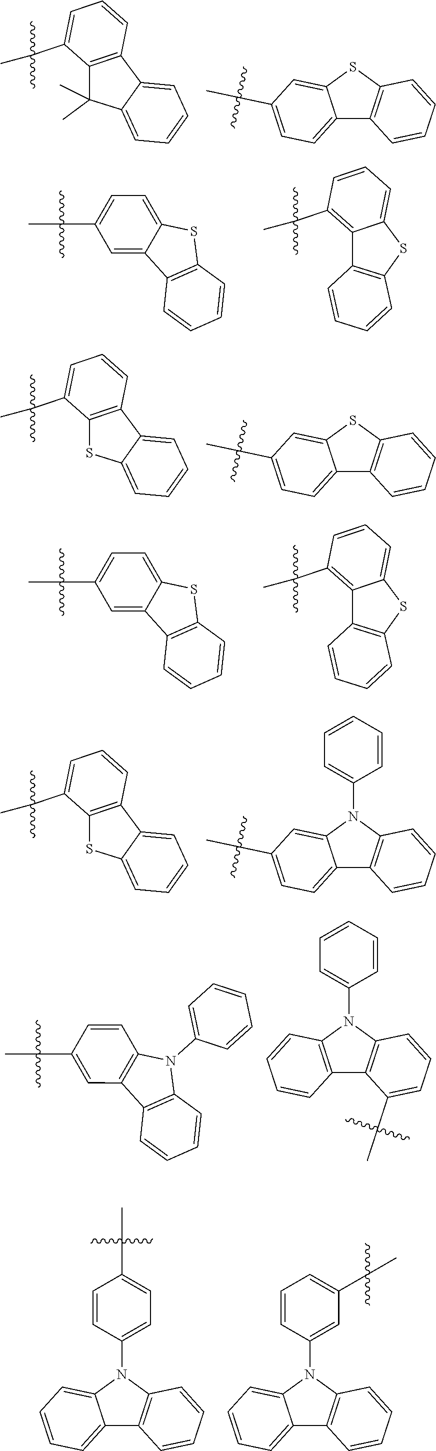

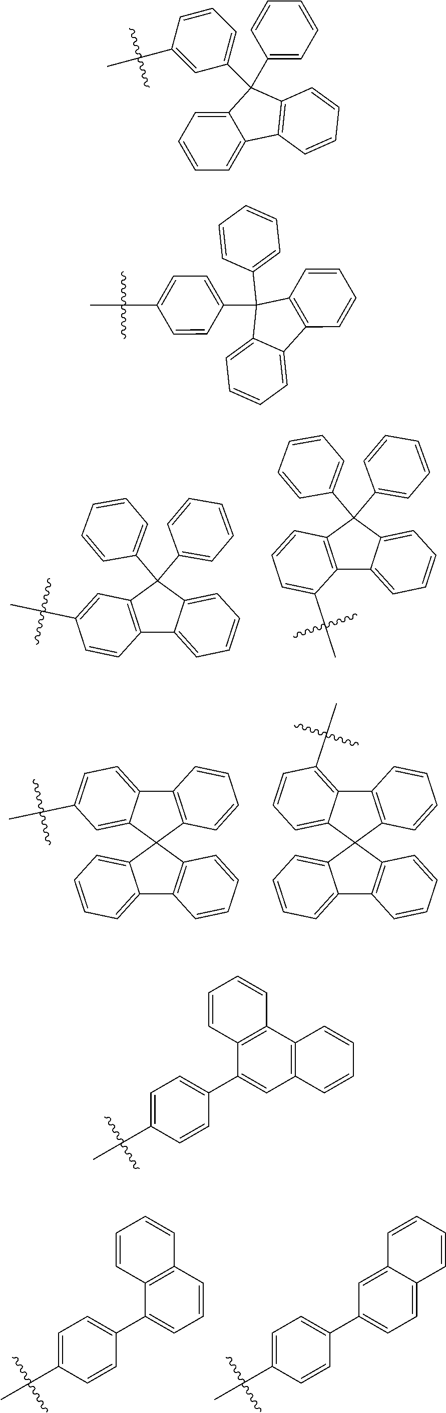

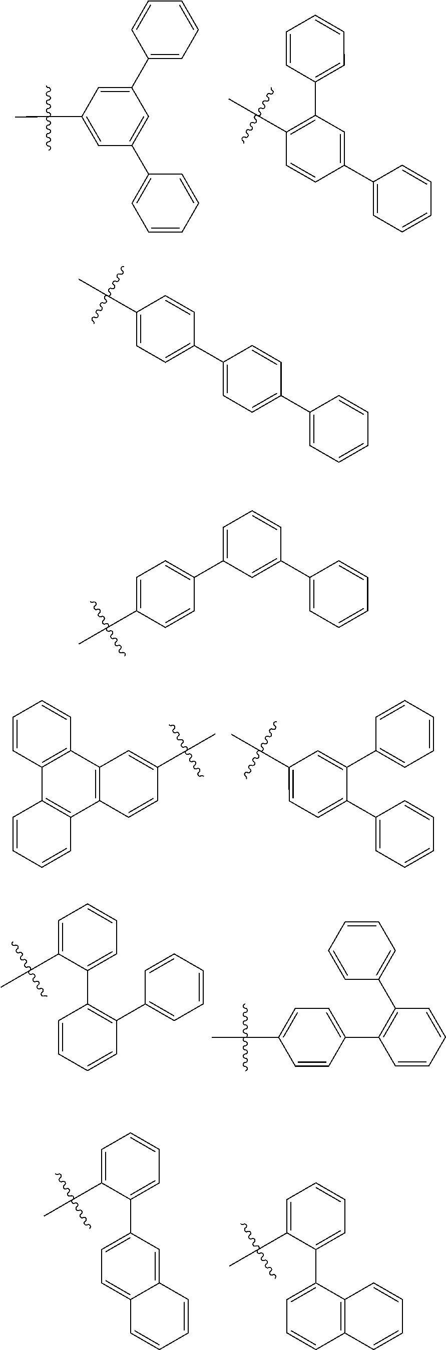

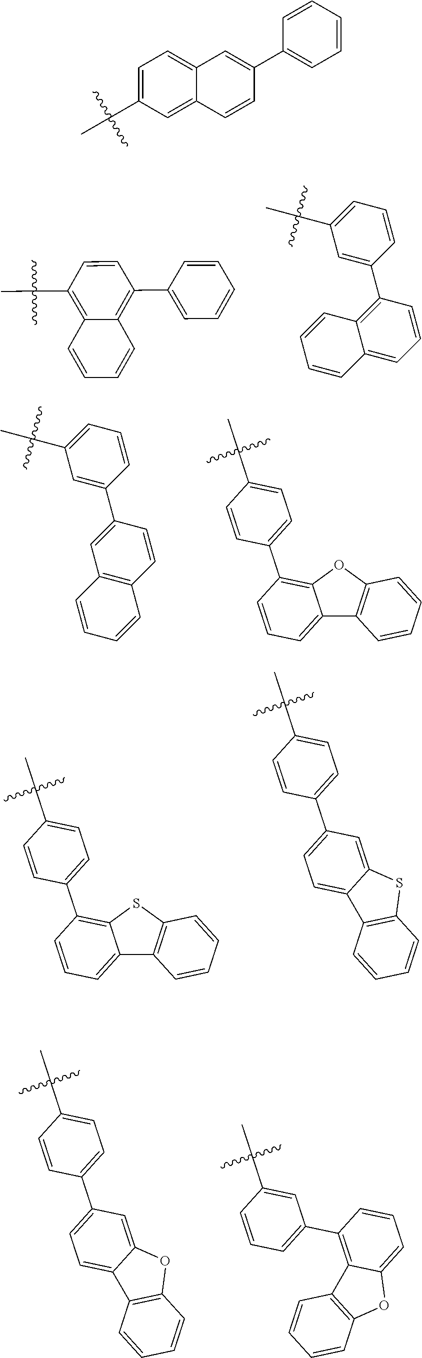

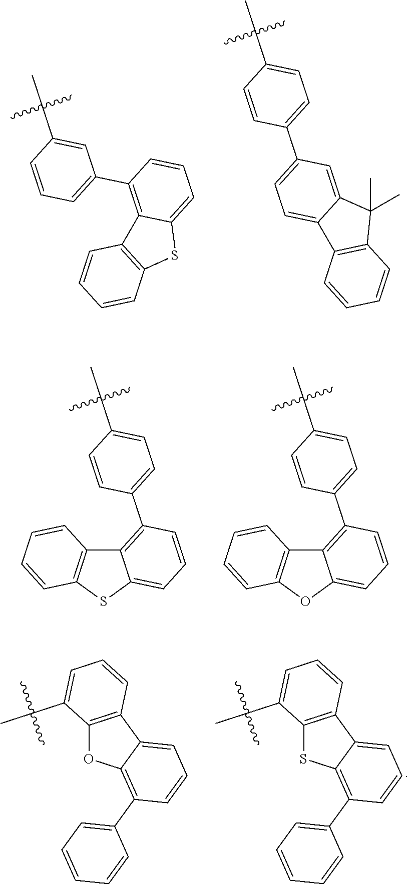

In some embodiments of the present application, L₃ is selected from a single bond, a substituted or unsubstituted phenylene, or a substituted or unsubstituted naphthylene.

Optionally, substituent(s) in L₃ are the same or different, and are respectively and independently selected from deuterium, fluorine, cyano, methyl, ethyl, n-propyl, isopropyl, tert-butyl or phenyl.

In particular, L₃ is selected from a single bond or the group consisting of:

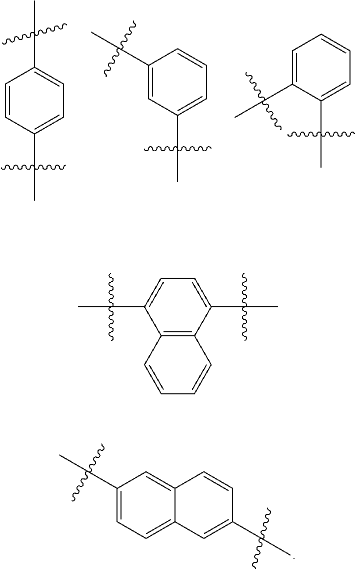 [SMILES: *c1ccc(*)cc1 ; *c1cccc(*)c1 ; *c1ccccc1* ; *c1ccc(*)c2ccccc12 ; *c1ccc2cc(*)ccc2c1]

In some embodiments of the present application, Ar₃ is selected from a substituted or unsubstituted aryl with 6 to 15 carbon atoms.

Optionally, substituent(s) in Ar₃ are the same or different, and are respectively and independently selected from deuterium, a halogen group, a cyano, an alkyl with 1 to 5 carbon atoms, a phenyl, or a pentadeuterophenyl.

In other embodiments of the present application, Ar₃ is selected from a substituted or unsubstituted phenyl, a substituted or unsubstituted naphthyl, a substituted or unsubstituted biphenyl, or a substituted or unsubstituted phenanthryl.

Optionally, the substituent(s) in Ar₃ are the same or different, and are respectively and independently selected from deuterium, fluorine, cyano, methyl, ethyl, n-propyl, isopropyl, tert-butyl, phenyl or pentadeuterophenyl.

Optionally, Ar₃ is selected from the group consisting of:

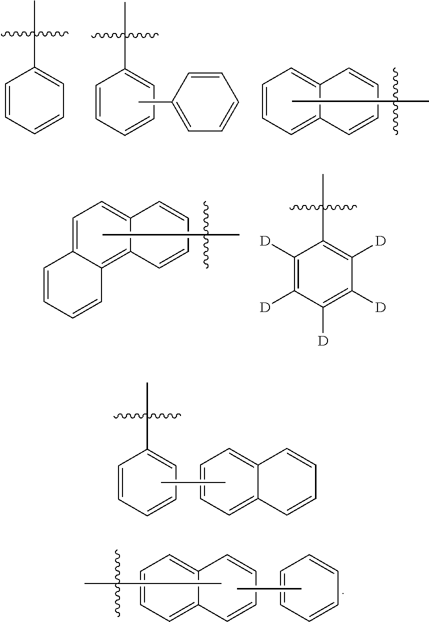

In particular, Ar₃ is selected from the group consisting of:

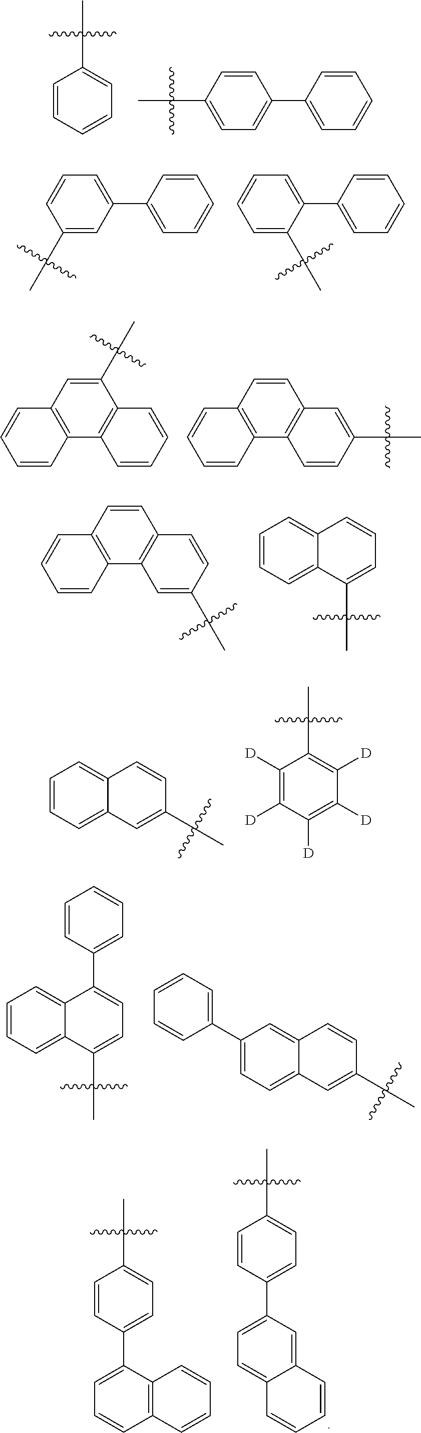

In some embodiments of the present application,

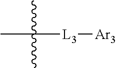

is selected from the group consisting of:

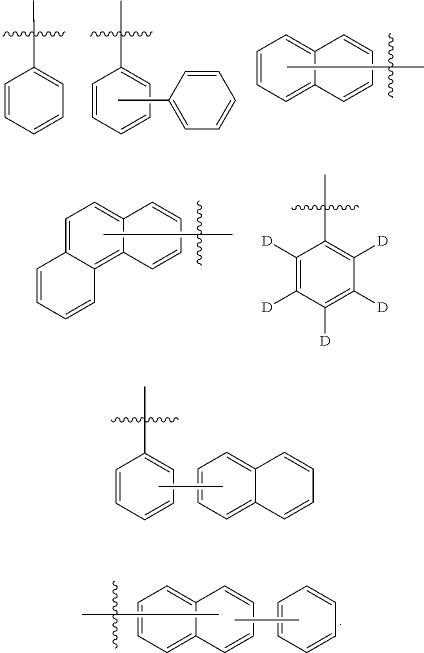

In particular,

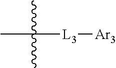

is selected from the group consisting of:

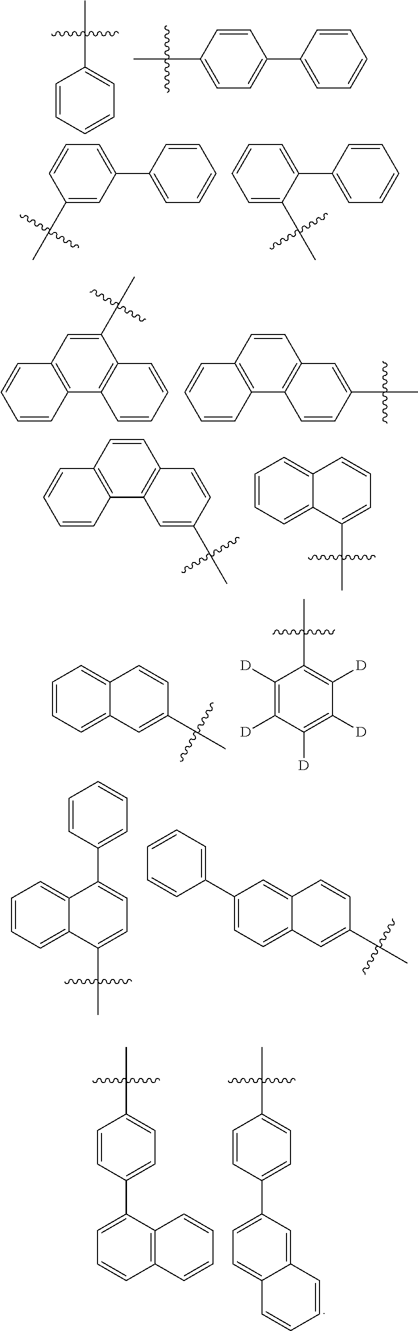

In the present application,

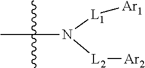

in the formula 1 is selected from the group consisting of:

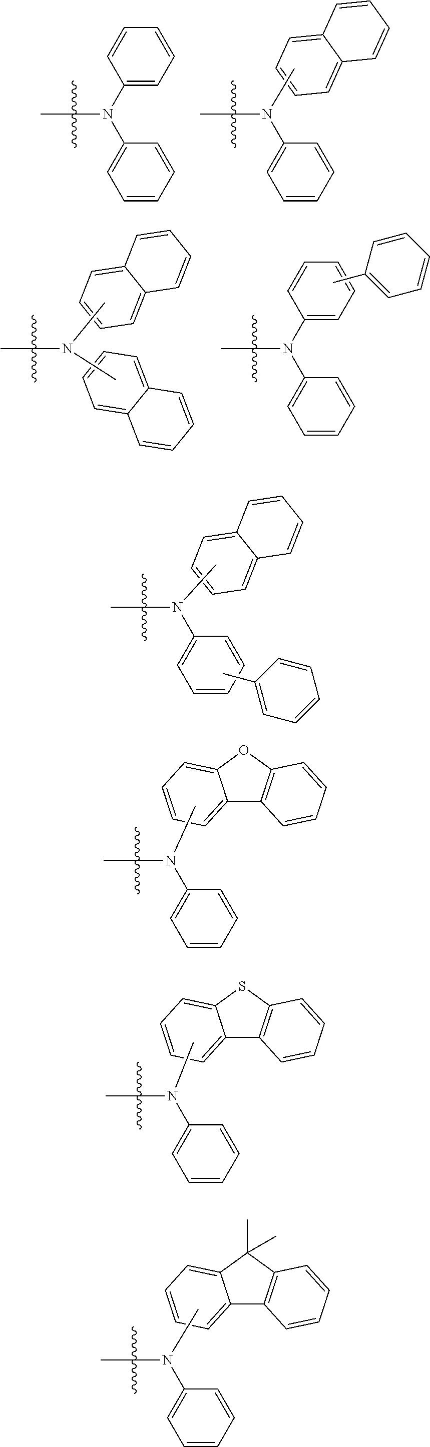

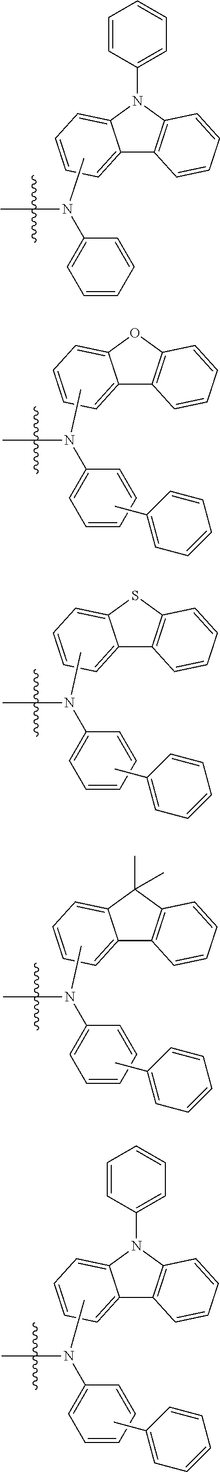

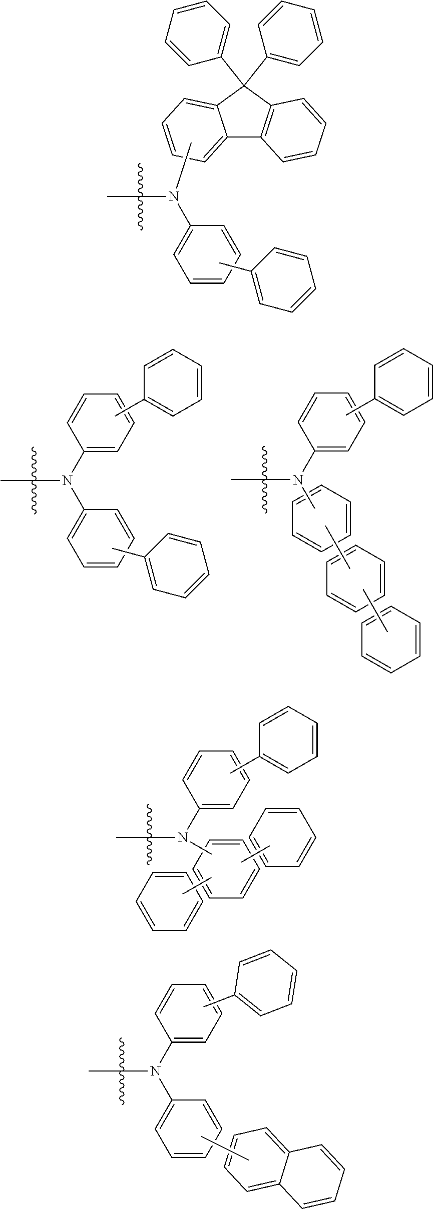

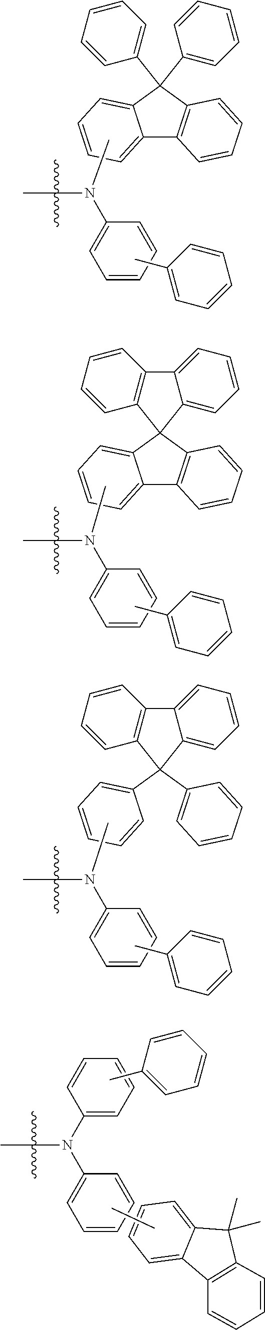

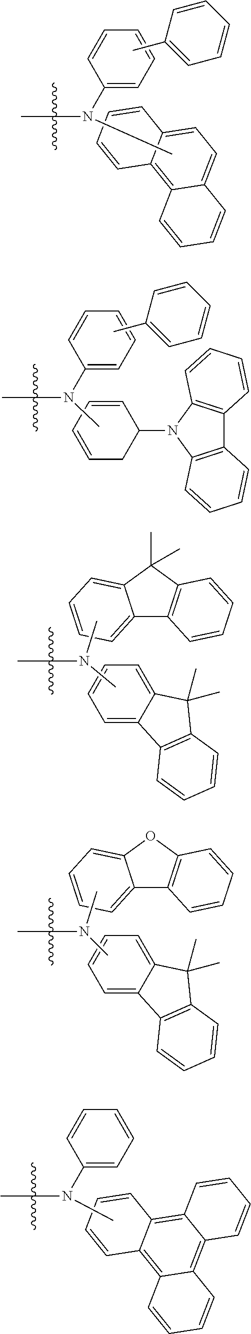

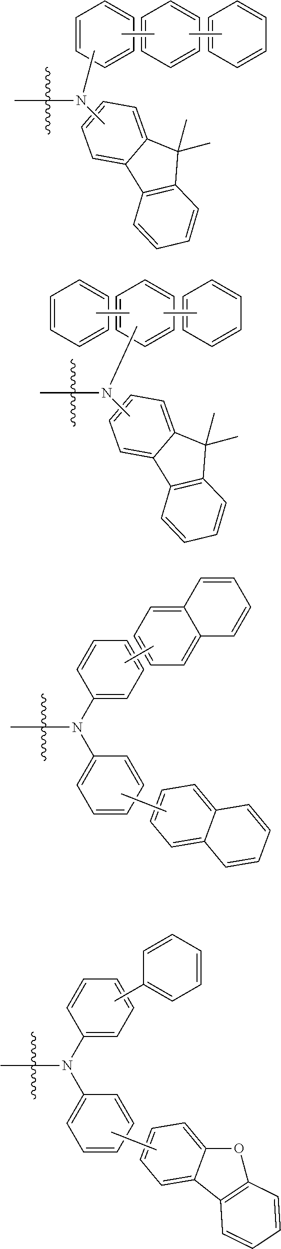

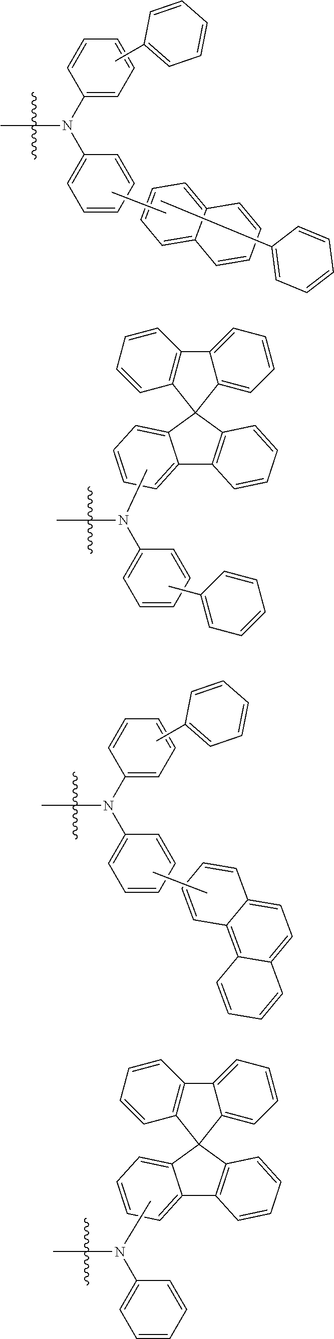

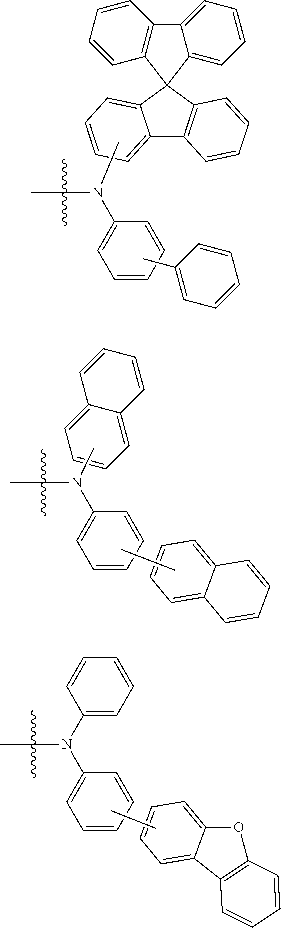

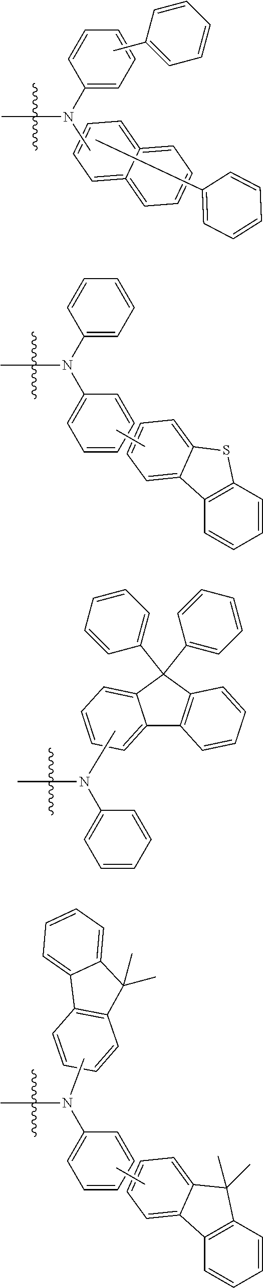

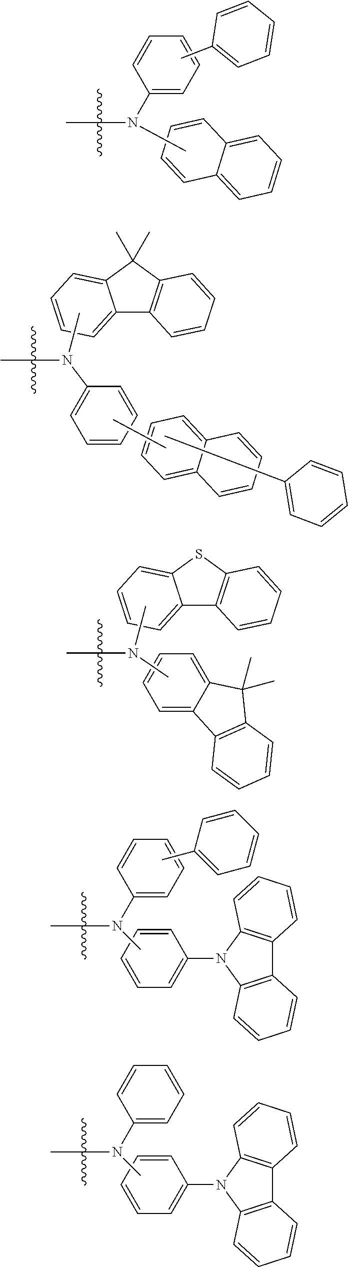

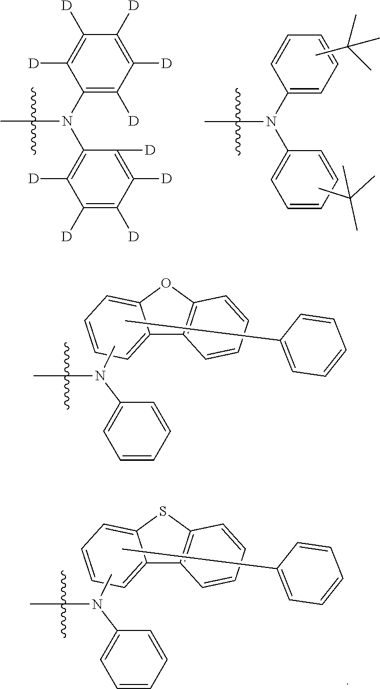

In particular

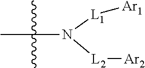

is selected from the group consisting of:

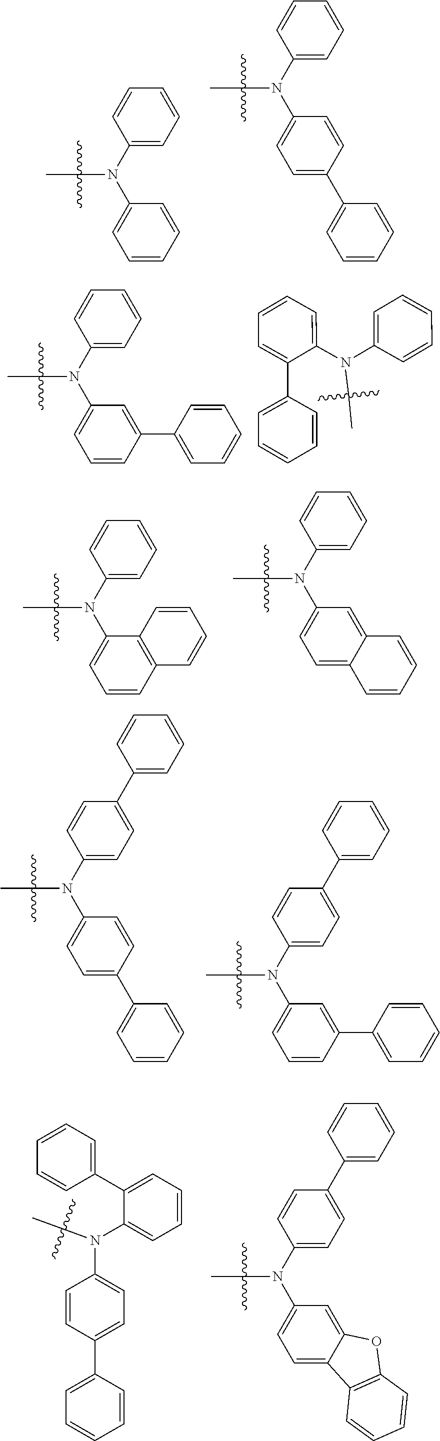

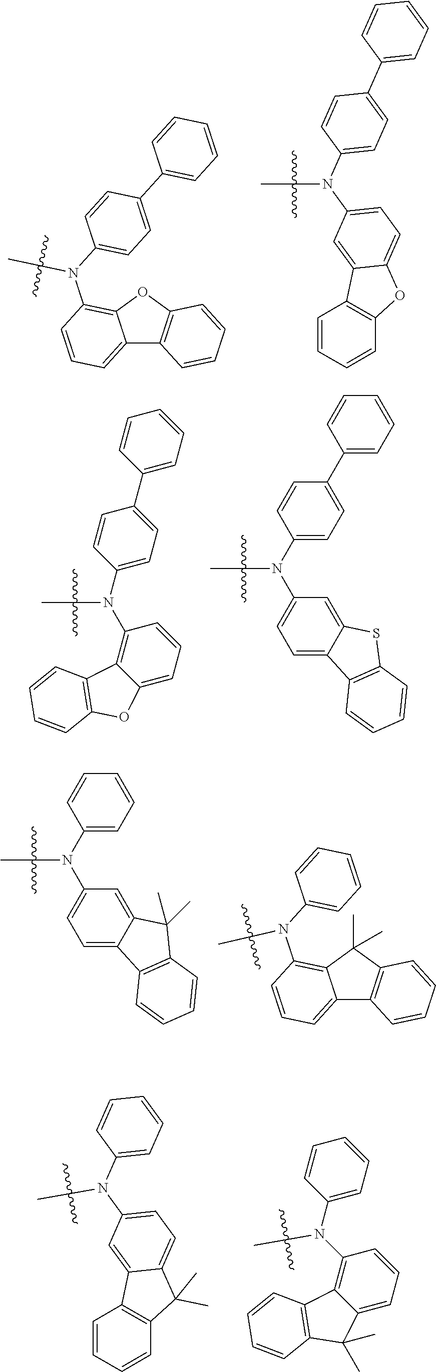

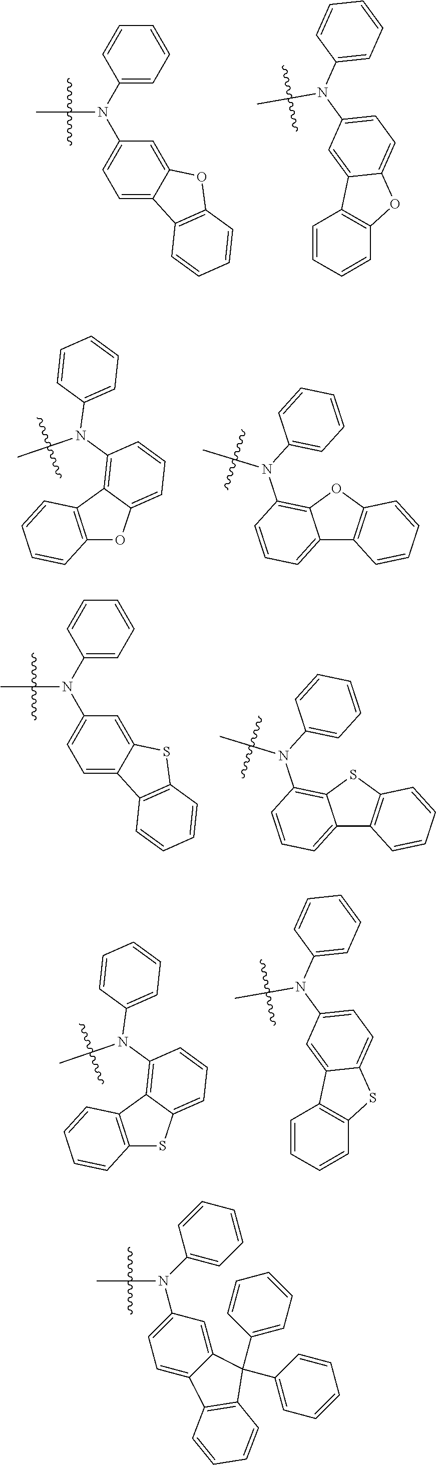

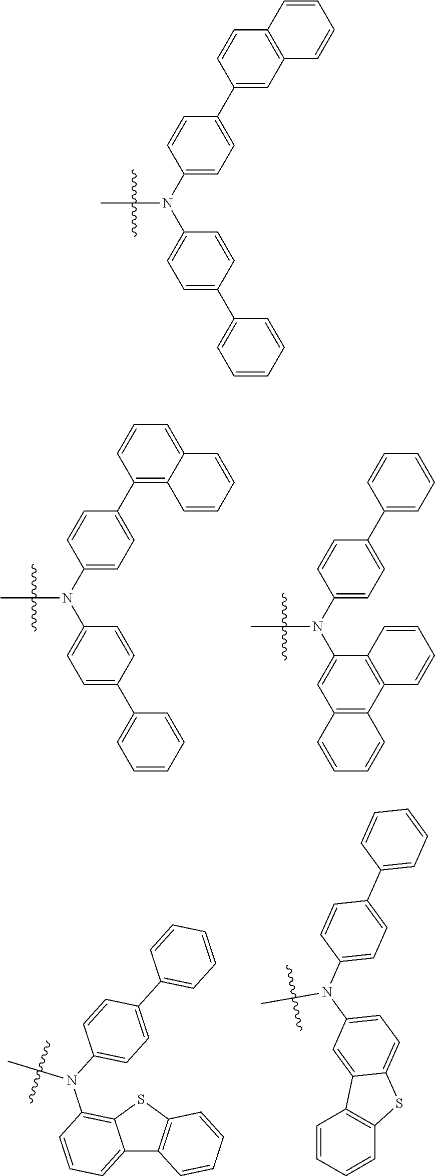

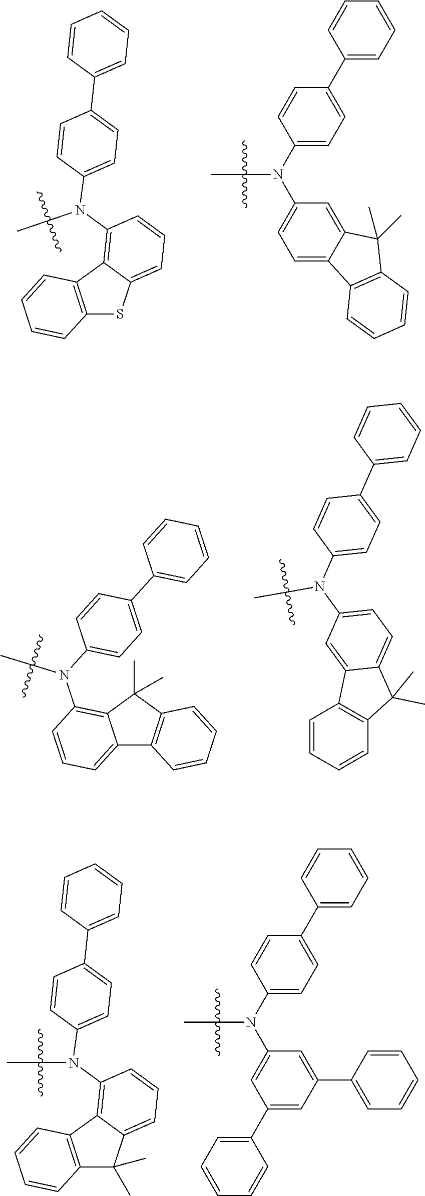 [SMILES: *N(c1ccc(-c2ccccc2)cc1)c1cccc2sc3ccccc3c12 ; *N(c1ccc(-c2ccccc2)cc1)c1ccc2c(c1)C(C)(C)c1ccccc1-2 ; *N(c1ccc(-c2ccccc2)cc1)c1cccc2c1C(C)(C)c1ccccc1-2 ; *N(c1ccc(-c2ccccc2)cc1)c1ccc2c(c1)-c1ccccc1C2(C)C ; *N(c1ccc(-c2ccccc2)cc1)c1cccc2c1-c1ccccc1C2(C)C ; *N(c1ccc(-c2ccccc2)cc1)c1cc(-c2ccccc2)cc(-c2ccccc2)c1]

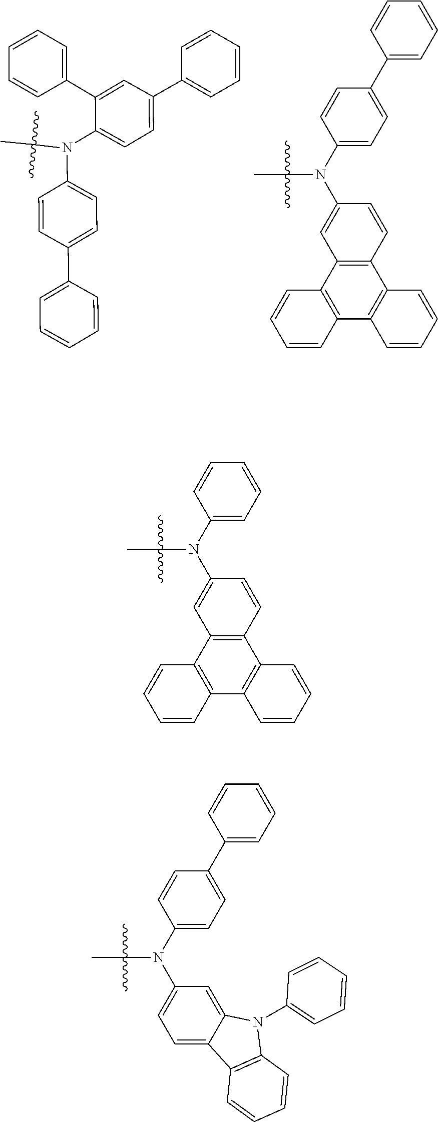 [SMILES: *N(c1ccc(-c2ccccc2)cc1)c1ccc(-c2ccccc2)cc1-c1ccccc1 ; *N(c1ccc(-c2ccccc2)cc1)c1ccc2c3ccccc3c3ccccc3c2c1 ; *N(c1ccccc1)c1ccc2c3ccccc3c3ccccc3c2c1 ; *N(c1ccc(-c2ccccc2)cc1)c1ccc2c3ccccc3n(-c3ccccc3)c2c1]

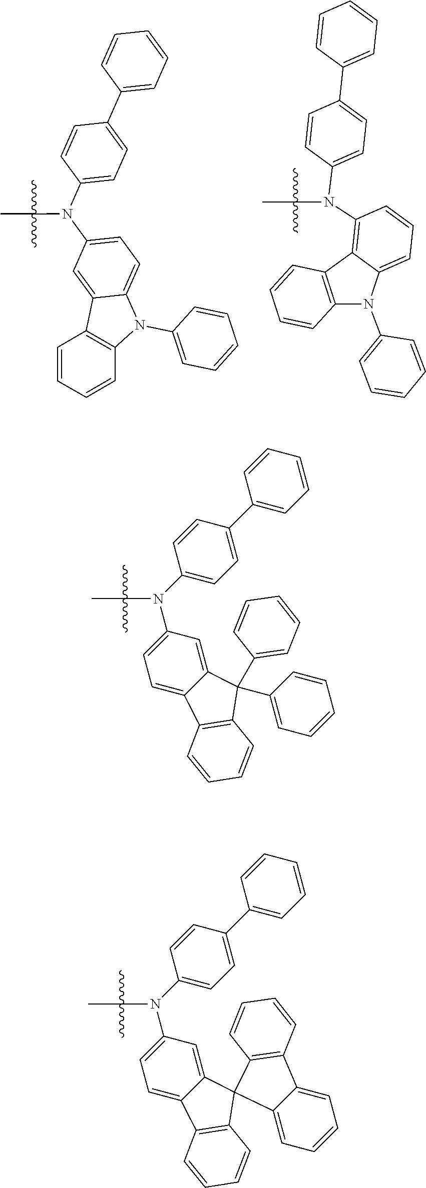 [SMILES: *N(c1ccc(-c2ccccc2)cc1)c1ccc2c(c1)c1ccccc1n2-c1ccccc1 ; *N(c1ccc(-c2ccccc2)cc1)c1cccc2c1c1ccccc1n2-c1ccccc1 ; *N(c1ccc(-c2ccccc2)cc1)c1ccc2c(c1)C(c1ccccc1)(c1ccccc1)c1ccccc1-2 ; *N(c1ccc(-c2ccccc2)cc1)c1ccc2c(c1)C1(c3ccccc3-c3ccccc31)c1ccccc1-2]

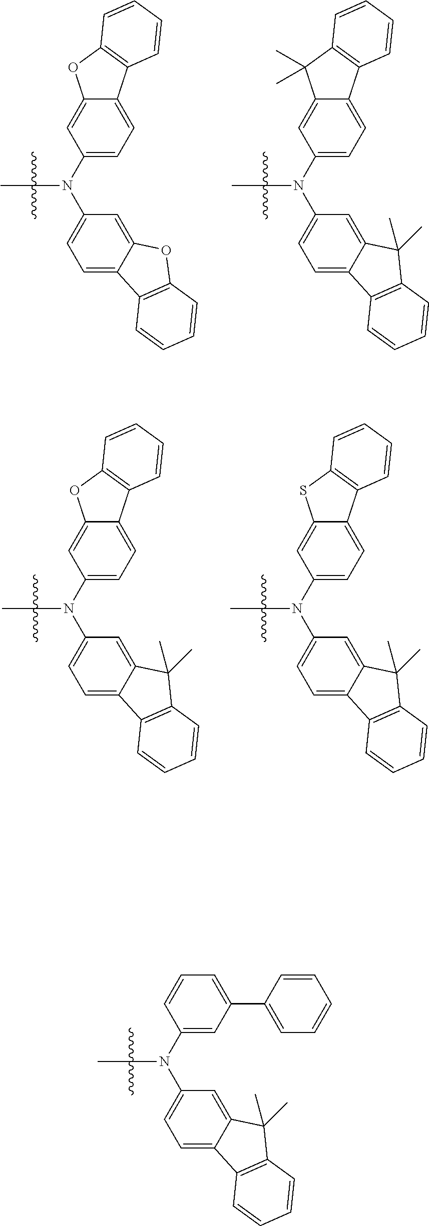 [SMILES: *N(c1ccc2c(c1)oc1ccccc12)c1ccc2c(c1)oc1ccccc12 ; *N(c1ccc2c(c1)C(C)(C)c1ccccc1-2)c1ccc2c(c1)C(C)(C)c1ccccc1-2 ; *N(c1ccc2c(c1)C(C)(C)c1ccccc1-2)c1ccc2c(c1)oc1ccccc12 ; *N(c1ccc2c(c1)C(C)(C)c1ccccc1-2)c1ccc2c(c1)sc1ccccc12 ; *N(c1cccc(-c2ccccc2)c1)c1ccc2c(c1)C(C)(C)c1ccccc1-2]

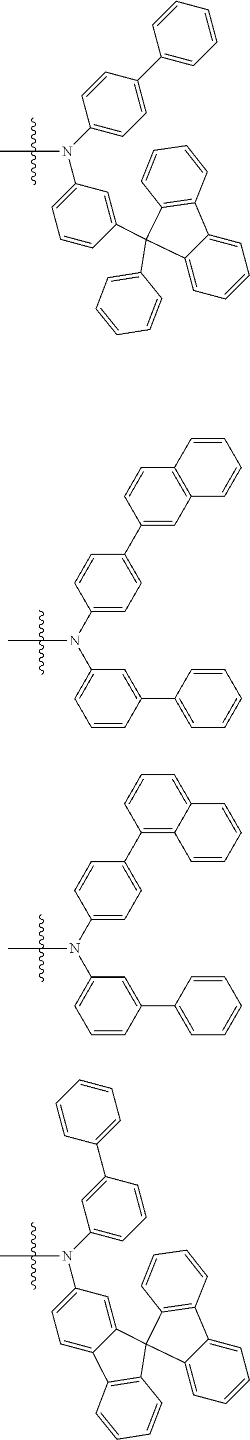

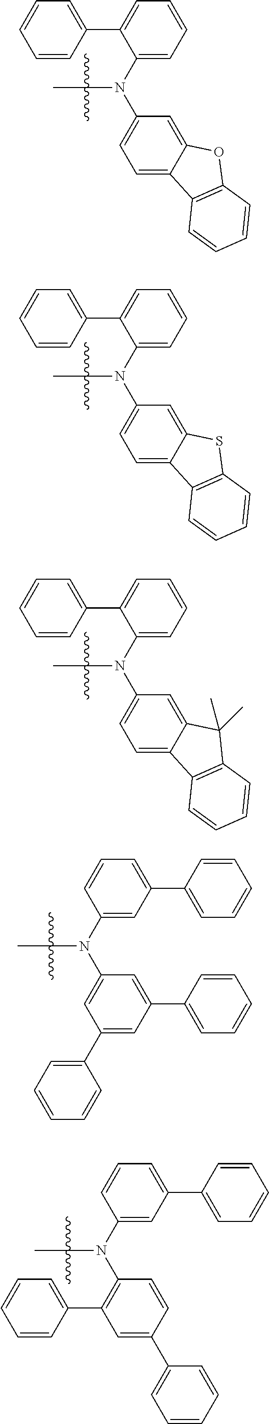

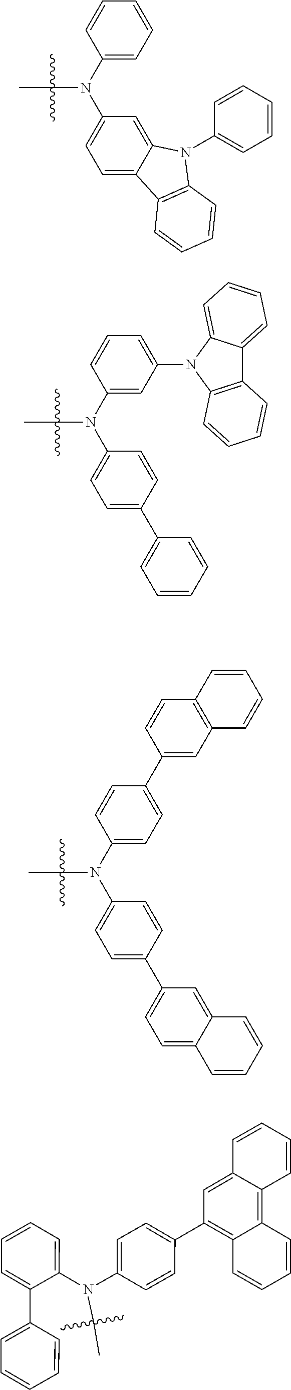 [SMILES: *N(c1ccccc1)c1ccc2c3ccccc3n(-c3ccccc3)c2c1 ; *N(c1ccc(-c2ccccc2)cc1)c1cccc(-n2c3ccccc3c3ccccc32)c1 ; *N(c1ccc(-c2ccc3ccccc3c2)cc1)c1ccc(-c2ccc3ccccc3c2)cc1 ; *N(c1ccc(-c2cc3ccccc3c3ccccc23)cc1)c1ccccc1-c1ccccc1]

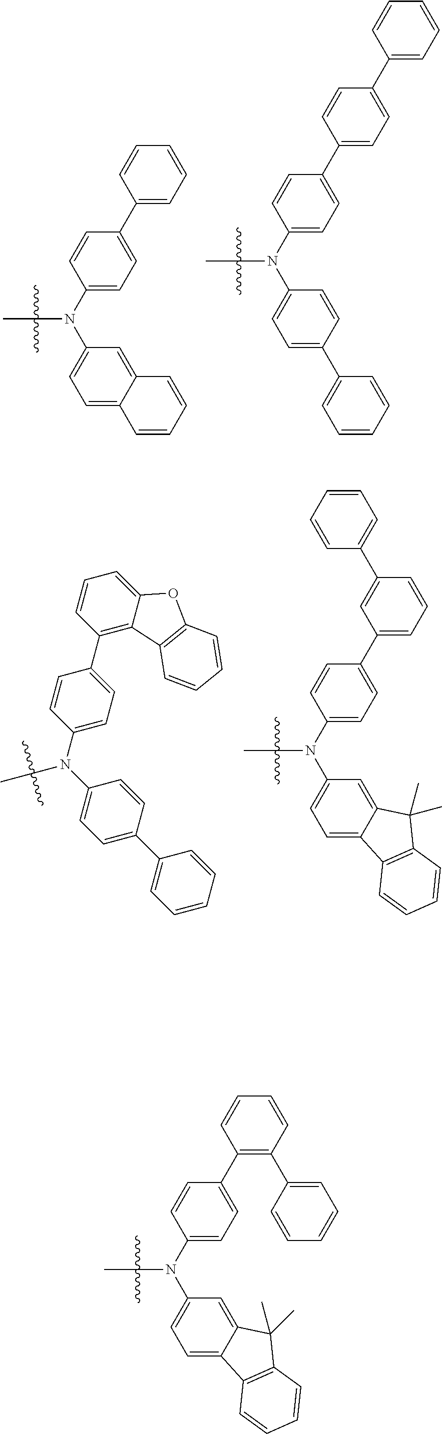 [SMILES: *N(c1ccc(-c2ccccc2)cc1)c1ccc2ccccc2c1 ; *N(c1ccc(-c2ccccc2)cc1)c1ccc(-c2ccc(-c3ccccc3)cc2)cc1 ; *N(c1ccc(-c2ccccc2)cc1)c1ccc(-c2cccc3oc4ccccc4c23)cc1 ; *N(c1ccc(-c2cccc(-c3ccccc3)c2)cc1)c1ccc2c(c1)C(C)(C)c1ccccc1-2 ; *N(c1ccc(-c2ccccc2-c2ccccc2)cc1)c1ccc2c(c1)C(C)(C)c1ccccc1-2]

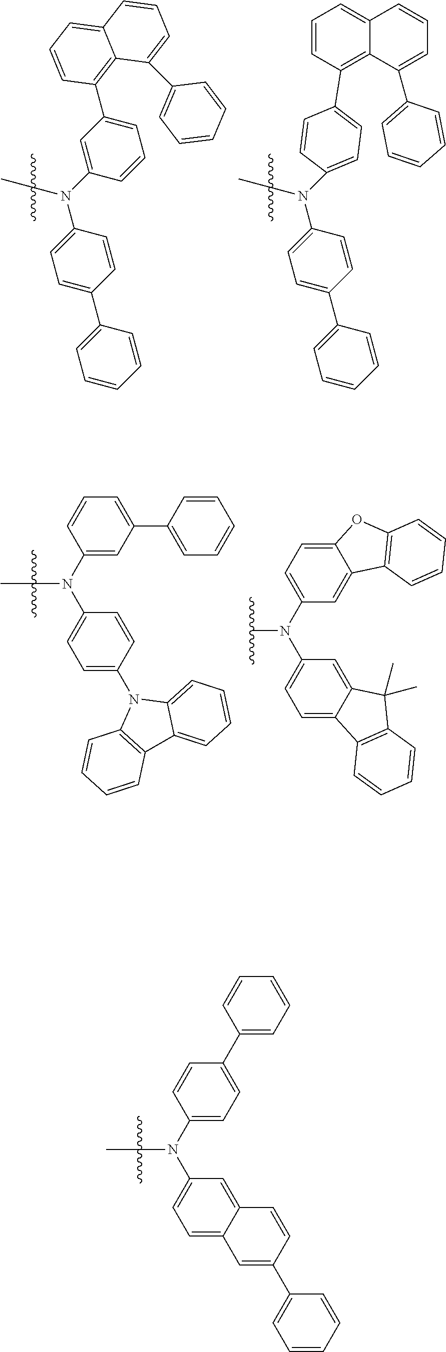 [SMILES: *N(c1ccc(-c2ccccc2)cc1)c1cccc(-c2cccc3cccc(-c4ccccc4)c23)c1 ; *N(c1ccc(-c2ccccc2)cc1)c1ccc(-c2cccc3cccc(-c4ccccc4)c23)cc1 ; *N(c1ccc(-n2c3ccccc3c3ccccc32)cc1)c1cccc(-c2ccccc2)c1 ; *N(c1ccc2c(c1)C(C)(C)c1ccccc1-2)c1ccc2oc3ccccc3c2c1 ; *N(c1ccc(-c2ccccc2)cc1)c1ccc2cc(-c3ccccc3)ccc2c1]

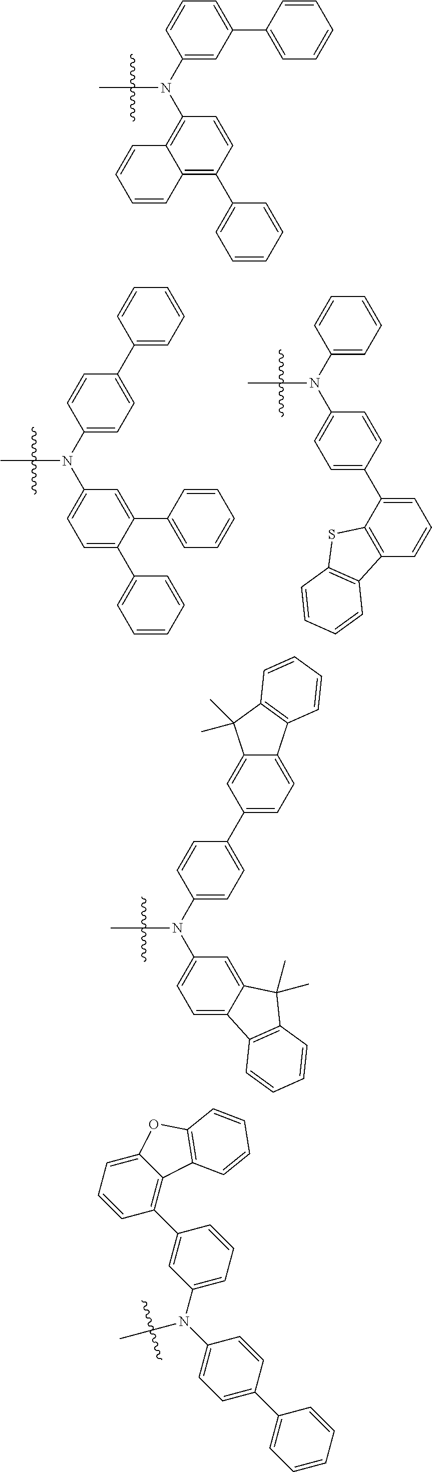

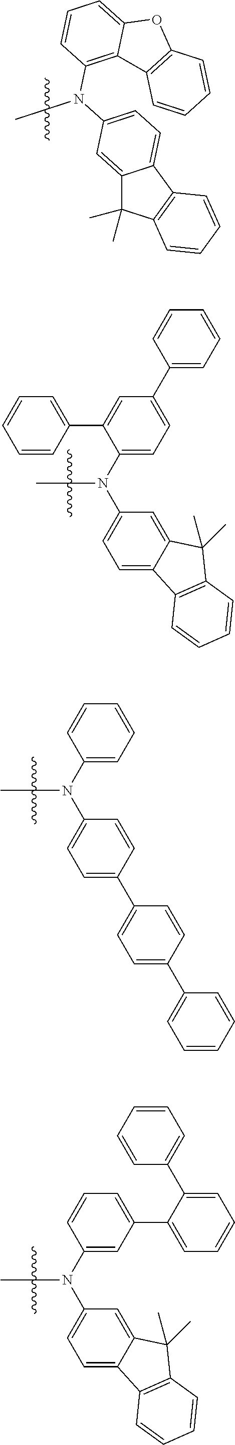 [SMILES: *N(c1ccc2c(c1)C(C)(C)c1ccccc1-2)c1cccc2oc3ccccc3c12 ; *N(c1ccc2c(c1)C(C)(C)c1ccccc1-2)c1ccc(-c2ccccc2)cc1-c1ccccc1 ; *N(c1ccccc1)c1ccc(-c2ccc(-c3ccccc3)cc2)cc1 ; *N(c1cccc(-c2ccccc2-c2ccccc2)c1)c1ccc2c(c1)C(C)(C)c1ccccc1-2]

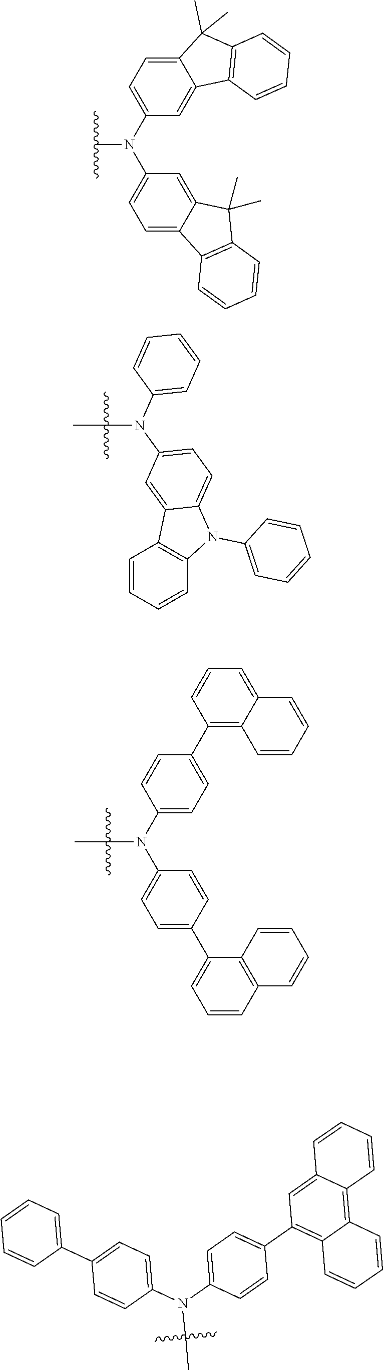 [SMILES: *N(c1ccc2c(c1)-c1ccccc1C2(C)C)c1ccc2c(c1)C(C)(C)c1ccccc1-2 ; *N(c1ccccc1)c1ccc2c(c1)c1ccccc1n2-c1ccccc1 ; *N(c1ccc(-c2cccc3ccccc23)cc1)c1ccc(-c2cccc3ccccc23)cc1 ; *N(c1ccc(-c2ccccc2)cc1)c1ccc(-c2cc3ccccc3c3ccccc23)cc1]

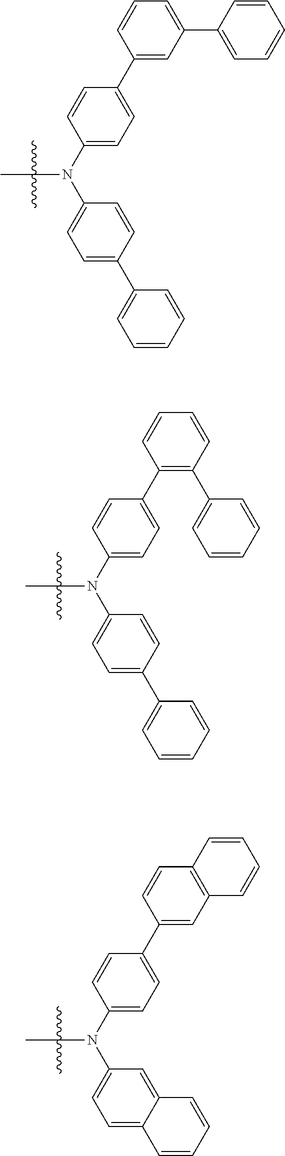 [SMILES: *N(c1ccc(-c2ccccc2)cc1)c1ccc(-c2cccc(-c3ccccc3)c2)cc1 ; *N(c1ccc(-c2ccccc2)cc1)c1ccc(-c2ccccc2-c2ccccc2)cc1 ; *N(c1ccc(-c2ccc3ccccc3c2)cc1)c1ccc2ccccc2c1]

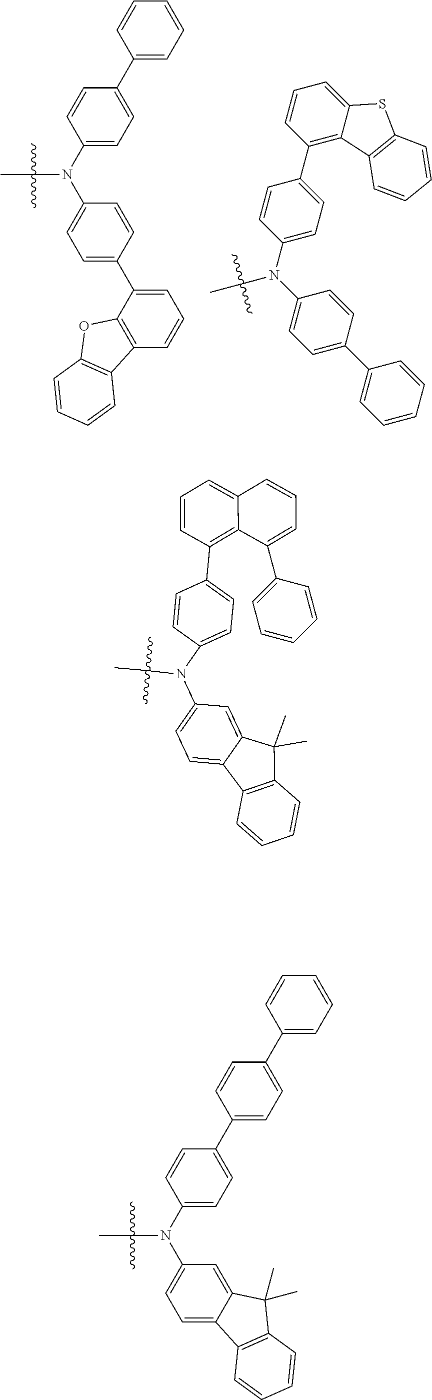 [SMILES: *N(c1ccc(-c2ccccc2)cc1)c1ccc(-c2cccc3c2oc2ccccc23)cc1 ; *N(c1ccc(-c2ccccc2)cc1)c1ccc(-c2cccc3sc4ccccc4c23)cc1 ; *N(c1ccc(-c2cccc3cccc(-c4ccccc4)c23)cc1)c1ccc2c(c1)C(C)(C)c1ccccc1-2 ; *N(c1ccc(-c2ccc(-c3ccccc3)cc2)cc1)c1ccc2c(c1)C(C)(C)c1ccccc1-2]

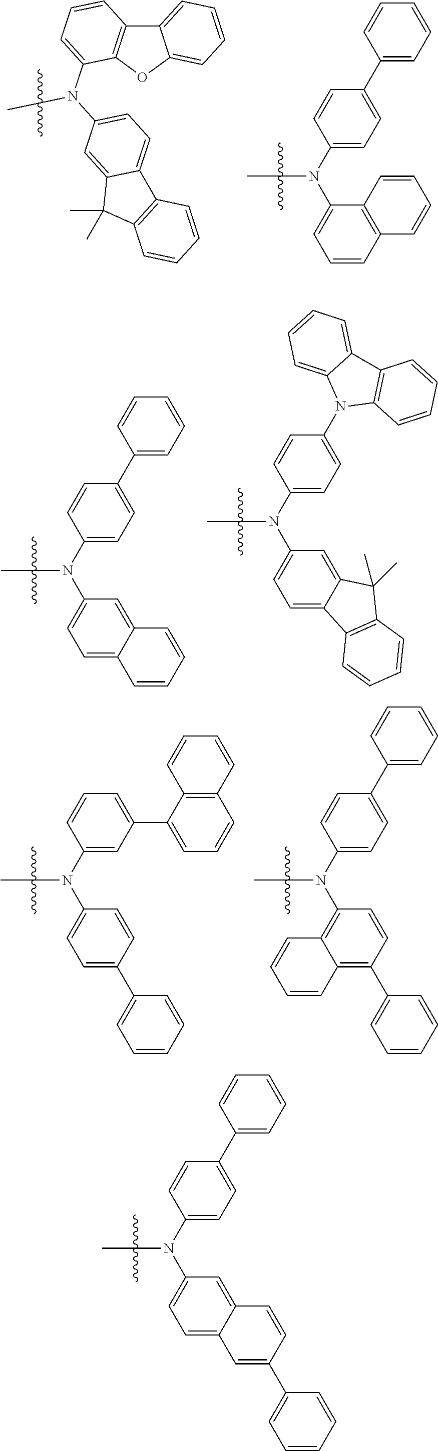

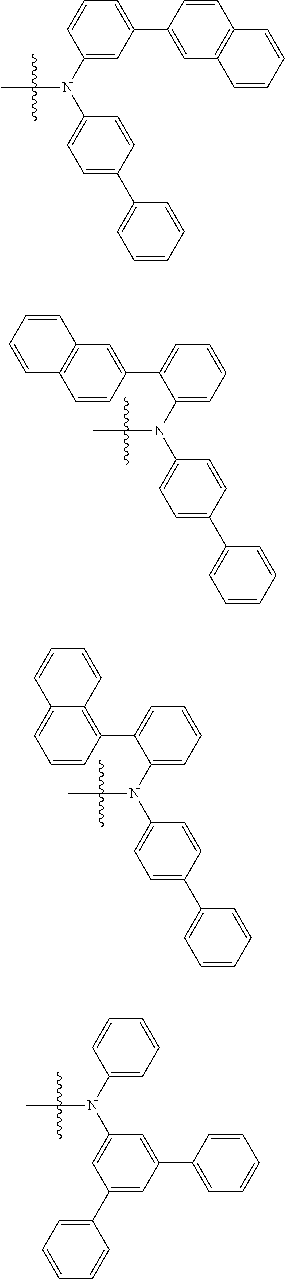 [SMILES: *N(c1ccc(-c2ccccc2)cc1)c1cccc(-c2ccc3ccccc3c2)c1 ; *N(c1ccc(-c2ccccc2)cc1)c1ccccc1-c1ccc2ccccc2c1 ; *N(c1ccc(-c2ccccc2)cc1)c1ccccc1-c1cccc2ccccc12 ; *N(c1ccccc1)c1cc(-c2ccccc2)cc(-c2ccccc2)c1]

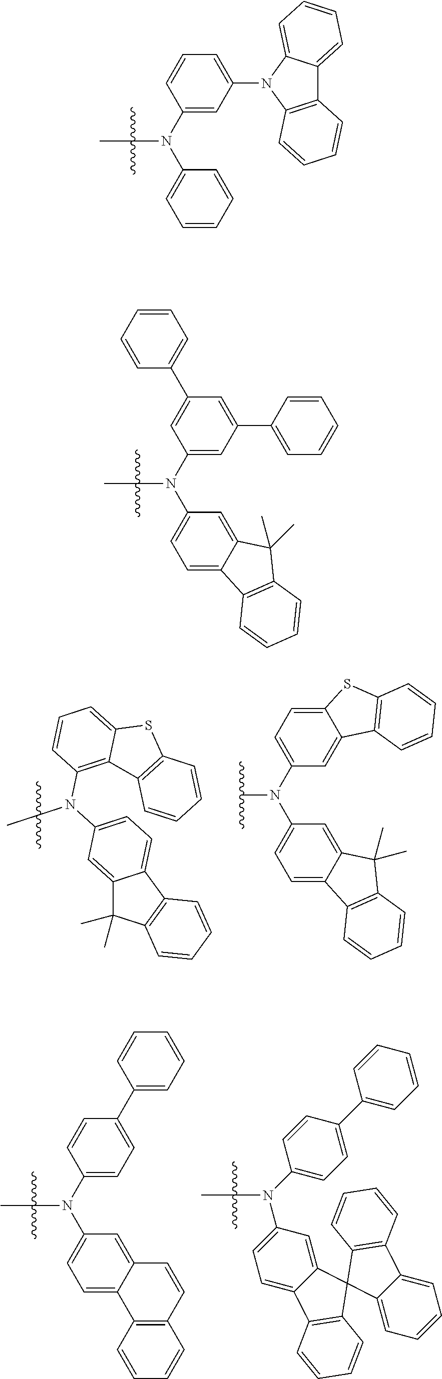

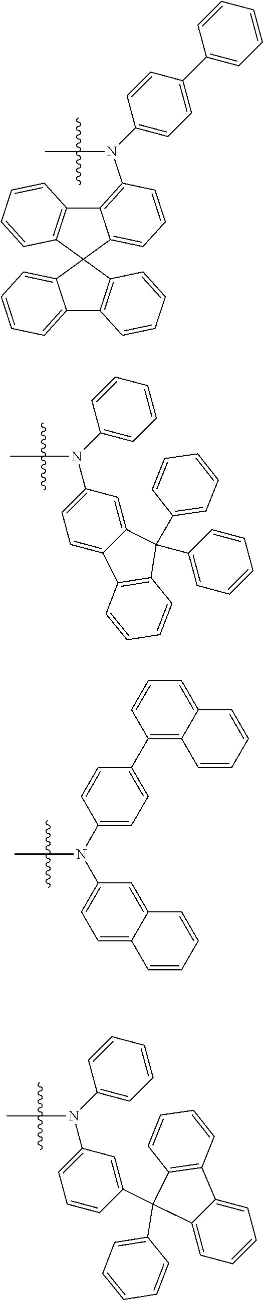

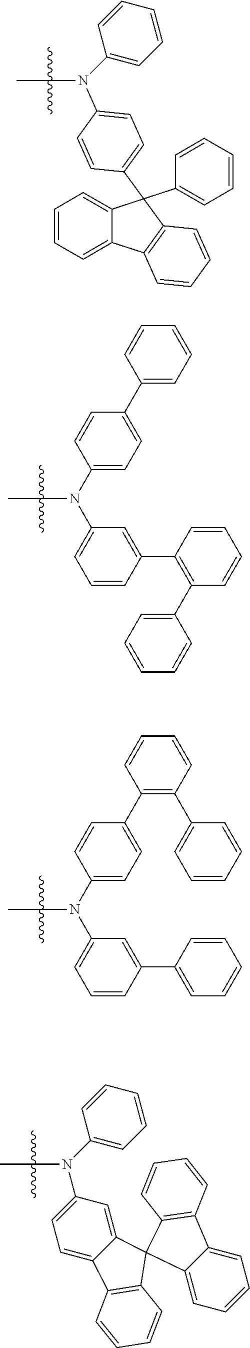

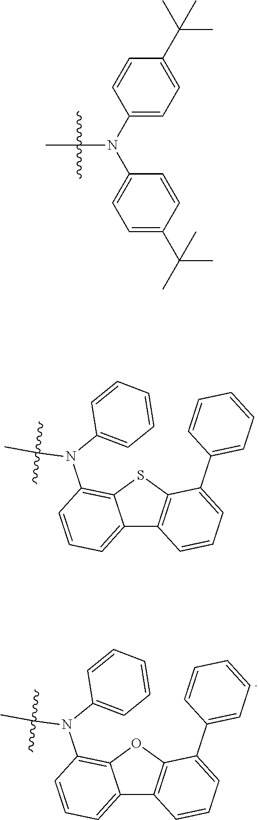 [SMILES: *N(c1ccc(C(C)(C)C)cc1)c1ccc(C(C)(C)C)cc1 ; *N(c1ccccc1)c1cccc2c1sc1c(-c3ccccc3)cccc12 ; *N(c1ccccc1)c1cccc2c1oc1c(-c3ccccc3)cccc12]

Optionally, the organic compound is selected from the group consisting of the following compounds:

[chemical structure]

[chemical structure]

[chemical structure]

[chemical structure]

[chemical structure]

[chemical structure]

[chemical structure]

[chemical structure]

[chemical structure]

[chemical structure]

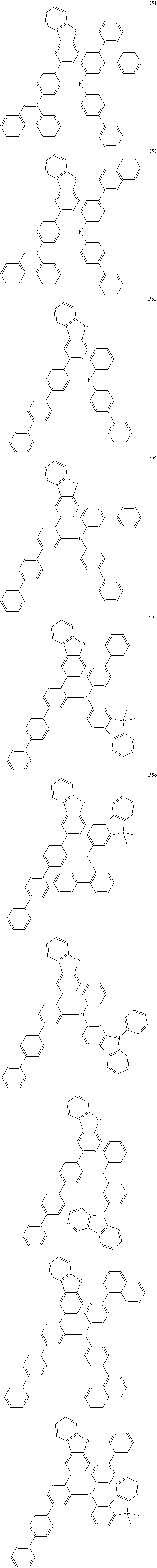

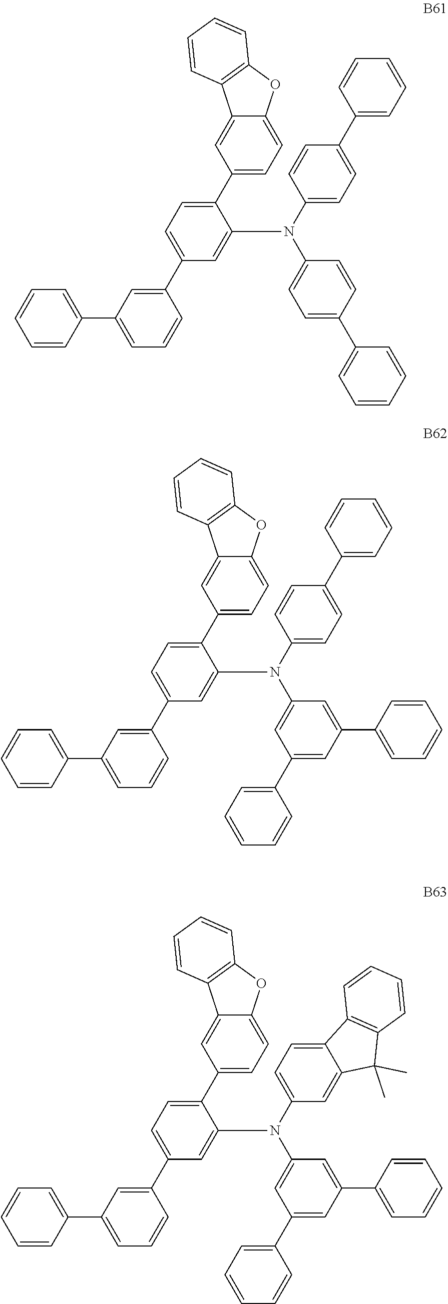 [SMILES: c1ccc(-c2ccc(N(c3ccc(-c4ccccc4)cc3)c3cc(-c4cccc(-c5ccccc5)c4)ccc3-c3ccc4oc5ccccc5c4c3)cc2)cc1 ; c1ccc(-c2ccc(N(c3cc(-c4ccccc4)cc(-c4ccccc4)c3)c3cc(-c4cccc(-c5ccccc5)c4)ccc3-c3ccc4oc5ccccc5c4c3)cc2)cc1 ; CC1(C)c2ccccc2-c2ccc(N(c3cc(-c4ccccc4)cc(-c4ccccc4)c3)c3cc(-c4cccc(-c5ccccc5)c4)ccc3-c3ccc4oc5ccccc5c4c3)cc21]

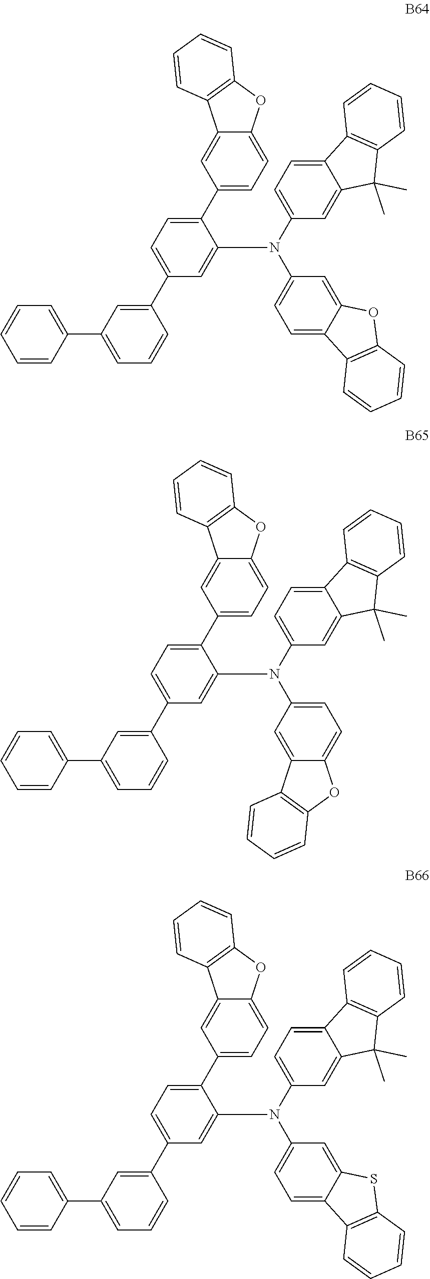 [SMILES: CC1(C)c2ccccc2-c2ccc(N(c3ccc4c(c3)oc3ccccc34)c3cc(-c4cccc(-c5ccccc5)c4)ccc3-c3ccc4oc5ccccc5c4c3)cc21 ; CC1(C)c2ccccc2-c2ccc(N(c3ccc4oc5ccccc5c4c3)c3cc(-c4cccc(-c5ccccc5)c4)ccc3-c3ccc4oc5ccccc5c4c3)cc21 ; CC1(C)c2ccccc2-c2ccc(N(c3ccc4c(c3)sc3ccccc34)c3cc(-c4cccc(-c5ccccc5)c4)ccc3-c3ccc4oc5ccccc5c4c3)cc21]

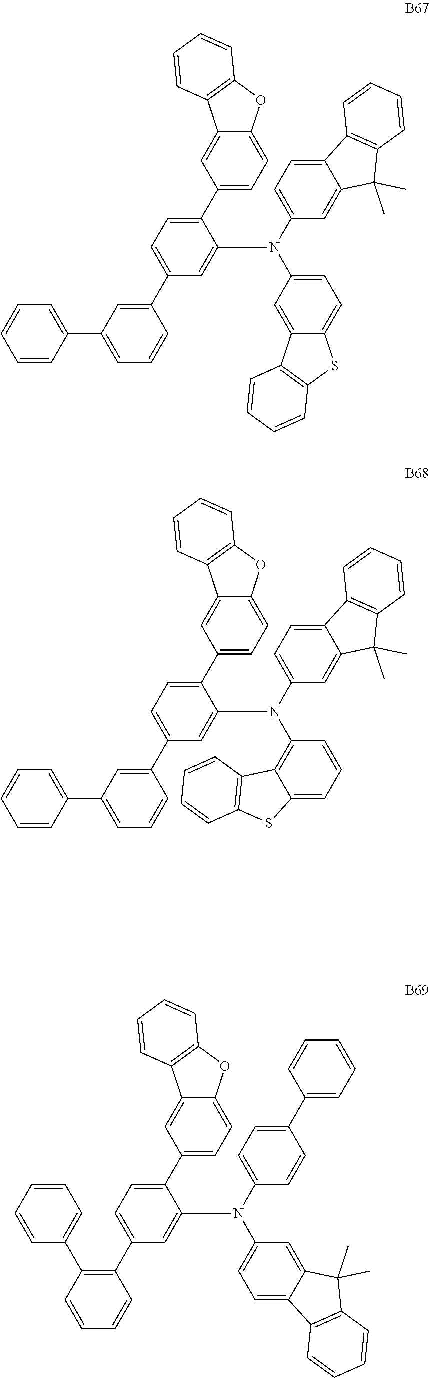 [SMILES: CC1(C)c2ccccc2-c2ccc(N(c3ccc4sc5ccccc5c4c3)c3cc(-c4cccc(-c5ccccc5)c4)ccc3-c3ccc4oc5ccccc5c4c3)cc21 ; CC1(C)c2ccccc2-c2ccc(N(c3cc(-c4cccc(-c5ccccc5)c4)ccc3-c3ccc4oc5ccccc5c4c3)c3cccc4sc5ccccc5c34)cc21 ; CC1(C)c2ccccc2-c2ccc(N(c3ccc(-c4ccccc4)cc3)c3cc(-c4ccccc4-c4ccccc4)ccc3-c3ccc4oc5ccccc5c4c3)cc21]

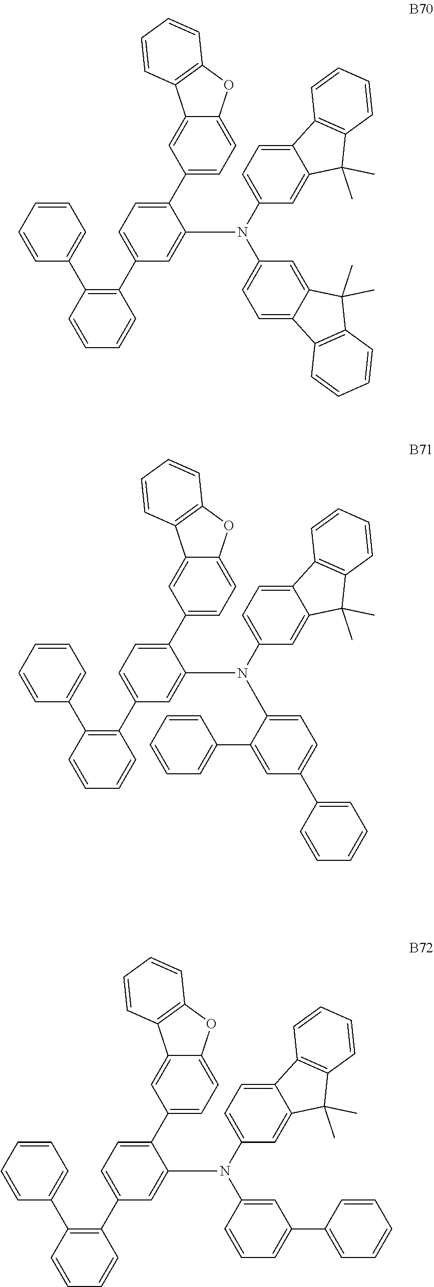 [SMILES: CC1(C)c2ccccc2-c2ccc(N(c3ccc4c(c3)C(C)(C)c3ccccc3-4)c3cc(-c4ccccc4-c4ccccc4)ccc3-c3ccc4oc5ccccc5c4c3)cc21 ; CC1(C)c2ccccc2-c2ccc(N(c3ccc(-c4ccccc4)cc3-c3ccccc3)c3cc(-c4ccccc4-c4ccccc4)ccc3-c3ccc4oc5ccccc5c4c3)cc21 ; CC1(C)c2ccccc2-c2ccc(N(c3cccc(-c4ccccc4)c3)c3cc(-c4ccccc4-c4ccccc4)ccc3-c3ccc4oc5ccccc5c4c3)cc21]

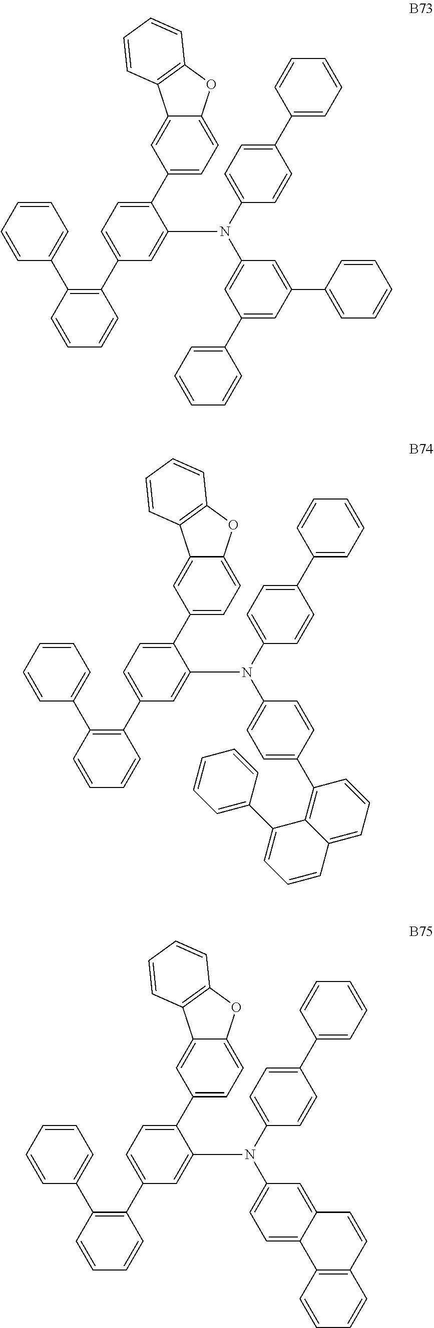 [SMILES: c1ccc(-c2ccc(N(c3cc(-c4ccccc4)cc(-c4ccccc4)c3)c3cc(-c4ccccc4-c4ccccc4)ccc3-c3ccc4oc5ccccc5c4c3)cc2)cc1 ; c1ccc(-c2ccc(N(c3ccc(-c4cccc5cccc(-c6ccccc6)c45)cc3)c3cc(-c4ccccc4-c4ccccc4)ccc3-c3ccc4oc5ccccc5c4c3)cc2)cc1 ; c1ccc(-c2ccc(N(c3ccc4c(ccc5ccccc54)c3)c3cc(-c4ccccc4-c4ccccc4)ccc3-c3ccc4oc5ccccc5c4c3)cc2)cc1]

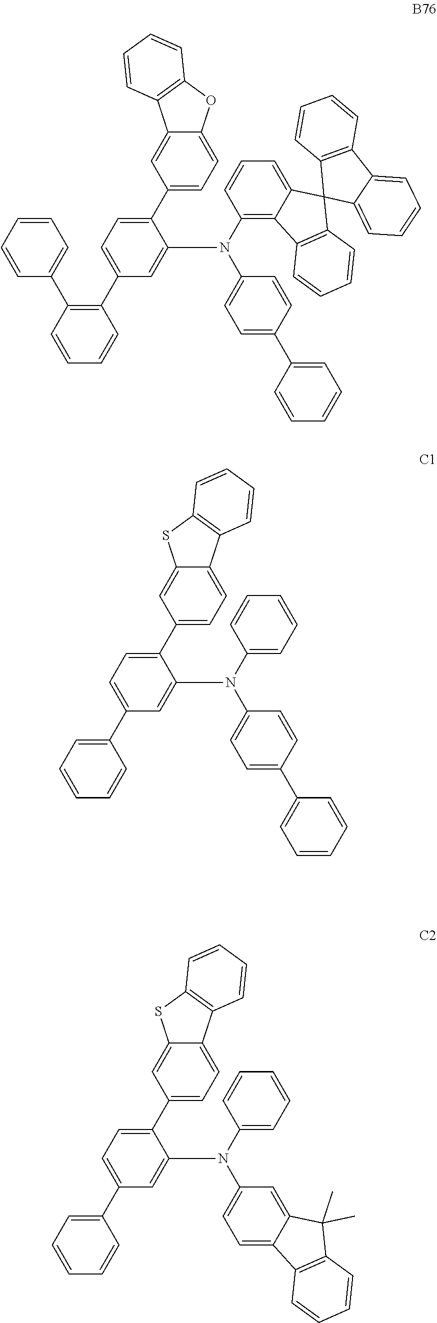 [SMILES: c1ccc(-c2ccc(N(c3cc(-c4ccccc4-c4ccccc4)ccc3-c3ccc4oc5ccccc5c4c3)c3cccc4c3-c3ccccc3C43c4ccccc4-c4ccccc43)cc2)cc1 ; c1ccc(-c2ccc(N(c3ccccc3)c3cc(-c4ccccc4)ccc3-c3ccc4c(c3)sc3ccccc34)cc2)cc1 ; CC1(C)c2ccccc2-c2ccc(N(c3ccccc3)c3cc(-c4ccccc4)ccc3-c3ccc4c(c3)sc3ccccc34)cc21]

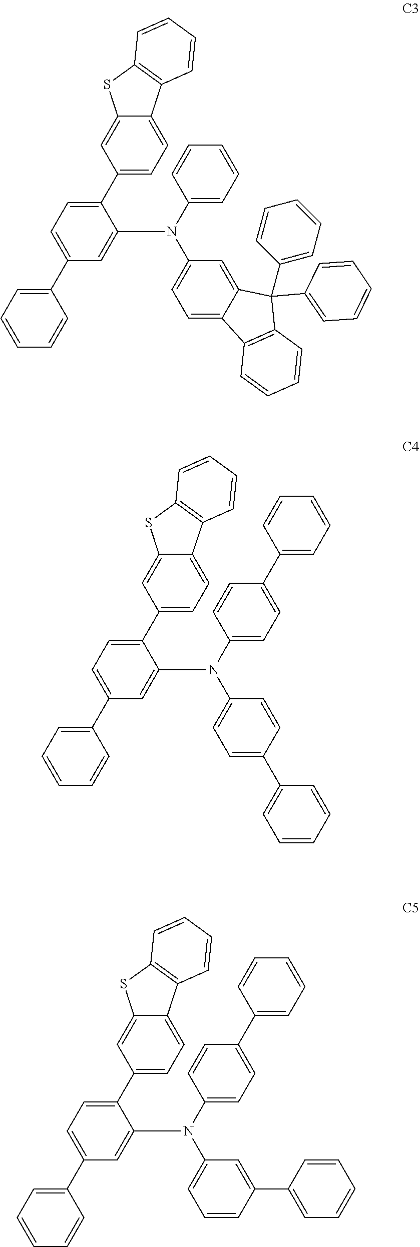 [SMILES: c1ccc(-c2ccc(-c3ccc4c(c3)sc3ccccc34)c(N(c3ccccc3)c3ccc4c(c3)C(c3ccccc3)(c3ccccc3)c3ccccc3-4)c2)cc1 ; c1ccc(-c2ccc(N(c3ccc(-c4ccccc4)cc3)c3cc(-c4ccccc4)ccc3-c3ccc4c(c3)sc3ccccc34)cc2)cc1 ; c1ccc(-c2ccc(N(c3cccc(-c4ccccc4)c3)c3cc(-c4ccccc4)ccc3-c3ccc4c(c3)sc3ccccc34)cc2)cc1]

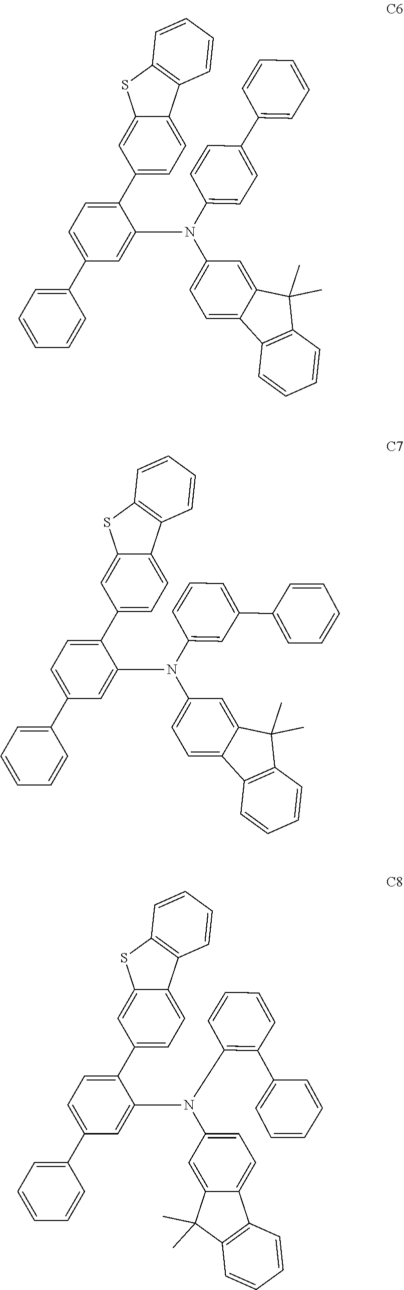 [SMILES: CC1(C)c2ccccc2-c2ccc(N(c3ccc(-c4ccccc4)cc3)c3cc(-c4ccccc4)ccc3-c3ccc4c(c3)sc3ccccc34)cc21 ; CC1(C)c2ccccc2-c2ccc(N(c3cccc(-c4ccccc4)c3)c3cc(-c4ccccc4)ccc3-c3ccc4c(c3)sc3ccccc34)cc21 ; CC1(C)c2ccccc2-c2ccc(N(c3ccccc3-c3ccccc3)c3cc(-c4ccccc4)ccc3-c3ccc4c(c3)sc3ccccc34)cc21]

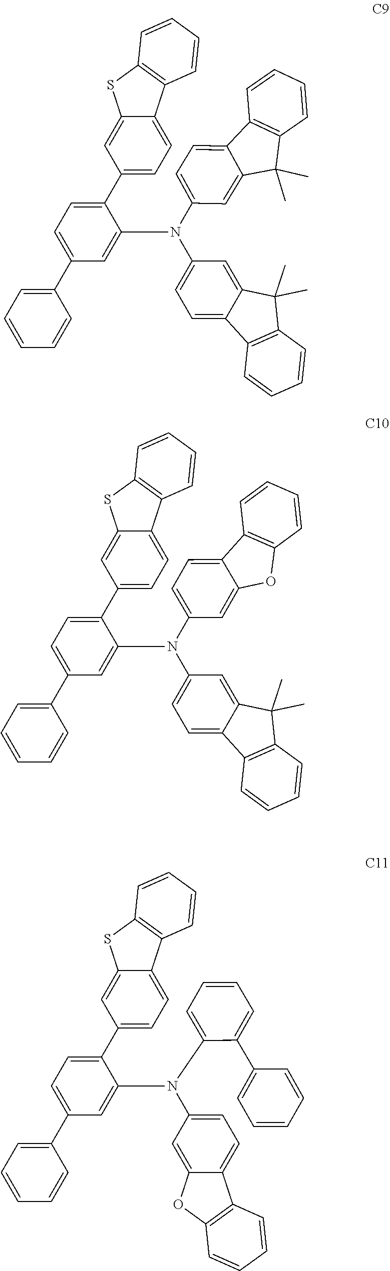 [SMILES: CC1(C)c2ccccc2-c2ccc(N(c3ccc4c(c3)C(C)(C)c3ccccc3-4)c3cc(-c4ccccc4)ccc3-c3ccc4c(c3)sc3ccccc34)cc21 ; CC1(C)c2ccccc2-c2ccc(N(c3ccc4c(c3)oc3ccccc34)c3cc(-c4ccccc4)ccc3-c3ccc4c(c3)sc3ccccc34)cc21 ; c1ccc(-c2ccc(-c3ccc4c(c3)sc3ccccc34)c(N(c3ccc4c(c3)oc3ccccc34)c3ccccc3-c3ccccc3)c2)cc1]

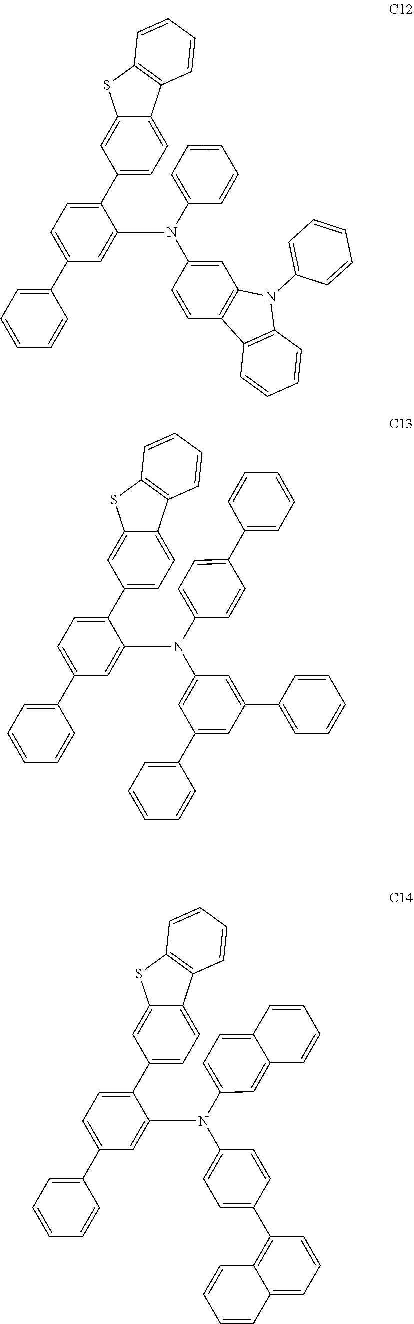 [SMILES: c1ccc(-c2ccc(-c3ccc4c(c3)sc3ccccc34)c(N(c3ccccc3)c3ccc4c5ccccc5n(-c5ccccc5)c4c3)c2)cc1 ; c1ccc(-c2ccc(N(c3cc(-c4ccccc4)cc(-c4ccccc4)c3)c3cc(-c4ccccc4)ccc3-c3ccc4c(c3)sc3ccccc34)cc2)cc1 ; c1ccc(-c2ccc(-c3ccc4c(c3)sc3ccccc34)c(N(c3ccc(-c4cccc5ccccc45)cc3)c3ccc4ccccc4c3)c2)cc1]

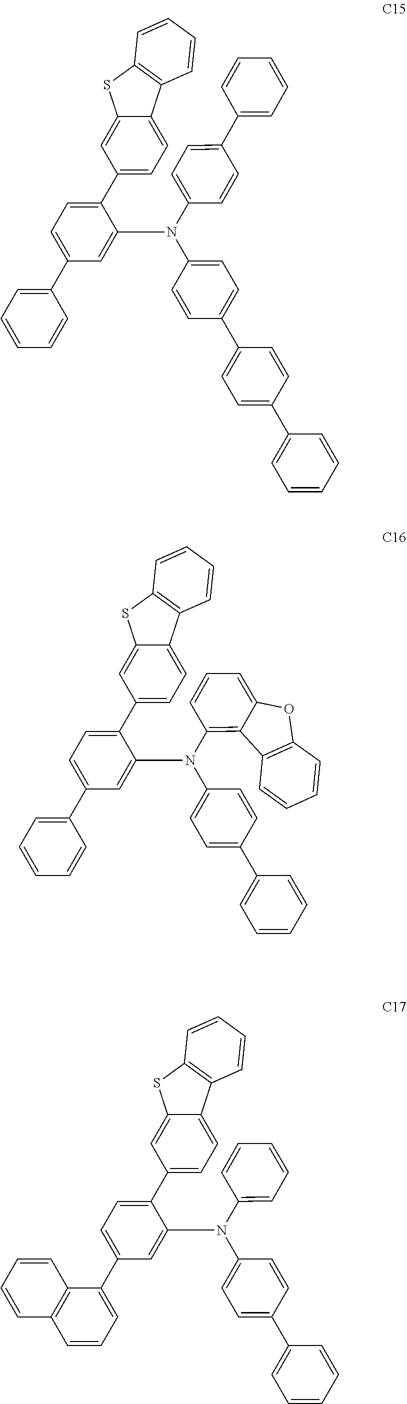 [SMILES: c1ccc(-c2ccc(-c3ccc(N(c4ccc(-c5ccccc5)cc4)c4cc(-c5ccccc5)ccc4-c4ccc5c(c4)sc4ccccc45)cc3)cc2)cc1 ; c1ccc(-c2ccc(N(c3cc(-c4ccccc4)ccc3-c3ccc4c(c3)sc3ccccc34)c3cccc4oc5ccccc5c34)cc2)cc1 ; c1ccc(-c2ccc(N(c3ccccc3)c3cc(-c4cccc5ccccc45)ccc3-c3ccc4c(c3)sc3ccccc34)cc2)cc1]

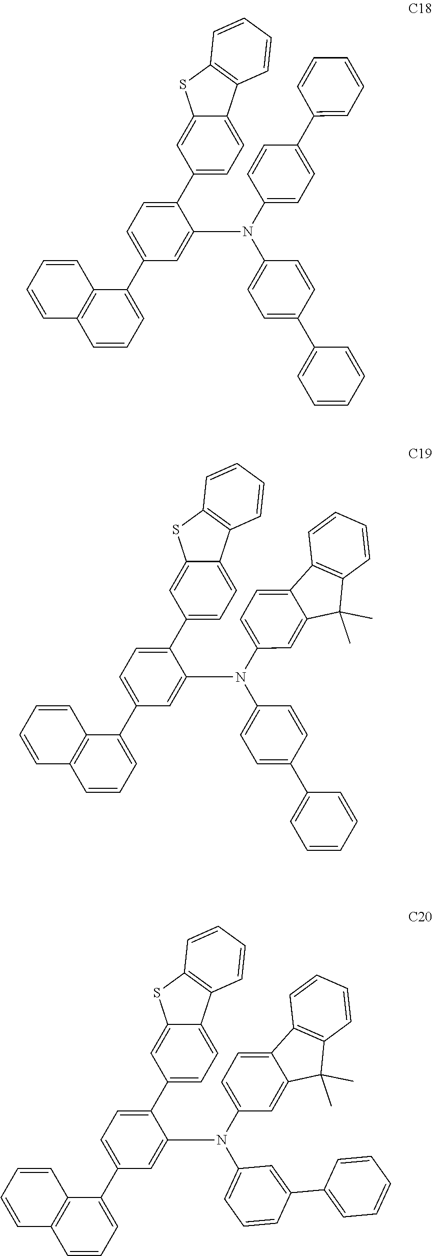 [SMILES: c1ccc(-c2ccc(N(c3ccc(-c4ccccc4)cc3)c3cc(-c4cccc5ccccc45)ccc3-c3ccc4c(c3)sc3ccccc34)cc2)cc1 ; CC1(C)c2ccccc2-c2ccc(N(c3ccc(-c4ccccc4)cc3)c3cc(-c4cccc5ccccc45)ccc3-c3ccc4c(c3)sc3ccccc34)cc21 ; CC1(C)c2ccccc2-c2ccc(N(c3cccc(-c4ccccc4)c3)c3cc(-c4cccc5ccccc45)ccc3-c3ccc4c(c3)sc3ccccc34)cc21]

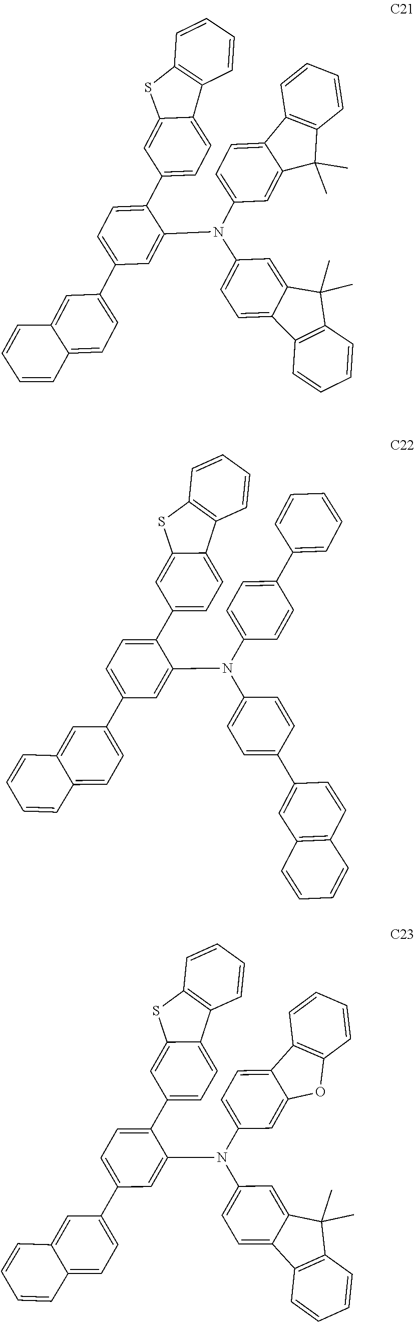 [SMILES: CC1(C)c2ccccc2-c2ccc(N(c3ccc4c(c3)C(C)(C)c3ccccc3-4)c3cc(-c4ccc5ccccc5c4)ccc3-c3ccc4c(c3)sc3ccccc34)cc21 ; c1ccc(-c2ccc(N(c3ccc(-c4ccc5ccccc5c4)cc3)c3cc(-c4ccc5ccccc5c4)ccc3-c3ccc4c(c3)sc3ccccc34)cc2)cc1 ; CC1(C)c2ccccc2-c2ccc(N(c3ccc4c(c3)oc3ccccc34)c3cc(-c4ccc5ccccc5c4)ccc3-c3ccc4c(c3)sc3ccccc34)cc21]

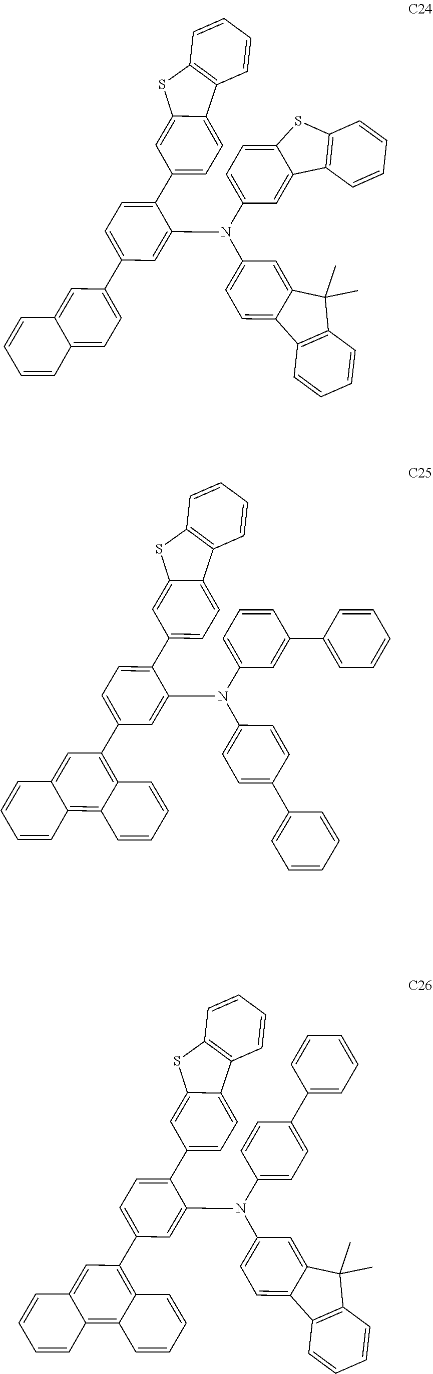 [SMILES: CC1(C)c2ccccc2-c2ccc(N(c3ccc4sc5ccccc5c4c3)c3cc(-c4ccc5ccccc5c4)ccc3-c3ccc4c(c3)sc3ccccc34)cc21 ; c1ccc(-c2ccc(N(c3cccc(-c4ccccc4)c3)c3cc(-c4cc5ccccc5c5ccccc45)ccc3-c3ccc4c(c3)sc3ccccc34)cc2)cc1 ; CC1(C)c2ccccc2-c2ccc(N(c3ccc(-c4ccccc4)cc3)c3cc(-c4cc5ccccc5c5ccccc45)ccc3-c3ccc4c(c3)sc3ccccc34)cc21]

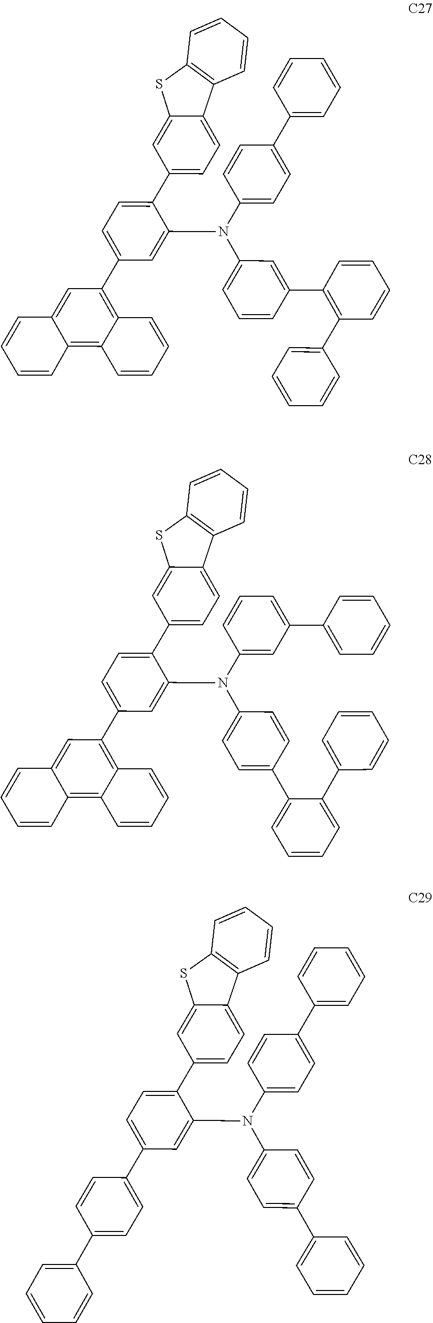 [SMILES: c1ccc(-c2ccc(N(c3cccc(-c4ccccc4-c4ccccc4)c3)c3cc(-c4cc5ccccc5c5ccccc45)ccc3-c3ccc4c(c3)sc3ccccc34)cc2)cc1 ; c1ccc(-c2cccc(N(c3ccc(-c4ccccc4-c4ccccc4)cc3)c3cc(-c4cc5ccccc5c5ccccc45)ccc3-c3ccc4c(c3)sc3ccccc34)c2)cc1 ; c1ccc(-c2ccc(-c3ccc(-c4ccc5c(c4)sc4ccccc45)c(N(c4ccc(-c5ccccc5)cc4)c4ccc(-c5ccccc5)cc4)c3)cc2)cc1]

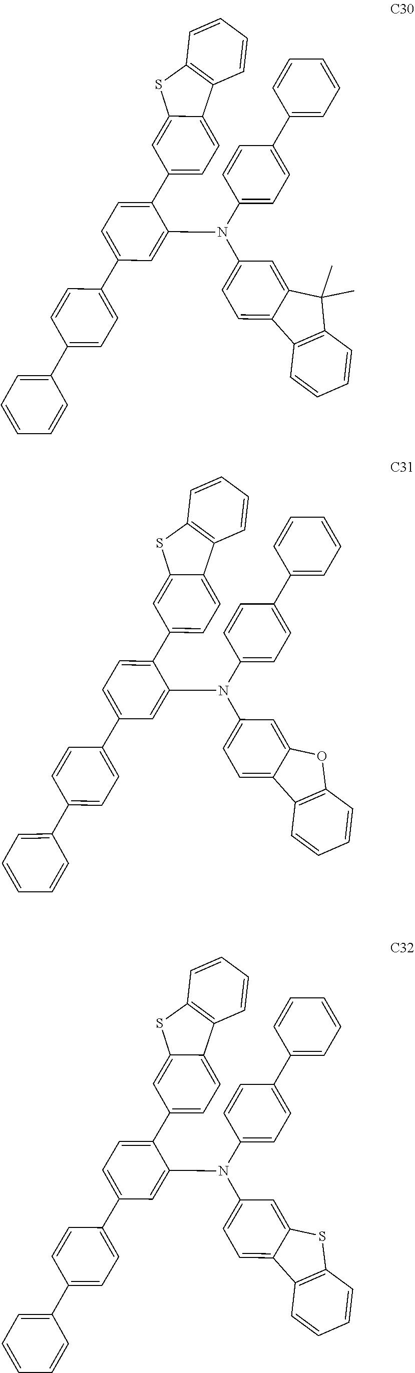 [SMILES: CC1(C)c2ccccc2-c2ccc(N(c3ccc(-c4ccccc4)cc3)c3cc(-c4ccc(-c5ccccc5)cc4)ccc3-c3ccc4c(c3)sc3ccccc34)cc21 ; c1ccc(-c2ccc(-c3ccc(-c4ccc5c(c4)sc4ccccc45)c(N(c4ccc(-c5ccccc5)cc4)c4ccc5c(c4)oc4ccccc45)c3)cc2)cc1 ; c1ccc(-c2ccc(-c3ccc(-c4ccc5c(c4)sc4ccccc45)c(N(c4ccc(-c5ccccc5)cc4)c4ccc5c(c4)sc4ccccc45)c3)cc2)cc1]

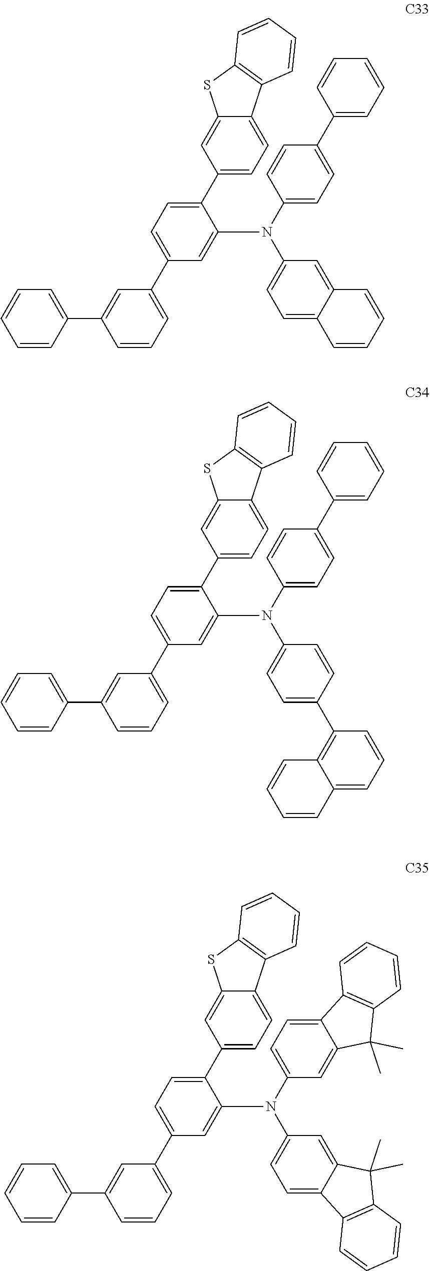 [SMILES: c1ccc(-c2ccc(N(c3ccc4ccccc4c3)c3cc(-c4cccc(-c5ccccc5)c4)ccc3-c3ccc4c(c3)sc3ccccc34)cc2)cc1 ; c1ccc(-c2ccc(N(c3ccc(-c4cccc5ccccc45)cc3)c3cc(-c4cccc(-c5ccccc5)c4)ccc3-c3ccc4c(c3)sc3ccccc34)cc2)cc1 ; CC1(C)c2ccccc2-c2ccc(N(c3ccc4c(c3)C(C)(C)c3ccccc3-4)c3cc(-c4cccc(-c5ccccc5)c4)ccc3-c3ccc4c(c3)sc3ccccc34)cc21]

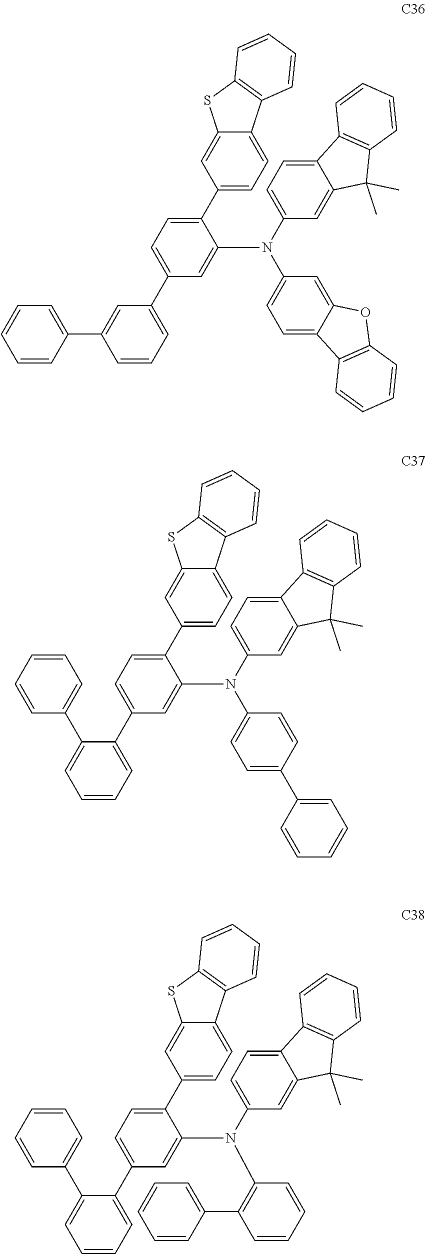 [SMILES: CC1(C)c2ccccc2-c2ccc(N(c3ccc4c(c3)oc3ccccc34)c3cc(-c4cccc(-c5ccccc5)c4)ccc3-c3ccc4c(c3)sc3ccccc34)cc21 ; CC1(C)c2ccccc2-c2ccc(N(c3ccc(-c4ccccc4)cc3)c3cc(-c4ccccc4-c4ccccc4)ccc3-c3ccc4c(c3)sc3ccccc34)cc21 ; CC1(C)c2ccccc2-c2ccc(N(c3ccccc3-c3ccccc3)c3cc(-c4ccccc4-c4ccccc4)ccc3-c3ccc4c(c3)sc3ccccc34)cc21]

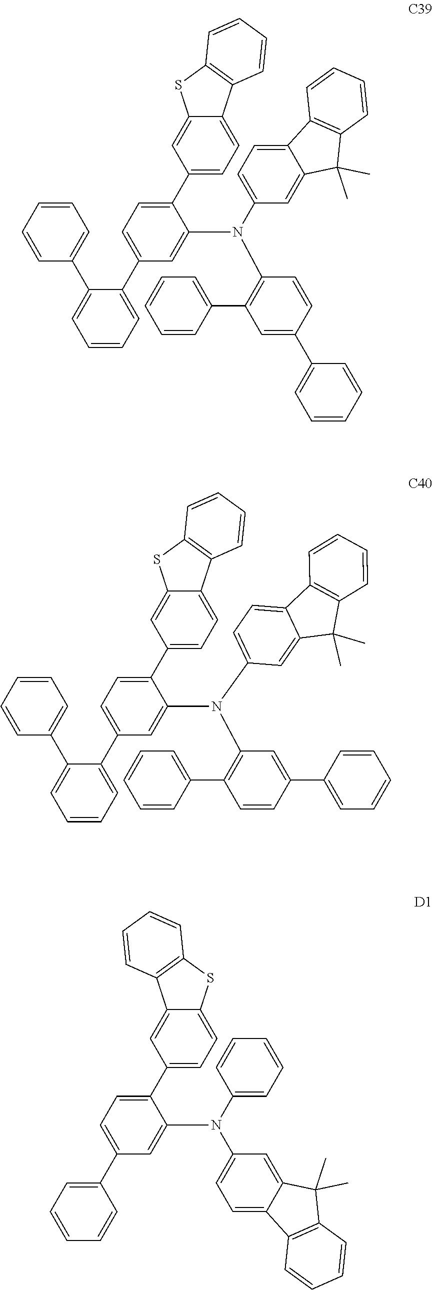 [SMILES: CC1(C)c2ccccc2-c2ccc(N(c3ccc(-c4ccccc4)cc3-c3ccccc3)c3cc(-c4ccccc4-c4ccccc4)ccc3-c3ccc4c(c3)sc3ccccc34)cc21 ; CC1(C)c2ccccc2-c2ccc(N(c3cc(-c4ccccc4)ccc3-c3ccccc3)c3cc(-c4ccccc4-c4ccccc4)ccc3-c3ccc4c(c3)sc3ccccc34)cc21 ; CC1(C)c2ccccc2-c2ccc(N(c3ccccc3)c3cc(-c4ccccc4)ccc3-c3ccc4sc5ccccc5c4c3)cc21]

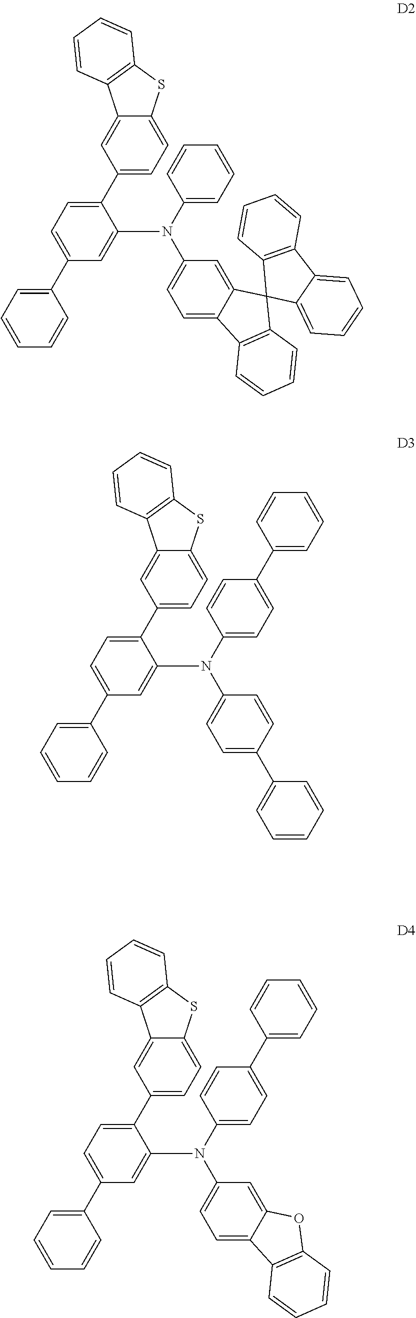 [SMILES: c1ccc(-c2ccc(-c3ccc4sc5ccccc5c4c3)c(N(c3ccccc3)c3ccc4c(c3)C3(c5ccccc5-c5ccccc53)c3ccccc3-4)c2)cc1 ; c1ccc(-c2ccc(N(c3ccc(-c4ccccc4)cc3)c3cc(-c4ccccc4)ccc3-c3ccc4sc5ccccc5c4c3)cc2)cc1 ; c1ccc(-c2ccc(N(c3ccc4c(c3)oc3ccccc34)c3cc(-c4ccccc4)ccc3-c3ccc4sc5ccccc5c4c3)cc2)cc1]

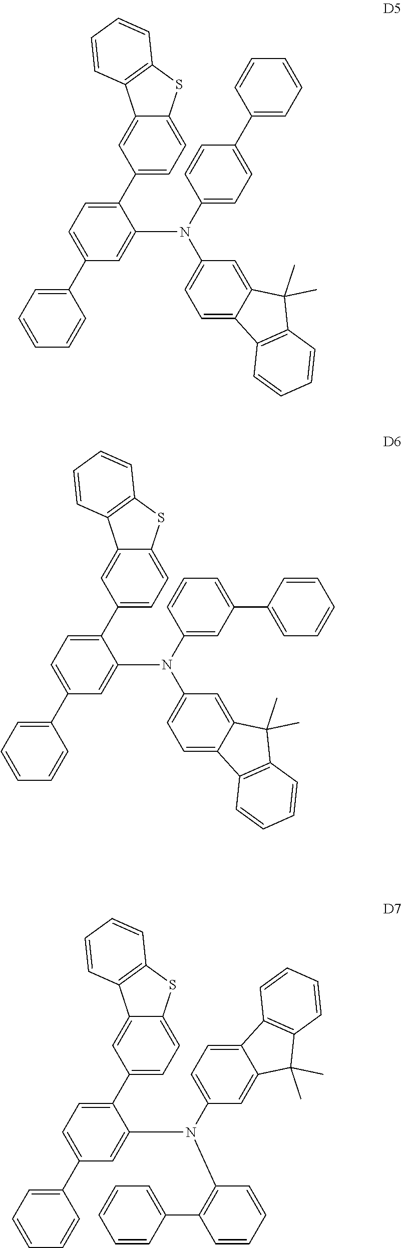 [SMILES: CC1(C)c2ccccc2-c2ccc(N(c3ccc(-c4ccccc4)cc3)c3cc(-c4ccccc4)ccc3-c3ccc4sc5ccccc5c4c3)cc21 ; CC1(C)c2ccccc2-c2ccc(N(c3cccc(-c4ccccc4)c3)c3cc(-c4ccccc4)ccc3-c3ccc4sc5ccccc5c4c3)cc21 ; CC1(C)c2ccccc2-c2ccc(N(c3ccccc3-c3ccccc3)c3cc(-c4ccccc4)ccc3-c3ccc4sc5ccccc5c4c3)cc21]

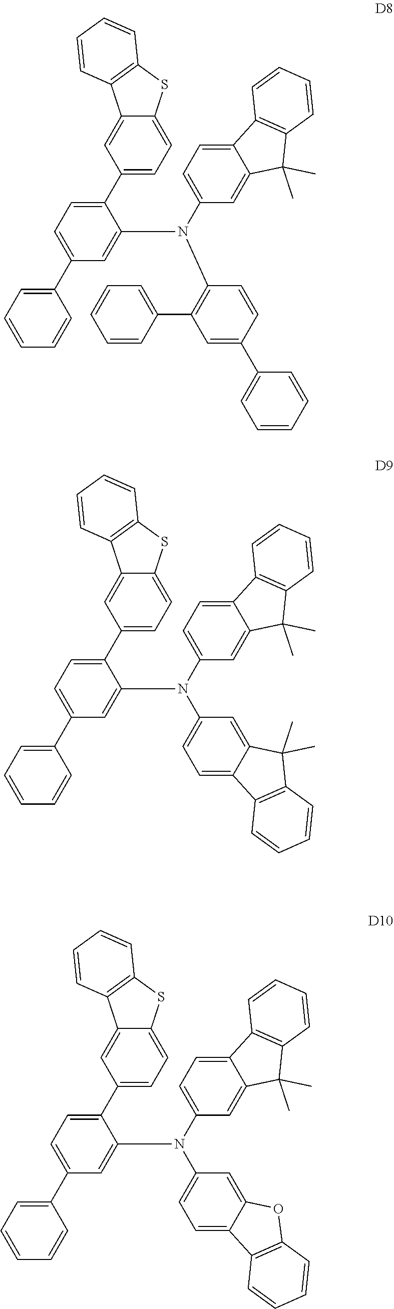 [SMILES: CC1(C)c2ccccc2-c2ccc(N(c3ccc(-c4ccccc4)cc3-c3ccccc3)c3cc(-c4ccccc4)ccc3-c3ccc4sc5ccccc5c4c3)cc21 ; CC1(C)c2ccccc2-c2ccc(N(c3ccc4c(c3)C(C)(C)c3ccccc3-4)c3cc(-c4ccccc4)ccc3-c3ccc4sc5ccccc5c4c3)cc21 ; CC1(C)c2ccccc2-c2ccc(N(c3ccc4c(c3)oc3ccccc34)c3cc(-c4ccccc4)ccc3-c3ccc4sc5ccccc5c4c3)cc21]

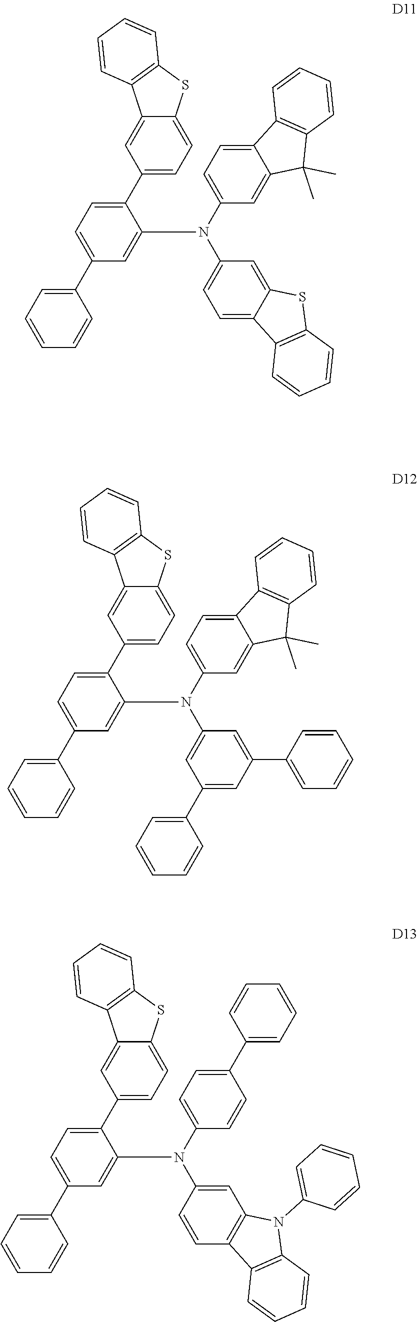 [SMILES: CC1(C)c2ccccc2-c2ccc(N(c3ccc4c(c3)sc3ccccc34)c3cc(-c4ccccc4)ccc3-c3ccc4sc5ccccc5c4c3)cc21 ; CC1(C)c2ccccc2-c2ccc(N(c3cc(-c4ccccc4)cc(-c4ccccc4)c3)c3cc(-c4ccccc4)ccc3-c3ccc4sc5ccccc5c4c3)cc21 ; c1ccc(-c2ccc(N(c3ccc4c5ccccc5n(-c5ccccc5)c4c3)c3cc(-c4ccccc4)ccc3-c3ccc4sc5ccccc5c4c3)cc2)cc1]

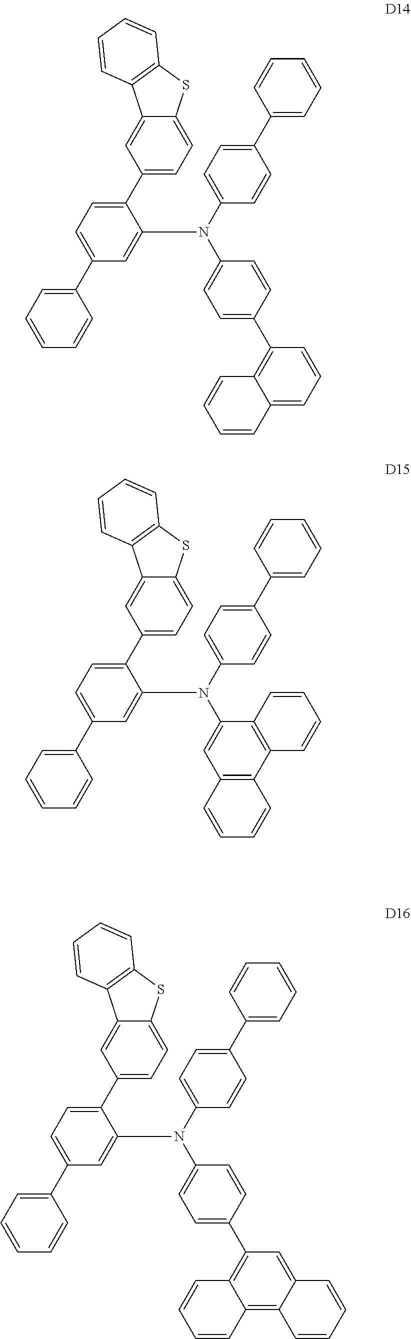 [SMILES: c1ccc(-c2ccc(N(c3ccc(-c4cccc5ccccc45)cc3)c3cc(-c4ccccc4)ccc3-c3ccc4sc5ccccc5c4c3)cc2)cc1 ; c1ccc(-c2ccc(N(c3cc(-c4ccccc4)ccc3-c3ccc4sc5ccccc5c4c3)c3cc4ccccc4c4ccccc34)cc2)cc1 ; c1ccc(-c2ccc(N(c3ccc(-c4cc5ccccc5c5ccccc45)cc3)c3cc(-c4ccccc4)ccc3-c3ccc4sc5ccccc5c4c3)cc2)cc1]

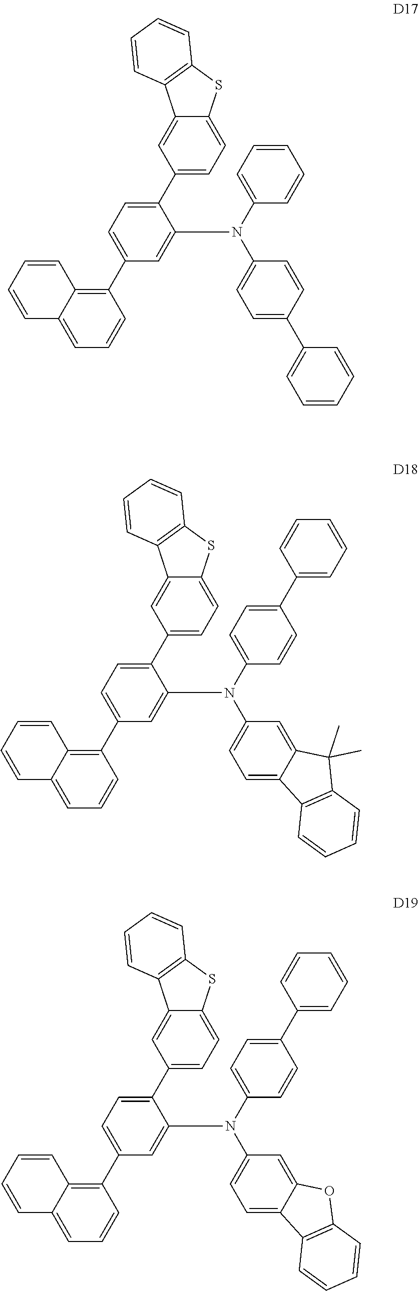 [SMILES: c1ccc(-c2ccc(N(c3ccccc3)c3cc(-c4cccc5ccccc45)ccc3-c3ccc4sc5ccccc5c4c3)cc2)cc1 ; CC1(C)c2ccccc2-c2ccc(N(c3ccc(-c4ccccc4)cc3)c3cc(-c4cccc5ccccc45)ccc3-c3ccc4sc5ccccc5c4c3)cc21 ; c1ccc(-c2ccc(N(c3ccc4c(c3)oc3ccccc34)c3cc(-c4cccc5ccccc45)ccc3-c3ccc4sc5ccccc5c4c3)cc2)cc1]

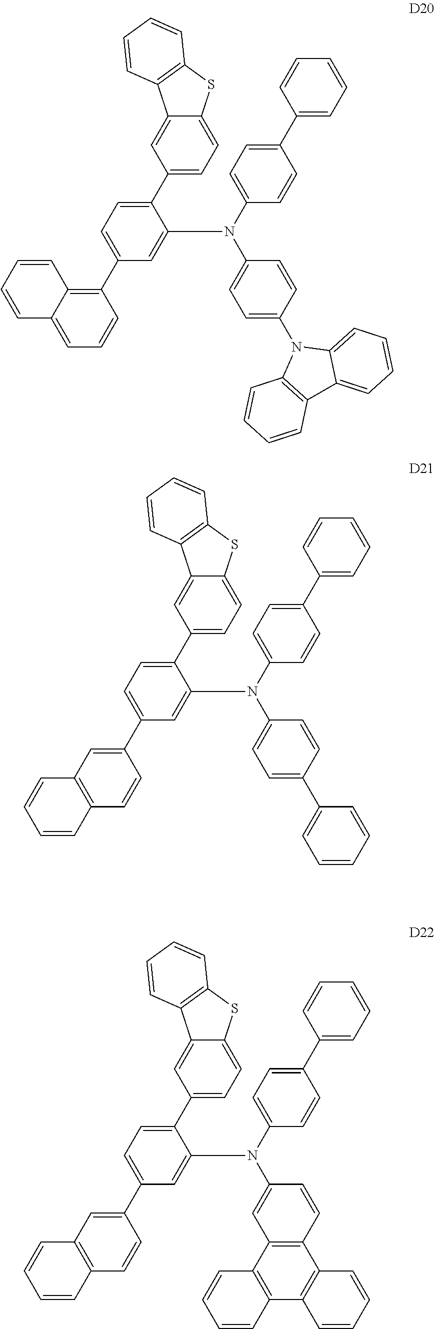 [SMILES: c1ccc(-c2ccc(N(c3ccc(-n4c5ccccc5c5ccccc54)cc3)c3cc(-c4cccc5ccccc45)ccc3-c3ccc4sc5ccccc5c4c3)cc2)cc1 ; c1ccc(-c2ccc(N(c3ccc(-c4ccccc4)cc3)c3cc(-c4ccc5ccccc5c4)ccc3-c3ccc4sc5ccccc5c4c3)cc2)cc1 ; c1ccc(-c2ccc(N(c3ccc4c5ccccc5c5ccccc5c4c3)c3cc(-c4ccc5ccccc5c4)ccc3-c3ccc4sc5ccccc5c4c3)cc2)cc1]

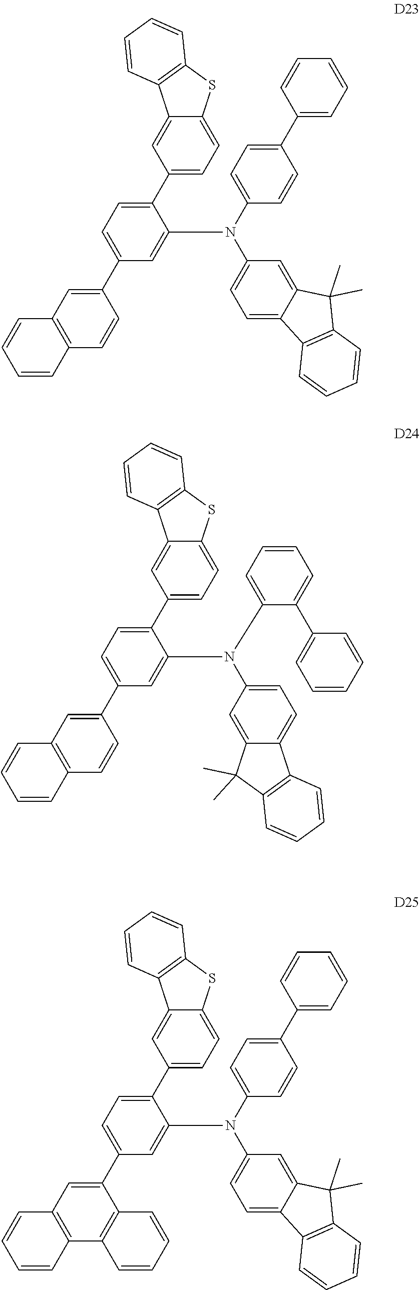 [SMILES: CC1(C)c2ccccc2-c2ccc(N(c3ccc(-c4ccccc4)cc3)c3cc(-c4ccc5ccccc5c4)ccc3-c3ccc4sc5ccccc5c4c3)cc21 ; CC1(C)c2ccccc2-c2ccc(N(c3ccccc3-c3ccccc3)c3cc(-c4ccc5ccccc5c4)ccc3-c3ccc4sc5ccccc5c4c3)cc21 ; CC1(C)c2ccccc2-c2ccc(N(c3ccc(-c4ccccc4)cc3)c3cc(-c4cc5ccccc5c5ccccc45)ccc3-c3ccc4sc5ccccc5c4c3)cc21]

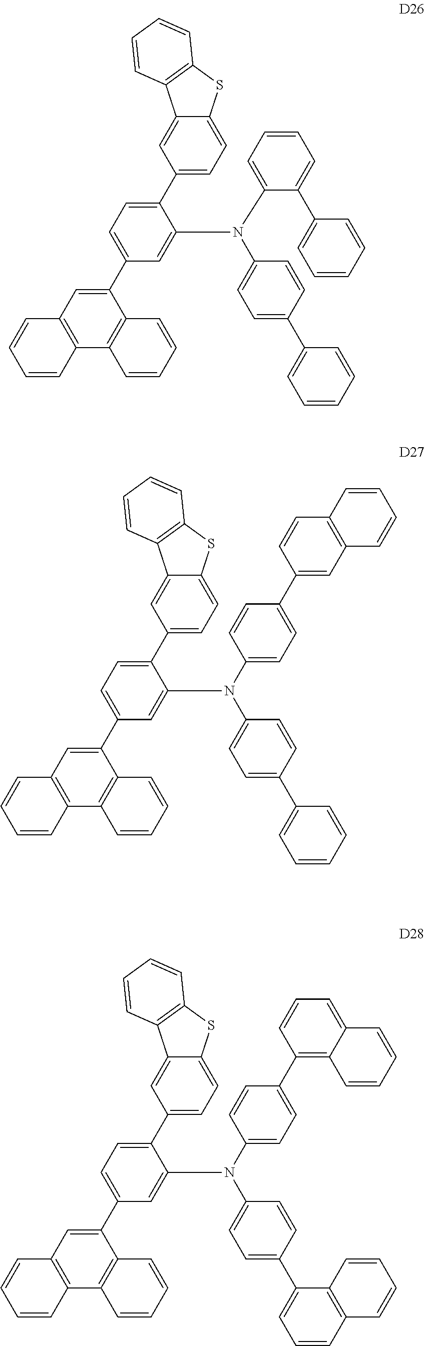 [SMILES: c1ccc(-c2ccc(N(c3ccccc3-c3ccccc3)c3cc(-c4cc5ccccc5c5ccccc45)ccc3-c3ccc4sc5ccccc5c4c3)cc2)cc1 ; c1ccc(-c2ccc(N(c3ccc(-c4ccc5ccccc5c4)cc3)c3cc(-c4cc5ccccc5c5ccccc45)ccc3-c3ccc4sc5ccccc5c4c3)cc2)cc1 ; c1ccc2c(-c3ccc(N(c4ccc(-c5cccc6ccccc56)cc4)c4cc(-c5cc6ccccc6c6ccccc56)ccc4-c4ccc5sc6ccccc6c5c4)cc3)cccc2c1]

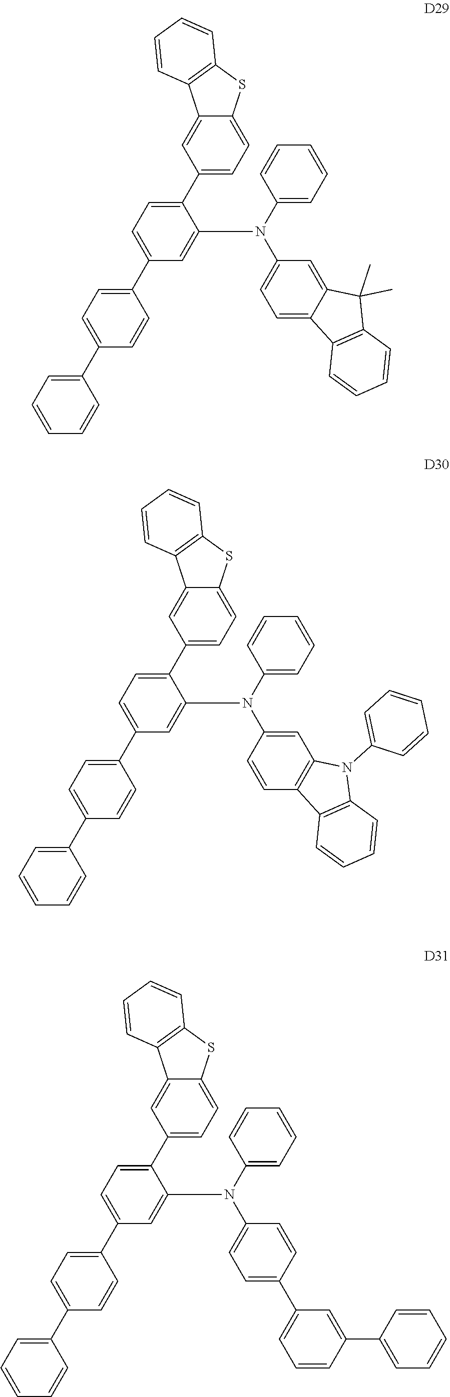 [SMILES: CC1(C)c2ccccc2-c2ccc(N(c3ccccc3)c3cc(-c4ccc(-c5ccccc5)cc4)ccc3-c3ccc4sc5ccccc5c4c3)cc21 ; c1ccc(-c2ccc(-c3ccc(-c4ccc5sc6ccccc6c5c4)c(N(c4ccccc4)c4ccc5c6ccccc6n(-c6ccccc6)c5c4)c3)cc2)cc1 ; c1ccc(-c2ccc(-c3ccc(-c4ccc5sc6ccccc6c5c4)c(N(c4ccccc4)c4ccc(-c5cccc(-c6ccccc6)c5)cc4)c3)cc2)cc1]

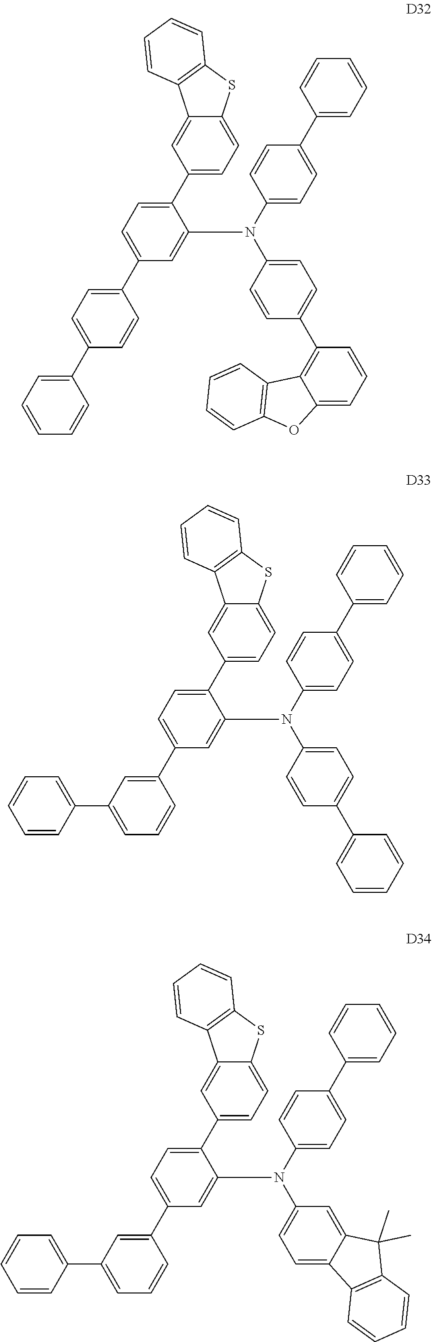 [SMILES: c1ccc(-c2ccc(-c3ccc(-c4ccc5sc6ccccc6c5c4)c(N(c4ccc(-c5ccccc5)cc4)c4ccc(-c5cccc6oc7ccccc7c56)cc4)c3)cc2)cc1 ; c1ccc(-c2ccc(N(c3ccc(-c4ccccc4)cc3)c3cc(-c4cccc(-c5ccccc5)c4)ccc3-c3ccc4sc5ccccc5c4c3)cc2)cc1 ; CC1(C)c2ccccc2-c2ccc(N(c3ccc(-c4ccccc4)cc3)c3cc(-c4cccc(-c5ccccc5)c4)ccc3-c3ccc4sc5ccccc5c4c3)cc21]

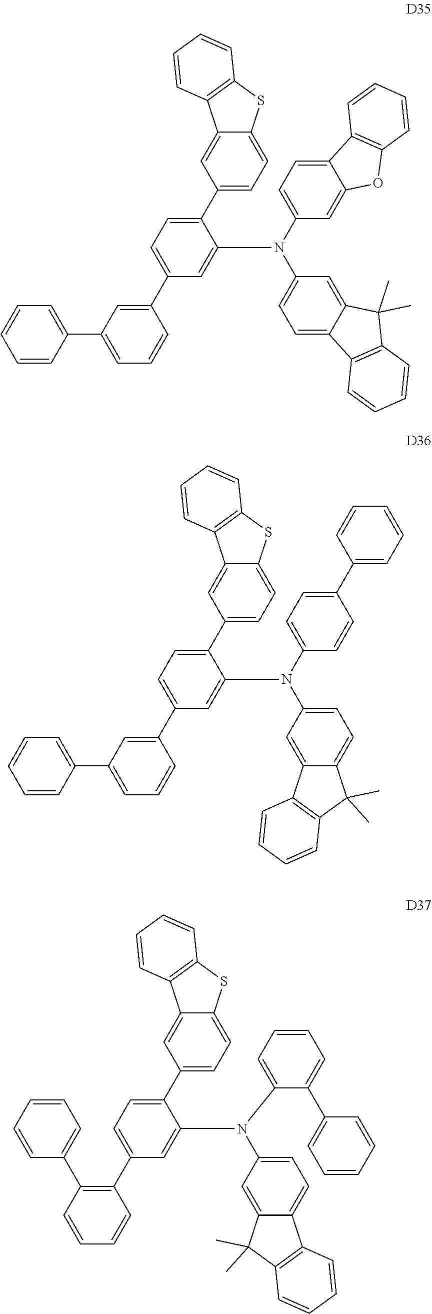 [SMILES: CC1(C)c2ccccc2-c2ccc(N(c3ccc4c(c3)oc3ccccc34)c3cc(-c4cccc(-c5ccccc5)c4)ccc3-c3ccc4sc5ccccc5c4c3)cc21 ; CC1(C)c2ccccc2-c2cc(N(c3ccc(-c4ccccc4)cc3)c3cc(-c4cccc(-c5ccccc5)c4)ccc3-c3ccc4sc5ccccc5c4c3)ccc21 ; CC1(C)c2ccccc2-c2ccc(N(c3ccccc3-c3ccccc3)c3cc(-c4ccccc4-c4ccccc4)ccc3-c3ccc4sc5ccccc5c4c3)cc21]

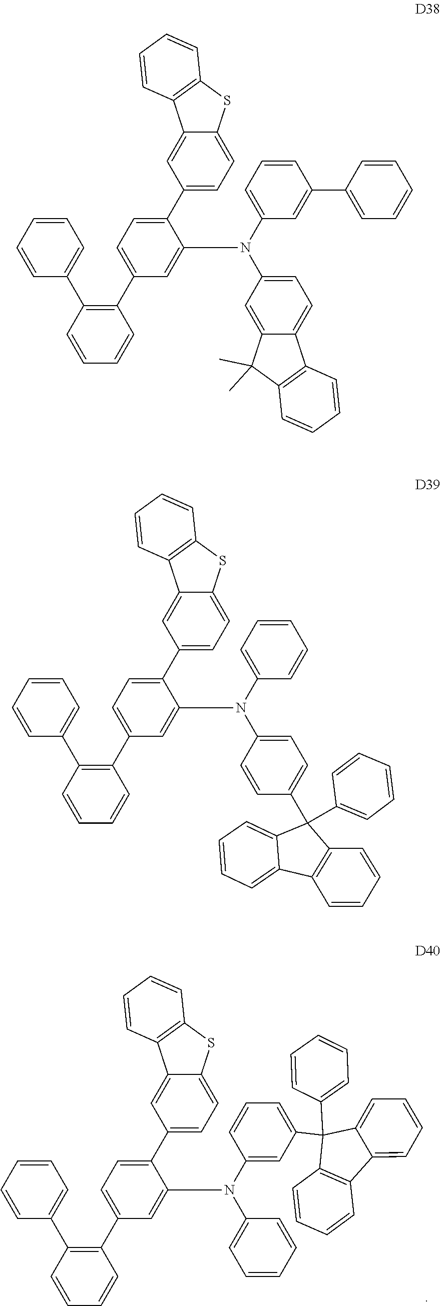 [SMILES: CC1(C)c2ccccc2-c2ccc(N(c3cccc(-c4ccccc4)c3)c3cc(-c4ccccc4-c4ccccc4)ccc3-c3ccc4sc5ccccc5c4c3)cc21 ; c1ccc(-c2ccccc2-c2ccc(-c3ccc4sc5ccccc5c4c3)c(N(c3ccccc3)c3ccc(C4(c5ccccc5)c5ccccc5-c5ccccc54)cc3)c2)cc1 ; c1ccc(-c2ccccc2-c2ccc(-c3ccc4sc5ccccc5c4c3)c(N(c3ccccc3)c3cccc(C4(c5ccccc5)c5ccccc5-c5ccccc54)c3)c2)cc1]

In a second aspect, the present application provides an organic electroluminescent device, including an anode and a cathode which are oppositely disposed, and a functional layer disposed between the anode and the cathode, where the functional layer includes the organic compound of the present application.

Optionally, the organic electroluminescent device is a red organic electroluminescent device.

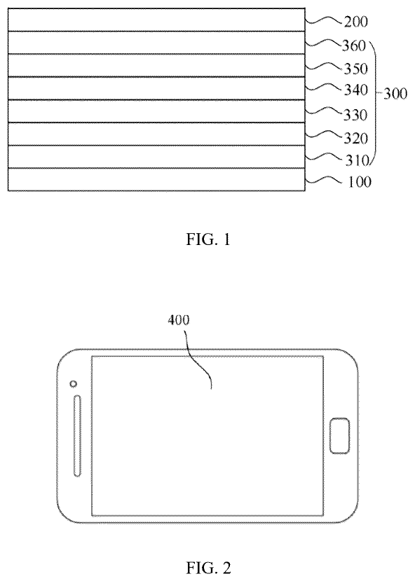

In some embodiments of the present application, an electronic component is an organic electroluminescent device. As shown in FIG. 1, the organic electroluminescent device may include an anode 100, a hole transport layer 320, a hole auxiliary layer 330, an organic light-emitting layer 340, an electron transport layer 350, and a cathode 200 which are sequentially stacked.

In some specific embodiments of the present application, the organic electroluminescent device is a red organic electroluminescent device.

Optionally, the anode 100 includes the following anode materials which are optionally materials having a large work function that facilitate hole injection into the functional layer.

Specific examples of the anode materials include metals such as nickel, platinum, vanadium, chromium, copper, zinc, and gold, or their alloys; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene](PEDT), polypyrrole, and polyaniline, but are not limited to this. A transparent electrode including indium tin oxide (ITO) as the anode is preferred.

Optionally, the hole transport layer 320 includes one or more hole transport materials, and the hole transport materials may be selected from carbazole multimers, carbazole-linked triarylamine compounds, or other types of compounds, which can be selected by those skilled in the art with reference to the prior art, and are not specially limited in the present application. In some embodiments of the present application, the hole transport layer 320 is HT-18.

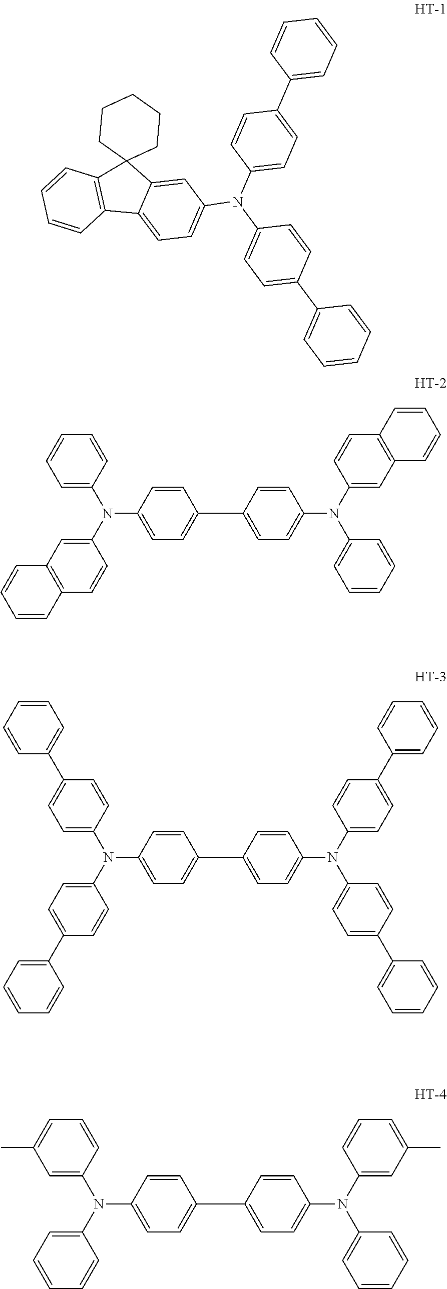

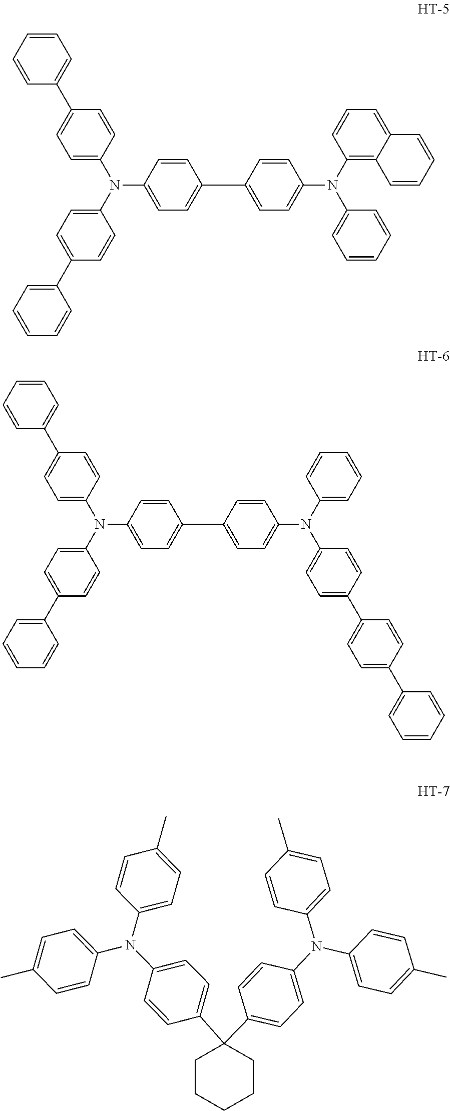

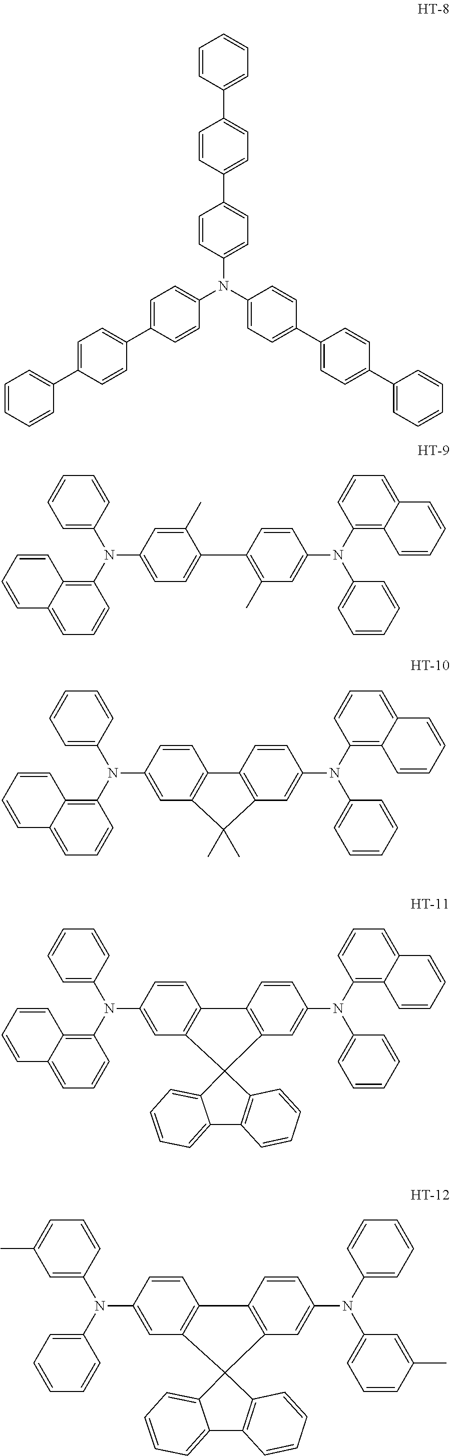

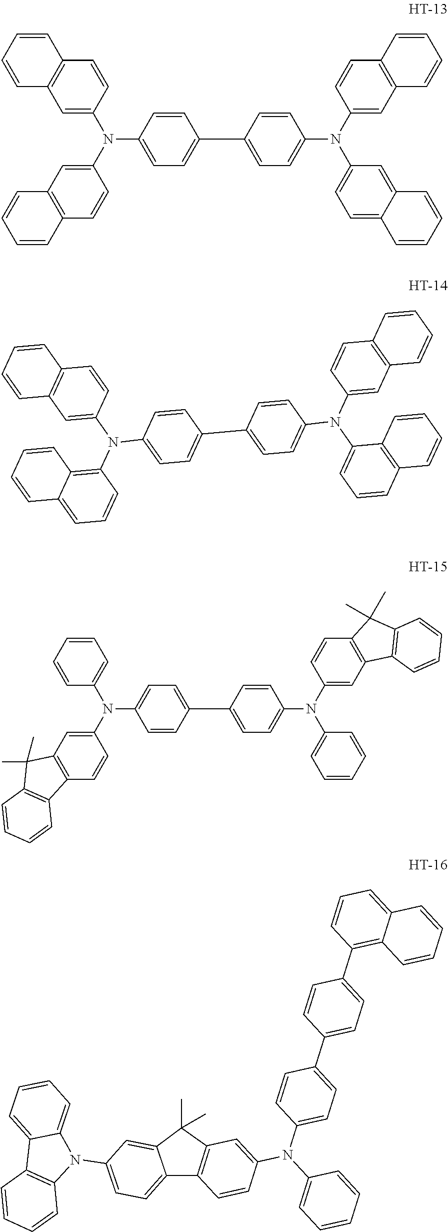 [SMILES: c1ccc2cc(N(c3ccc(-c4ccc(N(c5ccc6ccccc6c5)c5ccc6ccccc6c5)cc4)cc3)c3ccc4ccccc4c3)ccc2c1 ; c1ccc2cc(N(c3ccc(-c4ccc(N(c5ccc6ccccc6c5)c5cccc6ccccc56)cc4)cc3)c3cccc4ccccc34)ccc2c1 ; CC1(C)c2ccccc2-c2cc(N(c3ccccc3)c3ccc(-c4ccc(N(c5ccccc5)c5ccc6c(c5)C(C)(C)c5ccccc5-6)cc4)cc3)ccc21 ; CC1(C)c2cc(N(c3ccccc3)c3ccc(-c4ccc(-c5cccc6ccccc56)cc4)cc3)ccc2-c2ccc(-n3c4ccccc4c4ccccc43)cc21]

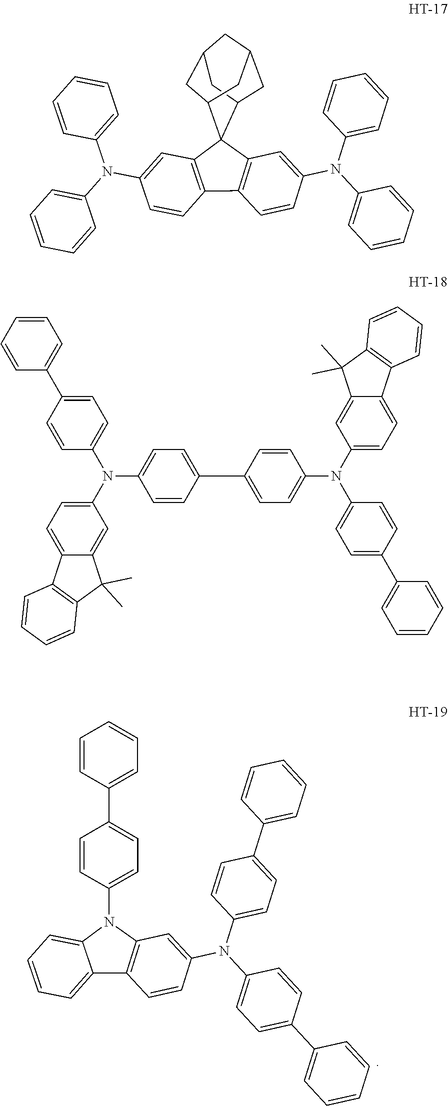 [SMILES: c1ccc(N(c2ccccc2)c2ccc3c(c2)C2(c4cc(N(c5ccccc5)c5ccccc5)ccc4-3)C3CC4CC(C3)CC2C4)cc1 ; CC1(C)c2ccccc2-c2ccc(N(c3ccc(-c4ccccc4)cc3)c3ccc(-c4ccc(N(c5ccc(-c6ccccc6)cc5)c5ccc6c(c5)C(C)(C)c5ccccc5-6)cc4)cc3)cc21 ; c1ccc(-c2ccc(N(c3ccc(-c4ccccc4)cc3)c3ccc4c5ccccc5n(-c5ccc(-c6ccccc6)cc5)c4c3)cc2)cc1]

In one embodiment of the present application, the hole auxiliary layer 330 is the organic compound of the present application.

Optionally, a hole injection layer 310 may also be arranged between the anode 100 and the hole transport layer 320 to enhance the ability to inject holes into the hole transport layer 320.

The hole injection layer 310 can be made of a benzidine derivative, a starburst arylamine compound, a phthalocyanine derivative or other materials, which is not specially limited in the present application. A material of the hole injection layer 310 may be selected from, for example, the following compounds or any combination of them;

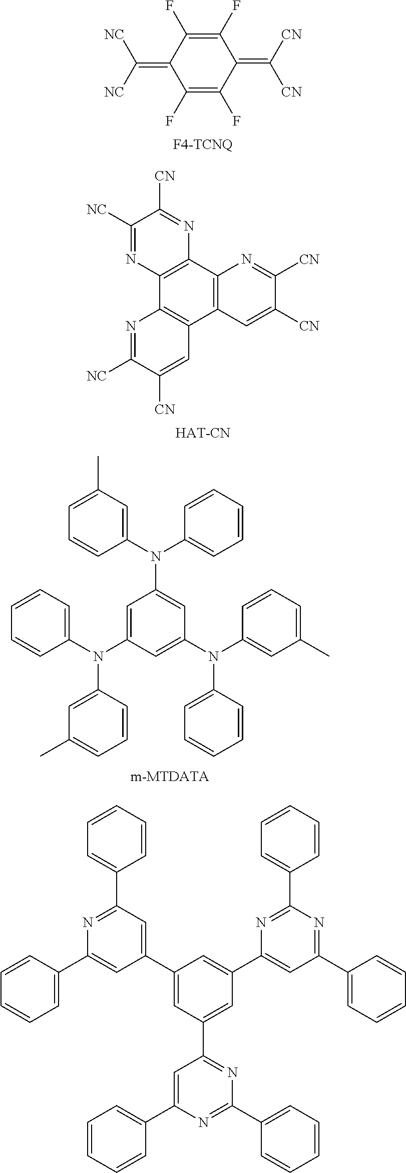 [SMILES: N#CC(C#N)=c1c(F)c(F)c(=C(C#N)C#N)c(F)c1F ; N#Cc1cc2c3cc(C#N)c(C#N)nc3c3nc(C#N)c(C#N)nc3c2nc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

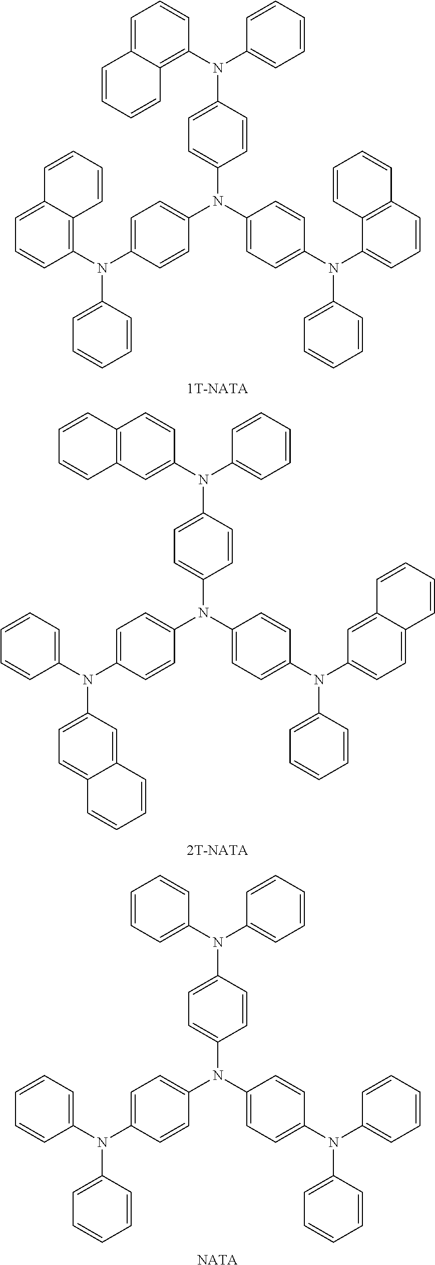

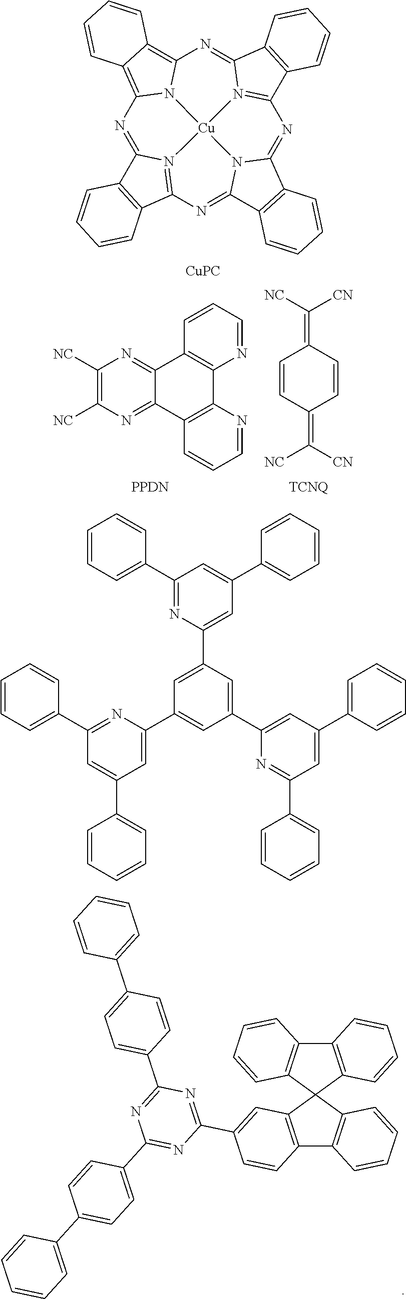

In some embodiments of the present application, the hole injection layer 310 is composed of F4-TCNQ and HT-18.

Optionally, the organic light-emitting layer 340 may be composed of a single light-emitting layer material, and may also include a host material and a doping material. Optionally, the organic light-emitting layer 340 is composed of the host material and the doping material, holes injected into the organic light-emitting layer 340 and electrons injected into the organic light-emitting layer 340 can be recombined in the organic light-emitting layer 340 to form excitons, the excitons transfer energy to the host material, the host material transfers energy to the doping material, and then the doping material can emit light.

The host material of the organic light-emitting layer 340 may be a metal chelated compound, a distyryl derivative, an aromatic amine derivative, a dibenzofuran derivative or other types of materials, which is not specially limited in the present application.

In some embodiments of the present application, the host material of the organic light-emitting layer 340 is RH-01.

A guest material of the organic light-emitting layer 340 may be a compound having a condensed aryl ring or its derivative, a compound having a heteroaryl ring or its derivative, an aromatic amine derivative, or other materials, which is not particularly limited in the present application. The guest material is also referred to as a doping material or a dopant. Specific examples of red phosphorescent dopants for red organic electroluminescent devices include, but are not limited to,

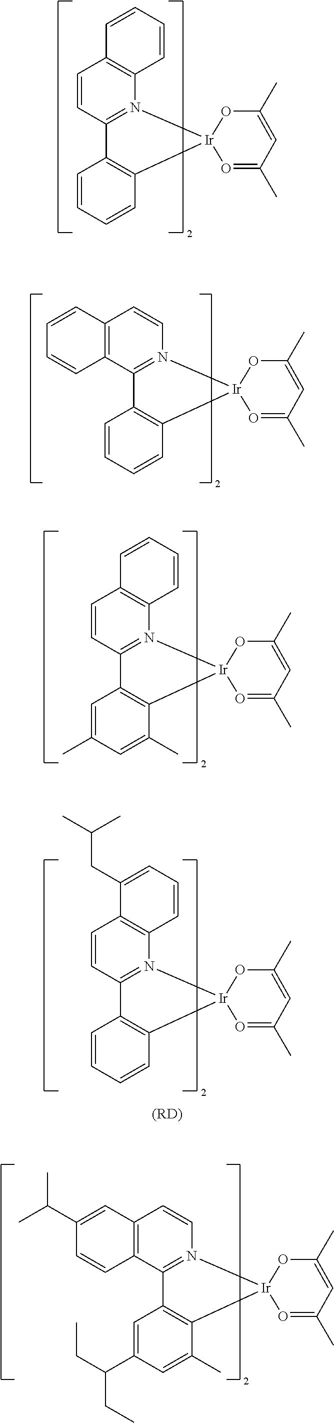

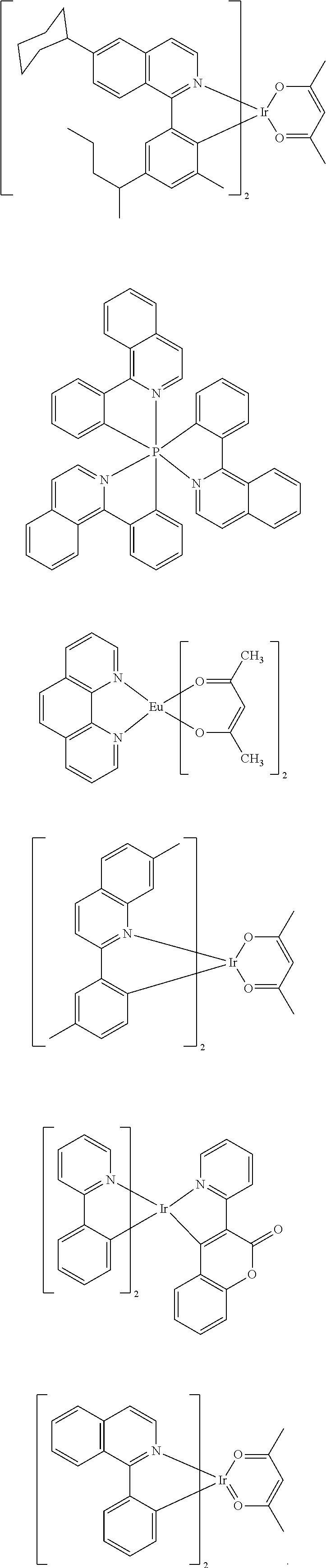

In a more particular embodiment, the host material of the organic light-emitting layer 340 is RH-01 and the guest material is RD.

The electron transport layer 350 may be of a single-layer structure or a multi-layer structure, and may include one or more electron transport materials, and the electron transport materials may be selected from, but are not limited to, ET-01, LiQ, a benzimidazole derivative, an oxadiazole derivative, a quinoxaline derivative, or other electron transport materials, which are not particularly limited in the present application. A material of the electron transport layer 350 includes, but is not limited to, the following compounds:

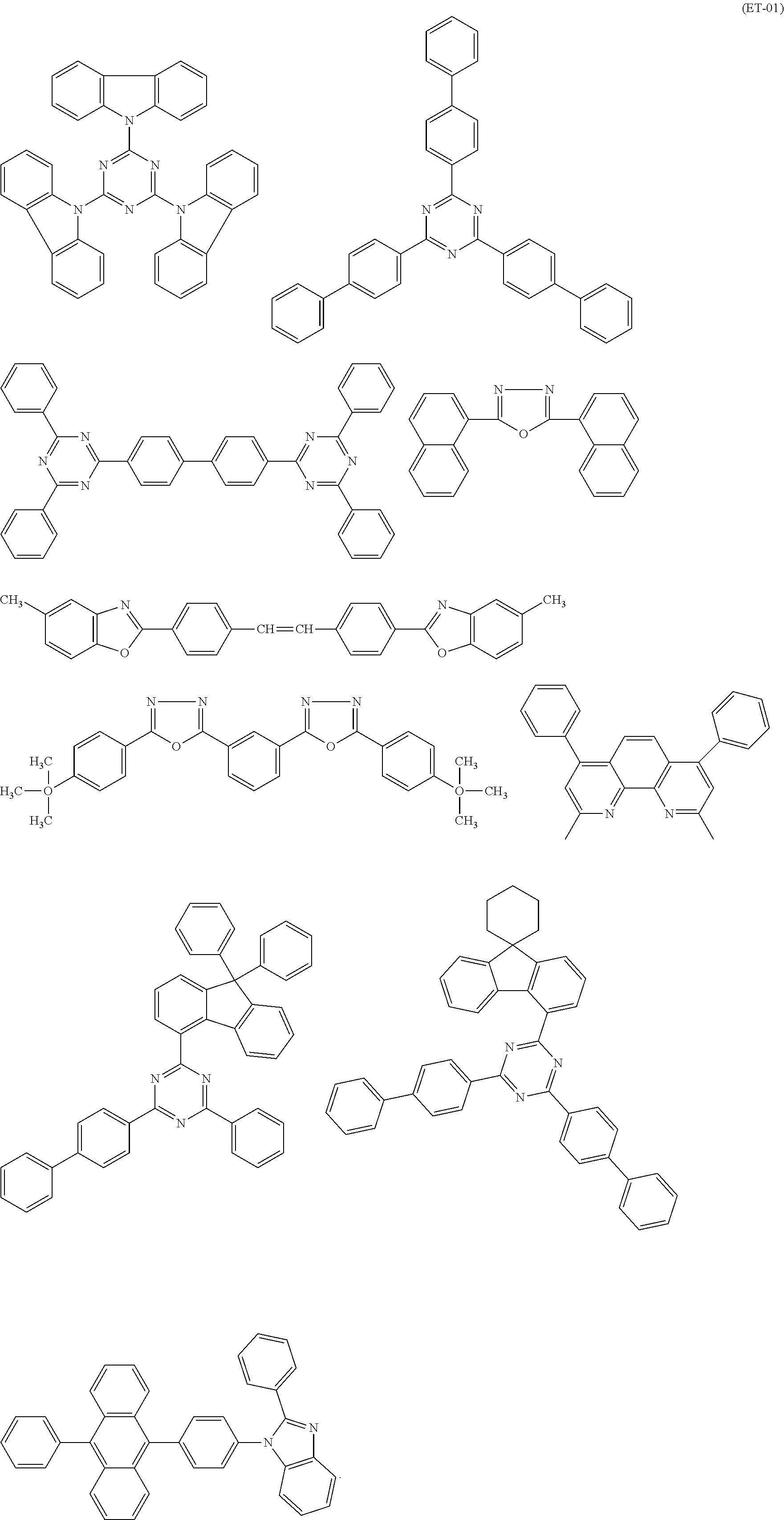

In some embodiments of the present application, the electron transport layer 350 is composed of ET-01 and LiQ.

In the present application, the cathode 200 may include a cathode material, which is a material with a small work function that facilitates electron injection into the functional layer.

Specific examples of the cathode material include, but are not limited to, metals such as magnesium, calcium, sodium, potassium, titanium, indium, yttrium, lithium, gadolinium, aluminum, silver, tin, and lead, or their alloys; or a multilayer material such as LiF/Al, Liq/Al, LiO₂/Al, LiF/Ca, LiF/Al, and BaF₂/Ca. Optionally, a metal electrode including magnesium and silver as the cathode is included.

In some embodiments of the present application, the electron injection layer 360 may include ytterbium (Yb).

According to a third aspect of the present application, provided is an electronic apparatus, including the organic electroluminescent device according to the second aspect of the present application.

According to one embodiment, as shown in FIG. 2, the electronic apparatus provided is an electronic apparatus 400 including the organic electroluminescent device described above. The electronic apparatus 400 may be, for example, a display device, a lighting device, an optical communication device, or other type of electronic devices, and may include, for example, but is not limited to, a computer screen, a mobile phone screen, a television, electronic paper, an emergency lighting lamp, an optical module, and the like.

A synthesis method of the organic compound of the present application is specifically described below in combination with the synthesis examples, but the present application is not limited in any way accordingly.

Compounds of which synthesis methods were not mentioned in the present application were all commercially available raw material products.

### Synthesis of Intermediate a0

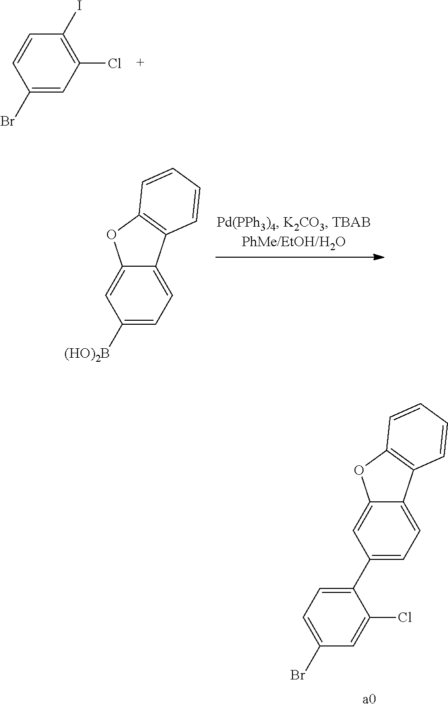 [SMILES: Clc1cc(Br)ccc1I ; OB(O)c1ccc2c(c1)oc1ccccc12 ; Clc1cc(Br)ccc1-c1ccc2c(c1)oc1ccccc12]

Under nitrogen protection, 4-bromo-2-chloroiodobenzene (7.5 g; 23.6 mmol), dibenzofuran-3-boronic acid (5.0 g; 23.6 mmol), tetrakis(triphenylphosphine)palladium (0.5 g; 0.5 mmol), potassium carbonate (6.5 g; 47.3 mmol), tetrabutylammonium bromide (1.5 g; 4.7 mmol), toluene (60 mL), ethanol (15 mL) and deionized water (15 mL) were added into a round bottom flask, and heated to 75° C. to 80° C., and a reaction was carried out under stirring for 16 h; the reaction solution was cooled to room temperature, deionized water was added, liquid separation was performed, and an organic phase was washed with water and dried over anhydrous magnesium sulfate, and a solvent was removed under reduced pressure; and the obtained crude product was purified by silica gel column chromatography using a dichloromethane/n-heptane solvent system to obtain Intermediate a0 (6.4 g; yield: 76%) as a white solid.

Referring to the synthesis method for the intermediate a0, by substituting Reactant A for dibenzofuran-3-boronic acid, intermediates shown in Table 1 below were synthesized:

TABLE 1

| Intermediate No. | Reactant A | Structure | Yield (%) |
|---|---|---|---|
| b0 | 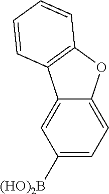 [SMILES: OB(O)c1ccc2oc3ccccc3c2c1] | 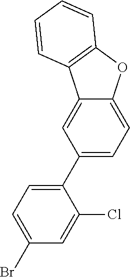 [SMILES: Clc1cc(Br)ccc1-c1ccc2oc3ccccc3c2c1] | 68 |
| c0 | 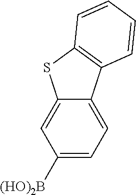 [SMILES: OB(O)c1ccc2c(c1)sc1ccccc12] | 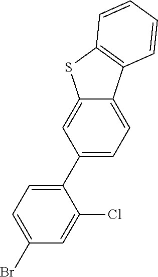 [SMILES: Clc1cc(Br)ccc1-c1ccc2c(c1)sc1ccccc12] | 70 |
| d0 | 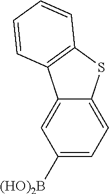 [SMILES: OB(O)c1ccc2sc3ccccc3c2c1] | 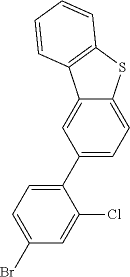 [SMILES: Clc1cc(Br)ccc1-c1ccc2sc3ccccc3c2c1] | 72 |

### Synthesis of Intermediate a1

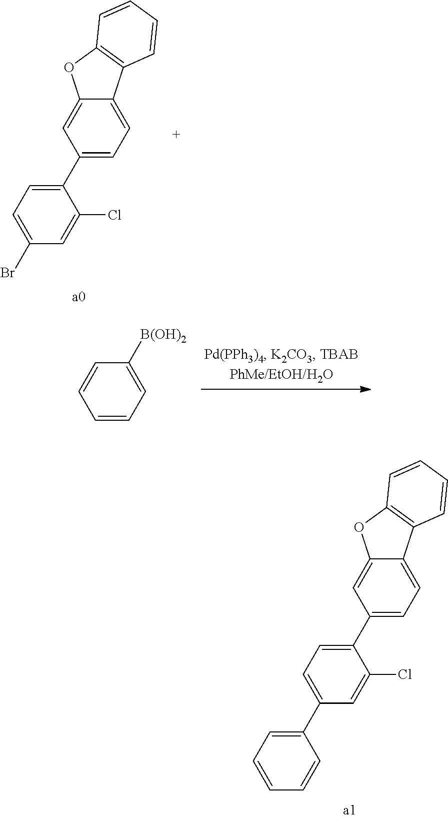 [SMILES: Clc1cc(Br)ccc1-c1ccc2c(c1)oc1ccccc12 ; OB(O)c1ccccc1 ; Clc1cc(-c2ccccc2)ccc1-c1ccc2c(c1)oc1ccccc12]

Under nitrogen protection, the Intermediate a0 (6.2 g; 17.3 mmol), phenylboronic acid (2.2 g; 18.2 mmol), tetrakis(triphenylphosphine)palladium (0.4 g; 0.3 mmol), potassium carbonate (4.8 g; 34.7 mmol), tetrabutylammonium bromide (1.1 g; 3.5 mmol), toluene (50 mL), ethanol (15 mL), and deionized water (15 mL) were added into a round bottom flask, and heated to 75° C. to 80° C., and a reaction was carried out under stirring for 16 h; the reaction solution was cooled to room temperature, deionized water was added, liquid separation was performed, and an organic phase was washed with water and dried over anhydrous magnesium sulfate, and a solvent was removed under reduced pressure; and the obtained crude product was purified by recrystallization using a dichloromethane/n-heptane solvent system to obtain Intermediate a1 (4.9 g; yield: 800%) as a white solid.

Referring to the synthesis method for the intermediate a1, by substituting a Reactant B for Intermediate a0 and a Reactant C for phenylboronic acid, intermediates shown in Table 2 below were synthesized:

TABLE 2

| Intermediate No. | Reactant B | Reactant C | Structure | Yield (%) |
|---|---|---|---|---|
| a2 | 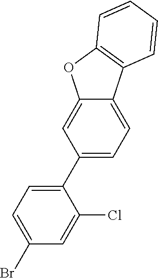 [SMILES: Clc1cc(Br)ccc1-c1ccc2c(c1)oc1ccccc12] | 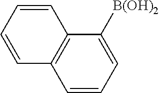 [SMILES: OB(O)c1cccc2ccccc12] | 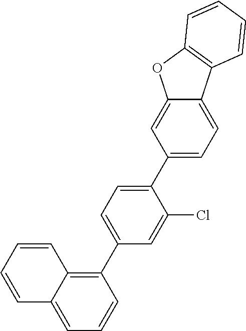 [SMILES: Clc1cc(-c2cccc3ccccc23)ccc1-c1ccc2c(c1)oc1ccccc12] | 72 |
| a3 | 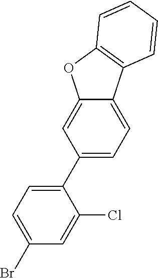 [SMILES: Clc1cc(Br)ccc1-c1ccc2c(c1)oc1ccccc12] | 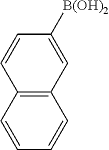 [SMILES: OB(O)c1ccc2ccccc2c1] | 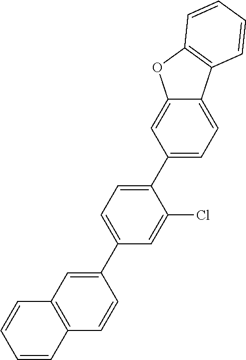 [SMILES: Clc1cc(-c2ccc3ccccc3c2)ccc1-c1ccc2c(c1)oc1ccccc12] | 82 |
| a4 | 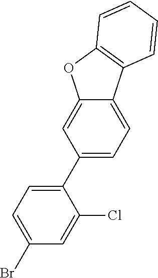 [SMILES: Clc1cc(Br)ccc1-c1ccc2c(c1)oc1ccccc12] | 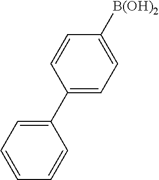 [SMILES: OB(O)c1ccc(-c2ccccc2)cc1] | 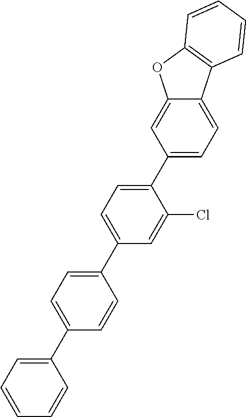 [SMILES: Clc1cc(-c2ccc(-c3ccccc3)cc2)ccc1-c1ccc2c(c1)oc1ccccc12] | 70 |
| a5 | 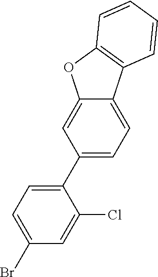 [SMILES: Clc1cc(Br)ccc1-c1ccc2c(c1)oc1ccccc12] | 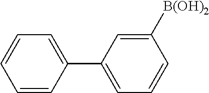 [SMILES: OB(O)c1cccc(-c2ccccc2)c1] | 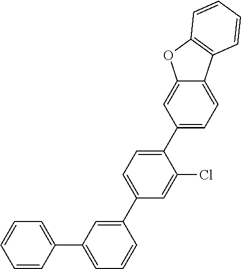 [SMILES: Clc1cc(-c2cccc(-c3ccccc3)c2)ccc1-c1ccc2c(c1)oc1ccccc12] | 75 |
| a6 | 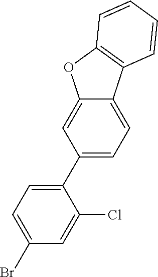 [SMILES: Clc1cc(Br)ccc1-c1ccc2c(c1)oc1ccccc12] | 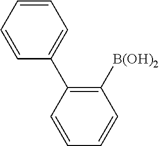 [SMILES: OB(O)c1ccccc1-c1ccccc1] | 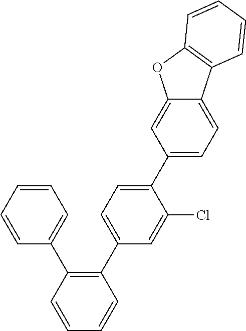 [SMILES: Clc1cc(-c2ccccc2-c2ccccc2)ccc1-c1ccc2c(c1)oc1ccccc12] | 69 |
| b1 | 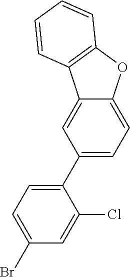 [SMILES: Clc1cc(Br)ccc1-c1ccc2oc3ccccc3c2c1] | 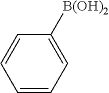 | 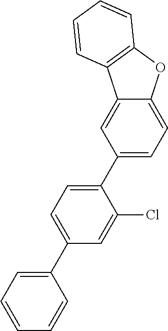 [SMILES: Clc1cc(-c2ccccc2)ccc1-c1ccc2oc3ccccc3c2c1] | 70 |
| b2 | 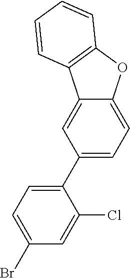 [SMILES: Clc1cc(Br)ccc1-c1ccc2oc3ccccc3c2c1] | 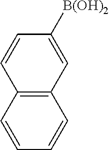 [SMILES: OB(O)c1ccc2ccccc2c1] | 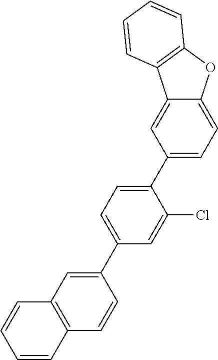 [SMILES: Clc1cc(-c2ccc3ccccc3c2)ccc1-c1ccc2oc3ccccc3c2c1] | 75 |
| b3 | 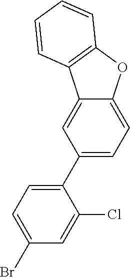 [SMILES: Clc1cc(Br)ccc1-c1ccc2oc3ccccc3c2c1] | 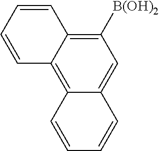 [SMILES: OB(O)c1cc2ccccc2c2ccccc12] | 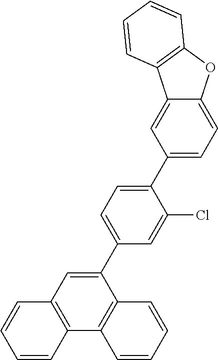 [SMILES: Clc1cc(-c2cc3ccccc3c3ccccc23)ccc1-c1ccc2oc3ccccc3c2c1] | 83 |
| b4 | 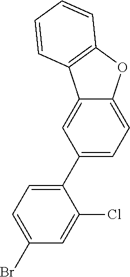 [SMILES: Clc1cc(Br)ccc1-c1ccc2oc3ccccc3c2c1] | 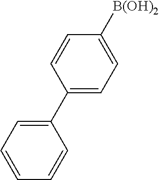 [SMILES: OB(O)c1ccc(-c2ccccc2)cc1] | 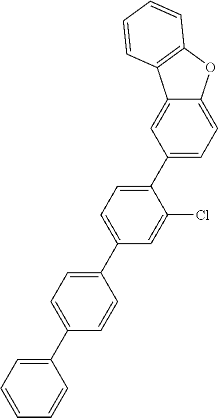 [SMILES: Clc1cc(-c2ccc(-c3ccccc3)cc2)ccc1-c1ccc2oc3ccccc3c2c1] | 77 |
| b5 | 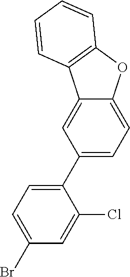 [SMILES: Clc1cc(Br)ccc1-c1ccc2oc3ccccc3c2c1] | 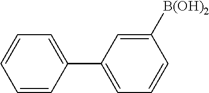 [SMILES: OB(O)c1cccc(-c2ccccc2)c1] | 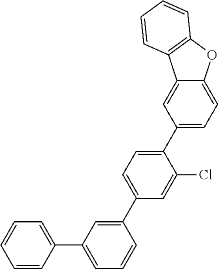 [SMILES: Clc1cc(-c2cccc(-c3ccccc3)c2)ccc1-c1ccc2oc3ccccc3c2c1] | 72 |
| b6 | 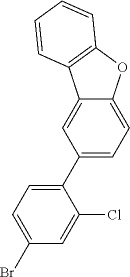 [SMILES: Clc1cc(Br)ccc1-c1ccc2oc3ccccc3c2c1] | 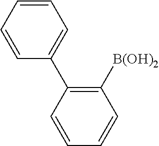 [SMILES: OB(O)c1ccccc1-c1ccccc1] | 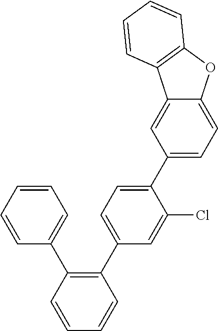 [SMILES: Clc1cc(-c2ccccc2-c2ccccc2)ccc1-c1ccc2oc3ccccc3c2c1] | 70 |
| c1 | 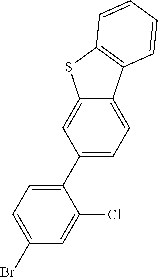 [SMILES: Clc1cc(Br)ccc1-c1ccc2c(c1)sc1ccccc12] | 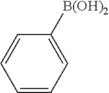 | 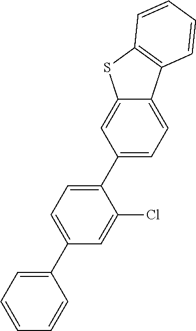 [SMILES: Clc1cc(-c2ccccc2)ccc1-c1ccc2c(c1)sc1ccccc12] | 81 |
| c2 | 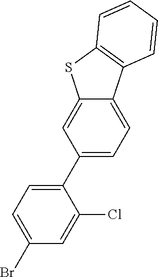 [SMILES: Clc1cc(Br)ccc1-c1ccc2c(c1)sc1ccccc12] | 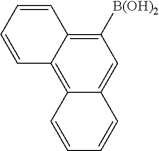 [SMILES: OB(O)c1cc2ccccc2c2ccccc12] | 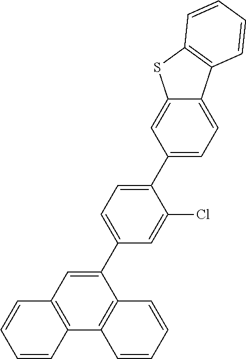 [SMILES: Clc1cc(-c2cc3ccccc3c3ccccc23)ccc1-c1ccc2c(c1)sc1ccccc12] | 83 |
| c3 | 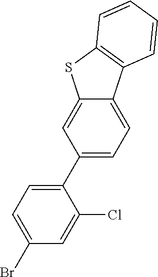 [SMILES: Clc1cc(Br)ccc1-c1ccc2c(c1)sc1ccccc12] | 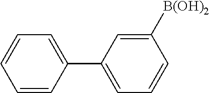 [SMILES: OB(O)c1cccc(-c2ccccc2)c1] | 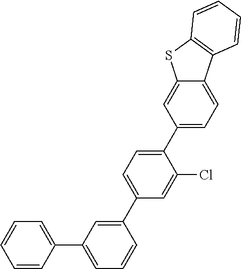 [SMILES: Clc1cc(-c2cccc(-c3ccccc3)c2)ccc1-c1ccc2c(c1)sc1ccccc12] | 76 |
| d1 | 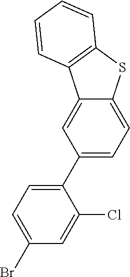 [SMILES: Clc1cc(Br)ccc1-c1ccc2sc3ccccc3c2c1] | 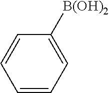 | 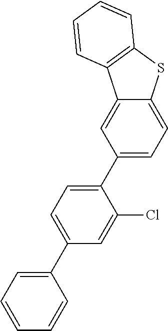 [SMILES: Clc1cc(-c2ccccc2)ccc1-c1ccc2sc3ccccc3c2c1] | 79 |
| d2 | 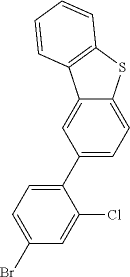 [SMILES: Clc1cc(Br)ccc1-c1ccc2sc3ccccc3c2c1] | 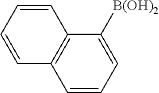 [SMILES: OB(O)c1cccc2ccccc12] | 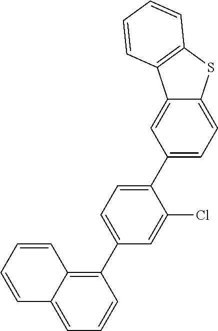 [SMILES: Clc1cc(-c2cccc3ccccc23)ccc1-c1ccc2sc3ccccc3c2c1] | 68 |
| d3 | 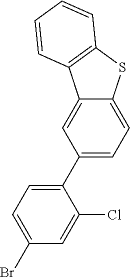 [SMILES: Clc1cc(Br)ccc1-c1ccc2sc3ccccc3c2c1] | 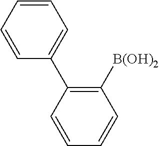 [SMILES: OB(O)c1ccccc1-c1ccccc1] | 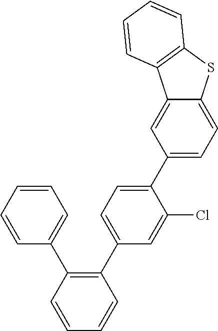 [SMILES: Clc1cc(-c2ccccc2-c2ccccc2)ccc1-c1ccc2sc3ccccc3c2c1] | 67 |
| d4 | 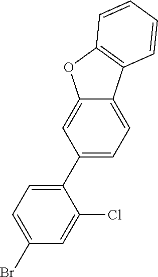 [SMILES: Clc1cc(Br)ccc1-c1ccc2c(c1)oc1ccccc12] | 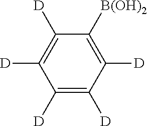 [SMILES: [2H]c1c([2H])c([2H])c(B(O)O)c([2H])c1[2H]] | 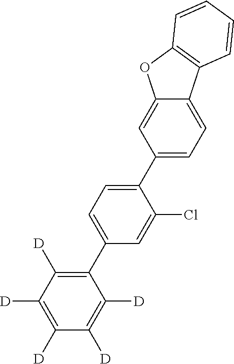 [SMILES: [2H]c1c([2H])c([2H])c(-c2ccc(-c3ccc4c(c3)oc3ccccc34)c(Cl)c2)c([2H])c1[2H]] | 73 |

### Synthesis of Compound A2

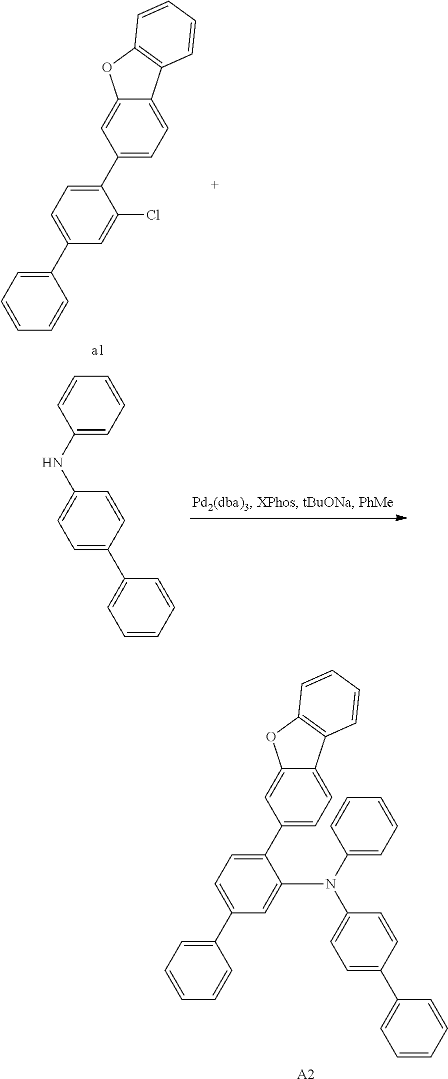 [SMILES: Clc1cc(-c2ccccc2)ccc1-c1ccc2c(c1)oc1ccccc12 ; c1ccc(Nc2ccc(-c3ccccc3)cc2)cc1 ; c1ccc(-c2ccc(N(c3ccccc3)c3cc(-c4ccccc4)ccc3-c3ccc4c(c3)oc3ccccc34)cc2)cc1 ; "A2"]

Under nitrogen protection, Intermediate a1 (4.0 g; 11.3 mmol), N-phenyl-4-benzidine (2.8 g; 11.5 mmol), tris(dibenzylideneacetone)dipalladium (0.1 g; 0.1 mmol), 2-dicyclohexylphosphino-2′,4′,6′-triisopropylbiphenyl (0.1 g; 0.2 mmol), sodium tert-butoxide (1.6 g; 16.9 mmol) and toluene (40 mL) were added into a round bottom flask, and heated to 100° C. to 105° C., and a reaction was carried out under stirring for 12 h; the reaction solution was cooled to room temperature, deionized water was added, liquid separation was performed, and an organic phase was washed with water and dried over anhydrous magnesium sulfate, and a solvent was removed under reduced pressure; and the obtained crude product was purified by silica gel column chromatography using a dichloromethane/n-heptane solvent system, followed by recrystallization using a toluene/n-heptane solvent system to obtain Compound A2 (4.6 g; yield: 72%) as a white solid.

Referring to the synthesis method for the Compound A2, by substituting Reactant D for Intermediate a1 and Reactant E for N-phenyl-4-benzidine, compounds shown in Table 3 below were synthesized:

TABLE 3

| Compound No. | Reactant D | Reactant E | Structure | Yield (%) |
|---|---|---|---|---|
| A10 | 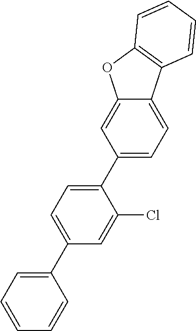 [SMILES: Clc1cc(-c2ccccc2)ccc1-c1ccc2c(c1)oc1ccccc12] | 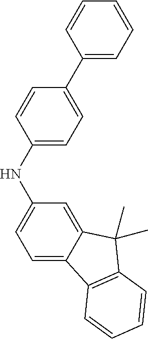 [SMILES: CC1(C)c2ccccc2-c2ccc(Nc3ccc(-c4ccccc4)cc3)cc21] | 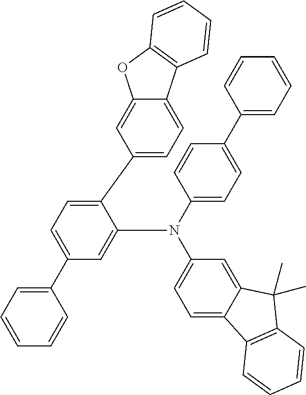 [SMILES: CC1(C)c2ccccc2-c2ccc(N(c3ccc(-c4ccccc4)cc3)c3cc(-c4ccccc4)ccc3-c3ccc4c(c3)oc3ccccc34)cc21] | 75 |
| A23 | 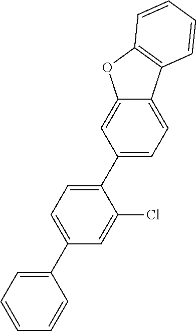 [SMILES: Clc1cc(-c2ccccc2)ccc1-c1ccc2c(c1)oc1ccccc12] | 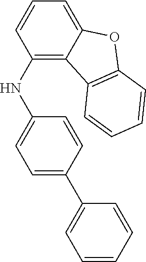 [SMILES: c1ccc(-c2ccc(Nc3cccc4oc5ccccc5c34)cc2)cc1] | 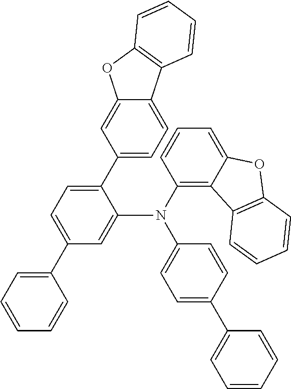 [SMILES: c1ccc(-c2ccc(N(c3cc(-c4ccccc4)ccc3-c3ccc4c(c3)oc3ccccc34)c3cccc4oc5ccccc5c34)cc2)cc1] | 52 |
| A32 | 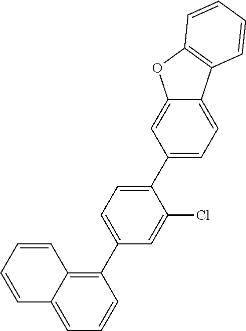 [SMILES: Clc1cc(-c2cccc3ccccc23)ccc1-c1ccc2c(c1)oc1ccccc12] | 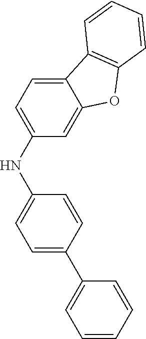 [SMILES: c1ccc(-c2ccc(Nc3ccc4c(c3)oc3ccccc34)cc2)cc1] | 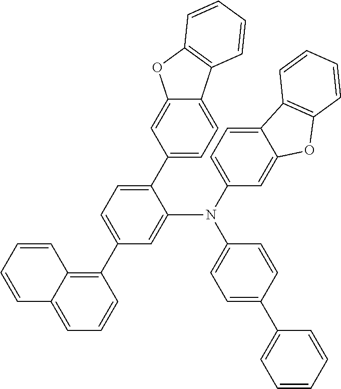 [SMILES: c1ccc(-c2ccc(N(c3ccc4c(c3)oc3ccccc34)c3cc(-c4cccc5ccccc45)ccc3-c3ccc4c(c3)oc3ccccc34)cc2)cc1] | 59 |
| A42 | 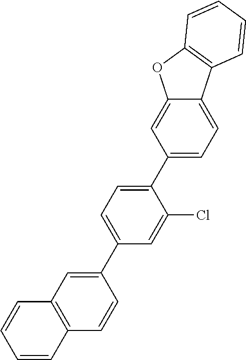 [SMILES: Clc1cc(-c2ccc3ccccc3c2)ccc1-c1ccc2c(c1)oc1ccccc12] | 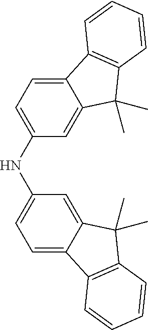 [SMILES: CC1(C)c2ccccc2-c2ccc(Nc3ccc4c(c3)C(C)(C)c3ccccc3-4)cc21] | 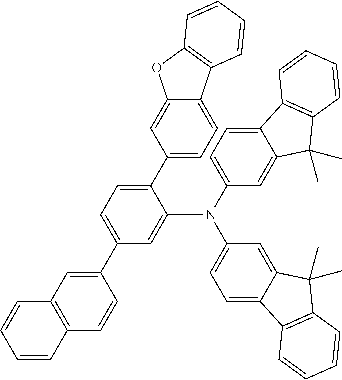 [SMILES: CC1(C)c2ccccc2-c2ccc(N(c3ccc4c(c3)C(C)(C)c3ccccc3-4)c3cc(-c4ccc5ccccc5c4)ccc3-c3ccc4c(c3)oc3ccccc34)cc21] | 64 |
| A56 | 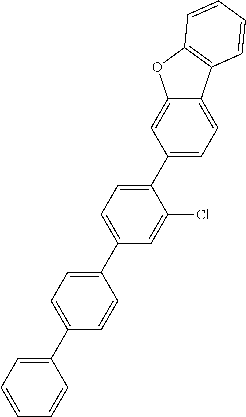 [SMILES: Clc1cc(-c2ccc(-c3ccccc3)cc2)ccc1-c1ccc2c(c1)oc1ccccc12] | 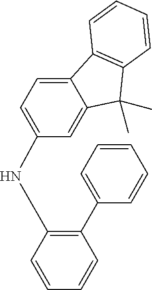 [SMILES: CC1(C)c2ccccc2-c2ccc(Nc3ccccc3-c3ccccc3)cc21] | 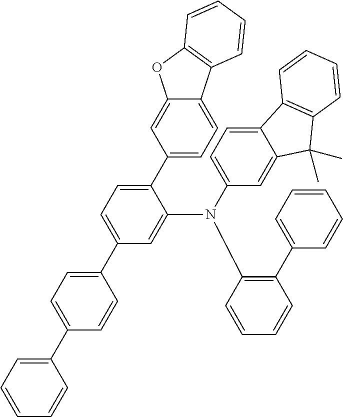 [SMILES: CC1(C)c2ccccc2-c2ccc(N(c3ccccc3-c3ccccc3)c3cc(-c4ccc(-c5ccccc5)cc4)ccc3-c3ccc4c(c3)oc3ccccc34)cc21] | 46 |
| A67 | 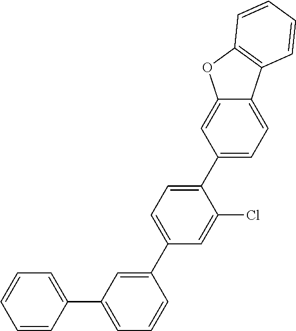 [SMILES: Clc1cc(-c2cccc(-c3ccccc3)c2)ccc1-c1ccc2c(c1)oc1ccccc12] | 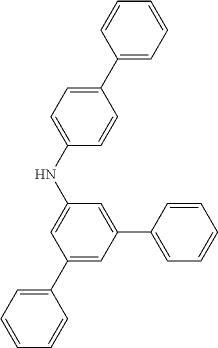 [SMILES: c1ccc(-c2ccc(Nc3cc(-c4ccccc4)cc(-c4ccccc4)c3)cc2)cc1] | 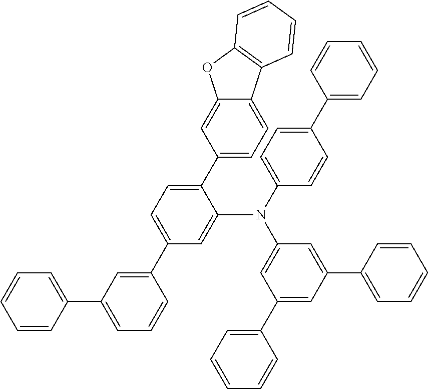 [SMILES: c1ccc(-c2ccc(N(c3cc(-c4ccccc4)cc(-c4ccccc4)c3)c3cc(-c4cccc(-c5ccccc5)c4)ccc3-c3ccc4c(c3)oc3ccccc34)cc2)cc1] | 74 |
| A76 | 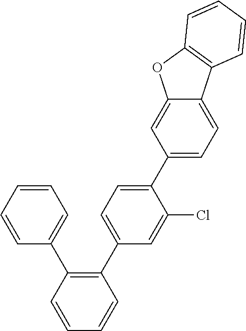 [SMILES: Clc1cc(-c2ccccc2-c2ccccc2)ccc1-c1ccc2c(c1)oc1ccccc12] | 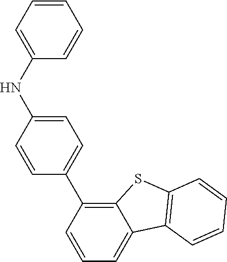 [SMILES: c1ccc(Nc2ccc(-c3cccc4c3sc3ccccc34)cc2)cc1] | 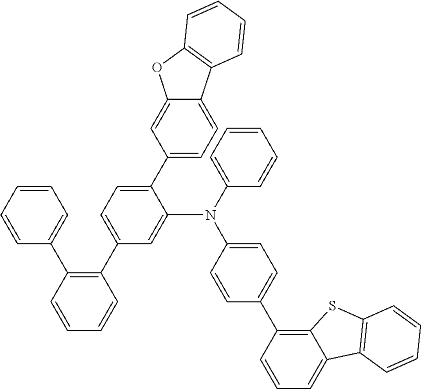 [SMILES: c1ccc(-c2ccccc2-c2ccc(-c3ccc4c(c3)oc3ccccc34)c(N(c3ccccc3)c3ccc(-c4cccc5c4sc4ccccc45)cc3)c2)cc1] | 60 |
| B4 | 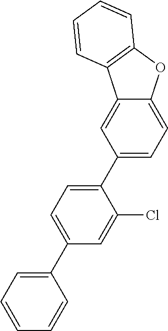 [SMILES: Clc1cc(-c2ccccc2)ccc1-c1ccc2oc3ccccc3c2c1] | 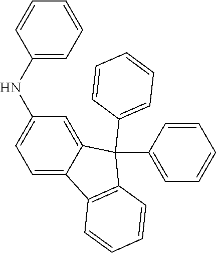 [SMILES: c1ccc(Nc2ccc3c(c2)C(c2ccccc2)(c2ccccc2)c2ccccc2-3)cc1] | 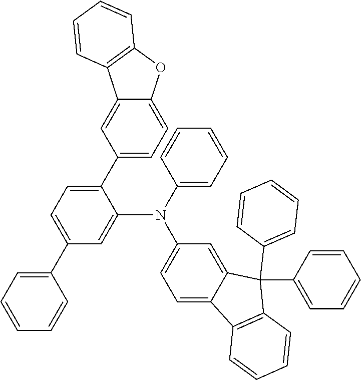 [SMILES: c1ccc(-c2ccc(-c3ccc4oc5ccccc5c4c3)c(N(c3ccccc3)c3ccc4c(c3)C(c3ccccc3)(c3ccccc3)c3ccccc3-4)c2)cc1] | 78 |
| B14 | 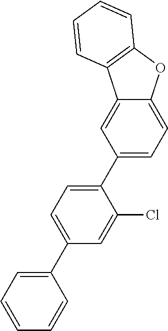 [SMILES: Clc1cc(-c2ccccc2)ccc1-c1ccc2oc3ccccc3c2c1] | 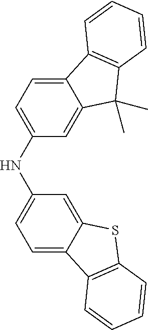 [SMILES: CC1(C)c2ccccc2-c2ccc(Nc3ccc4c(c3)sc3ccccc34)cc21] | 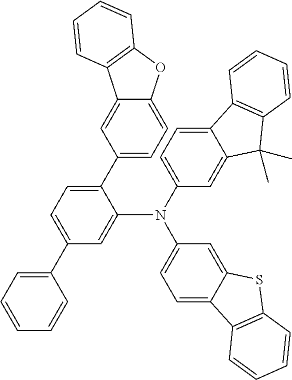 [SMILES: CC1(C)c2ccccc2-c2ccc(N(c3ccc4c(c3)sc3ccccc34)c3cc(-c4ccccc4)ccc3-c3ccc4oc5ccccc5c4c3)cc21] | 79 |
| B23 | 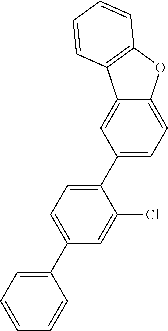 [SMILES: Clc1cc(-c2ccccc2)ccc1-c1ccc2oc3ccccc3c2c1] | 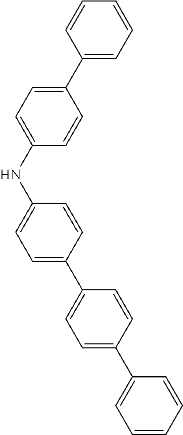 [SMILES: c1ccc(-c2ccc(Nc3ccc(-c4ccc(-c5ccccc5)cc4)cc3)cc2)cc1] | 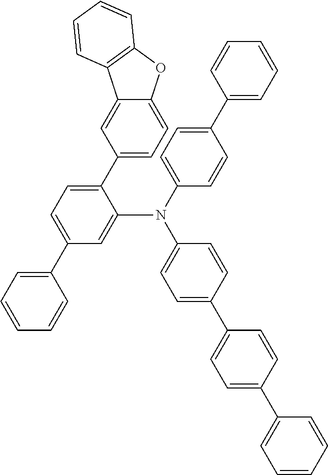 [SMILES: c1ccc(-c2ccc(-c3ccc(N(c4ccc(-c5ccccc5)cc4)c4cc(-c5ccccc5)ccc4-c4ccc5oc6ccccc6c5c4)cc3)cc2)cc1] | 66 |
| B39 | 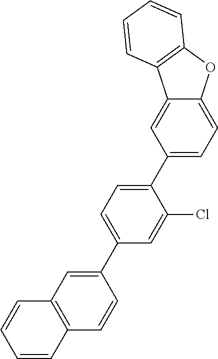 [SMILES: Clc1cc(-c2ccc3ccccc3c2)ccc1-c1ccc2oc3ccccc3c2c1] | 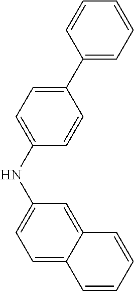 [SMILES: c1ccc(-c2ccc(Nc3ccc4ccccc4c3)cc2)cc1] | 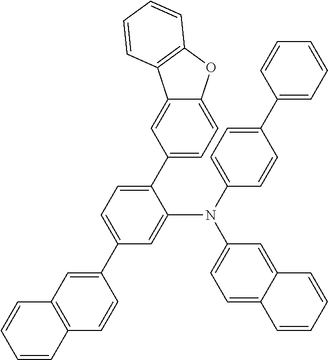 [SMILES: c1ccc(-c2ccc(N(c3ccc4ccccc4c3)c3cc(-c4ccc5ccccc5c4)ccc3-c3ccc4oc5ccccc5c4c3)cc2)cc1] | 56 |
| B46 | 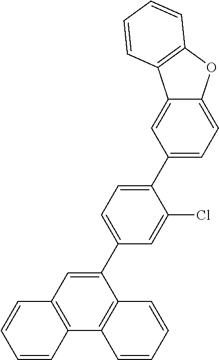 [SMILES: Clc1cc(-c2cc3ccccc3c3ccccc23)ccc1-c1ccc2oc3ccccc3c2c1] | 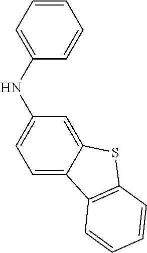 [SMILES: c1ccc(Nc2ccc3c(c2)sc2ccccc23)cc1] | 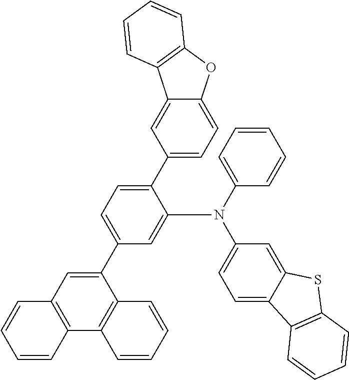 [SMILES: c1ccc(N(c2ccc3c(c2)sc2ccccc23)c2cc(-c3cc4ccccc4c4ccccc34)ccc2-c2ccc3oc4ccccc4c3c2)cc1] | 74 |
| B57 | 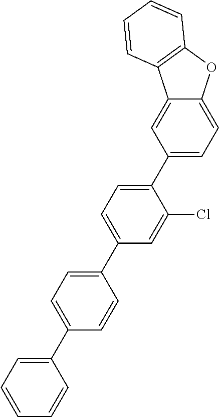 [SMILES: Clc1cc(-c2ccc(-c3ccccc3)cc2)ccc1-c1ccc2oc3ccccc3c2c1] | 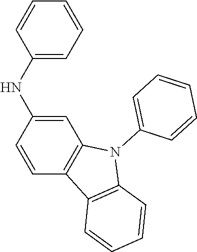 [SMILES: c1ccc(Nc2ccc3c4ccccc4n(-c4ccccc4)c3c2)cc1] | 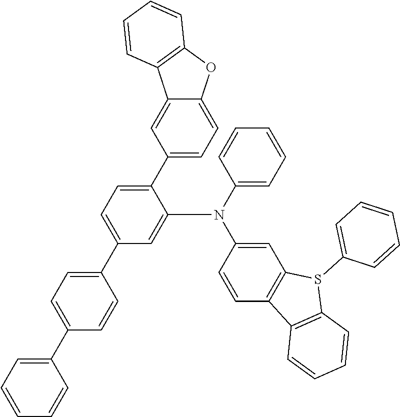 | 50 |
| B61 | 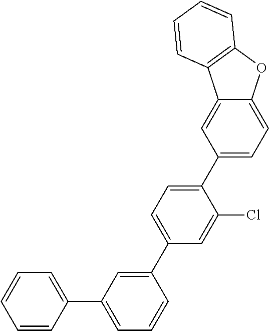 [SMILES: Clc1cc(-c2cccc(-c3ccccc3)c2)ccc1-c1ccc2oc3ccccc3c2c1] | 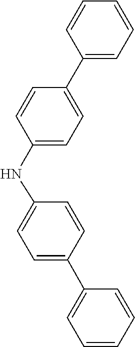 [SMILES: c1ccc(-c2ccc(Nc3ccc(-c4ccccc4)cc3)cc2)cc1] | 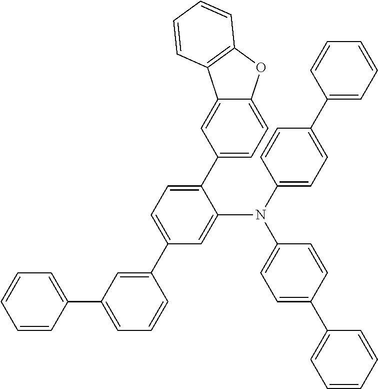 [SMILES: c1ccc(-c2ccc(N(c3ccc(-c4ccccc4)cc3)c3cc(-c4cccc(-c5ccccc5)c4)ccc3-c3ccc4oc5ccccc5c4c3)cc2)cc1] | 63 |
| B71 | 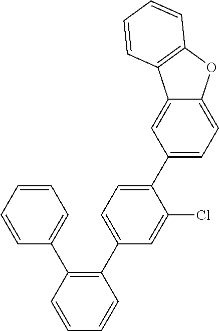 [SMILES: Clc1cc(-c2ccccc2-c2ccccc2)ccc1-c1ccc2oc3ccccc3c2c1] | 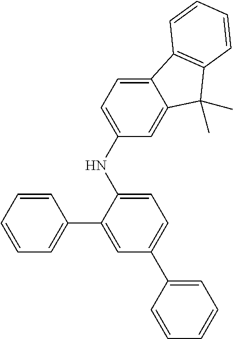 [SMILES: CC1(C)c2ccccc2-c2ccc(Nc3ccc(-c4ccccc4)cc3-c3ccccc3)cc21] | 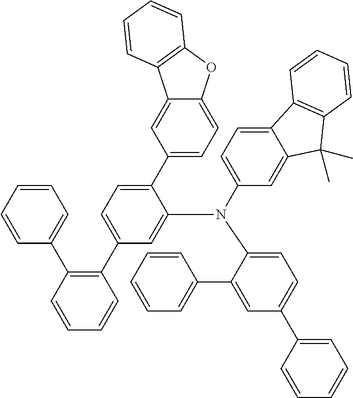 [SMILES: CC1(C)c2ccccc2-c2ccc(N(c3ccc(-c4ccccc4)cc3-c3ccccc3)c3cc(-c4ccccc4-c4ccccc4)ccc3-c3ccc4oc5ccccc5c4c3)cc21] | 45 |
| C2 | 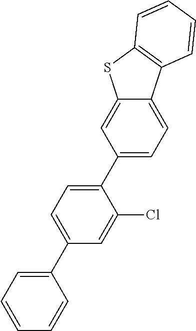 [SMILES: Clc1cc(-c2ccccc2)ccc1-c1ccc2c(c1)sc1ccccc12] | 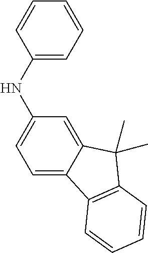 [SMILES: CC1(C)c2ccccc2-c2ccc(Nc3ccccc3)cc21] | 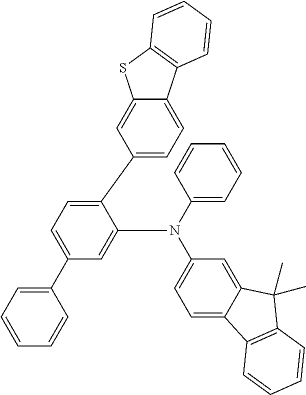 [SMILES: CC1(C)c2ccccc2-c2ccc(N(c3ccccc3)c3cc(-c4ccccc4)ccc3-c3ccc4c(c3)sc3ccccc34)cc21] | 59 |
| C14 | 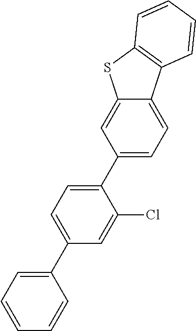 [SMILES: Clc1cc(-c2ccccc2)ccc1-c1ccc2c(c1)sc1ccccc12] | 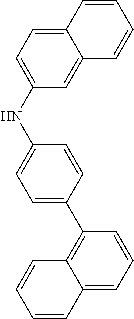 [SMILES: c1ccc2cc(Nc3ccc(-c4cccc5ccccc45)cc3)ccc2c1] | 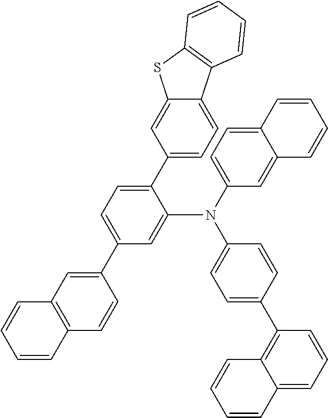 [SMILES: c1ccc2cc(-c3ccc(-c4ccc5c(c4)sc4ccccc45)c(N(c4ccc(-c5cccc6ccccc56)cc4)c4ccc5ccccc5c4)c3)ccc2c1] | 74 |
| C27 | 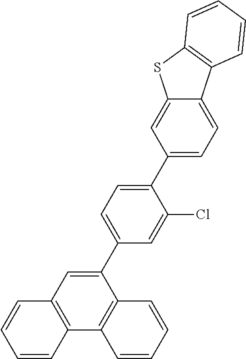 [SMILES: Clc1cc(-c2cc3ccccc3c3ccccc23)ccc1-c1ccc2c(c1)sc1ccccc12] | 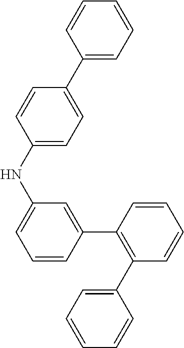 [SMILES: c1ccc(-c2ccc(Nc3cccc(-c4ccccc4-c4ccccc4)c3)cc2)cc1] | 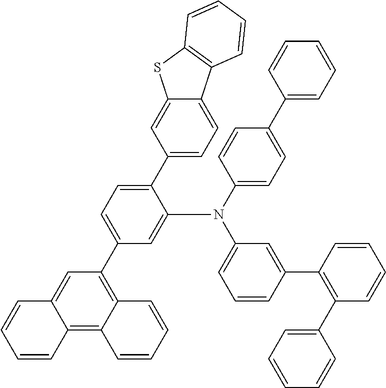 [SMILES: c1ccc(-c2ccc(N(c3cccc(-c4ccccc4-c4ccccc4)c3)c3cc(-c4cc5ccccc5c5ccccc45)ccc3-c3ccc4c(c3)sc3ccccc34)cc2)cc1] | 50 |
| C34 | 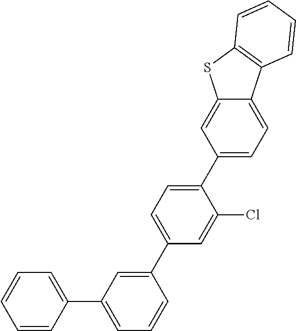 [SMILES: Clc1cc(-c2cccc(-c3ccccc3)c2)ccc1-c1ccc2c(c1)sc1ccccc12] | 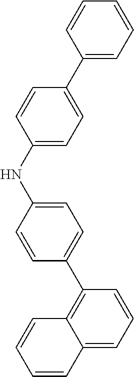 [SMILES: c1ccc(-c2ccc(Nc3ccc(-c4cccc5ccccc45)cc3)cc2)cc1] | 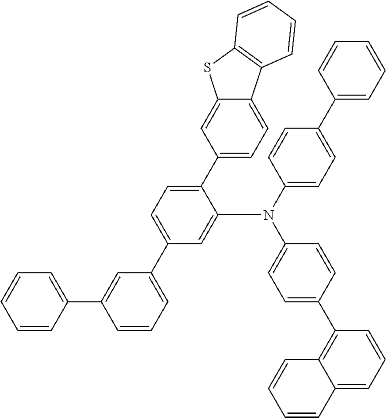 [SMILES: c1ccc(-c2ccc(N(c3ccc(-c4cccc5ccccc45)cc3)c3cc(-c4cccc(-c5ccccc5)c4)ccc3-c3ccc4c(c3)sc3ccccc34)cc2)cc1] | 71 |
| D2 | 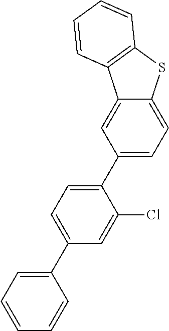 [SMILES: Clc1cc(-c2ccccc2)ccc1-c1ccc2sc3ccccc3c2c1] | 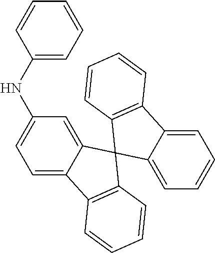 [SMILES: c1ccc(Nc2ccc3c(c2)C2(c4ccccc4-c4ccccc42)c2ccccc2-3)cc1] | 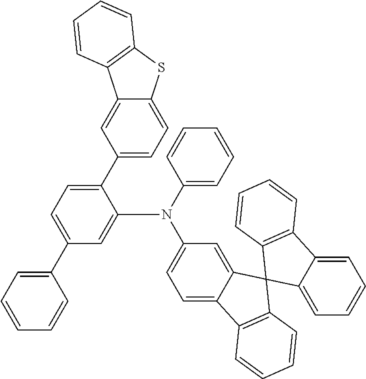 [SMILES: c1ccc(-c2ccc(-c3ccc4sc5ccccc5c4c3)c(N(c3ccccc3)c3ccc4c(c3)C3(c5ccccc5-c5ccccc53)c3ccccc3-4)c2)cc1] | 60 |
| D16 | 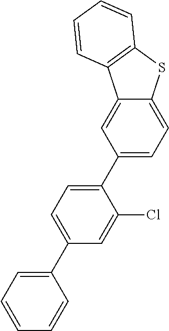 [SMILES: Clc1cc(-c2ccccc2)ccc1-c1ccc2sc3ccccc3c2c1] | 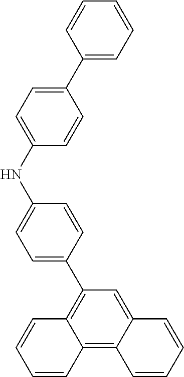 [SMILES: c1ccc(-c2ccc(Nc3ccc(-c4cc5ccccc5c5ccccc45)cc3)cc2)cc1] | 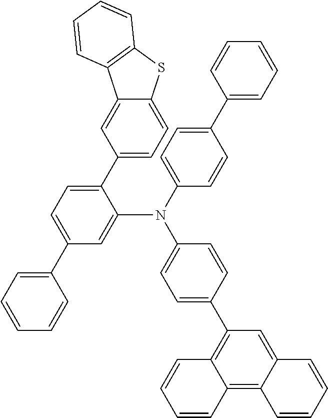 [SMILES: c1ccc(-c2ccc(N(c3ccc(-c4cc5ccccc5c5ccccc45)cc3)c3cc(-c4ccccc4)ccc3-c3ccc4sc5ccccc5c4c3)cc2)cc1] | 75 |
| D20 | 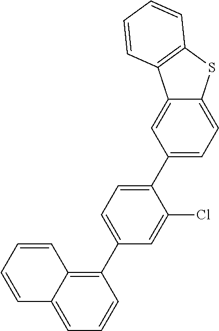 [SMILES: Clc1cc(-c2cccc3ccccc23)ccc1-c1ccc2sc3ccccc3c2c1] | 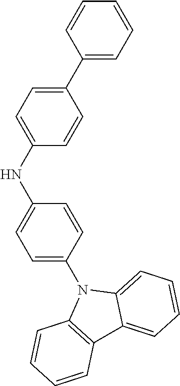 [SMILES: c1ccc(-c2ccc(Nc3ccc(-n4c5ccccc5c5ccccc54)cc3)cc2)cc1] | 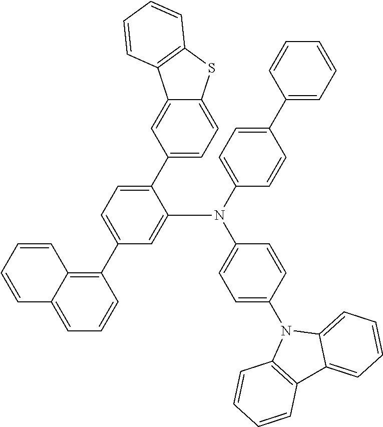 [SMILES: c1ccc(-c2ccc(N(c3ccc(-n4c5ccccc5c5ccccc54)cc3)c3cc(-c4cccc5ccccc45)ccc3-c3ccc4sc5ccccc5c4c3)cc2)cc1] | 73 |
| D38 | 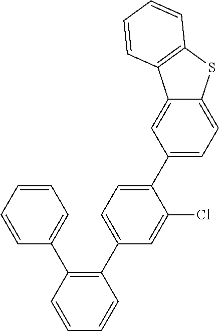 [SMILES: Clc1cc(-c2ccccc2-c2ccccc2)ccc1-c1ccc2sc3ccccc3c2c1] | 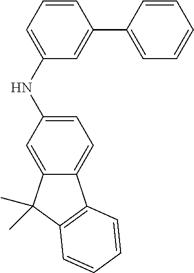 [SMILES: CC1(C)c2ccccc2-c2ccc(Nc3cccc(-c4ccccc4)c3)cc21] | 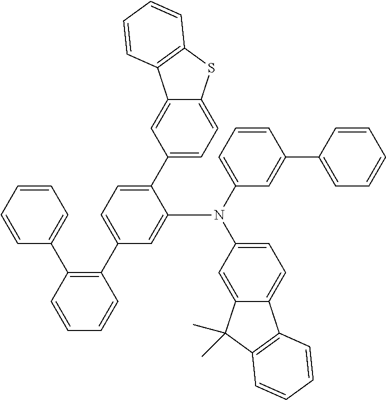 [SMILES: CC1(C)c2ccccc2-c2ccc(N(c3cccc(-c4ccccc4)c3)c3cc(-c4ccccc4-c4ccccc4)ccc3-c3ccc4sc5ccccc5c4c3)cc21] | 66 |
| A77 | 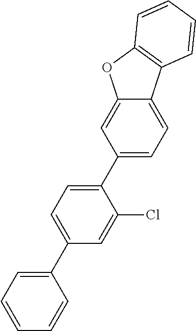 [SMILES: Clc1cc(-c2ccccc2)ccc1-c1ccc2c(c1)oc1ccccc12] | 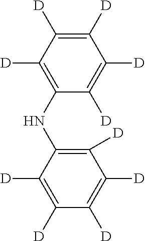 [SMILES: [2H]c1c([2H])c([2H])c(Nc2c([2H])c([2H])c([2H])c([2H])c2[2H])c([2H])c1[2H]] | 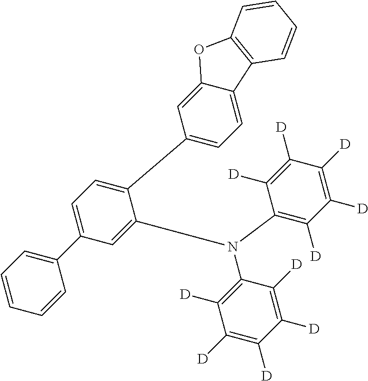 [SMILES: [2H]c1c([2H])c([2H])c(N(c2cc(-c3ccccc3)ccc2-c2ccc3c(c2)oc2ccccc23)c2c([2H])c([2H])c([2H])c([2H])c2[2H])c([2H])c1[2H]] | 54 |
| A78 | 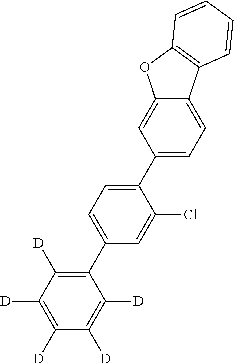 [SMILES: [2H]c1c([2H])c([2H])c(-c2ccc(-c3ccc4c(c3)oc3ccccc34)c(Cl)c2)c([2H])c1[2H]] | 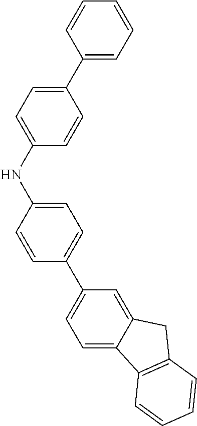 [SMILES: c1ccc(-c2ccc(Nc3ccc(-c4ccc5c(c4)Cc4ccccc4-5)cc3)cc2)cc1] | 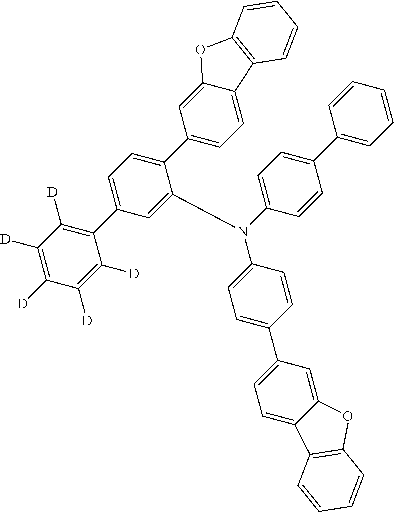 [SMILES: [2H]c1c([2H])c([2H])c(-c2ccc(-c3ccc4c(c3)oc3ccccc34)c(N(c3ccc(-c4ccccc4)cc3)c3ccc(-c4ccc5c(c4)oc4ccccc45)cc3)c2)c([2H])c1[2H]] | 49 |
| A79 | 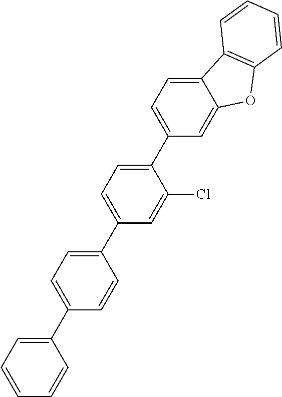 [SMILES: Clc1cc(-c2ccc(-c3ccccc3)cc2)ccc1-c1ccc2c(c1)oc1ccccc12] | 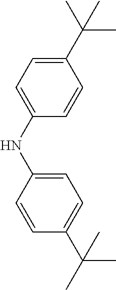 [SMILES: CC(C)(C)c1ccc(Nc2ccc(C(C)(C)C)cc2)cc1] | 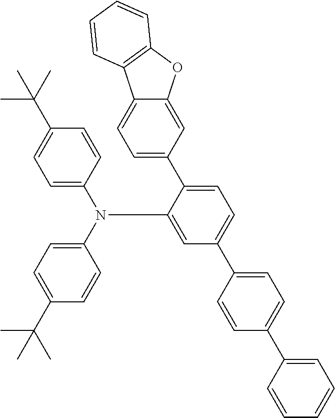 [SMILES: CC(C)(C)c1ccc(N(c2ccc(C(C)(C)C)cc2)c2cc(-c3ccc(-c4ccccc4)cc3)ccc2-c2ccc3c(c2)oc2ccccc23)cc1] | 69 |
| B6 | 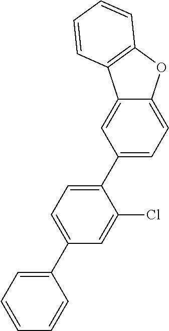 [SMILES: Clc1cc(-c2ccccc2)ccc1-c1ccc2oc3ccccc3c2c1] | 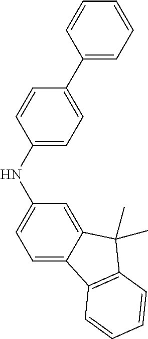 [SMILES: CC1(C)c2ccccc2-c2ccc(Nc3ccc(-c4ccccc4)cc3)cc21] | 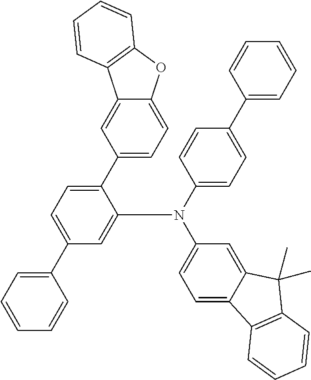 [SMILES: CC1(C)c2ccccc2-c2ccc(N(c3ccc(-c4ccccc4)cc3)c3cc(-c4ccccc4)ccc3-c3ccc4oc5ccccc5c4c3)cc21] | 52 |

Mass spectrum data of some of the compounds are shown in Table 4 below.

TABLE 4

| Compound | Mass spectrum data | Compound | Mass spectrum data |
|---|---|---|---|
| Compound A2 | m/z = 564.2 (M + H)⁺ | Compound B46 | m/z = 694.2 (M + H)⁺ |
| Compound A10 | m/z = 680.3 (M + H)⁺ | Compound B57 | m/z = 729.3 (M + H)⁺ |
| Compound A23 | m/z = 654.2 (M + H)⁺ | Compound B61 | m/z = 716.3 (M + H)⁺ |
| Compound A32 | m/z = 704.3 (M + H)⁺ | Compound B71 | m/z = 832.4 (M + H)⁺ |
| Compound A42 | m/z = 770.3 (M + H)⁺ | Compound C2 | m/z = 620.2 (M + H)⁺ |
| Compound A56 | m/z = 756.3 (M + H)⁺ | Compound C14 | m/z = 680.2 (M + H)⁺ |
| Compound A67 | m/z = 792.3 (M + H)⁺ | Compound C27 | m/z = 832.3 (M + H)⁺ |
| Compound A76 | m/z = 746.3 (M + H)⁺ | Compound C34 | m/z = 782.3 (M + H)⁺ |
| Compound B4 | m/z = 728.3 (M + H)⁺ | Compound D2 | m/z = 742.3 (M + H)⁺ |
| Compound B14 | m/z = 710.3 (M + H)⁺ | Compound D16 | m/z = 756.3 (M + H)⁺ |
| Compound B23 | m/z = 716.3 (M + H)⁺ | Compound D20 | m/z = 795.3 (M + H)⁺ |
| Compound B39 | m/z = 664.3 (M + H)⁺ | Compound D38 | m/z = 772.3 (M + H)⁺ |
| Compound A77 | m/z = 498.3 (M + H)⁺ | Compound A78 | m/z = 735.3 (M + H)⁺ |
| Compound A79 | m/z = 676.4 (M + H)⁺ | Compound B6 | m/z = 680.3 (M + H)⁺ |

NMR data of some of the compounds are shown in Table 5 below.

TABLE 5

| Compound | NMR data |
|---|---|
| Compound | ¹HNMR (400 MHz, CD₂Cl₂): 7.82 (s, 1H), 7.75 (d, 1H), |
| B6 | 7.71 (s, 1H), 7.67-7.62 (m, 3H), 7.56 (d, 1H), 7.51-6.15 |
| | (m, 19H), 7.12 (d, 2H), 6.93 (s, 1H), 6.86 (d, 2H), 1.12 |
| | (s, 6H). |

### Manufacture of Organic Electroluminescent Devices

### Example 1: Red Organic Electroluminescent Device

An anode was prepared the following process: an ITO/Ag/ITO glass substrate (manufactured by Corning) with a thickness of 110 Å/900 Å/90 Å was cut into a size of 40 mm×40 mm×0.7 mm, and prepared into an experimental substrate with cathode, anode, and insulation layer patterns by using a photoetching process, and surface treatment was performed with UV ozone and O₂:N₂ plasma to increase the work function of the anode (the experimental substrate) and remove scum.

F4-TCNQ and HT-18 was co-evaporated on the experimental substrate (the anode) at an evaporation rate ratio of 2%:98% to form a hole injection layer (HIL) with a thickness of 100 Å, and HT-18 was evaporated on the hole injection layer to form a hole transport layer (HTL) with a thickness of 1220 Å.

Compound A2 was vacuum-evaporated on the hole transport layer to form a hole auxiliary layer of 850 Å.

RH-01 and RD were co-evaporated on the hole auxiliary layer at a film thickness ratio of 96%:4% to form an organic light-emitting layer (R-EML) of 310 Å.

ET-01 and LiQ were co-evaporated on the organic light-emitting layer at a ratio of 1:1 to form an electron transport layer (ETL) of 330 Å, Yb was evaporated on the electron transport layer to form an electron injection layer (EIL) having a thickness of 12 Å, and then magnesium (Mg) and silver (Ag) were mixed and vacuum-evaporated on the electron injection layer at an evaporation rate of 1:9 to form a cathode having a thickness of 130 Å.

Finally, HT-19 was evaporated on the cathode to form an organic capping layer (CPL) with a thickness of 720 Å, thus completing manufacture of an organic light-emitting device.

### Examples 2 to 28

An organic electroluminescent device was manufactured by the same method as that in Example 1, except that compounds shown in Table 6 below were substituted for the compound A2 when the hole auxiliary layer was formed.

### Comparative Examples 1 to 4

An organic electroluminescent device was manufactured by the same method as that in Example 1, except that Compound A, Compound B, Compound C, and Compound D in Table 6 below were respectively substituted for the Compound A2 when the hole auxiliary layer was formed.

Structures of other materials used in above Examples and Comparative examples are shown below,

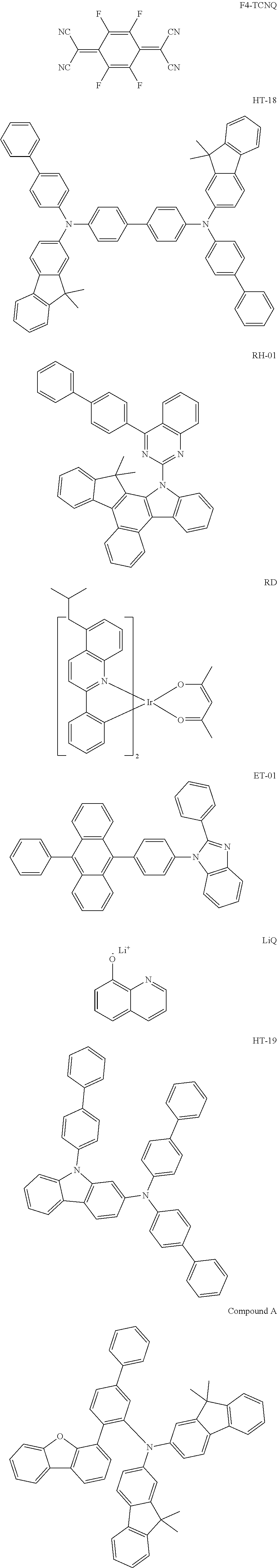

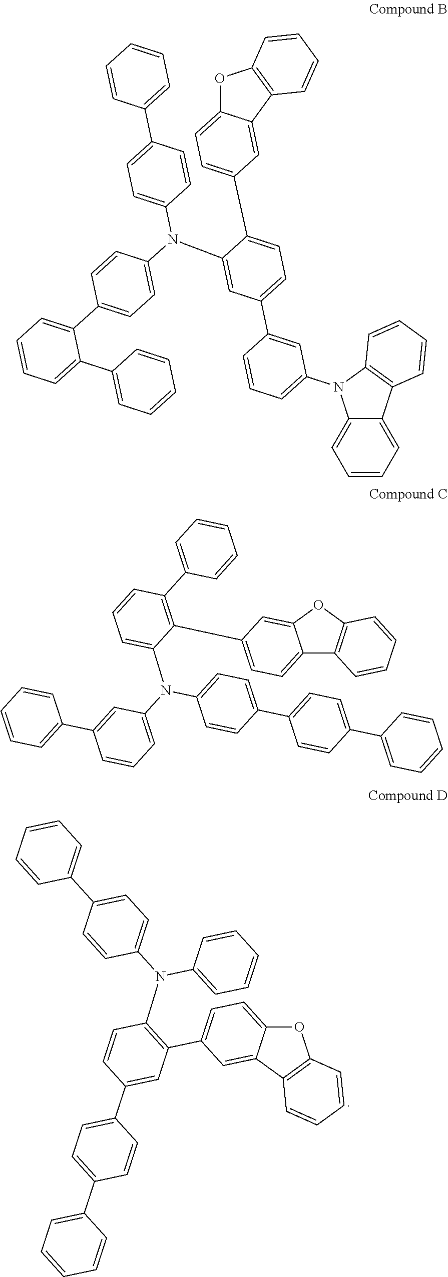 [SMILES: c1ccc(-c2ccc(N(c3ccc(-c4ccccc4-c4ccccc4)cc3)c3cc(-c4cccc(-n5c6ccccc6c6ccccc65)c4)ccc3-c3ccc4oc5ccccc5c4c3)cc2)cc1 ; c1ccc(-c2ccc(-c3ccc(N(c4cccc(-c5ccccc5)c4)c4cccc(-c5ccccc5)c4-c4ccc5c(c4)oc4ccccc45)cc3)cc2)cc1 ; c1ccc(-c2ccc(-c3ccc(N(c4ccccc4)c4ccc(-c5ccccc5)cc4)c(-c4ccc5oc6ccccc6c5c4)c3)cc2)cc1]

The IVL (current, voltage, efficiency, etc.) of the devices in Examples 1 to 28 and Comparative examples 1 to 4 was tested at a current density of 10 mA/cm² and the T95 service life of the devices in Examples 1 to 28 and Comparative examples 1 to 4 was tested at a current density of 20 mA/cm², and the test results are shown in Table 6 below.

TABLE 6
Device performance test results

| | | | | | | T95 |
|---|---|---|---|---|---|---|
| | Hole | Operating | Current | Chromaticity | Chromaticity | service |
| Example | auxiliary | voltage | efficiency | coordinate | coordinate | life |
| No. | layer | Volt (V) | Cd/A | CIEx | CIEy | (hrs) |
| Example 1 | Compound | 3.48 | 62.80 | 0.68 | 0.32 | 503 |
| | A2 | | | | | |
| Example 2 | Compound | 3.47 | 61.90 | 0.68 | 0.32 | 503 |
| | A10 | | | | | |
| Example 3 | Compound | 3.42 | 62.49 | 0.68 | 0.32 | 506 |
| | A23 | | | | | |
| Example 4 | Compound | 3.43 | 61.80 | 0.68 | 0.32 | 502 |
| | A42 | | | | | |
| Example 5 | Compound | 3.45 | 62.50 | 0.68 | 0.32 | 501 |
| | A56 | | | | | |
| Example 6 | Compound | 3.45 | 61.89 | 0.68 | 0.32 | 499 |
| | A67 | | | | | |
| Example 7 | Compound | 3.42 | 62.84 | 0.68 | 0.32 | 500 |
| | A32 | | | | | |
| Example 8 | Compound | 3.43 | 62.40 | 0.68 | 0.32 | 499 |
| | A76 | | | | | |
| Example 9 | Compound | 3.43 | 61.77 | 0.68 | 0.32 | 506 |
| | B4 | | | | | |
| Example 10 | Compound | 3.43 | 61.75 | 0.68 | 0.32 | 510 |
| | B14 | | | | | |
| Example 11 | Compound | 3.45 | 61.79 | 0.68 | 0.32 | 510 |
| | B23 | | | | | |
| Example 12 | Compound | 3.44 | 62.37 | 0.68 | 0.32 | 507 |
| | B39 | | | | | |
| Example 13 | Compound | 3.43 | 62.20 | 0.68 | 0.32 | 500 |
| | A77 | | | | | |
| Example 14 | Compound | 3.46 | 62.18 | 0.68 | 0.32 | 515 |
| | A78 | | | | | |
| Example 15 | Compound | 3.43 | 62.66 | 0.68 | 0.32 | 510 |
| | A79 | | | | | |
| Example 16 | Compound | 3.47 | 62.52 | 0.68 | 0.32 | 502 |
| | B46 | | | | | |
| Example 17 | Compound | 3.46 | 61.70 | 0.68 | 0.32 | 503 |
| | B57 | | | | | |
| Example 18 | Compound | 3.48 | 61.86 | 0.68 | 0.32 | 506 |
| | B61 | | | | | |
| Example 19 | Compound | 3.43 | 62.70 | 0.68 | 0.32 | 508 |
| | B71 | | | | | |
| Example 20 | Compound | 3.48 | 62.60 | 0.68 | 0.32 | 478 |
| | C2 | | | | | |
| Example 21 | Compound | 3.43 | 62.10 | 0.68 | 0.32 | 480 |
| | C14 | | | | | |
| Example 22 | Compound | 3.47 | 61.82 | 0.68 | 0.32 | 482 |
| | C27 | | | | | |
| Example 23 | Compound | 3.47 | 62.01 | 0.68 | 0.32 | 479 |
| | C34 | | | | | |
| Example 24 | Compound | 3.42 | 63.03 | 0.68 | 0.32 | 479 |
| | D2 | | | | | |
| Example 25 | Compound | 3.45 | 62.63 | 0.68 | 0.32 | 483 |
| | D16 | | | | | |
| Example 26 | Compound | 3.44 | 62.00 | 0.68 | 0.32 | 481 |
| | D20 | | | | | |
| Example 27 | Compound | 3.42 | 61.93 | 0.68 | 0.32 | 479 |
| | D38 | | | | | |
| Example 28 | Compound | 3.43 | 63.00 | 0.68 | 0.32 | 506 |
| | B6 | | | | | |
| Comparative | Compound | 3.70 | 55.40 | 0.68 | 0.32 | 410 |
| example 1 | A | | | | | |
| Comparative | Compound | 3.71 | 53.37 | 0.68 | 0.32 | 397 |
| example 2 | B | | | | | |
| Comparative | Compound | 3.67 | 54.31 | 0.68 | 0.32 | 403 |
| example 3 | C | | | | | |
| Comparative | Compound | 3.49 | 51.10 | 0.68 | 0.32 | 415 |
| example 4 | D | | | | | |

As can been seen from the results of Table 6 above, the organic electroluminescent devices in Examples 1 to 28 have improved performance compared with the organic electroluminescent devices in Comparative examples 1 to 4. Specifically, the driving voltages of the organic electroluminescent devices in Examples 1 to 28 are close to those of Comparative examples, the current efficiency of the organic electroluminescent devices in Examples 1 to 28 is improved by at least 11.4%, and the service life of the organic electroluminescent devices in Examples 1 to 28 is improved by at least 15.2%. Thus, when the organic compound of the present application is used as a hole auxiliary layer of an organic electroluminescent device, the efficiency and service life are improved while maintaining a low operating voltage.

The devices in the Examples of the present application have significantly lower driving voltages, as well as improved current efficiency and service life compared with Comparative examples 1 to 3.

Compared with the compound A, when the compounds of the present application are used, the current efficiency and service life are significantly improved, and the reason may be that in the compounds of the present application, the dibenzofuran/dibenzothiophene group is linked to a benzene ring of aniline via a specific position, which maintains a high coverage of an HOMO orbit of the molecule, giving the compound molecule a moderate distortion, thus enhancing the steric aromatic conjugation effect and the hole mobility of the compounds.

Compared with the compound B, when the compounds of the present application are used, the current efficiency and service life are significantly improved, and the reason may be that the compounds of the present application have less bulky aryl linked to a 5-position of phenyl of aniline, maintaining a high coverage of the HOMO orbit of the molecule.

Compared with the compound C, an aryl substituent is linked at a para-position instead of an ortho-position of the dibenzofuran/dibenzothiophene group in aniline in the compounds of the present application, so that the dibenzofuran/dibenzothiophene group can better maintain a high coincidence of intermolecular orbitals through steric conjugation effects.

The devices in the Examples of the present application have significantly improved luminous efficiency and service life characteristics while the driving voltages are close compared with Comparative example 4. The reason may be that the aryl substituent is linked to the 5-position of aniline in the compounds of the present application compared with compound D, thus maintaining a deeper HOMO energy level and a high T1 energy level.

1. An organic compound, having a structure as shown in a formula 1: 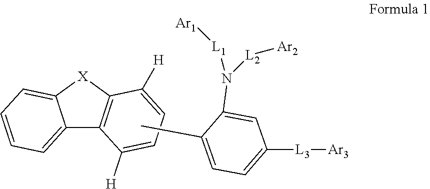
wherein X is O or S;
L₁ and L₂ are the same or different, and are respectively and independently selected from a single bond, a substituted or unsubstituted arylene with 6 to 30 carbon atoms, or a substituted or unsubstituted heteroarylene with 3 to 30 carbon atoms;
Ar₁ and Ar₂ are the same or different, and are respectively and independently selected from a substituted or unsubstituted aryl with 6 to 40 carbon atoms, or a substituted or unsubstituted heteroaryl with 3 to 40 carbon atoms;
L₃ is selected from a single bond, or a substituted or unsubstituted arylene with 6 to 30 carbon atoms;
Ar₃ is selected from a substituted or unsubstituted aryl with 6 to 30 carbon atoms;
substituent(s) in L₃ and Ar₃ are the same or different, and are respectively and independently selected from deuterium, a halogen group, a cyano, an alkyl with 1 to 10 carbon atoms, a cycloalkyl with 3 to 20 carbon atoms, an aryl with 6 to 20 carbon atoms, or a deuterated aryl with 6 to 20 carbon atoms;
substituent(s) in L₁, L₂, Ar₁ and Ar₂ are the same or different, and are respectively and independently selected from deuterium, a halogen group, a cyano, an alkyl with 1 to 10 carbon atoms, a cycloalkyl with 3 to 20 carbon atoms, a heteroaryl with 12 to 20 carbon atoms, an aryl with 6 to 20 carbon atoms, a deuterated aryl with 6 to 20 carbon atoms, a haloaryl with 6 to 20 carbon atoms, a trialkylsilyl with 3 to 12 carbon atoms, a triarylsilyl with 18 to 24 carbon atoms, a haloalkyl with 1 to 10 carbon atoms, or a deuterated alkyl with 1 to 10 carbon atoms; and
optionally, in Ar₁ and Ar₂, any two adjacent substituents form a ring.

2. The organic compound according to claim 1, wherein L₁ and L₂ are the same or different, and are respectively and independently selected from a single bond, a substituted or unsubstituted arylene with 6 to 15 carbon atoms, or a substituted or unsubstituted heteroarylene with 12 to 20 carbon atoms; and
optionally, substituent(s) in L₁ and L₂ are the same or different, and are respectively and independently selected from deuterium, a halogen group, a cyano, an alkyl with 1 to 5 carbon atoms or a phenyl.

3. The organic compound according to claim 1, wherein L₁ and L₂ are the same or different, and are respectively and independently selected from a single bond, a substituted or unsubstituted phenylene, a substituted or unsubstituted naphthylene, a substituted or unsubstituted biphenylene, a substituted or unsubstituted carbazolylene, a substituted or unsubstituted dibenzofurylene, or a substituted or unsubstituted dibenzothenylene; and
optionally, substituent(s) in L₁ and L₂ are the same or different, and are respectively and independently selected from deuterium, fluorine, cyano, methyl, ethyl, n-propyl, isopropyl, tert-butyl or phenyl.

4. The organic compound according to claim 1, wherein Ar₁ and Ar₂ are the same or different, and are respectively and independently selected from a substituted or unsubstituted aryl with 6 to 25 carbon atoms, or a substituted or unsubstituted heteroaryl with 12 to 20 carbon atoms;
optionally, substituent(s) in Ar₁ and Ar₂ are the same or different, and are respectively and independently selected from deuterium, a halogen group, a cyano, an alkyl with 1 to 5 carbon atoms, a phenyl, or a pentadeuterophenyl; and
optionally, in Ar₁ and Ar₂, any two adjacent substituents form a fluorene ring.

5. The organic compound according to claim 1, wherein Ar₁ and Ar₂ are the same or different, and are respectively and independently selected from a substituted or unsubstituted terphenyl, a substituted or unsubstituted phenyl, a substituted or unsubstituted naphthyl, a substituted or unsubstituted biphenyl, a substituted or unsubstituted phenanthryl, a substituted or unsubstituted fluorenyl, a substituted or unsubstituted triphenylene, a substituted or unsubstituted carbazolyl, a substituted or unsubstituted dibenzofuranyl, a substituted or unsubstituted dibenzothienyl, or a substituted or unsubstituted spirobifluorenyl; and
optionally, substituent(s) in Ar₁ and Ar₂ are the same or different, and are respectively and independently selected from deuterium, fluorine, cyano, methyl, ethyl, n-propyl, isopropyl, tert-butyl, phenyl or pentadeuterophenyl.

6. The organic compound according to claim 1, wherein L₃ is selected from a single bond, a substituted or unsubstituted phenylene, or a substituted or unsubstituted naphthylene; and
optionally, substituent(s) in L₃ are the same or different, and are respectively and independently selected from deuterium, fluorine, cyano, methyl, ethyl, n-propyl, isopropyl, tert-butyl or phenyl.

7. The organic compound according to claim 1, wherein Ar₃ is selected from a substituted or unsubstituted phenyl, a substituted or unsubstituted naphthyl, a substituted or unsubstituted biphenyl, or a substituted or unsubstituted phenanthryl; and
optionally, substituent(s) in Ar₃ are the same or different, and are respectively and independently selected from deuterium, fluorine, cyano, methyl, ethyl, n-propyl, isopropyl, tert-butyl, phenyl or pentadeuterophenyl.

8. The organic compound according to claim 1, wherein 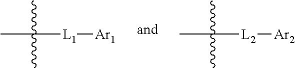
are the same or different, and are respectively and independently selected from the group consisting of:

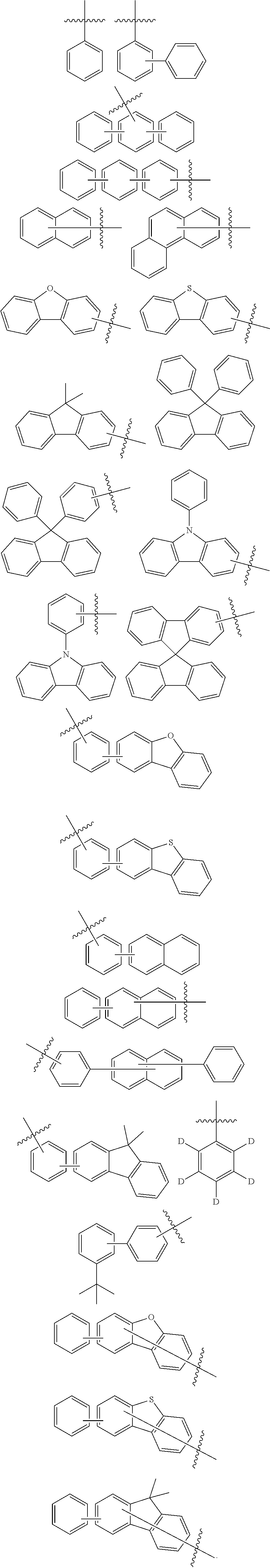

9. The organic compound according to claim 1, wherein 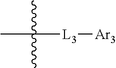
is selected from the group consisting of:

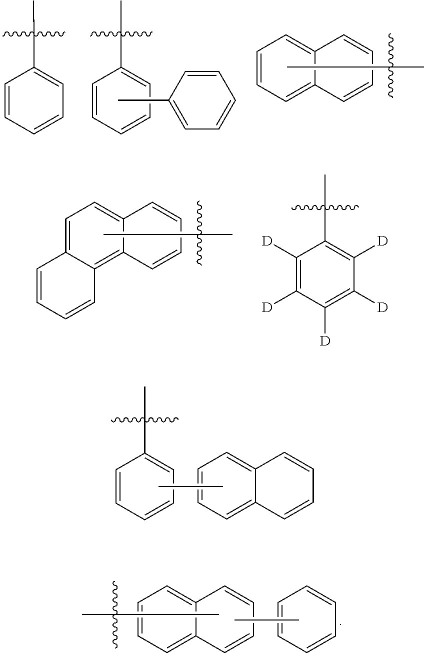

10. The organic compound according to claim 1, wherein 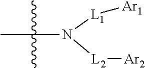
in the formula 1 is selected from the group consisting of:

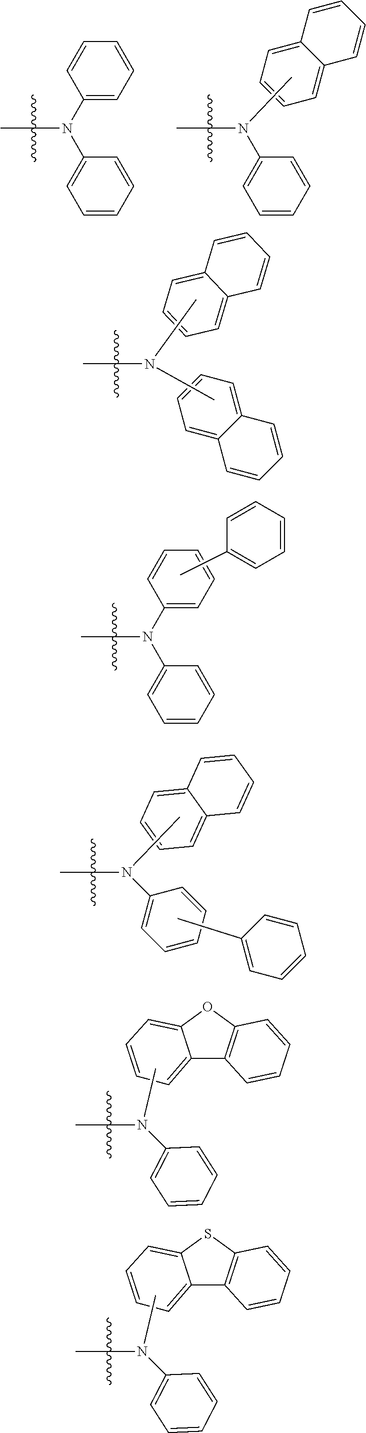

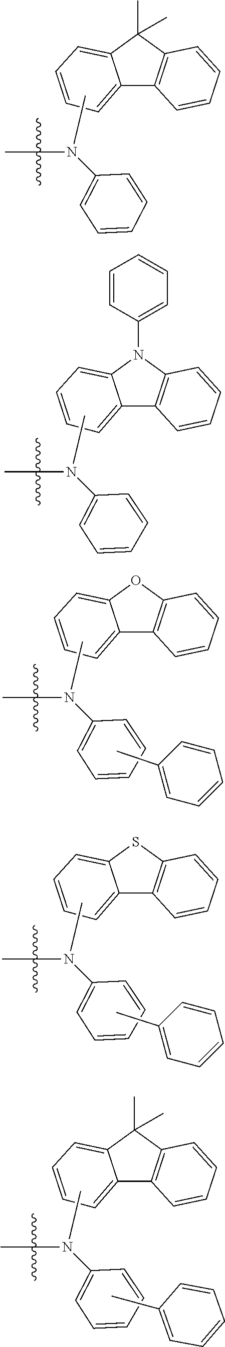

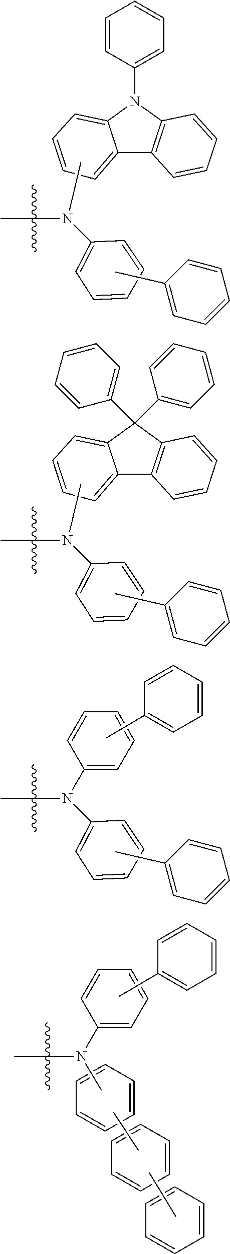

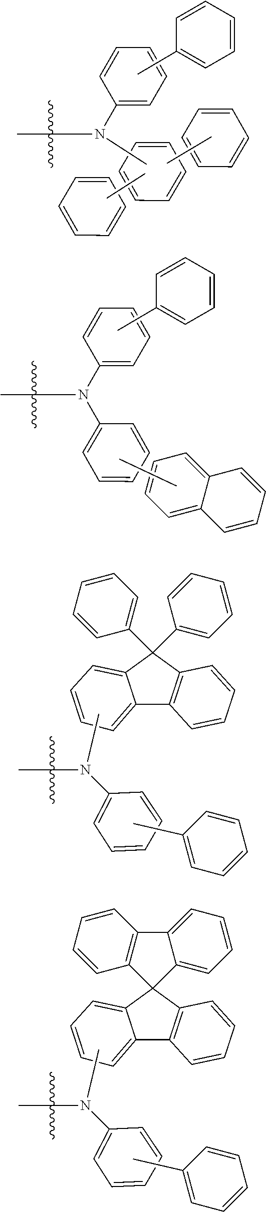

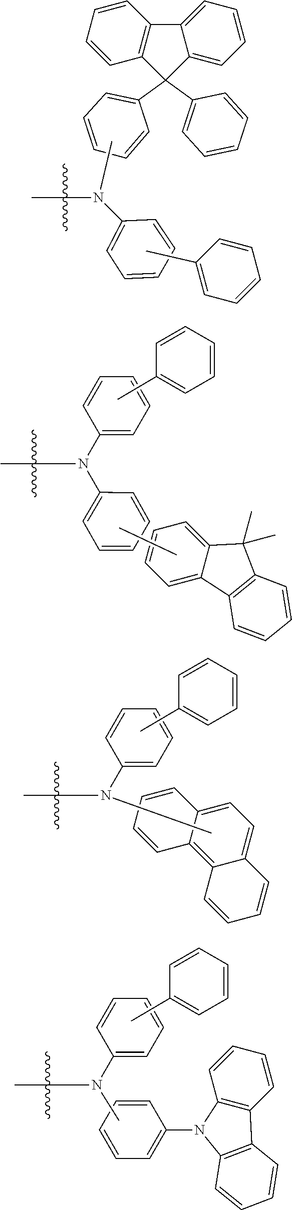

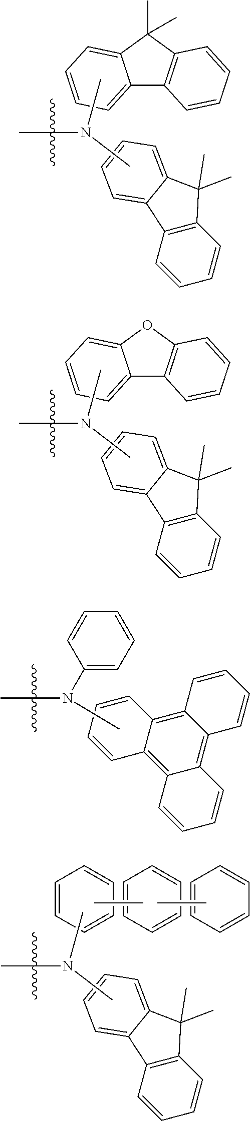

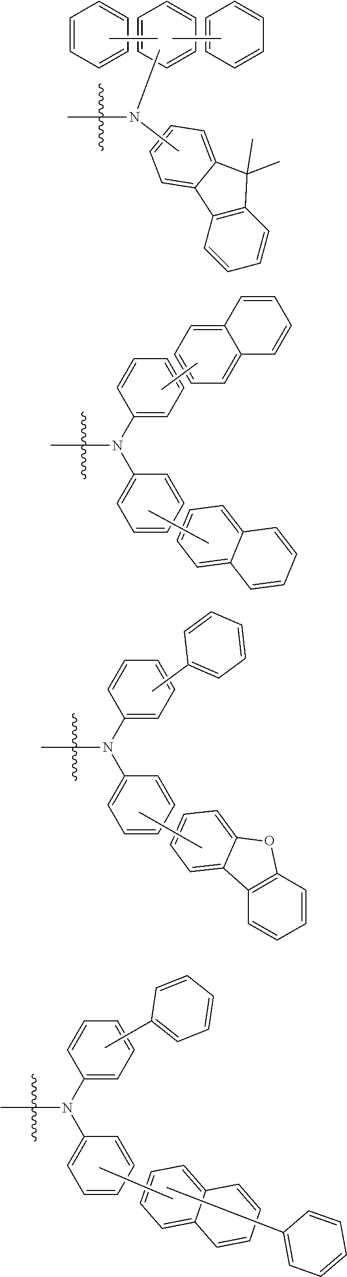

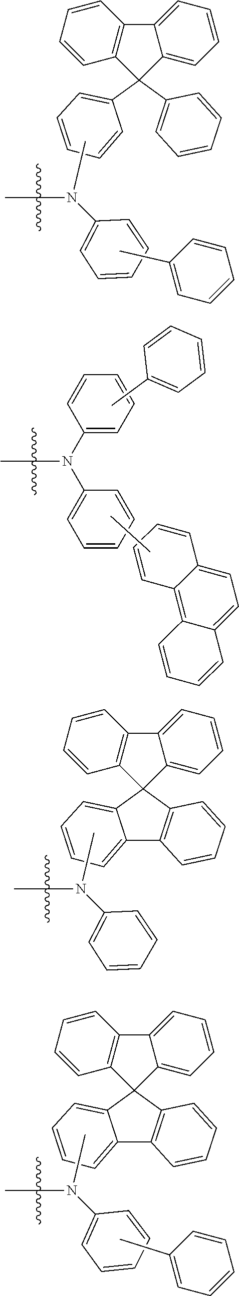

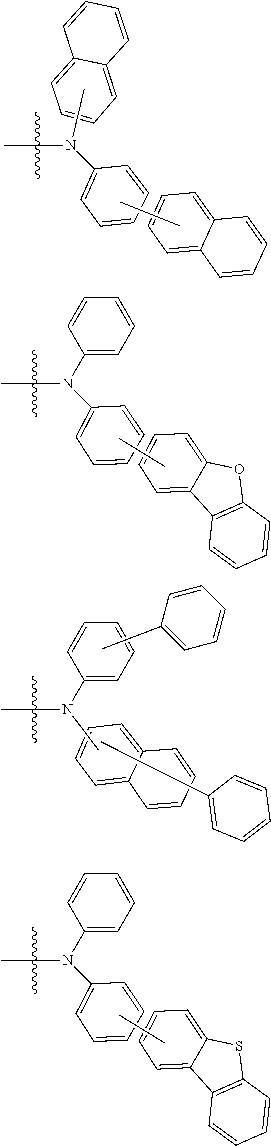

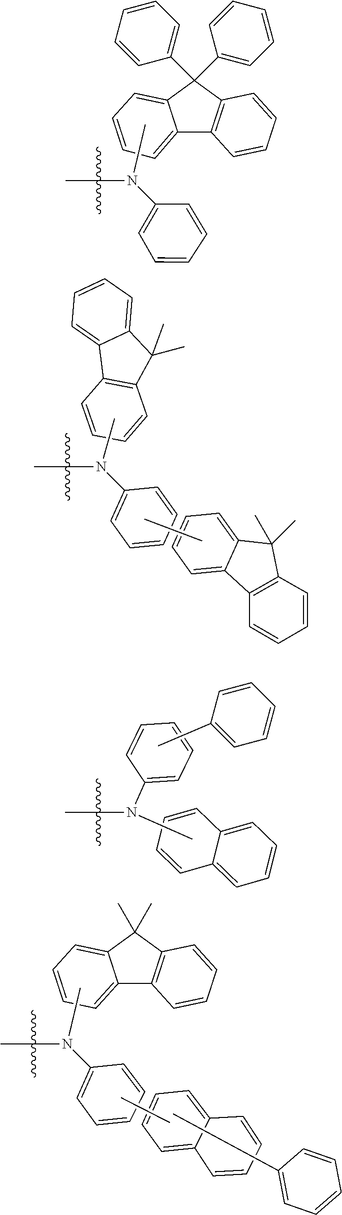

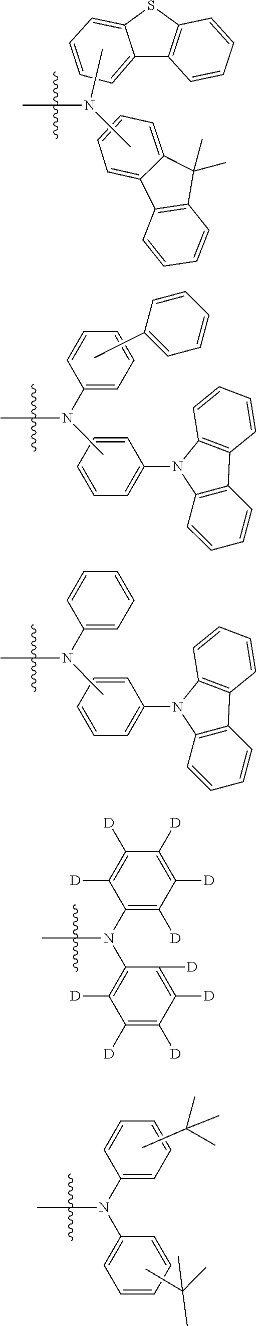

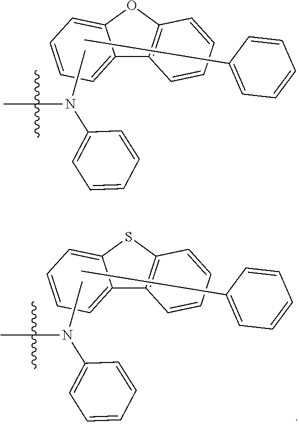

11. The organic compound according to claim 1, wherein the organic compound is selected from the group consisting of the following compounds:

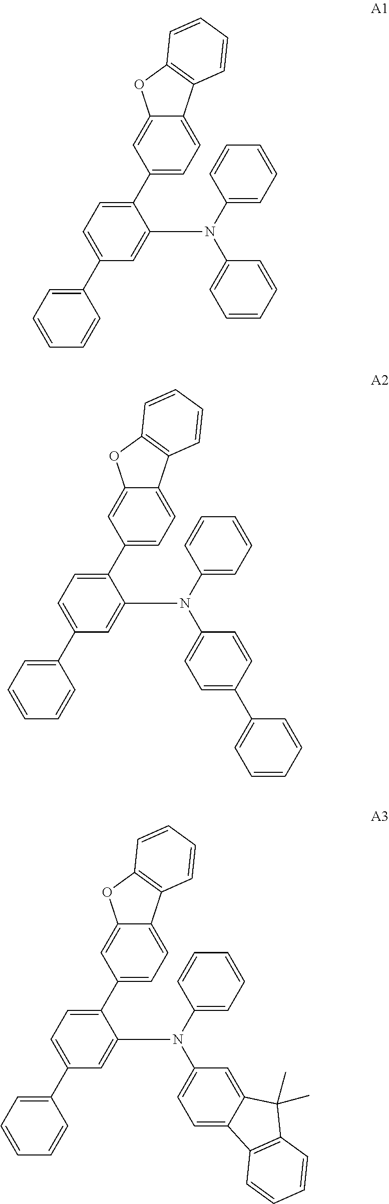 [SMILES: c1ccc(-c2ccc(-c3ccc4c(c3)oc3ccccc34)c(N(c3ccccc3)c3ccccc3)c2)cc1 ; c1ccc(-c2ccc(N(c3ccccc3)c3cc(-c4ccccc4)ccc3-c3ccc4c(c3)oc3ccccc34)cc2)cc1 ; CC1(C)c2ccccc2-c2ccc(N(c3ccccc3)c3cc(-c4ccccc4)ccc3-c3ccc4c(c3)oc3ccccc34)cc21]

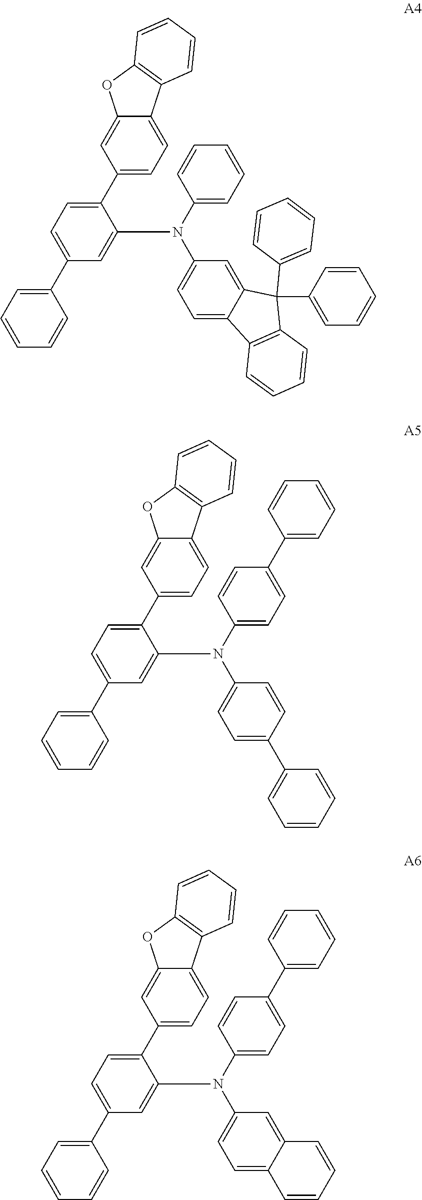 [SMILES: c1ccc(-c2ccc(-c3ccc4c(c3)oc3ccccc34)c(N(c3ccccc3)c3ccc4c(c3)C(c3ccccc3)(c3ccccc3)c3ccccc3-4)c2)cc1 ; c1ccc(-c2ccc(N(c3ccc(-c4ccccc4)cc3)c3cc(-c4ccccc4)ccc3-c3ccc4c(c3)oc3ccccc34)cc2)cc1 ; c1ccc(-c2ccc(N(c3ccc4ccccc4c3)c3cc(-c4ccccc4)ccc3-c3ccc4c(c3)oc3ccccc34)cc2)cc1]

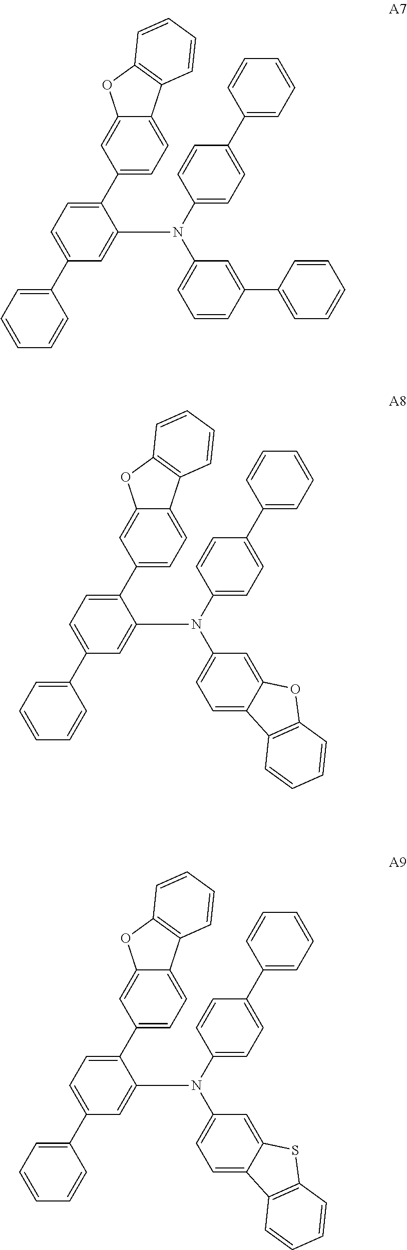 [SMILES: c1ccc(-c2ccc(N(c3cccc(-c4ccccc4)c3)c3cc(-c4ccccc4)ccc3-c3ccc4c(c3)oc3ccccc34)cc2)cc1 ; c1ccc(-c2ccc(N(c3ccc4c(c3)oc3ccccc34)c3cc(-c4ccccc4)ccc3-c3ccc4c(c3)oc3ccccc34)cc2)cc1 ; c1ccc(-c2ccc(N(c3ccc4c(c3)sc3ccccc34)c3cc(-c4ccccc4)ccc3-c3ccc4c(c3)oc3ccccc34)cc2)cc1]

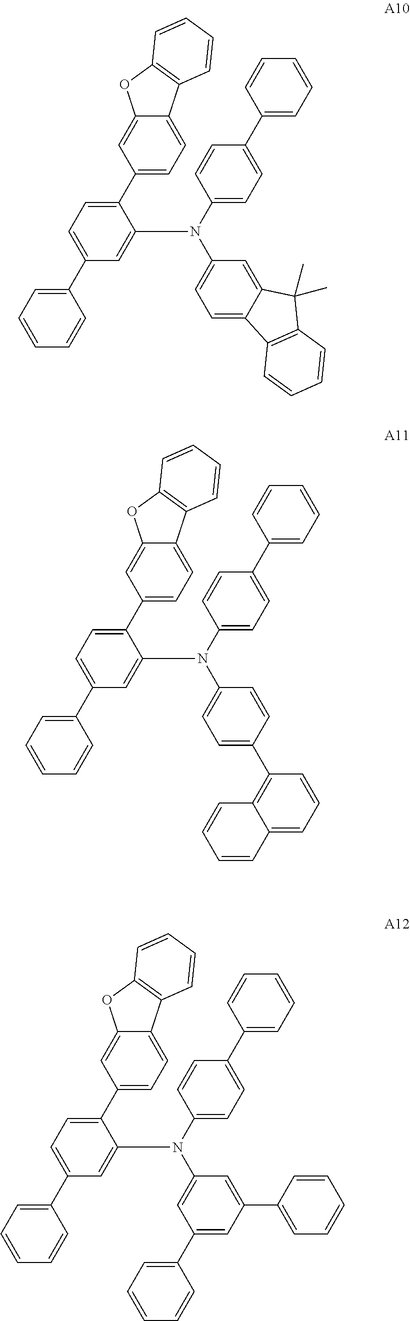 [SMILES: CC1(C)c2ccccc2-c2ccc(N(c3ccc(-c4ccccc4)cc3)c3cc(-c4ccccc4)ccc3-c3ccc4c(c3)oc3ccccc34)cc21 ; c1ccc(-c2ccc(N(c3ccc(-c4cccc5ccccc45)cc3)c3cc(-c4ccccc4)ccc3-c3ccc4c(c3)oc3ccccc34)cc2)cc1 ; c1ccc(-c2ccc(N(c3cc(-c4ccccc4)cc(-c4ccccc4)c3)c3cc(-c4ccccc4)ccc3-c3ccc4c(c3)oc3ccccc34)cc2)cc1]

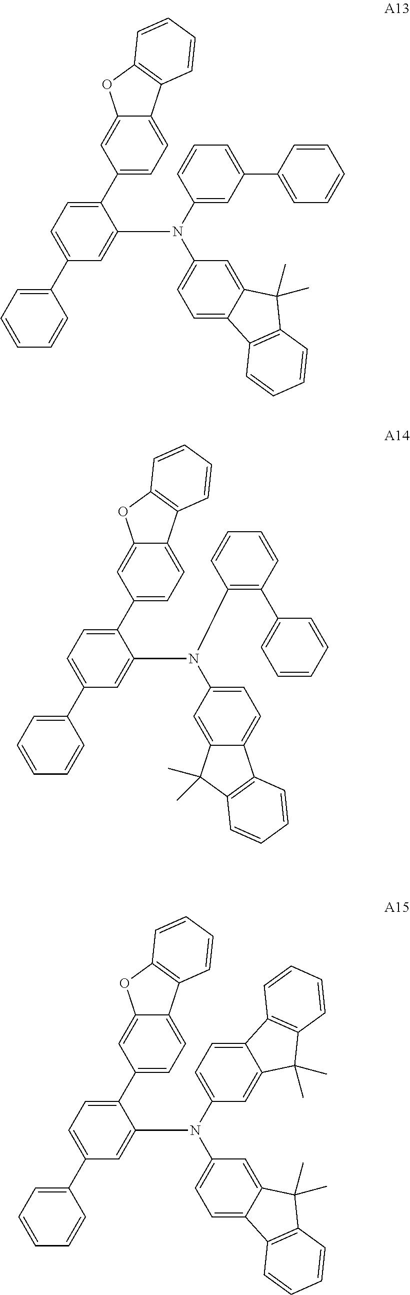 [SMILES: CC1(C)c2ccccc2-c2ccc(N(c3cccc(-c4ccccc4)c3)c3cc(-c4ccccc4)ccc3-c3ccc4c(c3)oc3ccccc34)cc21 ; CC1(C)c2ccccc2-c2ccc(N(c3ccccc3-c3ccccc3)c3cc(-c4ccccc4)ccc3-c3ccc4c(c3)oc3ccccc34)cc21 ; CC1(C)c2ccccc2-c2ccc(N(c3ccc4c(c3)C(C)(C)c3ccccc3-4)c3cc(-c4ccccc4)ccc3-c3ccc4c(c3)oc3ccccc34)cc21]

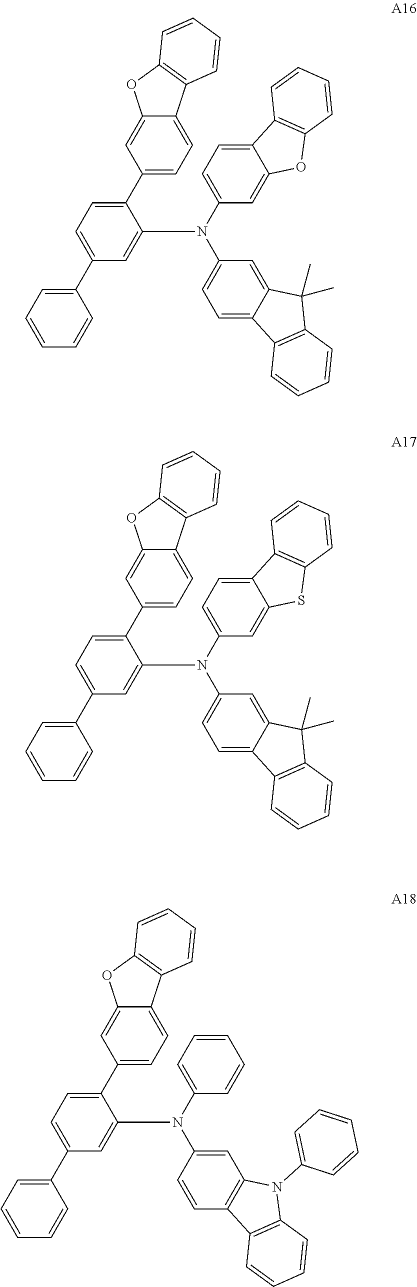 [SMILES: CC1(C)c2ccccc2-c2ccc(N(c3ccc4c(c3)oc3ccccc34)c3cc(-c4ccccc4)ccc3-c3ccc4c(c3)oc3ccccc34)cc21 ; CC1(C)c2ccccc2-c2ccc(N(c3ccc4c(c3)sc3ccccc34)c3cc(-c4ccccc4)ccc3-c3ccc4c(c3)oc3ccccc34)cc21 ; c1ccc(-c2ccc(-c3ccc4c(c3)oc3ccccc34)c(N(c3ccccc3)c3ccc4c5ccccc5n(-c5ccccc5)c4c3)c2)cc1]

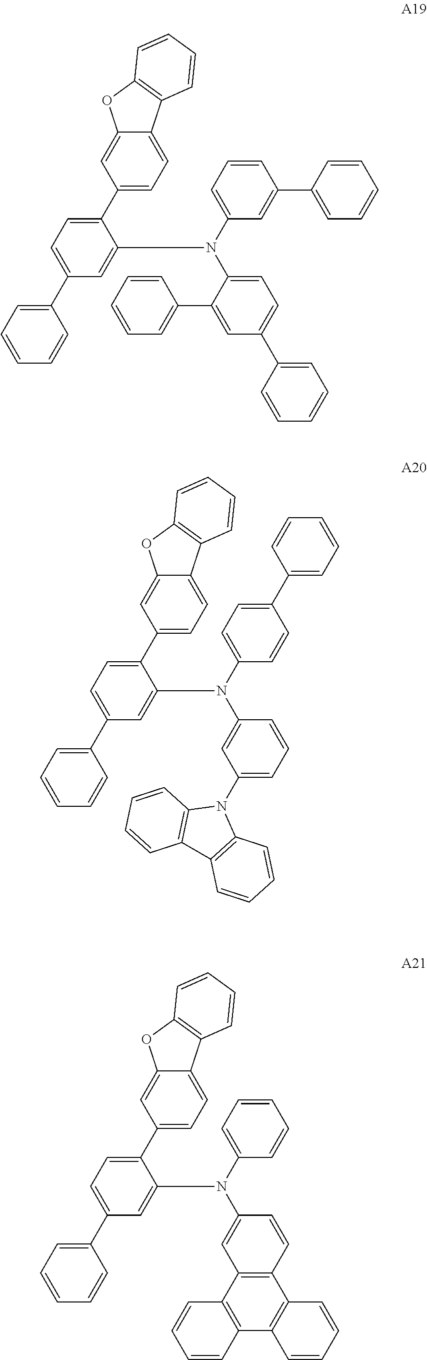 [SMILES: c1ccc(-c2cccc(N(c3ccc(-c4ccccc4)cc3-c3ccccc3)c3cc(-c4ccccc4)ccc3-c3ccc4c(c3)oc3ccccc34)c2)cc1 ; c1ccc(-c2ccc(N(c3cccc(-n4c5ccccc5c5ccccc54)c3)c3cc(-c4ccccc4)ccc3-c3ccc4c(c3)oc3ccccc34)cc2)cc1 ; c1ccc(-c2ccc(-c3ccc4c(c3)oc3ccccc34)c(N(c3ccccc3)c3ccc4c5ccccc5c5ccccc5c4c3)c2)cc1]

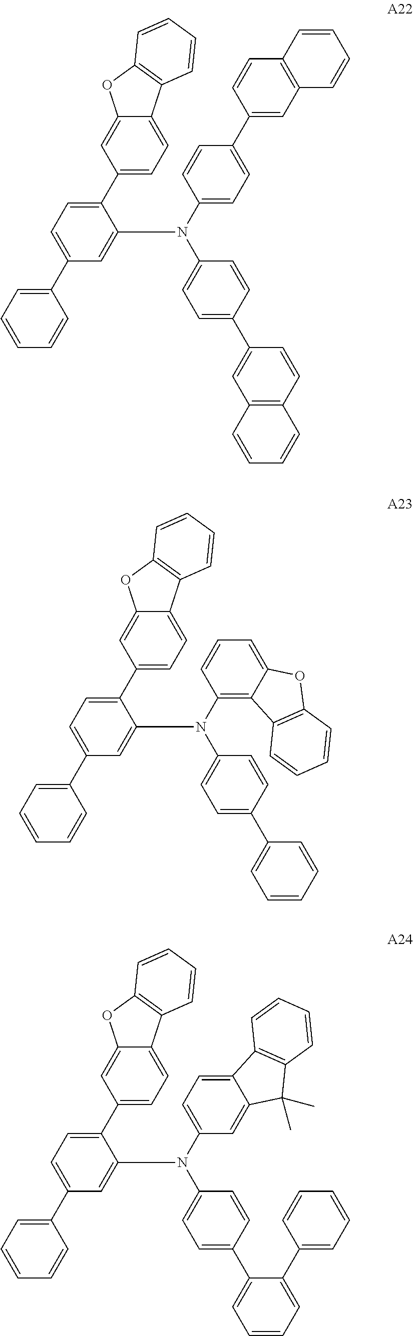 [SMILES: c1ccc(-c2ccc(-c3ccc4c(c3)oc3ccccc34)c(N(c3ccc(-c4ccc5ccccc5c4)cc3)c3ccc(-c4ccc5ccccc5c4)cc3)c2)cc1 ; c1ccc(-c2ccc(N(c3cc(-c4ccccc4)ccc3-c3ccc4c(c3)oc3ccccc34)c3cccc4oc5ccccc5c34)cc2)cc1 ; CC1(C)c2ccccc2-c2ccc(N(c3ccc(-c4ccccc4-c4ccccc4)cc3)c3cc(-c4ccccc4)ccc3-c3ccc4c(c3)oc3ccccc34)cc21]

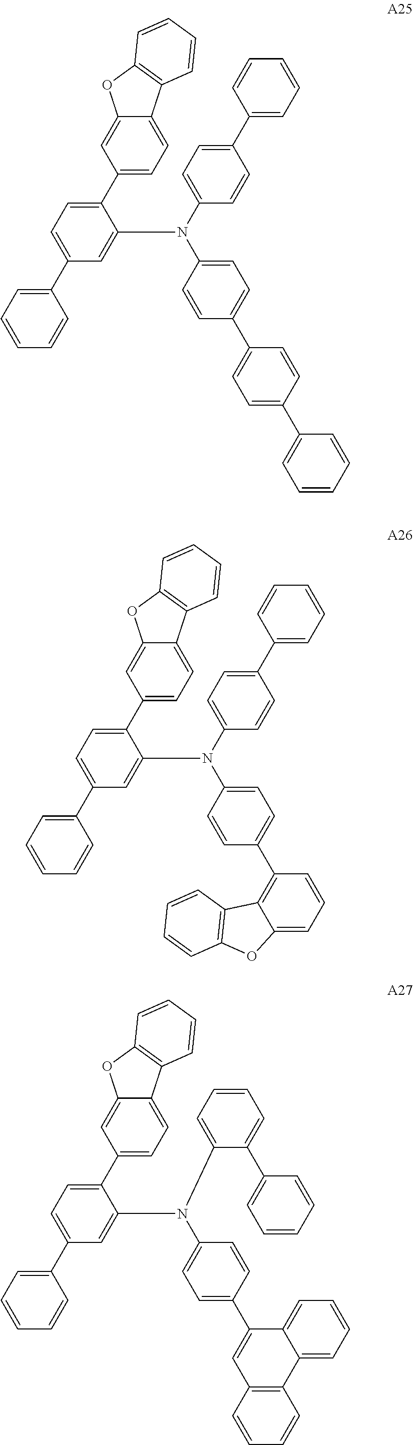 [SMILES: c1ccc(-c2ccc(-c3ccc(N(c4ccc(-c5ccccc5)cc4)c4cc(-c5ccccc5)ccc4-c4ccc5c(c4)oc4ccccc45)cc3)cc2)cc1 ; c1ccc(-c2ccc(N(c3ccc(-c4cccc5oc6ccccc6c45)cc3)c3cc(-c4ccccc4)ccc3-c3ccc4c(c3)oc3ccccc34)cc2)cc1 ; c1ccc(-c2ccc(-c3ccc4c(c3)oc3ccccc34)c(N(c3ccc(-c4cc5ccccc5c5ccccc45)cc3)c3ccccc3-c3ccccc3)c2)cc1]

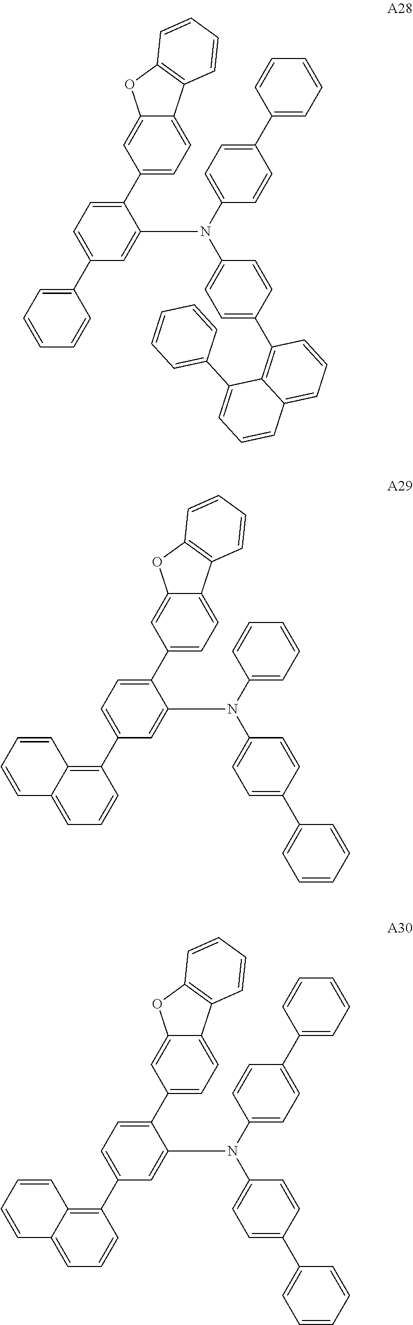 [SMILES: c1ccc(-c2ccc(N(c3ccc(-c4cccc5cccc(-c6ccccc6)c45)cc3)c3cc(-c4ccccc4)ccc3-c3ccc4c(c3)oc3ccccc34)cc2)cc1 ; c1ccc(-c2ccc(N(c3ccccc3)c3cc(-c4cccc5ccccc45)ccc3-c3ccc4c(c3)oc3ccccc34)cc2)cc1 ; c1ccc(-c2ccc(N(c3ccc(-c4ccccc4)cc3)c3cc(-c4cccc5ccccc45)ccc3-c3ccc4c(c3)oc3ccccc34)cc2)cc1]

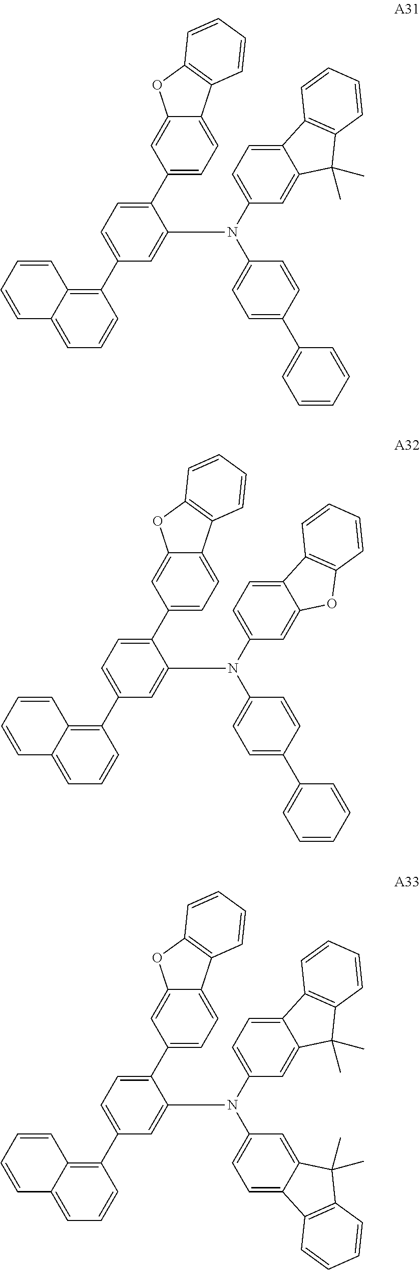 [SMILES: CC1(C)c2ccccc2-c2ccc(N(c3ccc(-c4ccccc4)cc3)c3cc(-c4cccc5ccccc45)ccc3-c3ccc4c(c3)oc3ccccc34)cc21 ; c1ccc(-c2ccc(N(c3ccc4c(c3)oc3ccccc34)c3cc(-c4cccc5ccccc45)ccc3-c3ccc4c(c3)oc3ccccc34)cc2)cc1 ; CC1(C)c2ccccc2-c2ccc(N(c3ccc4c(c3)C(C)(C)c3ccccc3-4)c3cc(-c4cccc5ccccc45)ccc3-c3ccc4c(c3)oc3ccccc34)cc21]

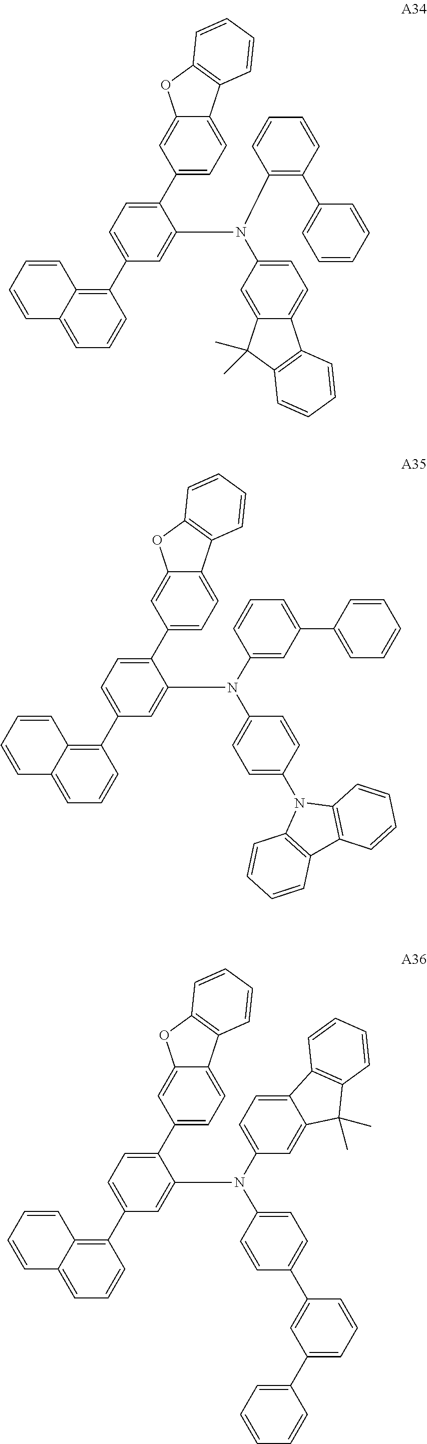 [SMILES: CC1(C)c2ccccc2-c2ccc(N(c3ccccc3-c3ccccc3)c3cc(-c4cccc5ccccc45)ccc3-c3ccc4c(c3)oc3ccccc34)cc21 ; c1ccc(-c2cccc(N(c3ccc(-n4c5ccccc5c5ccccc54)cc3)c3cc(-c4cccc5ccccc45)ccc3-c3ccc4c(c3)oc3ccccc34)c2)cc1 ; CC1(C)c2ccccc2-c2ccc(N(c3ccc(-c4cccc(-c5ccccc5)c4)cc3)c3cc(-c4cccc5ccccc45)ccc3-c3ccc4c(c3)oc3ccccc34)cc21]

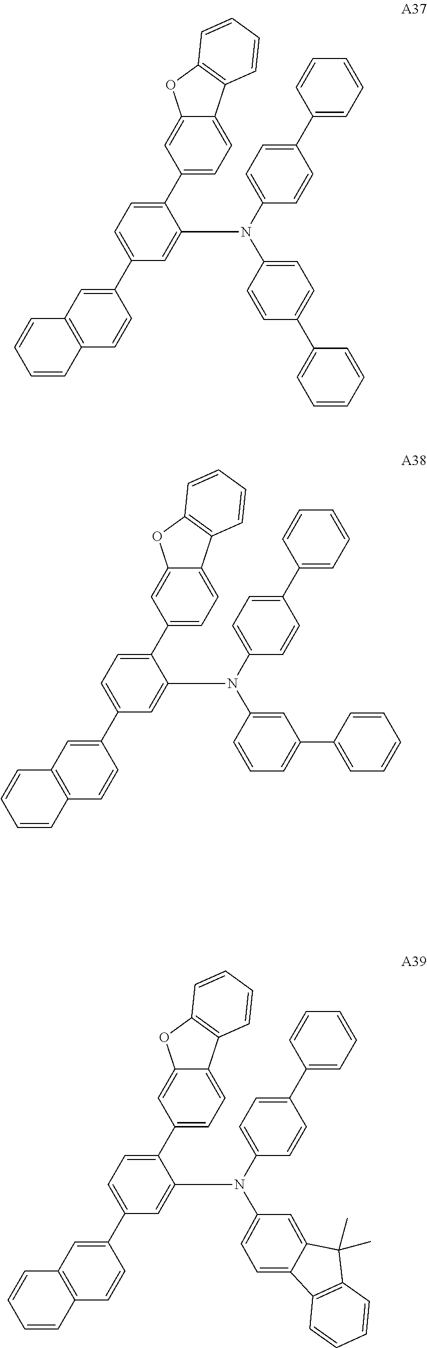 [SMILES: c1ccc(-c2ccc(N(c3ccc(-c4ccccc4)cc3)c3cc(-c4ccc5ccccc5c4)ccc3-c3ccc4c(c3)oc3ccccc34)cc2)cc1 ; c1ccc(-c2ccc(N(c3cccc(-c4ccccc4)c3)c3cc(-c4ccc5ccccc5c4)ccc3-c3ccc4c(c3)oc3ccccc34)cc2)cc1 ; CC1(C)c2ccccc2-c2ccc(N(c3ccc(-c4ccccc4)cc3)c3cc(-c4ccc5ccccc5c4)ccc3-c3ccc4c(c3)oc3ccccc34)cc21]

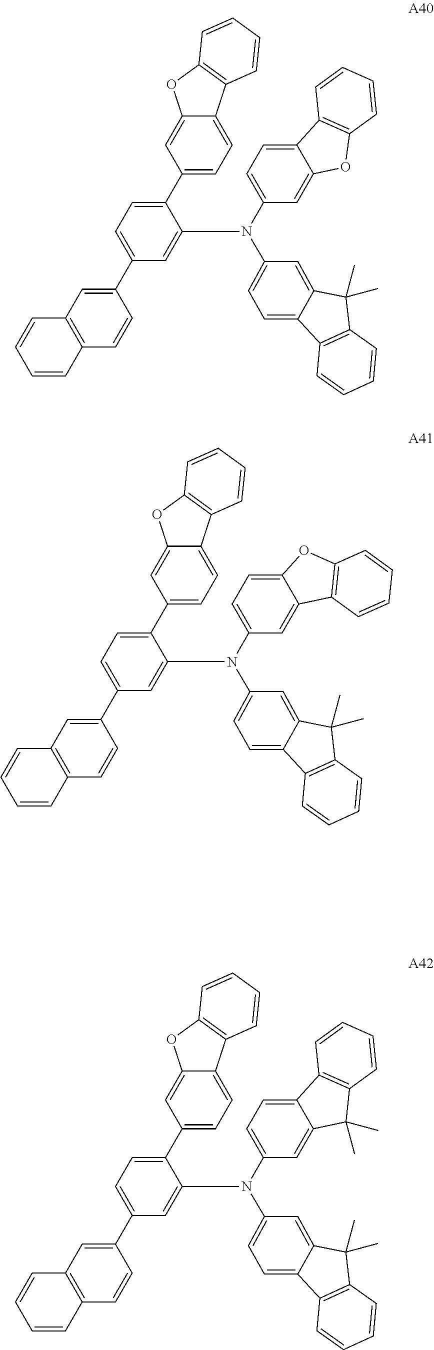 [SMILES: CC1(C)c2ccccc2-c2ccc(N(c3ccc4c(c3)oc3ccccc34)c3cc(-c4ccc5ccccc5c4)ccc3-c3ccc4c(c3)oc3ccccc34)cc21 ; CC1(C)c2ccccc2-c2ccc(N(c3ccc4oc5ccccc5c4c3)c3cc(-c4ccc5ccccc5c4)ccc3-c3ccc4c(c3)oc3ccccc34)cc21 ; CC1(C)c2ccccc2-c2ccc(N(c3ccc4c(c3)C(C)(C)c3ccccc3-4)c3cc(-c4ccc5ccccc5c4)ccc3-c3ccc4c(c3)oc3ccccc34)cc21]

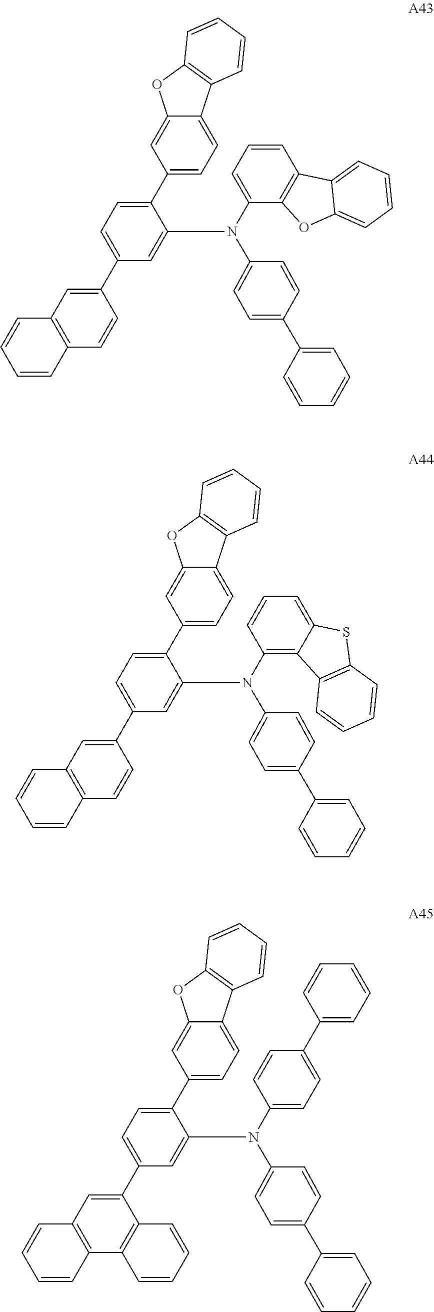 [SMILES: c1ccc(-c2ccc(N(c3cc(-c4ccc5ccccc5c4)ccc3-c3ccc4c(c3)oc3ccccc34)c3cccc4c3oc3ccccc34)cc2)cc1 ; c1ccc(-c2ccc(N(c3cc(-c4ccc5ccccc5c4)ccc3-c3ccc4c(c3)oc3ccccc34)c3cccc4sc5ccccc5c34)cc2)cc1 ; c1ccc(-c2ccc(N(c3ccc(-c4ccccc4)cc3)c3cc(-c4cc5ccccc5c5ccccc45)ccc3-c3ccc4c(c3)oc3ccccc34)cc2)cc1]

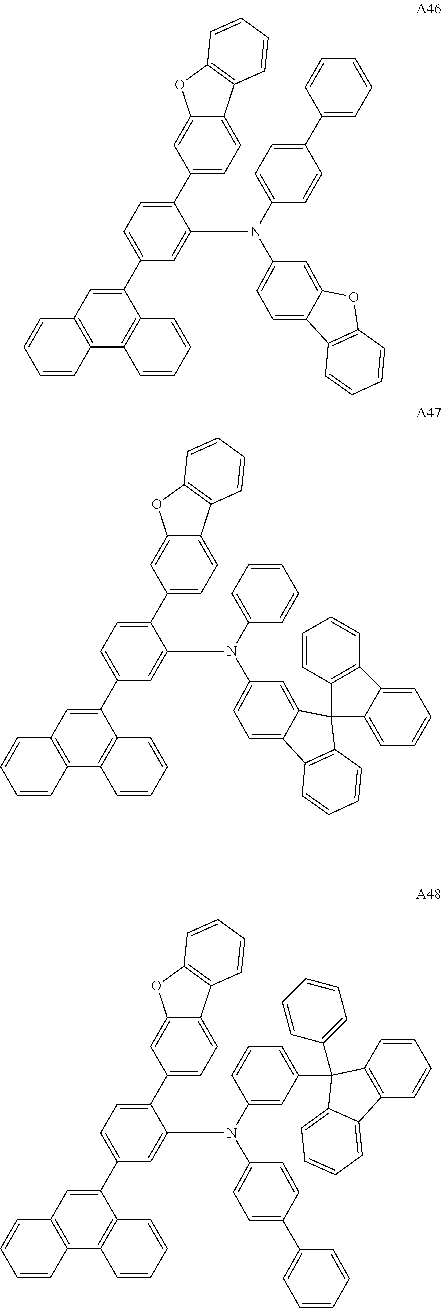 [SMILES: c1ccc(-c2ccc(N(c3ccc4c(c3)oc3ccccc34)c3cc(-c4cc5ccccc5c5ccccc45)ccc3-c3ccc4c(c3)oc3ccccc34)cc2)cc1 ; c1ccc(N(c2ccc3c(c2)C2(c4ccccc4-c4ccccc42)c2ccccc2-3)c2cc(-c3cc4ccccc4c4ccccc34)ccc2-c2ccc3c(c2)oc2ccccc23)cc1 ; c1ccc(-c2ccc(N(c3cccc(C4(c5ccccc5)c5ccccc5-c5ccccc54)c3)c3cc(-c4cc5ccccc5c5ccccc45)ccc3-c3ccc4c(c3)oc3ccccc34)cc2)cc1]

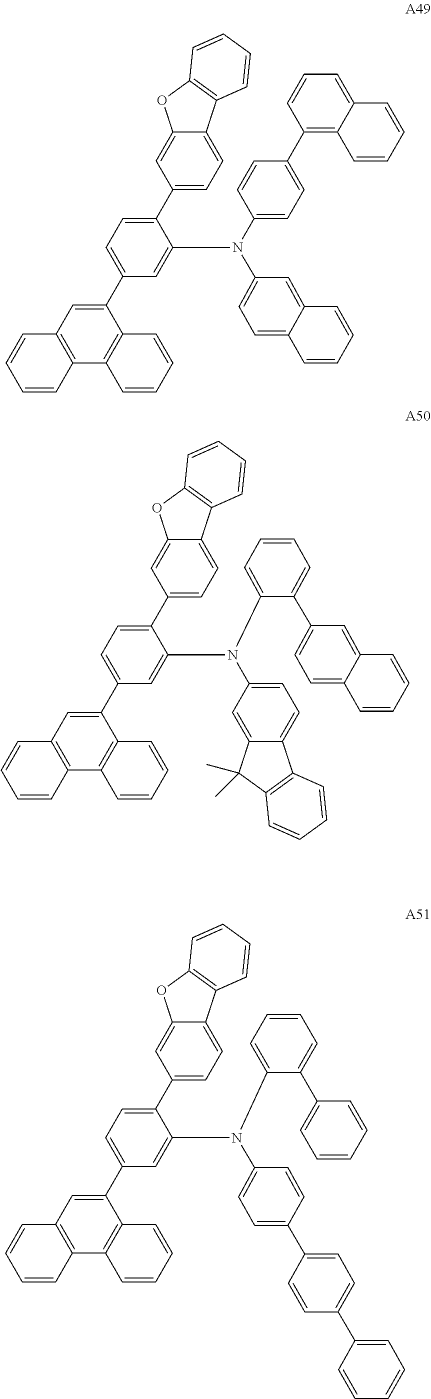 [SMILES: c1ccc2cc(N(c3ccc(-c4cccc5ccccc45)cc3)c3cc(-c4cc5ccccc5c5ccccc45)ccc3-c3ccc4c(c3)oc3ccccc34)ccc2c1 ; CC1(C)c2ccccc2-c2ccc(N(c3ccccc3-c3ccc4ccccc4c3)c3cc(-c4cc5ccccc5c5ccccc45)ccc3-c3ccc4c(c3)oc3ccccc34)cc21 ; c1ccc(-c2ccc(-c3ccc(N(c4ccccc4-c4ccccc4)c4cc(-c5cc6ccccc6c6ccccc56)ccc4-c4ccc5c(c4)oc4ccccc45)cc3)cc2)cc1]

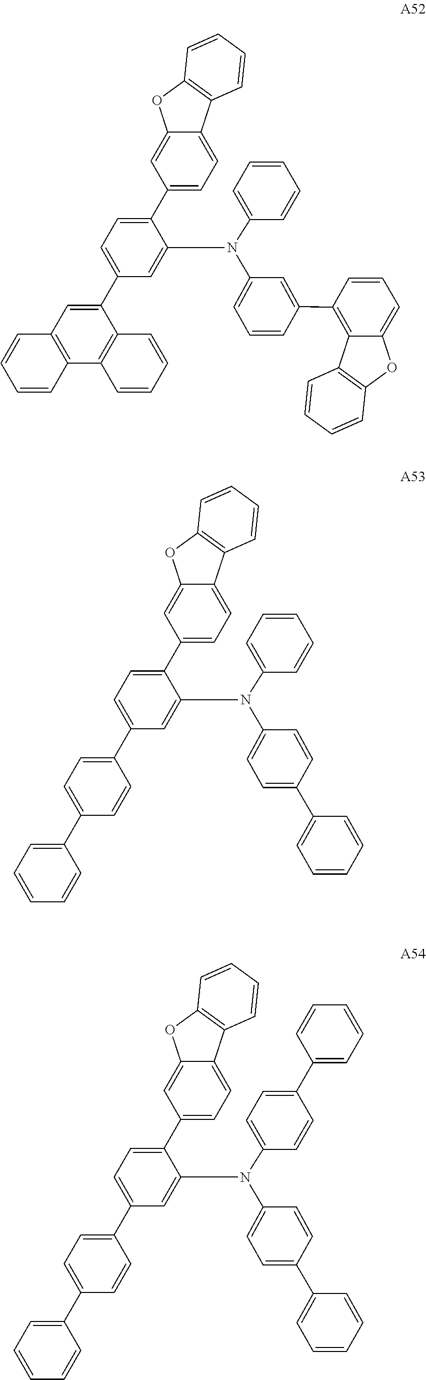 [SMILES: c1ccc(N(c2cccc(-c3cccc4oc5ccccc5c34)c2)c2cc(-c3cc4ccccc4c4ccccc34)ccc2-c2ccc3c(c2)oc2ccccc23)cc1 ; c1ccc(-c2ccc(-c3ccc(-c4ccc5c(c4)oc4ccccc45)c(N(c4ccccc4)c4ccc(-c5ccccc5)cc4)c3)cc2)cc1 ; c1ccc(-c2ccc(-c3ccc(-c4ccc5c(c4)oc4ccccc45)c(N(c4ccc(-c5ccccc5)cc4)c4ccc(-c5ccccc5)cc4)c3)cc2)cc1]

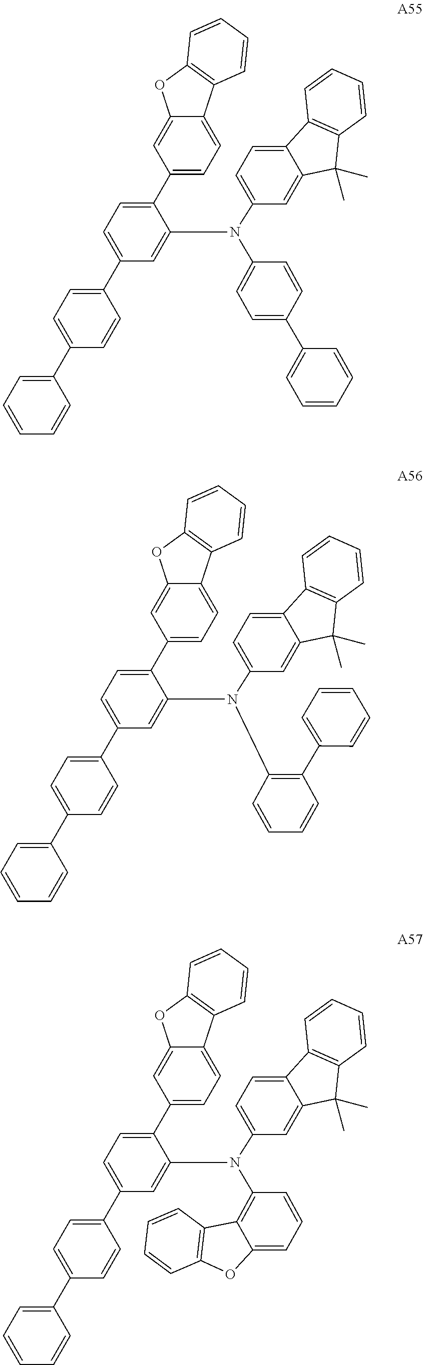 [SMILES: CC1(C)c2ccccc2-c2ccc(N(c3ccc(-c4ccccc4)cc3)c3cc(-c4ccc(-c5ccccc5)cc4)ccc3-c3ccc4c(c3)oc3ccccc34)cc21 ; CC1(C)c2ccccc2-c2ccc(N(c3ccccc3-c3ccccc3)c3cc(-c4ccc(-c5ccccc5)cc4)ccc3-c3ccc4c(c3)oc3ccccc34)cc21 ; CC1(C)c2ccccc2-c2ccc(N(c3cc(-c4ccc(-c5ccccc5)cc4)ccc3-c3ccc4c(c3)oc3ccccc34)c3cccc4oc5ccccc5c34)cc21]

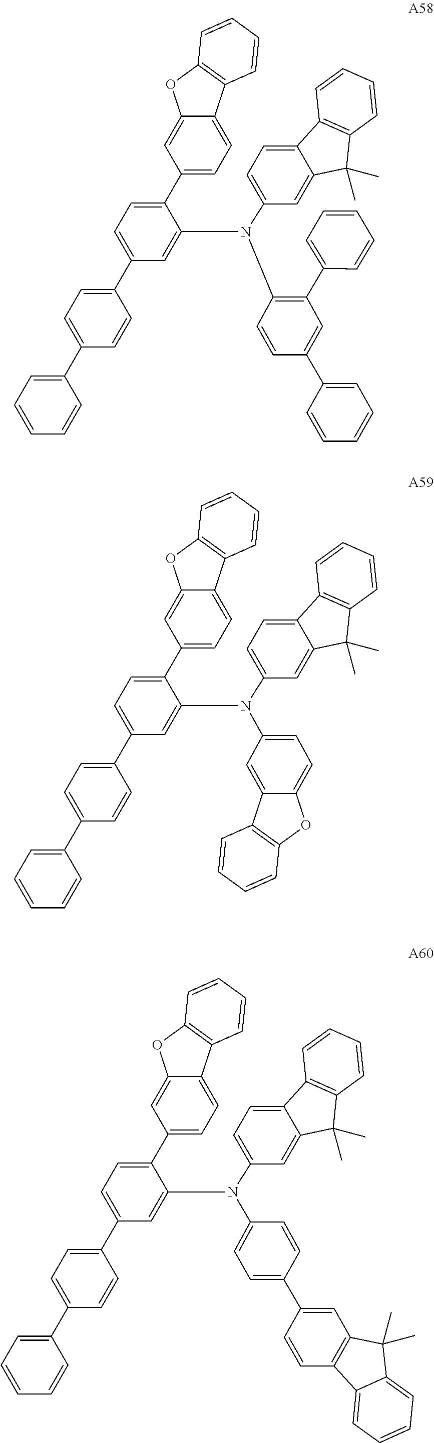 [SMILES: CC1(C)c2ccccc2-c2ccc(N(c3ccc(-c4ccccc4)cc3-c3ccccc3)c3cc(-c4ccc(-c5ccccc5)cc4)ccc3-c3ccc4c(c3)oc3ccccc34)cc21 ; CC1(C)c2ccccc2-c2ccc(N(c3ccc4oc5ccccc5c4c3)c3cc(-c4ccc(-c5ccccc5)cc4)ccc3-c3ccc4c(c3)oc3ccccc34)cc21 ; CC1(C)c2ccccc2-c2ccc(-c3ccc(N(c4ccc5c(c4)C(C)(C)c4ccccc4-5)c4cc(-c5ccc(-c6ccccc6)cc5)ccc4-c4ccc5c(c4)oc4ccccc45)cc3)cc21]

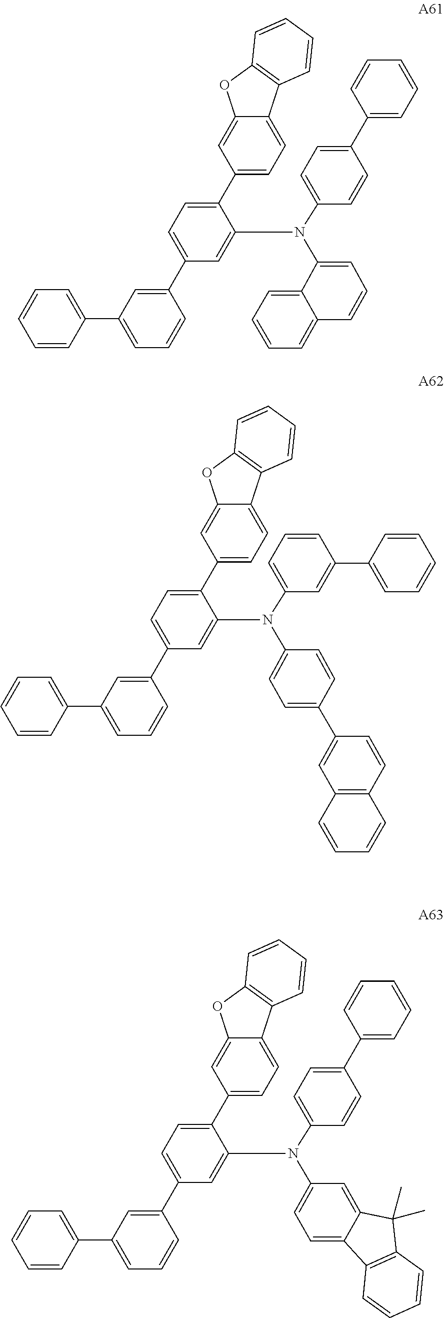 [SMILES: c1ccc(-c2ccc(N(c3cc(-c4cccc(-c5ccccc5)c4)ccc3-c3ccc4c(c3)oc3ccccc34)c3cccc4ccccc34)cc2)cc1 ; c1ccc(-c2cccc(-c3ccc(-c4ccc5c(c4)oc4ccccc45)c(N(c4ccc(-c5ccc6ccccc6c5)cc4)c4cccc(-c5ccccc5)c4)c3)c2)cc1 ; CC1(C)c2ccccc2-c2ccc(N(c3ccc(-c4ccccc4)cc3)c3cc(-c4cccc(-c5ccccc5)c4)ccc3-c3ccc4c(c3)oc3ccccc34)cc21]

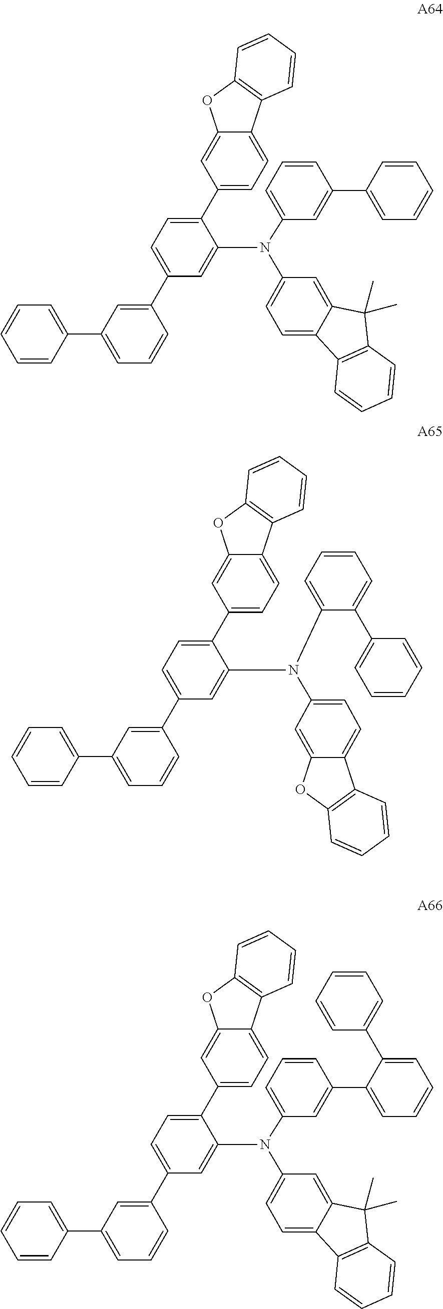 [SMILES: CC1(C)c2ccccc2-c2ccc(N(c3cccc(-c4ccccc4)c3)c3cc(-c4cccc(-c5ccccc5)c4)ccc3-c3ccc4c(c3)oc3ccccc34)cc21 ; c1ccc(-c2cccc(-c3ccc(-c4ccc5c(c4)oc4ccccc45)c(N(c4ccc5c(c4)oc4ccccc45)c4ccccc4-c4ccccc4)c3)c2)cc1 ; CC1(C)c2ccccc2-c2ccc(N(c3cccc(-c4ccccc4-c4ccccc4)c3)c3cc(-c4cccc(-c5ccccc5)c4)ccc3-c3ccc4c(c3)oc3ccccc34)cc21]

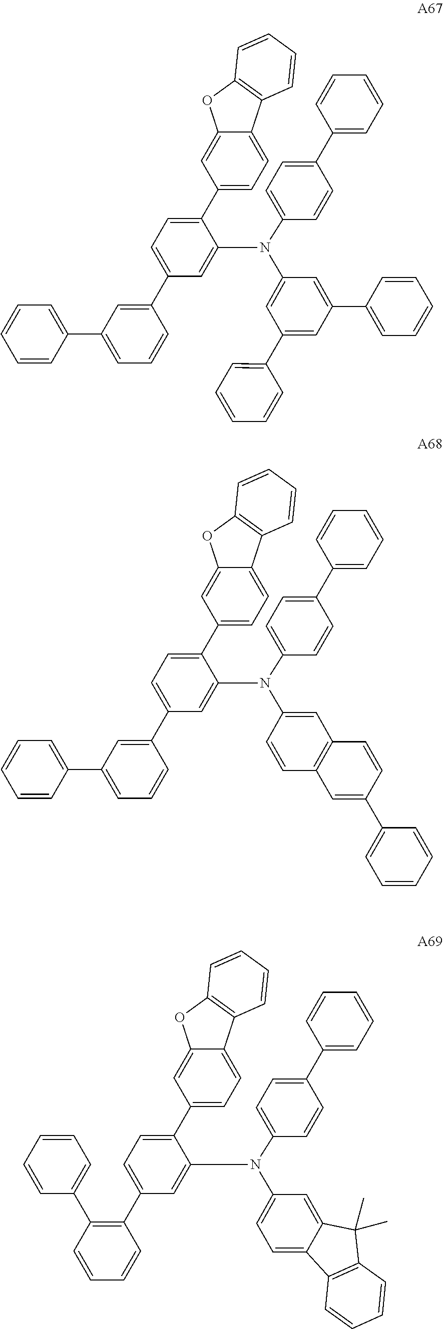 [SMILES: c1ccc(-c2ccc(N(c3cc(-c4ccccc4)cc(-c4ccccc4)c3)c3cc(-c4cccc(-c5ccccc5)c4)ccc3-c3ccc4c(c3)oc3ccccc34)cc2)cc1 ; c1ccc(-c2ccc(N(c3ccc4cc(-c5ccccc5)ccc4c3)c3cc(-c4cccc(-c5ccccc5)c4)ccc3-c3ccc4c(c3)oc3ccccc34)cc2)cc1 ; CC1(C)c2ccccc2-c2ccc(N(c3ccc(-c4ccccc4)cc3)c3cc(-c4ccccc4-c4ccccc4)ccc3-c3ccc4c(c3)oc3ccccc34)cc21]

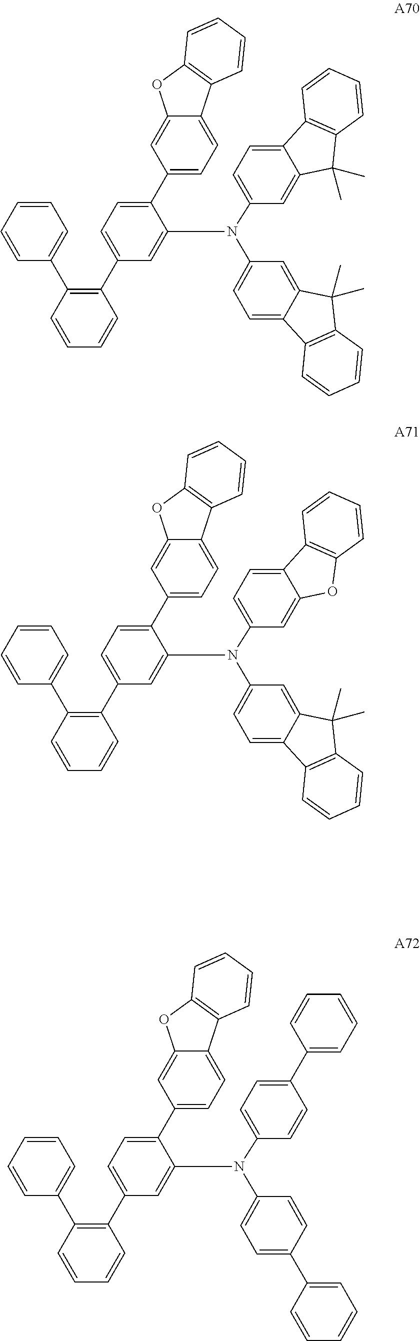 [SMILES: CC1(C)c2ccccc2-c2ccc(N(c3ccc4c(c3)C(C)(C)c3ccccc3-4)c3cc(-c4ccccc4-c4ccccc4)ccc3-c3ccc4c(c3)oc3ccccc34)cc21 ; CC1(C)c2ccccc2-c2ccc(N(c3ccc4c(c3)oc3ccccc34)c3cc(-c4ccccc4-c4ccccc4)ccc3-c3ccc4c(c3)oc3ccccc34)cc21 ; c1ccc(-c2ccc(N(c3ccc(-c4ccccc4)cc3)c3cc(-c4ccccc4-c4ccccc4)ccc3-c3ccc4c(c3)oc3ccccc34)cc2)cc1]

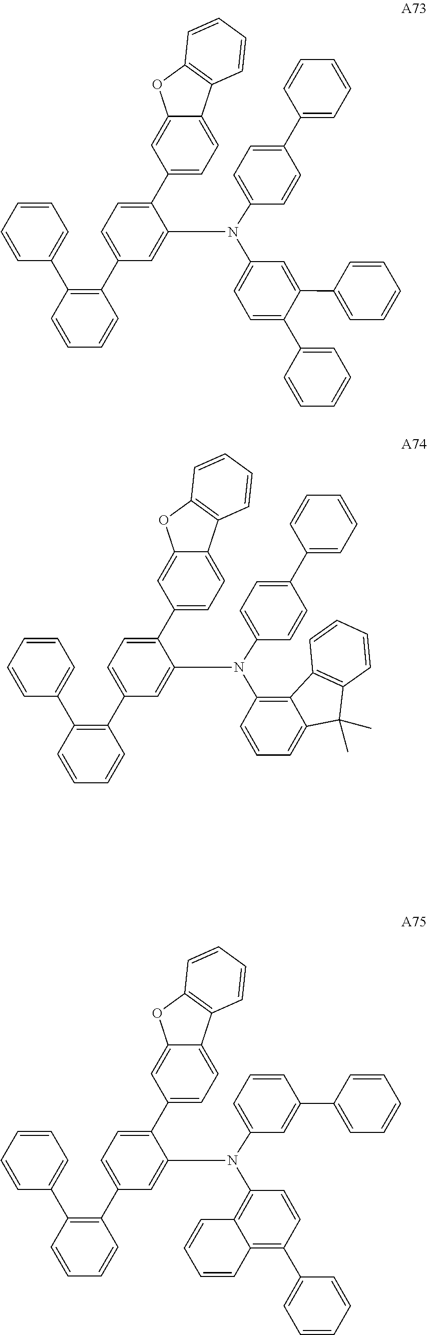 [SMILES: c1ccc(-c2ccc(N(c3ccc(-c4ccccc4)c(-c4ccccc4)c3)c3cc(-c4ccccc4-c4ccccc4)ccc3-c3ccc4c(c3)oc3ccccc34)cc2)cc1 ; CC1(C)c2ccccc2-c2c(N(c3ccc(-c4ccccc4)cc3)c3cc(-c4ccccc4-c4ccccc4)ccc3-c3ccc4c(c3)oc3ccccc34)cccc21 ; c1ccc(-c2cccc(N(c3cc(-c4ccccc4-c4ccccc4)ccc3-c3ccc4c(c3)oc3ccccc34)c3ccc(-c4ccccc4)c4ccccc34)c2)cc1]

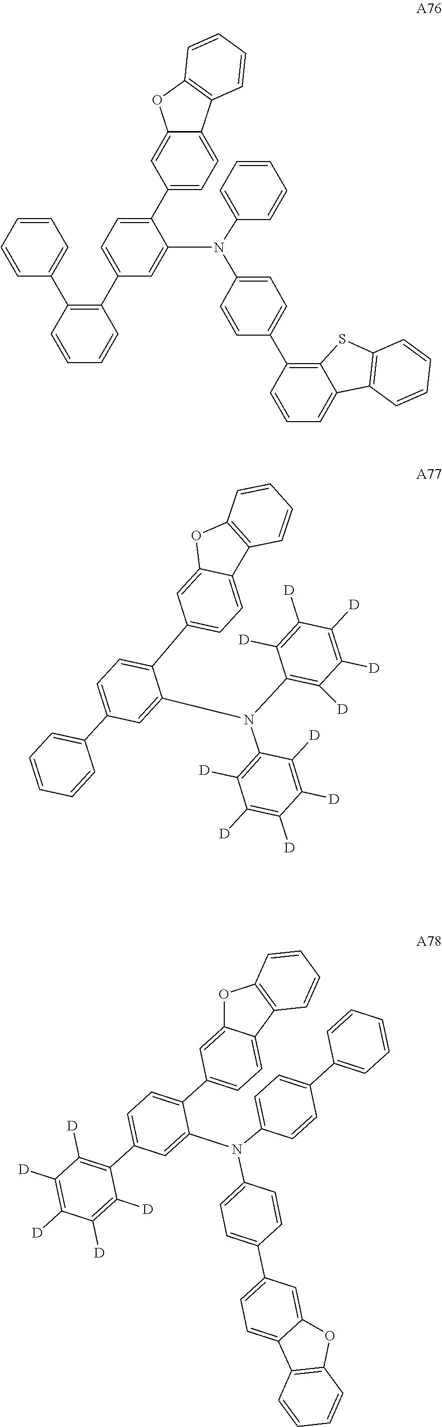 [SMILES: c1ccc(-c2ccccc2-c2ccc(-c3ccc4c(c3)oc3ccccc34)c(N(c3ccccc3)c3ccc(-c4cccc5c4sc4ccccc45)cc3)c2)cc1 ; [2H]c1c([2H])c([2H])c(N(c2cc(-c3ccccc3)ccc2-c2ccc3c(c2)oc2ccccc23)c2c([2H])c([2H])c([2H])c([2H])c2[2H])c([2H])c1[2H] ; [2H]c1c([2H])c([2H])c(-c2ccc(-c3ccc4c(c3)oc3ccccc34)c(N(c3ccc(-c4ccccc4)cc3)c3ccc(-c4ccc5c(c4)oc4ccccc45)cc3)c2)c([2H])c1[2H]]

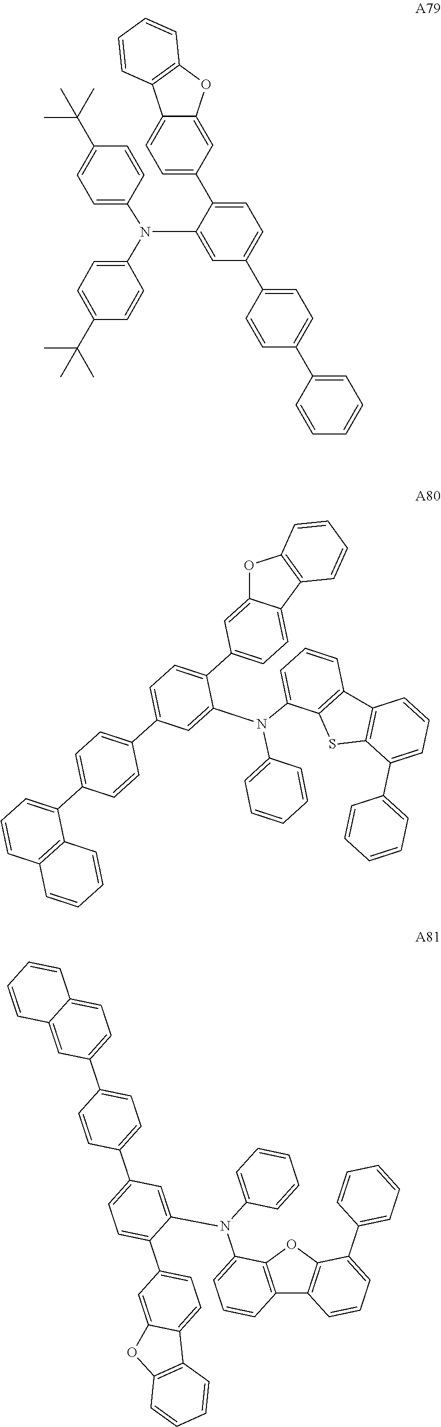 [SMILES: CC(C)(C)c1ccc(N(c2ccc(C(C)(C)C)cc2)c2cc(-c3ccc(-c4ccccc4)cc3)ccc2-c2ccc3c(c2)oc2ccccc23)cc1 ; c1ccc(-c2cccc3c2sc2c(N(c4ccccc4)c4cc(-c5ccc(-c6cccc7ccccc67)cc5)ccc4-c4ccc5c(c4)oc4ccccc45)cccc23)cc1 ; c1ccc(-c2cccc3c2oc2c(N(c4ccccc4)c4cc(-c5ccc(-c6ccc7ccccc7c6)cc5)ccc4-c4ccc5c(c4)oc4ccccc45)cccc23)cc1]

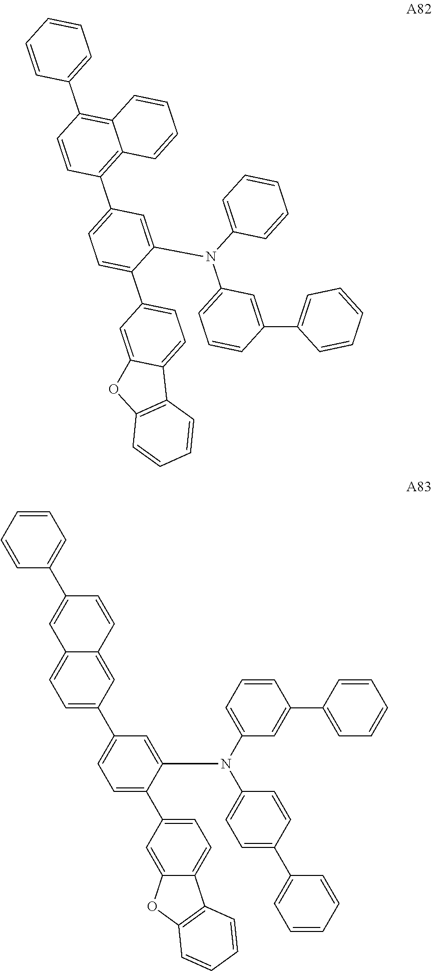 [SMILES: c1ccc(-c2cccc(N(c3ccccc3)c3cc(-c4ccc(-c5ccccc5)c5ccccc45)ccc3-c3ccc4c(c3)oc3ccccc34)c2)cc1 ; c1ccc(-c2ccc(N(c3cccc(-c4ccccc4)c3)c3cc(-c4ccc5cc(-c6ccccc6)ccc5c4)ccc3-c3ccc4c(c3)oc3ccccc34)cc2)cc1]

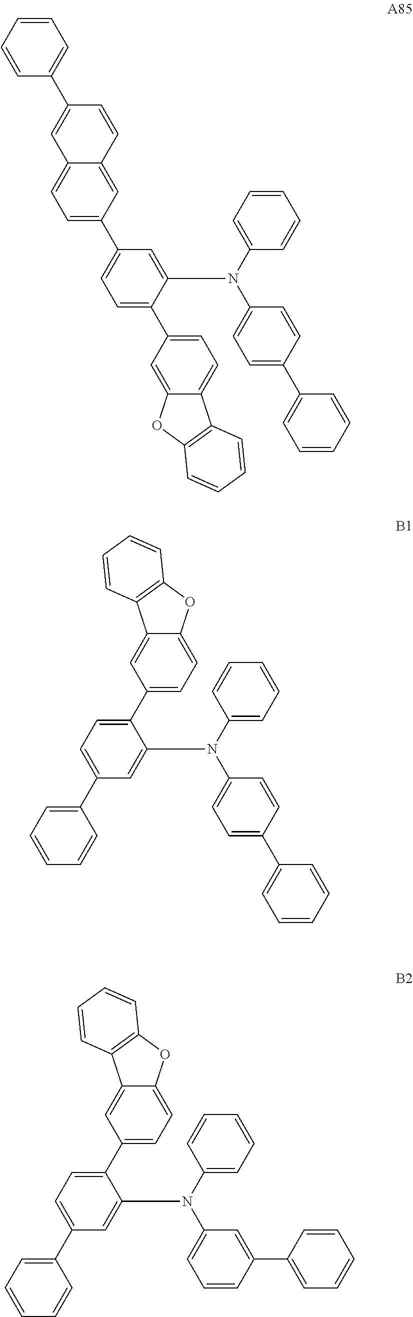 [SMILES: c1ccc(-c2ccc(N(c3ccccc3)c3cc(-c4ccc5cc(-c6ccccc6)ccc5c4)ccc3-c3ccc4c(c3)oc3ccccc34)cc2)cc1 ; c1ccc(-c2ccc(N(c3ccccc3)c3cc(-c4ccccc4)ccc3-c3ccc4oc5ccccc5c4c3)cc2)cc1 ; c1ccc(-c2cccc(N(c3ccccc3)c3cc(-c4ccccc4)ccc3-c3ccc4oc5ccccc5c4c3)c2)cc1]

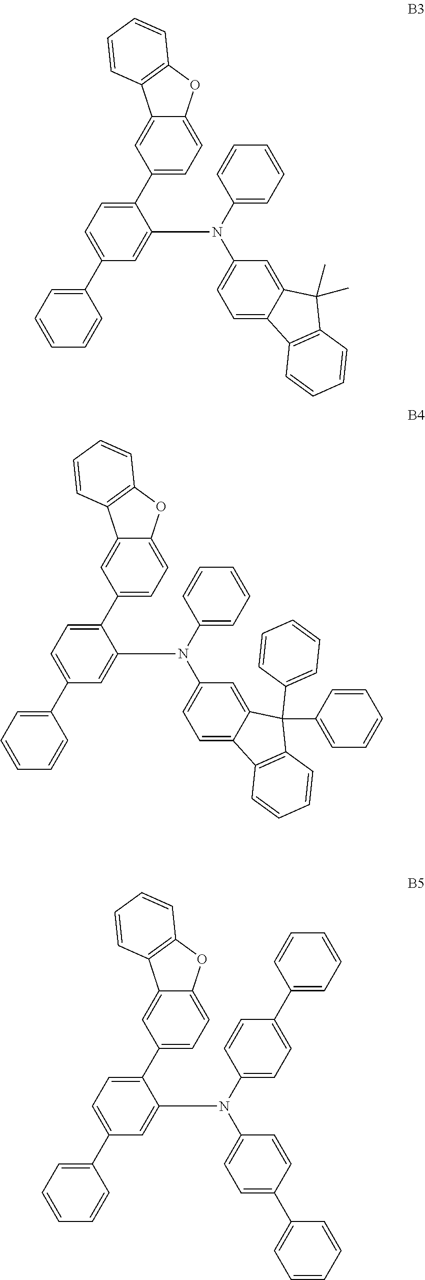 [SMILES: CC1(C)c2ccccc2-c2ccc(N(c3ccccc3)c3cc(-c4ccccc4)ccc3-c3ccc4oc5ccccc5c4c3)cc21 ; c1ccc(-c2ccc(-c3ccc4oc5ccccc5c4c3)c(N(c3ccccc3)c3ccc4c(c3)C(c3ccccc3)(c3ccccc3)c3ccccc3-4)c2)cc1 ; c1ccc(-c2ccc(N(c3ccc(-c4ccccc4)cc3)c3cc(-c4ccccc4)ccc3-c3ccc4oc5ccccc5c4c3)cc2)cc1]

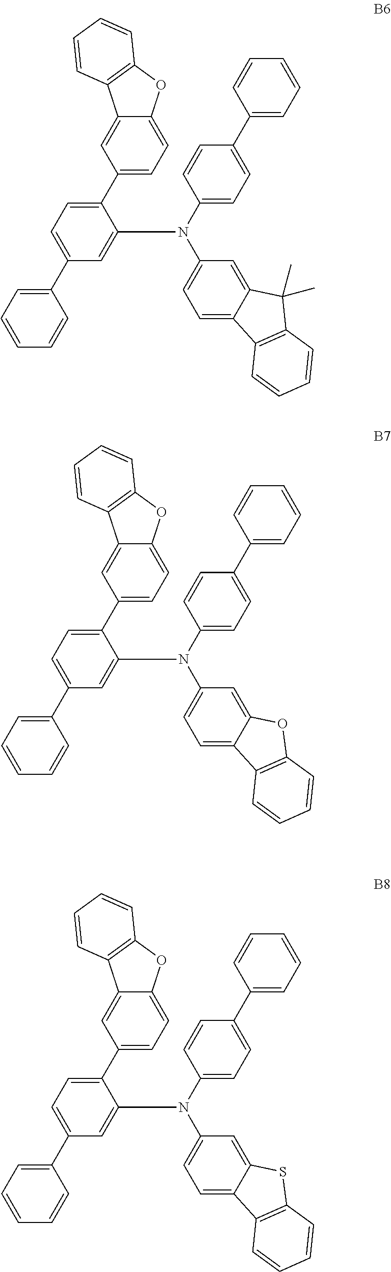 [SMILES: CC1(C)c2ccccc2-c2ccc(N(c3ccc(-c4ccccc4)cc3)c3cc(-c4ccccc4)ccc3-c3ccc4oc5ccccc5c4c3)cc21 ; c1ccc(-c2ccc(N(c3ccc4c(c3)oc3ccccc34)c3cc(-c4ccccc4)ccc3-c3ccc4oc5ccccc5c4c3)cc2)cc1 ; c1ccc(-c2ccc(N(c3ccc4c(c3)sc3ccccc34)c3cc(-c4ccccc4)ccc3-c3ccc4oc5ccccc5c4c3)cc2)cc1]

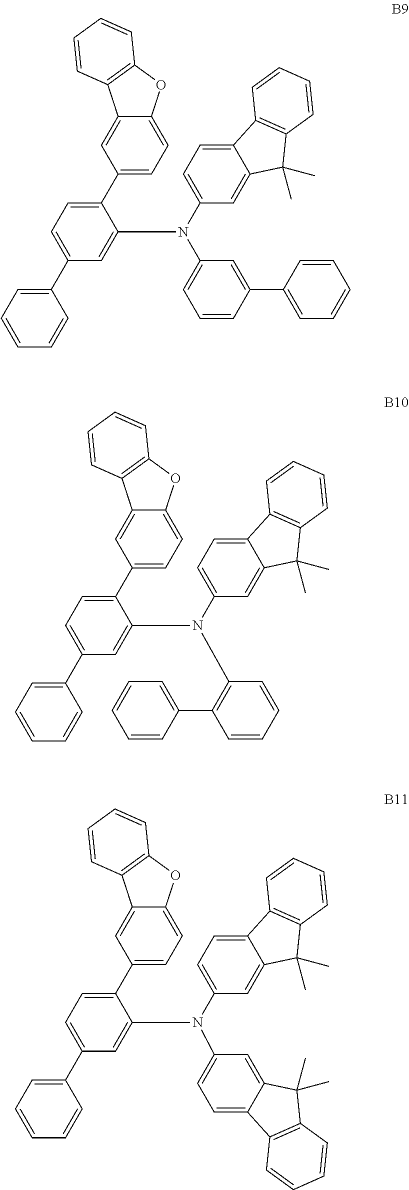 [SMILES: CC1(C)c2ccccc2-c2ccc(N(c3cccc(-c4ccccc4)c3)c3cc(-c4ccccc4)ccc3-c3ccc4oc5ccccc5c4c3)cc21 ; CC1(C)c2ccccc2-c2ccc(N(c3ccccc3-c3ccccc3)c3cc(-c4ccccc4)ccc3-c3ccc4oc5ccccc5c4c3)cc21 ; CC1(C)c2ccccc2-c2ccc(N(c3ccc4c(c3)C(C)(C)c3ccccc3-4)c3cc(-c4ccccc4)ccc3-c3ccc4oc5ccccc5c4c3)cc21]

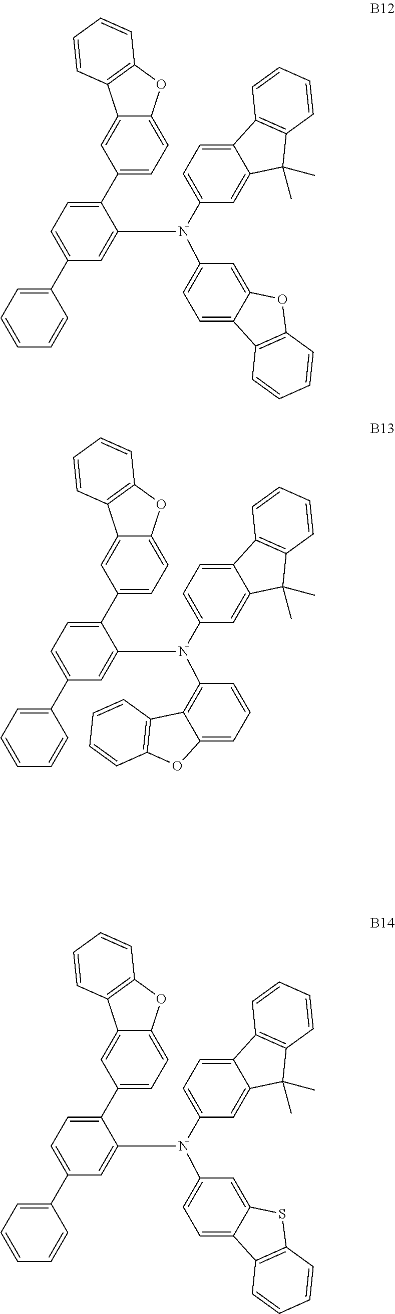 [SMILES: CC1(C)c2ccccc2-c2ccc(N(c3ccc4c(c3)oc3ccccc34)c3cc(-c4ccccc4)ccc3-c3ccc4oc5ccccc5c4c3)cc21 ; CC1(C)c2ccccc2-c2ccc(N(c3cc(-c4ccccc4)ccc3-c3ccc4oc5ccccc5c4c3)c3cccc4oc5ccccc5c34)cc21 ; CC1(C)c2ccccc2-c2ccc(N(c3ccc4c(c3)sc3ccccc34)c3cc(-c4ccccc4)ccc3-c3ccc4oc5ccccc5c4c3)cc21]

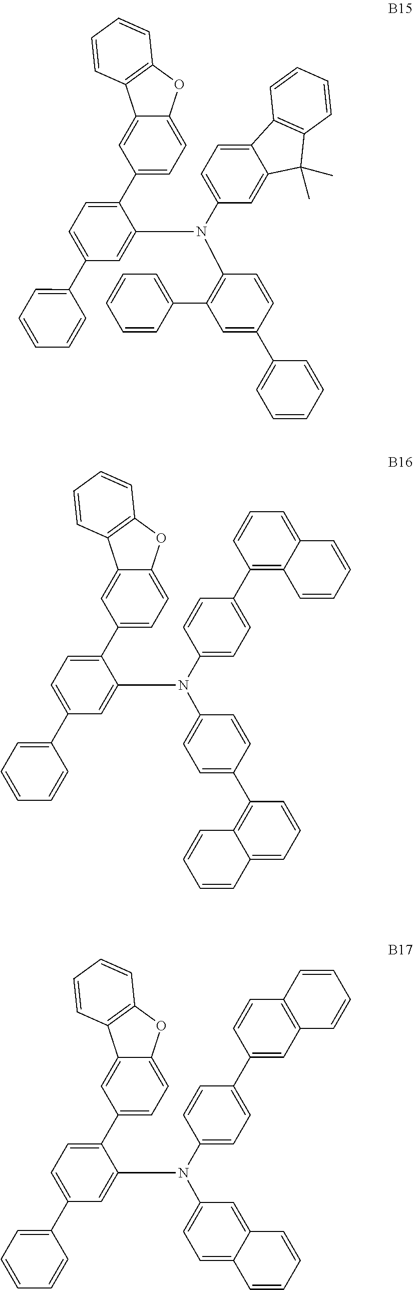 [SMILES: CC1(C)c2ccccc2-c2ccc(N(c3ccc(-c4ccccc4)cc3-c3ccccc3)c3cc(-c4ccccc4)ccc3-c3ccc4oc5ccccc5c4c3)cc21 ; c1ccc(-c2ccc(-c3ccc4oc5ccccc5c4c3)c(N(c3ccc(-c4cccc5ccccc45)cc3)c3ccc(-c4cccc5ccccc45)cc3)c2)cc1 ; c1ccc(-c2ccc(-c3ccc4oc5ccccc5c4c3)c(N(c3ccc(-c4ccc5ccccc5c4)cc3)c3ccc4ccccc4c3)c2)cc1]

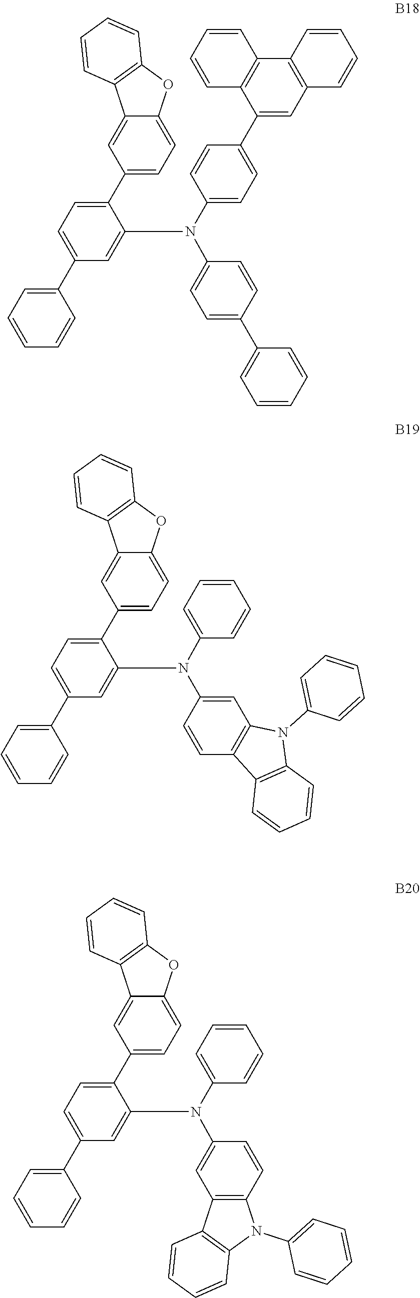 [SMILES: c1ccc(-c2ccc(N(c3ccc(-c4cc5ccccc5c5ccccc45)cc3)c3cc(-c4ccccc4)ccc3-c3ccc4oc5ccccc5c4c3)cc2)cc1 ; c1ccc(-c2ccc(-c3ccc4oc5ccccc5c4c3)c(N(c3ccccc3)c3ccc4c5ccccc5n(-c5ccccc5)c4c3)c2)cc1 ; c1ccc(-c2ccc(-c3ccc4oc5ccccc5c4c3)c(N(c3ccccc3)c3ccc4c(c3)c3ccccc3n4-c3ccccc3)c2)cc1]

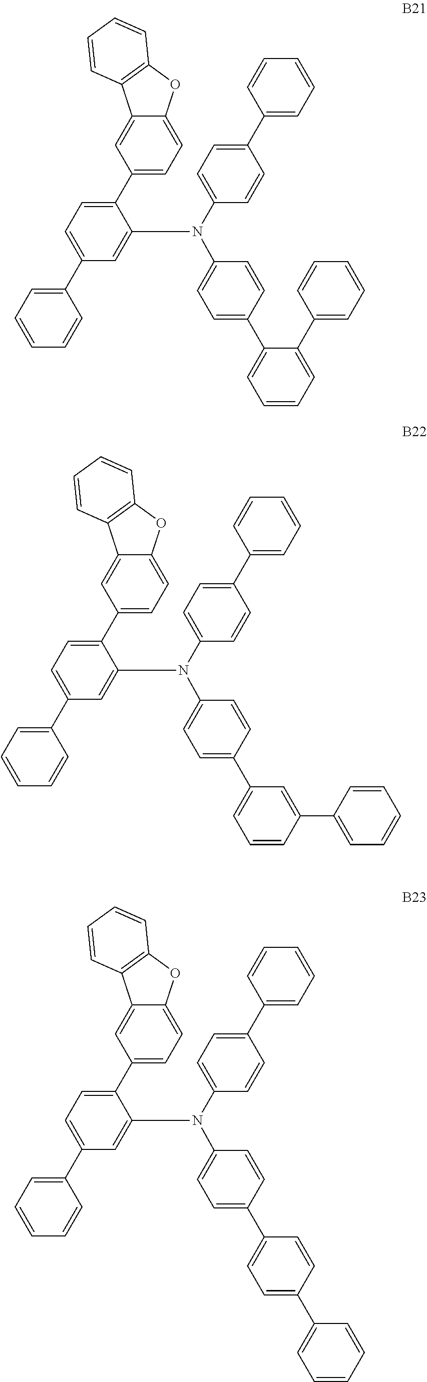 [SMILES: c1ccc(-c2ccc(N(c3ccc(-c4ccccc4-c4ccccc4)cc3)c3cc(-c4ccccc4)ccc3-c3ccc4oc5ccccc5c4c3)cc2)cc1 ; c1ccc(-c2ccc(N(c3ccc(-c4cccc(-c5ccccc5)c4)cc3)c3cc(-c4ccccc4)ccc3-c3ccc4oc5ccccc5c4c3)cc2)cc1 ; c1ccc(-c2ccc(-c3ccc(N(c4ccc(-c5ccccc5)cc4)c4cc(-c5ccccc5)ccc4-c4ccc5oc6ccccc6c5c4)cc3)cc2)cc1]

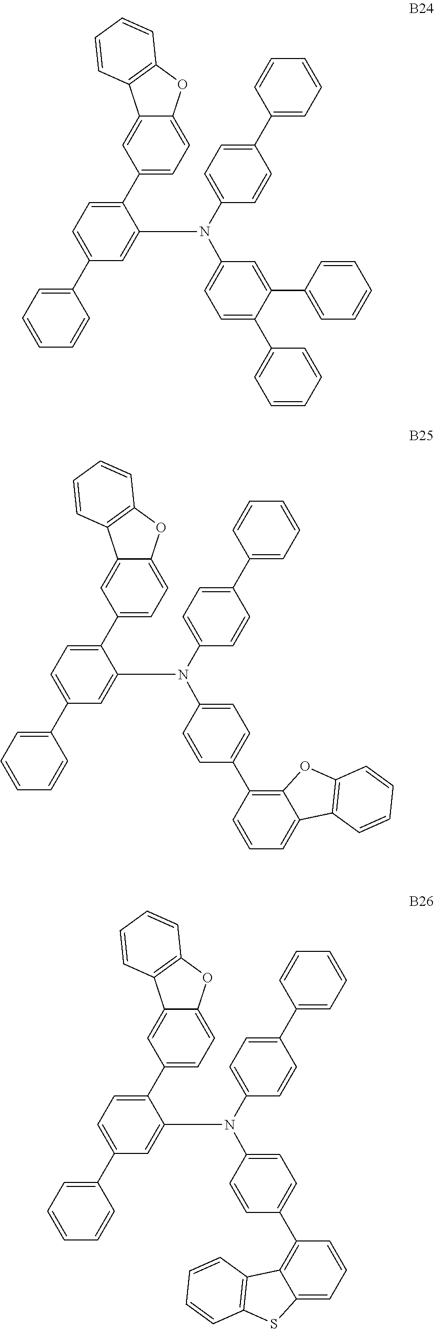 [SMILES: c1ccc(-c2ccc(N(c3ccc(-c4ccccc4)c(-c4ccccc4)c3)c3cc(-c4ccccc4)ccc3-c3ccc4oc5ccccc5c4c3)cc2)cc1 ; c1ccc(-c2ccc(N(c3ccc(-c4cccc5c4oc4ccccc45)cc3)c3cc(-c4ccccc4)ccc3-c3ccc4oc5ccccc5c4c3)cc2)cc1 ; c1ccc(-c2ccc(N(c3ccc(-c4cccc5sc6ccccc6c45)cc3)c3cc(-c4ccccc4)ccc3-c3ccc4oc5ccccc5c4c3)cc2)cc1]

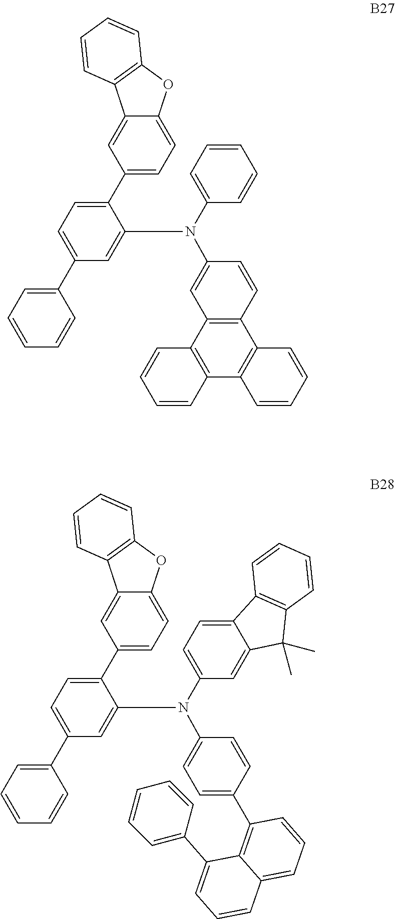 [SMILES: c1ccc(-c2ccc(-c3ccc4oc5ccccc5c4c3)c(N(c3ccccc3)c3ccc4c5ccccc5c5ccccc5c4c3)c2)cc1 ; CC1(C)c2ccccc2-c2ccc(N(c3ccc(-c4cccc5cccc(-c6ccccc6)c45)cc3)c3cc(-c4ccccc4)ccc3-c3ccc4oc5ccccc5c4c3)cc21]

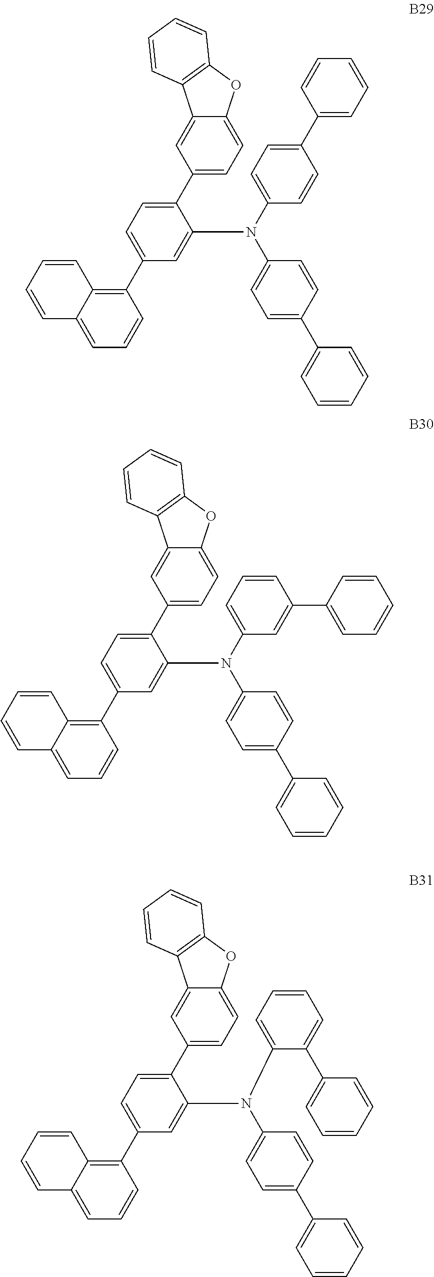 [SMILES: c1ccc(-c2ccc(N(c3ccc(-c4ccccc4)cc3)c3cc(-c4cccc5ccccc45)ccc3-c3ccc4oc5ccccc5c4c3)cc2)cc1 ; c1ccc(-c2ccc(N(c3cccc(-c4ccccc4)c3)c3cc(-c4cccc5ccccc45)ccc3-c3ccc4oc5ccccc5c4c3)cc2)cc1 ; c1ccc(-c2ccc(N(c3ccccc3-c3ccccc3)c3cc(-c4cccc5ccccc45)ccc3-c3ccc4oc5ccccc5c4c3)cc2)cc1]

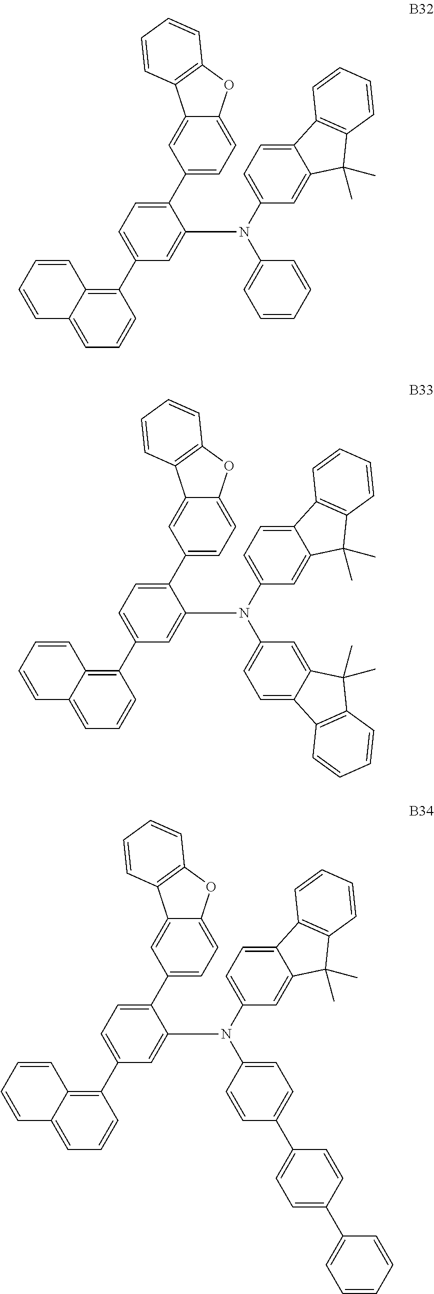 [SMILES: CC1(C)c2ccccc2-c2ccc(N(c3ccccc3)c3cc(-c4cccc5ccccc45)ccc3-c3ccc4oc5ccccc5c4c3)cc21 ; CC1(C)c2ccccc2-c2ccc(N(c3ccc4c(c3)C(C)(C)c3ccccc3-4)c3cc(-c4cccc5ccccc45)ccc3-c3ccc4oc5ccccc5c4c3)cc21 ; CC1(C)c2ccccc2-c2ccc(N(c3ccc(-c4ccc(-c5ccccc5)cc4)cc3)c3cc(-c4cccc5ccccc45)ccc3-c3ccc4oc5ccccc5c4c3)cc21]

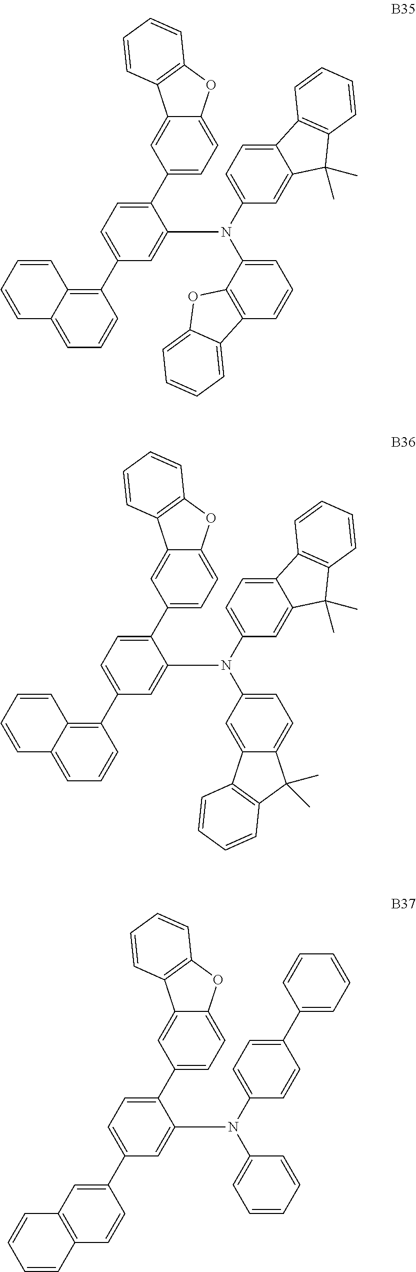 [SMILES: CC1(C)c2ccccc2-c2ccc(N(c3cc(-c4cccc5ccccc45)ccc3-c3ccc4oc5ccccc5c4c3)c3cccc4c3oc3ccccc34)cc21 ; CC1(C)c2ccccc2-c2cc(N(c3ccc4c(c3)C(C)(C)c3ccccc3-4)c3cc(-c4cccc5ccccc45)ccc3-c3ccc4oc5ccccc5c4c3)ccc21 ; c1ccc(-c2ccc(N(c3ccccc3)c3cc(-c4ccc5ccccc5c4)ccc3-c3ccc4oc5ccccc5c4c3)cc2)cc1]

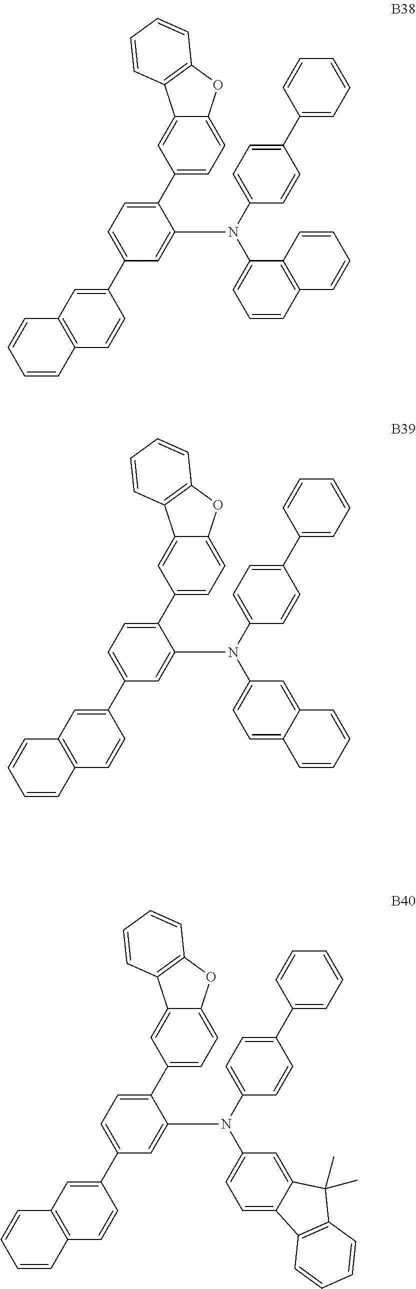 [SMILES: c1ccc(-c2ccc(N(c3cc(-c4ccc5ccccc5c4)ccc3-c3ccc4oc5ccccc5c4c3)c3cccc4ccccc34)cc2)cc1 ; c1ccc(-c2ccc(N(c3ccc4ccccc4c3)c3cc(-c4ccc5ccccc5c4)ccc3-c3ccc4oc5ccccc5c4c3)cc2)cc1 ; CC1(C)c2ccccc2-c2ccc(N(c3ccc(-c4ccccc4)cc3)c3cc(-c4ccc5ccccc5c4)ccc3-c3ccc4oc5ccccc5c4c3)cc21]

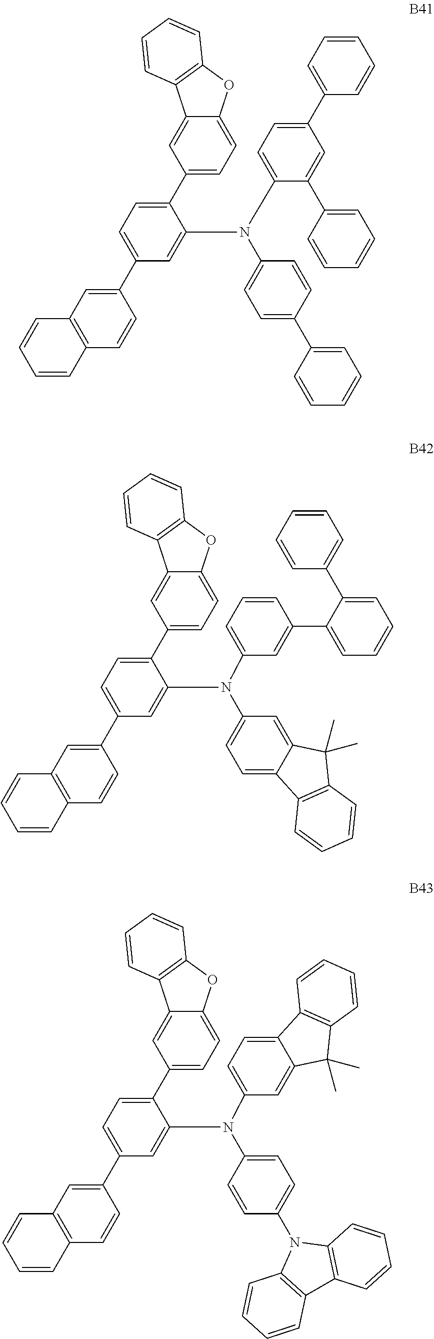 [SMILES: c1ccc(-c2ccc(N(c3ccc(-c4ccccc4)cc3-c3ccccc3)c3cc(-c4ccc5ccccc5c4)ccc3-c3ccc4oc5ccccc5c4c3)cc2)cc1 ; CC1(C)c2ccccc2-c2ccc(N(c3cccc(-c4ccccc4-c4ccccc4)c3)c3cc(-c4ccc5ccccc5c4)ccc3-c3ccc4oc5ccccc5c4c3)cc21 ; CC1(C)c2ccccc2-c2ccc(N(c3ccc(-n4c5ccccc5c5ccccc54)cc3)c3cc(-c4ccc5ccccc5c4)ccc3-c3ccc4oc5ccccc5c4c3)cc21]

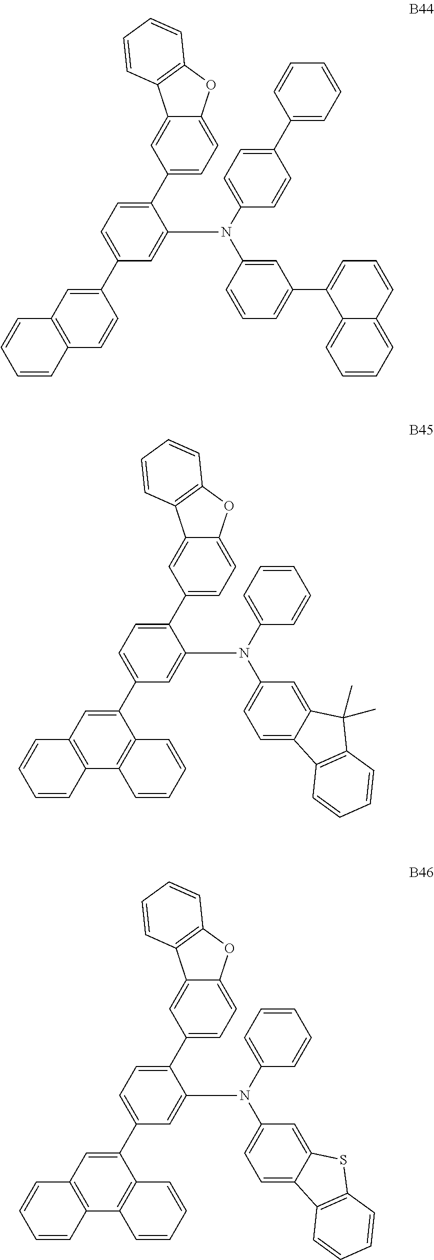 [SMILES: c1ccc(-c2ccc(N(c3cccc(-c4cccc5ccccc45)c3)c3cc(-c4ccc5ccccc5c4)ccc3-c3ccc4oc5ccccc5c4c3)cc2)cc1 ; CC1(C)c2ccccc2-c2ccc(N(c3ccccc3)c3cc(-c4cc5ccccc5c5ccccc45)ccc3-c3ccc4oc5ccccc5c4c3)cc21 ; c1ccc(N(c2ccc3c(c2)sc2ccccc23)c2cc(-c3cc4ccccc4c4ccccc34)ccc2-c2ccc3oc4ccccc4c3c2)cc1]

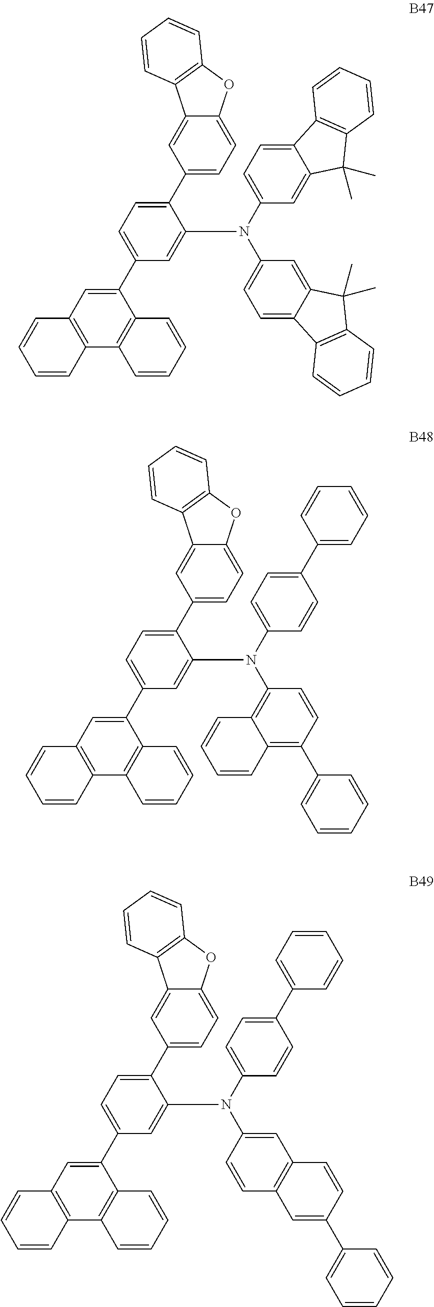 [SMILES: CC1(C)c2ccccc2-c2ccc(N(c3ccc4c(c3)C(C)(C)c3ccccc3-4)c3cc(-c4cc5ccccc5c5ccccc45)ccc3-c3ccc4oc5ccccc5c4c3)cc21 ; c1ccc(-c2ccc(N(c3cc(-c4cc5ccccc5c5ccccc45)ccc3-c3ccc4oc5ccccc5c4c3)c3ccc(-c4ccccc4)c4ccccc34)cc2)cc1 ; c1ccc(-c2ccc(N(c3ccc4cc(-c5ccccc5)ccc4c3)c3cc(-c4cc5ccccc5c5ccccc45)ccc3-c3ccc4oc5ccccc5c4c3)cc2)cc1]

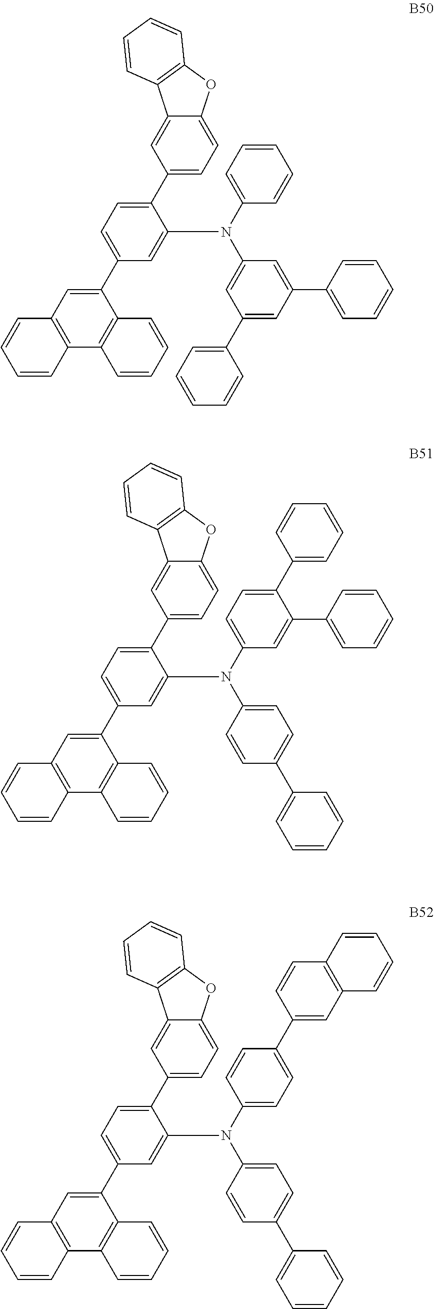 [SMILES: c1ccc(-c2cc(-c3ccccc3)cc(N(c3ccccc3)c3cc(-c4cc5ccccc5c5ccccc45)ccc3-c3ccc4oc5ccccc5c4c3)c2)cc1 ; c1ccc(-c2ccc(N(c3ccc(-c4ccccc4)c(-c4ccccc4)c3)c3cc(-c4cc5ccccc5c5ccccc45)ccc3-c3ccc4oc5ccccc5c4c3)cc2)cc1 ; c1ccc(-c2ccc(N(c3ccc(-c4ccc5ccccc5c4)cc3)c3cc(-c4cc5ccccc5c5ccccc45)ccc3-c3ccc4oc5ccccc5c4c3)cc2)cc1]

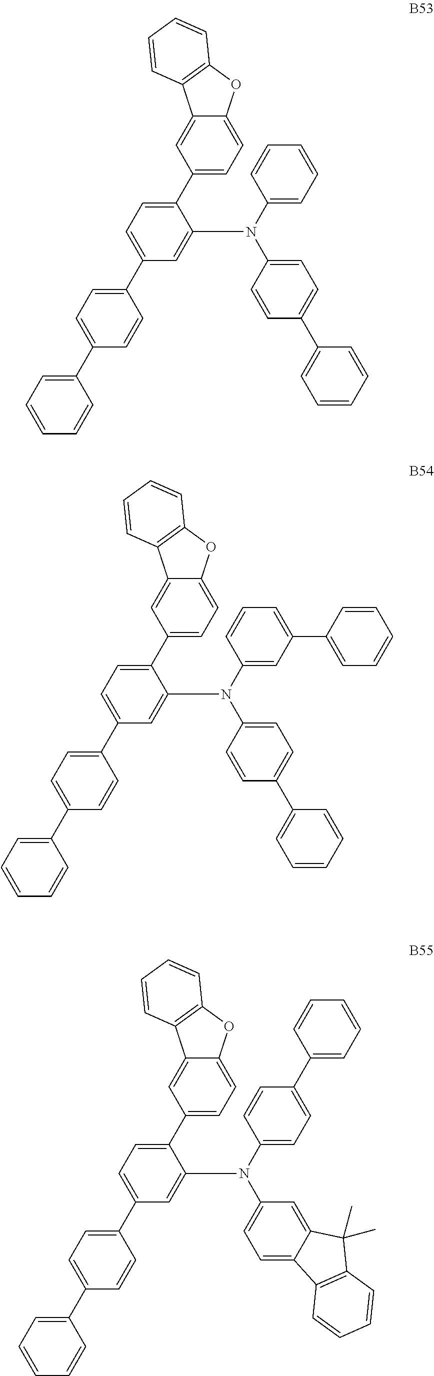 [SMILES: c1ccc(-c2ccc(-c3ccc(-c4ccc5oc6ccccc6c5c4)c(N(c4ccccc4)c4ccc(-c5ccccc5)cc4)c3)cc2)cc1 ; c1ccc(-c2ccc(-c3ccc(-c4ccc5oc6ccccc6c5c4)c(N(c4ccc(-c5ccccc5)cc4)c4cccc(-c5ccccc5)c4)c3)cc2)cc1 ; CC1(C)c2ccccc2-c2ccc(N(c3ccc(-c4ccccc4)cc3)c3cc(-c4ccc(-c5ccccc5)cc4)ccc3-c3ccc4oc5ccccc5c4c3)cc21]

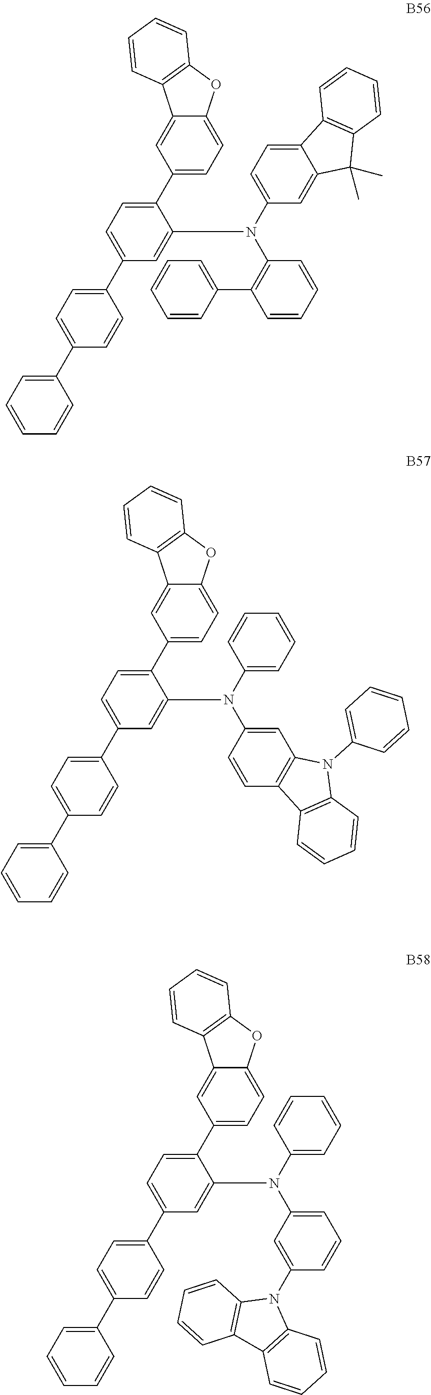 [SMILES: CC1(C)c2ccccc2-c2ccc(N(c3ccccc3-c3ccccc3)c3cc(-c4ccc(-c5ccccc5)cc4)ccc3-c3ccc4oc5ccccc5c4c3)cc21 ; c1ccc(-c2ccc(-c3ccc(-c4ccc5oc6ccccc6c5c4)c(N(c4ccccc4)c4ccc5c6ccccc6n(-c6ccccc6)c5c4)c3)cc2)cc1 ; c1ccc(-c2ccc(-c3ccc(-c4ccc5oc6ccccc6c5c4)c(N(c4ccccc4)c4cccc(-n5c6ccccc6c6ccccc65)c4)c3)cc2)cc1]

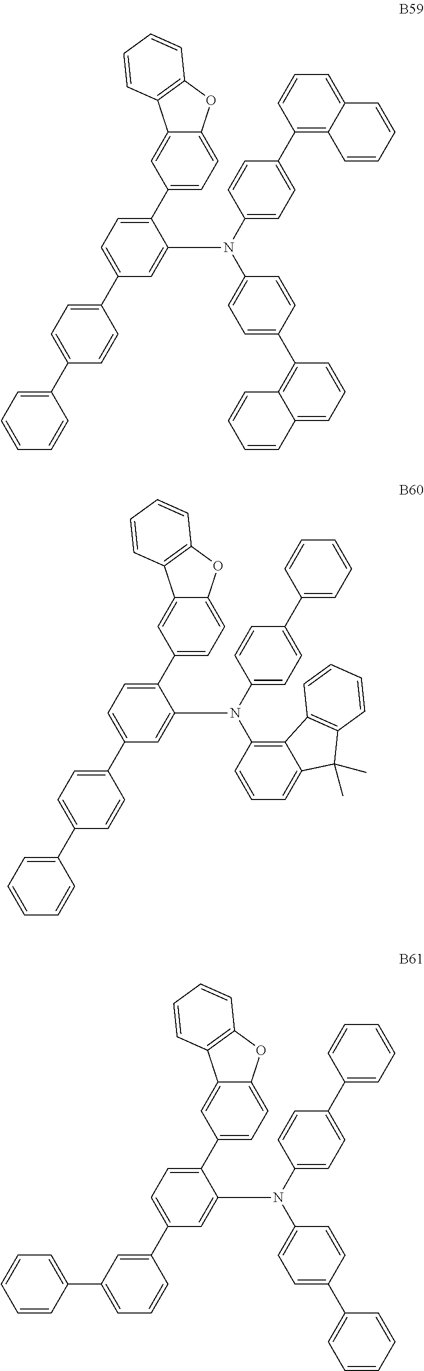 [SMILES: c1ccc(-c2ccc(-c3ccc(-c4ccc5oc6ccccc6c5c4)c(N(c4ccc(-c5cccc6ccccc56)cc4)c4ccc(-c5cccc6ccccc56)cc4)c3)cc2)cc1 ; CC1(C)c2ccccc2-c2c(N(c3ccc(-c4ccccc4)cc3)c3cc(-c4ccc(-c5ccccc5)cc4)ccc3-c3ccc4oc5ccccc5c4c3)cccc21 ; c1ccc(-c2ccc(N(c3ccc(-c4ccccc4)cc3)c3cc(-c4cccc(-c5ccccc5)c4)ccc3-c3ccc4oc5ccccc5c4c3)cc2)cc1]

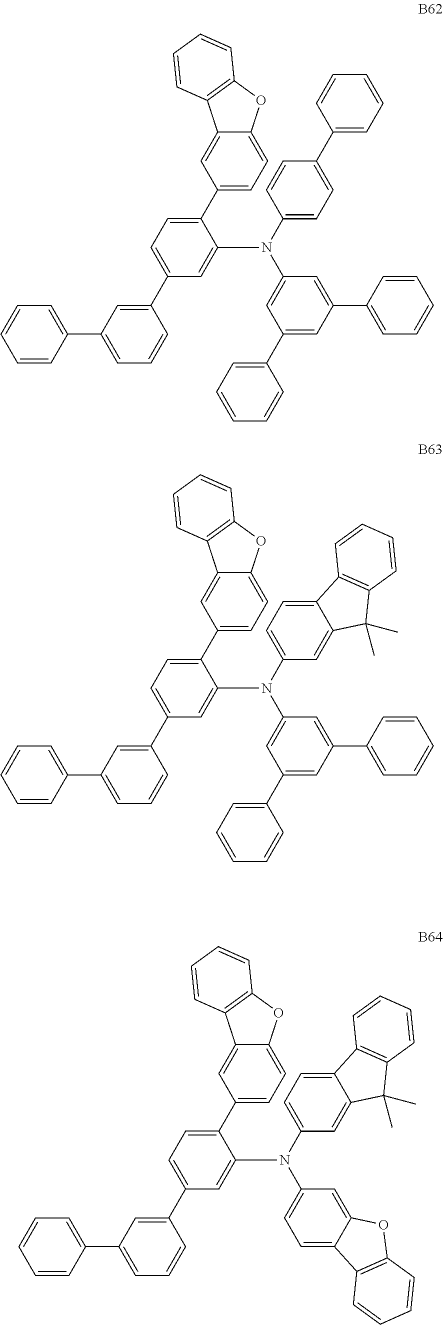 [SMILES: c1ccc(-c2ccc(N(c3cc(-c4ccccc4)cc(-c4ccccc4)c3)c3cc(-c4cccc(-c5ccccc5)c4)ccc3-c3ccc4oc5ccccc5c4c3)cc2)cc1 ; CC1(C)c2ccccc2-c2ccc(N(c3cc(-c4ccccc4)cc(-c4ccccc4)c3)c3cc(-c4cccc(-c5ccccc5)c4)ccc3-c3ccc4oc5ccccc5c4c3)cc21 ; CC1(C)c2ccccc2-c2ccc(N(c3ccc4c(c3)oc3ccccc34)c3cc(-c4cccc(-c5ccccc5)c4)ccc3-c3ccc4oc5ccccc5c4c3)cc21]

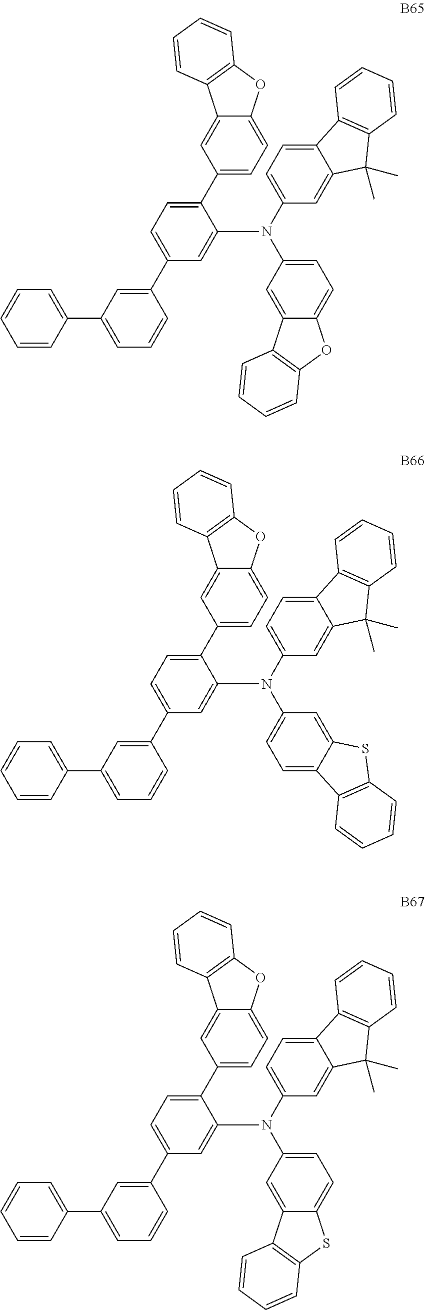 [SMILES: CC1(C)c2ccccc2-c2ccc(N(c3ccc4oc5ccccc5c4c3)c3cc(-c4cccc(-c5ccccc5)c4)ccc3-c3ccc4oc5ccccc5c4c3)cc21 ; CC1(C)c2ccccc2-c2ccc(N(c3ccc4c(c3)sc3ccccc34)c3cc(-c4cccc(-c5ccccc5)c4)ccc3-c3ccc4oc5ccccc5c4c3)cc21 ; CC1(C)c2ccccc2-c2ccc(N(c3ccc4sc5ccccc5c4c3)c3cc(-c4cccc(-c5ccccc5)c4)ccc3-c3ccc4oc5ccccc5c4c3)cc21]

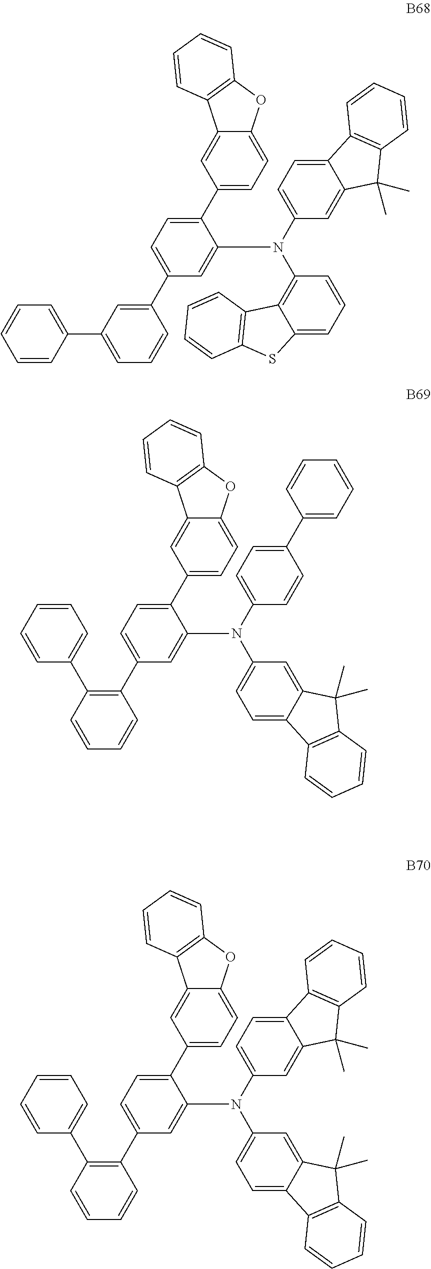 [SMILES: CC1(C)c2ccccc2-c2ccc(N(c3cc(-c4cccc(-c5ccccc5)c4)ccc3-c3ccc4oc5ccccc5c4c3)c3cccc4sc5ccccc5c34)cc21 ; CC1(C)c2ccccc2-c2ccc(N(c3ccc(-c4ccccc4)cc3)c3cc(-c4ccccc4-c4ccccc4)ccc3-c3ccc4oc5ccccc5c4c3)cc21 ; CC1(C)c2ccccc2-c2ccc(N(c3ccc4c(c3)C(C)(C)c3ccccc3-4)c3cc(-c4ccccc4-c4ccccc4)ccc3-c3ccc4oc5ccccc5c4c3)cc21]

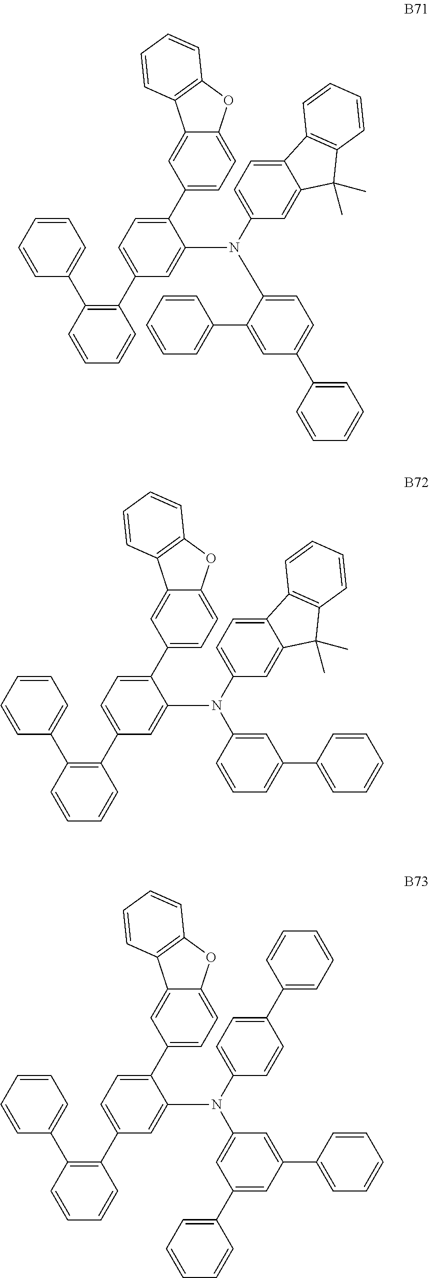 [SMILES: CC1(C)c2ccccc2-c2ccc(N(c3ccc(-c4ccccc4)cc3-c3ccccc3)c3cc(-c4ccccc4-c4ccccc4)ccc3-c3ccc4oc5ccccc5c4c3)cc21 ; CC1(C)c2ccccc2-c2ccc(N(c3cccc(-c4ccccc4)c3)c3cc(-c4ccccc4-c4ccccc4)ccc3-c3ccc4oc5ccccc5c4c3)cc21 ; c1ccc(-c2ccc(N(c3cc(-c4ccccc4)cc(-c4ccccc4)c3)c3cc(-c4ccccc4-c4ccccc4)ccc3-c3ccc4oc5ccccc5c4c3)cc2)cc1]

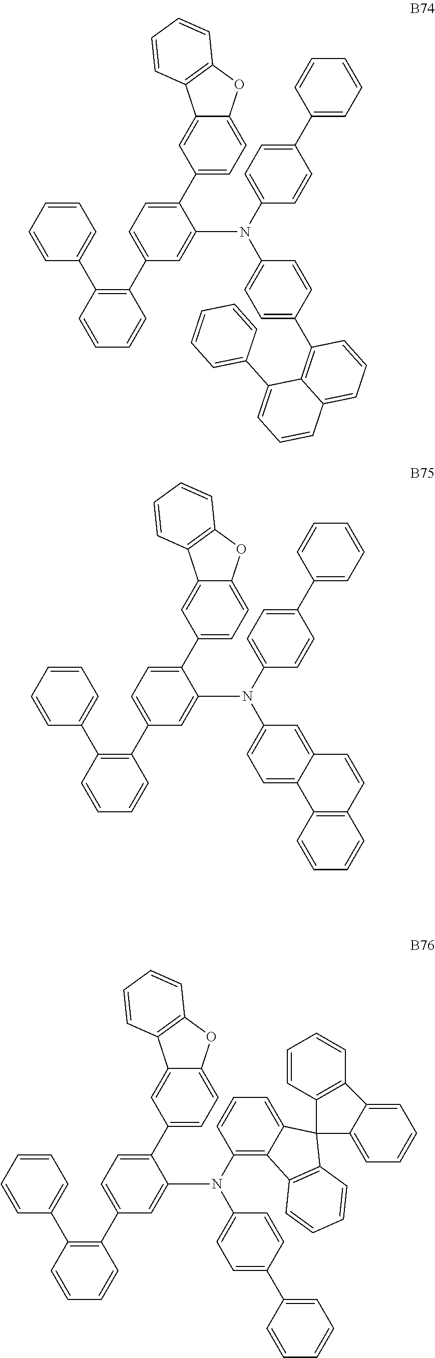 [SMILES: c1ccc(-c2ccc(N(c3ccc(-c4cccc5cccc(-c6ccccc6)c45)cc3)c3cc(-c4ccccc4-c4ccccc4)ccc3-c3ccc4oc5ccccc5c4c3)cc2)cc1 ; c1ccc(-c2ccc(N(c3ccc4c(ccc5ccccc54)c3)c3cc(-c4ccccc4-c4ccccc4)ccc3-c3ccc4oc5ccccc5c4c3)cc2)cc1 ; c1ccc(-c2ccc(N(c3cc(-c4ccccc4-c4ccccc4)ccc3-c3ccc4oc5ccccc5c4c3)c3cccc4c3-c3ccccc3C43c4ccccc4-c4ccccc43)cc2)cc1]

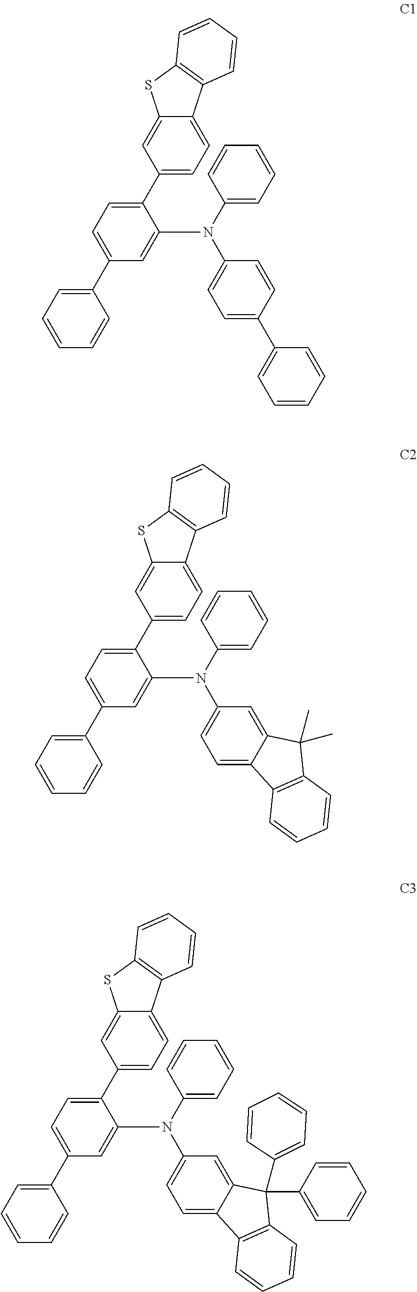 [SMILES: c1ccc(-c2ccc(N(c3ccccc3)c3cc(-c4ccccc4)ccc3-c3ccc4c(c3)sc3ccccc34)cc2)cc1 ; CC1(C)c2ccccc2-c2ccc(N(c3ccccc3)c3cc(-c4ccccc4)ccc3-c3ccc4c(c3)sc3ccccc34)cc21 ; c1ccc(-c2ccc(-c3ccc4c(c3)sc3ccccc34)c(N(c3ccccc3)c3ccc4c(c3)C(c3ccccc3)(c3ccccc3)c3ccccc3-4)c2)cc1]

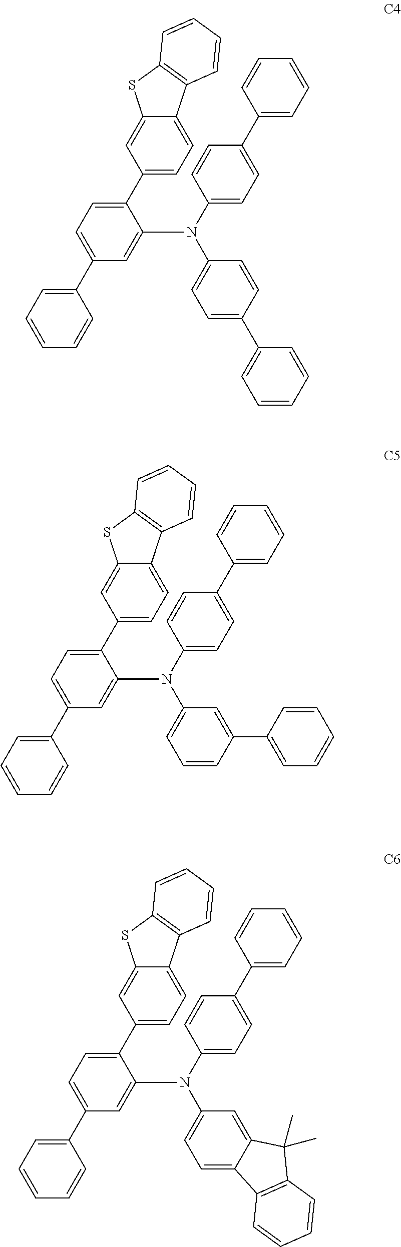 [SMILES: c1ccc(-c2ccc(N(c3ccc(-c4ccccc4)cc3)c3cc(-c4ccccc4)ccc3-c3ccc4c(c3)sc3ccccc34)cc2)cc1 ; c1ccc(-c2ccc(N(c3cccc(-c4ccccc4)c3)c3cc(-c4ccccc4)ccc3-c3ccc4c(c3)sc3ccccc34)cc2)cc1 ; CC1(C)c2ccccc2-c2ccc(N(c3ccc(-c4ccccc4)cc3)c3cc(-c4ccccc4)ccc3-c3ccc4c(c3)sc3ccccc34)cc21]

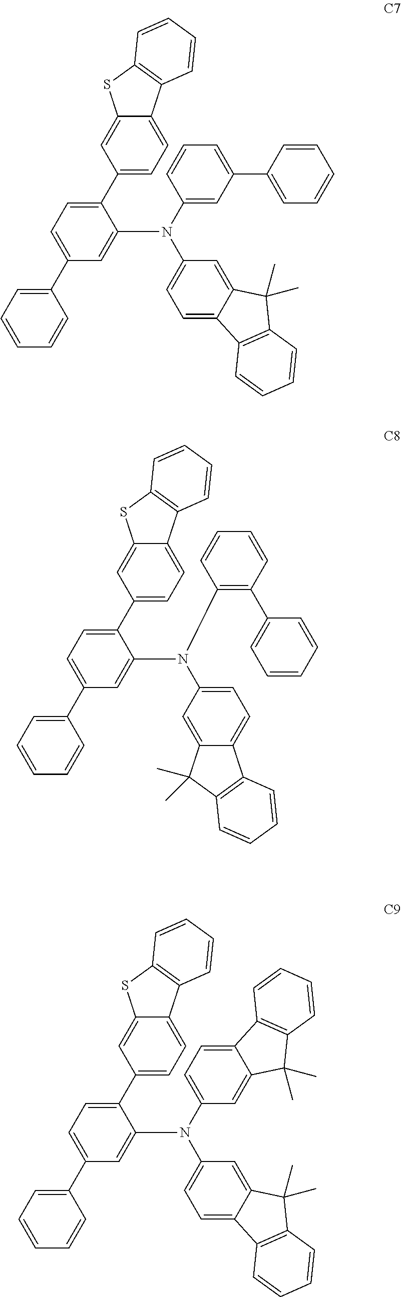 [SMILES: CC1(C)c2ccccc2-c2ccc(N(c3cccc(-c4ccccc4)c3)c3cc(-c4ccccc4)ccc3-c3ccc4c(c3)sc3ccccc34)cc21 ; CC1(C)c2ccccc2-c2ccc(N(c3ccccc3-c3ccccc3)c3cc(-c4ccccc4)ccc3-c3ccc4c(c3)sc3ccccc34)cc21 ; CC1(C)c2ccccc2-c2ccc(N(c3ccc4c(c3)C(C)(C)c3ccccc3-4)c3cc(-c4ccccc4)ccc3-c3ccc4c(c3)sc3ccccc34)cc21]

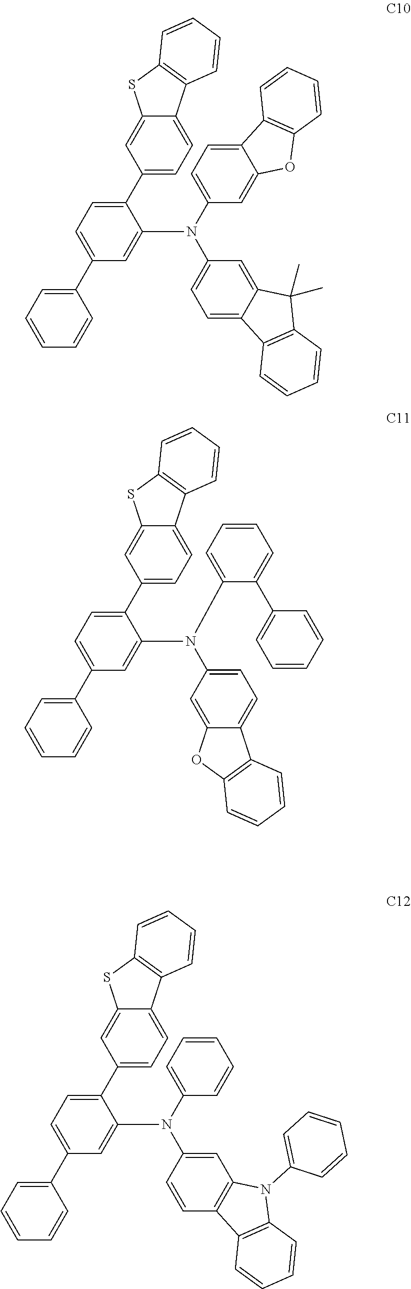 [SMILES: CC1(C)c2ccccc2-c2ccc(N(c3ccc4c(c3)oc3ccccc34)c3cc(-c4ccccc4)ccc3-c3ccc4c(c3)sc3ccccc34)cc21 ; c1ccc(-c2ccc(-c3ccc4c(c3)sc3ccccc34)c(N(c3ccc4c(c3)oc3ccccc34)c3ccccc3-c3ccccc3)c2)cc1 ; c1ccc(-c2ccc(-c3ccc4c(c3)sc3ccccc34)c(N(c3ccccc3)c3ccc4c5ccccc5n(-c5ccccc5)c4c3)c2)cc1]

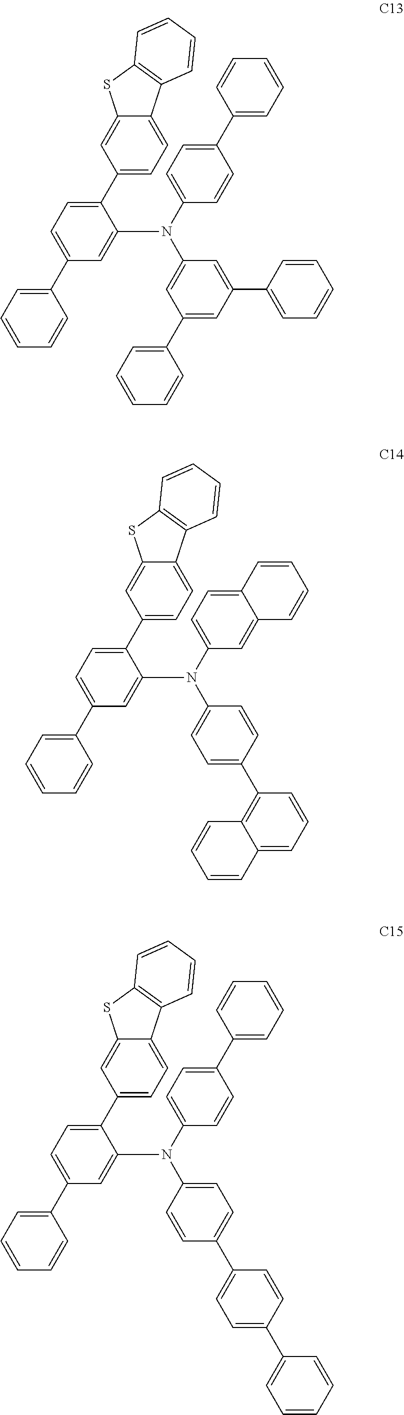 [SMILES: c1ccc(-c2ccc(N(c3cc(-c4ccccc4)cc(-c4ccccc4)c3)c3cc(-c4ccccc4)ccc3-c3ccc4c(c3)sc3ccccc34)cc2)cc1 ; c1ccc(-c2ccc(-c3ccc4c(c3)sc3ccccc34)c(N(c3ccc(-c4cccc5ccccc45)cc3)c3ccc4ccccc4c3)c2)cc1 ; c1ccc(-c2ccc(-c3ccc(N(c4ccc(-c5ccccc5)cc4)c4cc(-c5ccccc5)ccc4-c4ccc5c(c4)sc4ccccc45)cc3)cc2)cc1]

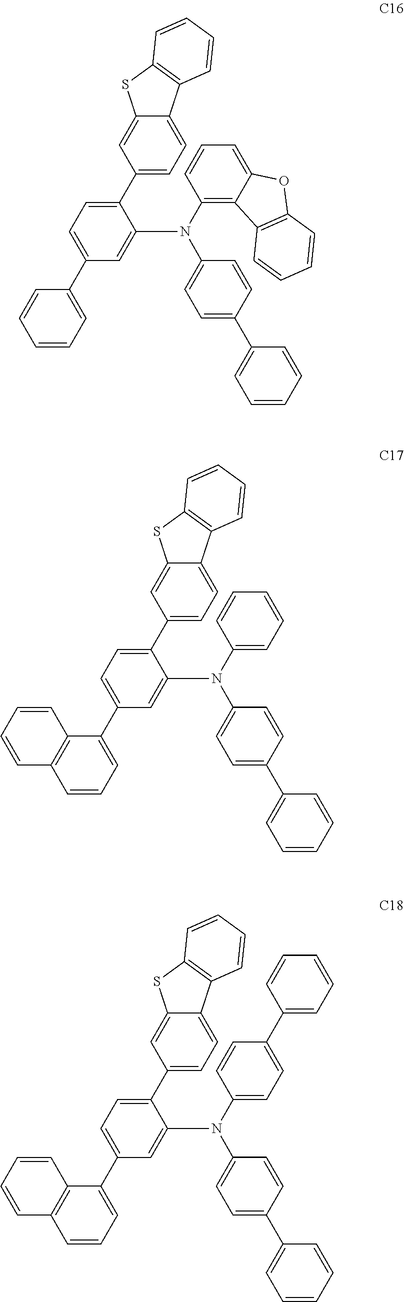 [SMILES: c1ccc(-c2ccc(N(c3cc(-c4ccccc4)ccc3-c3ccc4c(c3)sc3ccccc34)c3cccc4oc5ccccc5c34)cc2)cc1 ; c1ccc(-c2ccc(N(c3ccccc3)c3cc(-c4cccc5ccccc45)ccc3-c3ccc4c(c3)sc3ccccc34)cc2)cc1 ; c1ccc(-c2ccc(N(c3ccc(-c4ccccc4)cc3)c3cc(-c4cccc5ccccc45)ccc3-c3ccc4c(c3)sc3ccccc34)cc2)cc1]

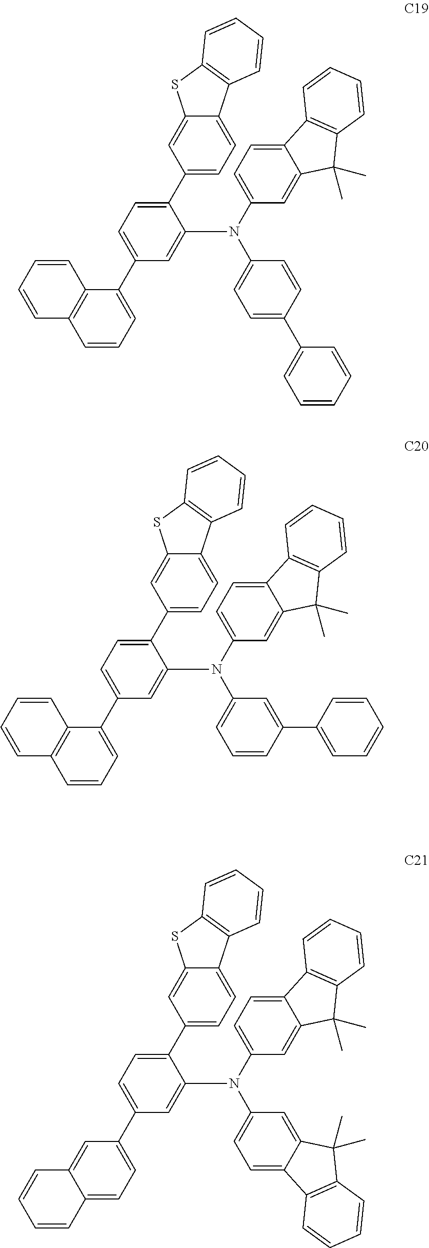 [SMILES: CC1(C)c2ccccc2-c2ccc(N(c3ccc(-c4ccccc4)cc3)c3cc(-c4cccc5ccccc45)ccc3-c3ccc4c(c3)sc3ccccc34)cc21 ; CC1(C)c2ccccc2-c2ccc(N(c3cccc(-c4ccccc4)c3)c3cc(-c4cccc5ccccc45)ccc3-c3ccc4c(c3)sc3ccccc34)cc21 ; CC1(C)c2ccccc2-c2ccc(N(c3ccc4c(c3)C(C)(C)c3ccccc3-4)c3cc(-c4ccc5ccccc5c4)ccc3-c3ccc4c(c3)sc3ccccc34)cc21]

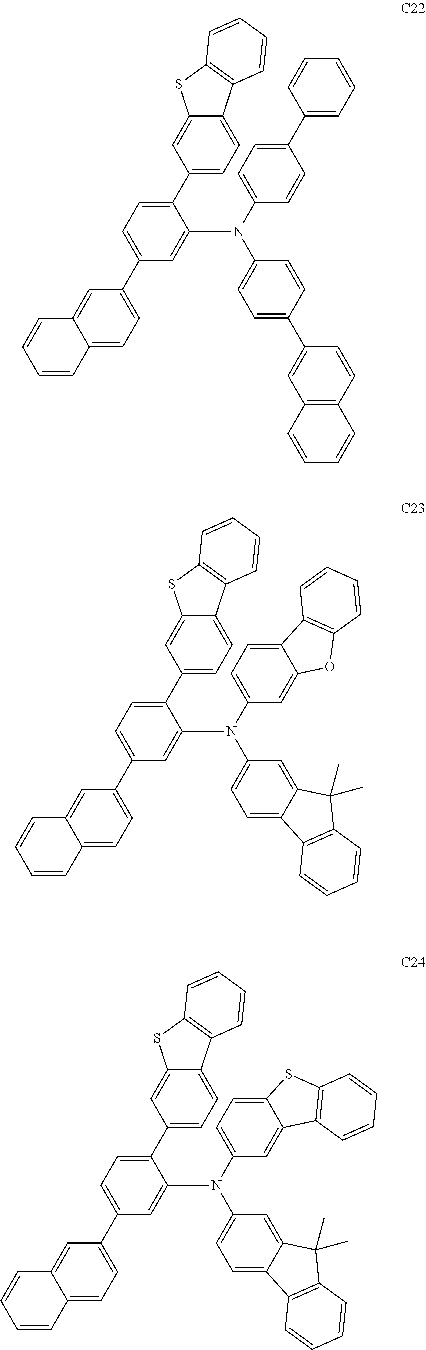 [SMILES: c1ccc(-c2ccc(N(c3ccc(-c4ccc5ccccc5c4)cc3)c3cc(-c4ccc5ccccc5c4)ccc3-c3ccc4c(c3)sc3ccccc34)cc2)cc1 ; CC1(C)c2ccccc2-c2ccc(N(c3ccc4c(c3)oc3ccccc34)c3cc(-c4ccc5ccccc5c4)ccc3-c3ccc4c(c3)sc3ccccc34)cc21 ; CC1(C)c2ccccc2-c2ccc(N(c3ccc4sc5ccccc5c4c3)c3cc(-c4ccc5ccccc5c4)ccc3-c3ccc4c(c3)sc3ccccc34)cc21]

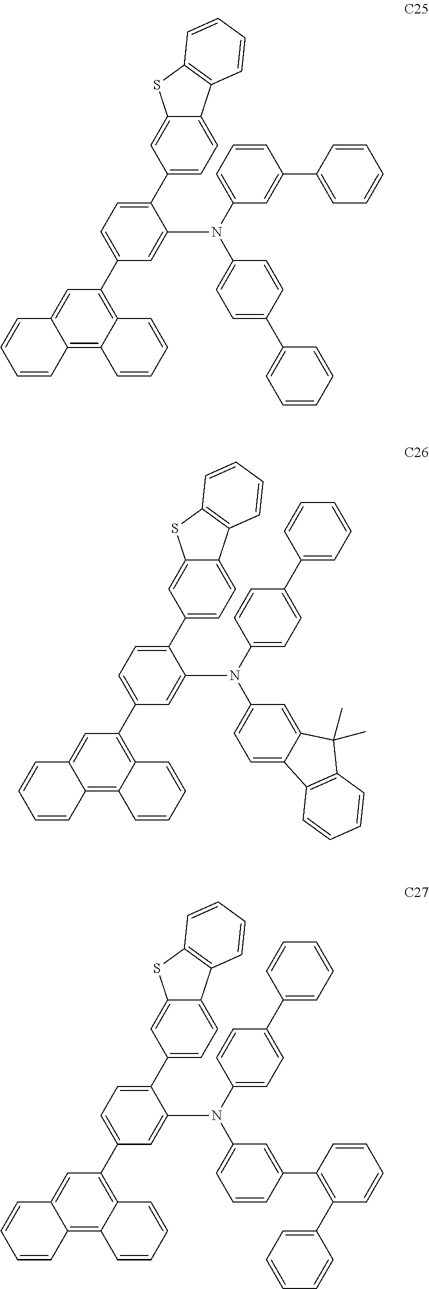 [SMILES: c1ccc(-c2ccc(N(c3cccc(-c4ccccc4)c3)c3cc(-c4cc5ccccc5c5ccccc45)ccc3-c3ccc4c(c3)sc3ccccc34)cc2)cc1 ; CC1(C)c2ccccc2-c2ccc(N(c3ccc(-c4ccccc4)cc3)c3cc(-c4cc5ccccc5c5ccccc45)ccc3-c3ccc4c(c3)sc3ccccc34)cc21 ; c1ccc(-c2ccc(N(c3cccc(-c4ccccc4-c4ccccc4)c3)c3cc(-c4cc5ccccc5c5ccccc45)ccc3-c3ccc4c(c3)sc3ccccc34)cc2)cc1]

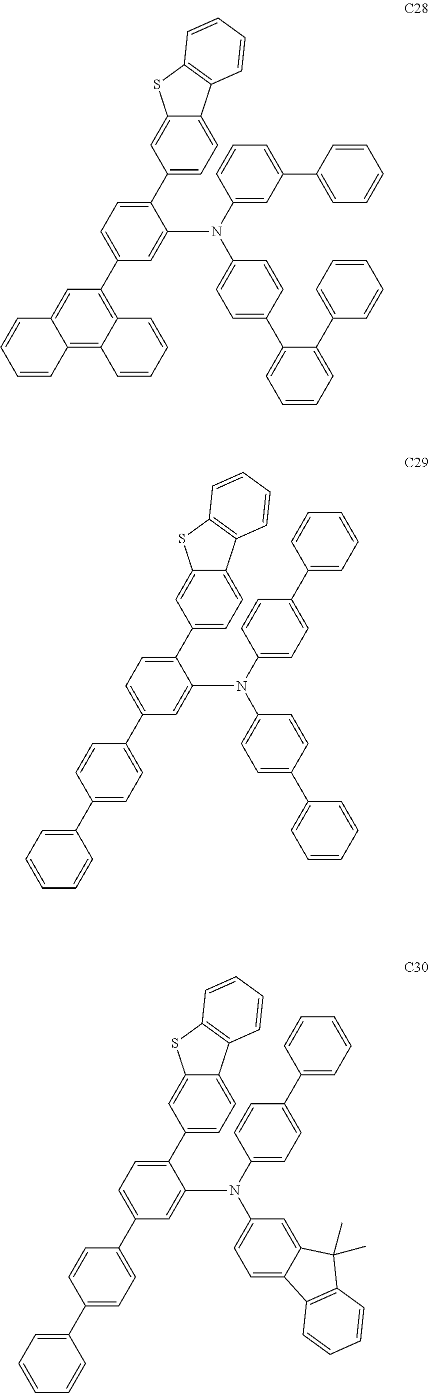 [SMILES: c1ccc(-c2cccc(N(c3ccc(-c4ccccc4-c4ccccc4)cc3)c3cc(-c4cc5ccccc5c5ccccc45)ccc3-c3ccc4c(c3)sc3ccccc34)c2)cc1 ; c1ccc(-c2ccc(-c3ccc(-c4ccc5c(c4)sc4ccccc45)c(N(c4ccc(-c5ccccc5)cc4)c4ccc(-c5ccccc5)cc4)c3)cc2)cc1 ; CC1(C)c2ccccc2-c2ccc(N(c3ccc(-c4ccccc4)cc3)c3cc(-c4ccc(-c5ccccc5)cc4)ccc3-c3ccc4c(c3)sc3ccccc34)cc21]

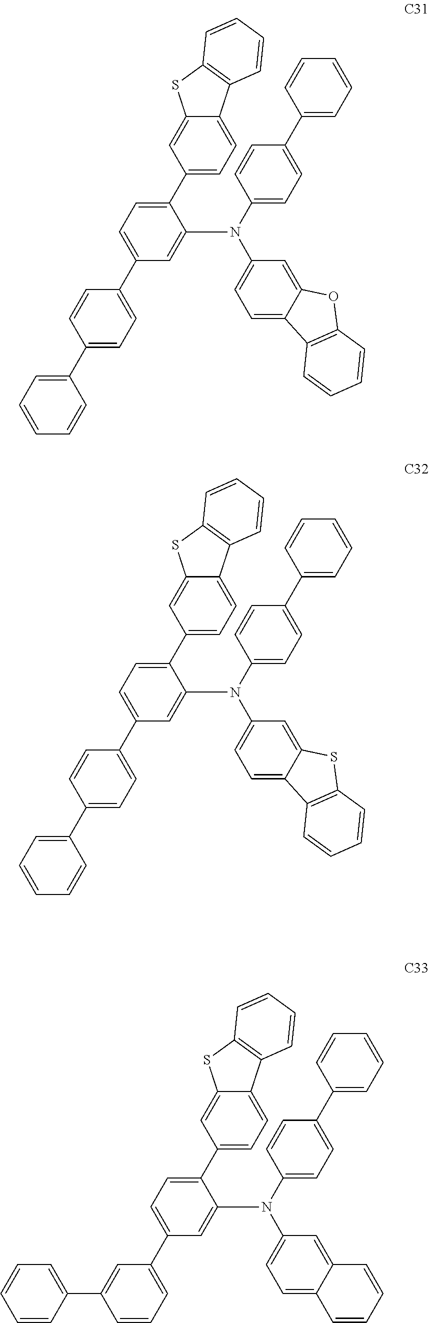 [SMILES: c1ccc(-c2ccc(-c3ccc(-c4ccc5c(c4)sc4ccccc45)c(N(c4ccc(-c5ccccc5)cc4)c4ccc5c(c4)oc4ccccc45)c3)cc2)cc1 ; c1ccc(-c2ccc(-c3ccc(-c4ccc5c(c4)sc4ccccc45)c(N(c4ccc(-c5ccccc5)cc4)c4ccc5c(c4)sc4ccccc45)c3)cc2)cc1 ; c1ccc(-c2ccc(N(c3ccc4ccccc4c3)c3cc(-c4cccc(-c5ccccc5)c4)ccc3-c3ccc4c(c3)sc3ccccc34)cc2)cc1]

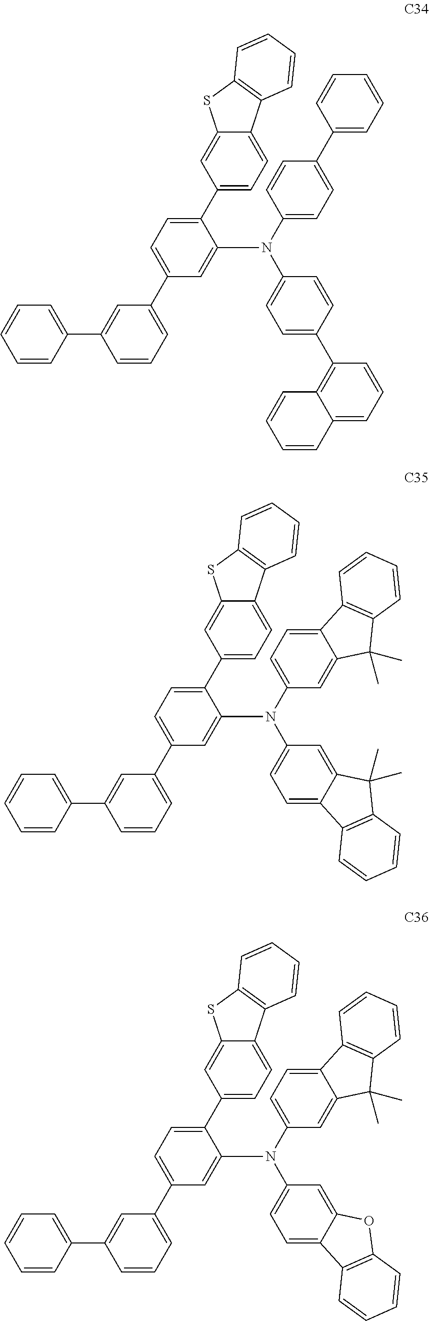 [SMILES: c1ccc(-c2ccc(N(c3ccc(-c4cccc5ccccc45)cc3)c3cc(-c4cccc(-c5ccccc5)c4)ccc3-c3ccc4c(c3)sc3ccccc34)cc2)cc1 ; CC1(C)c2ccccc2-c2ccc(N(c3ccc4c(c3)C(C)(C)c3ccccc3-4)c3cc(-c4cccc(-c5ccccc5)c4)ccc3-c3ccc4c(c3)sc3ccccc34)cc21 ; CC1(C)c2ccccc2-c2ccc(N(c3ccc4c(c3)oc3ccccc34)c3cc(-c4cccc(-c5ccccc5)c4)ccc3-c3ccc4c(c3)sc3ccccc34)cc21]

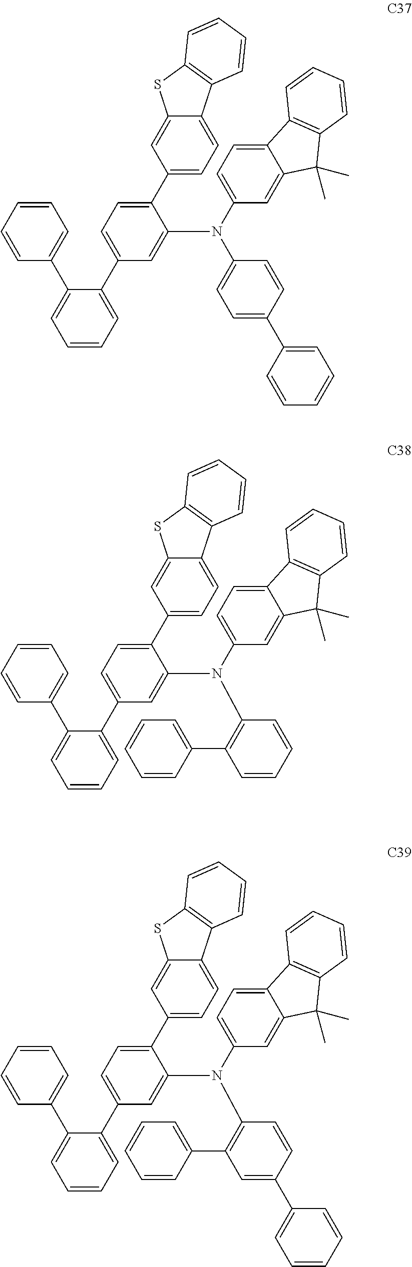 [SMILES: CC1(C)c2ccccc2-c2ccc(N(c3ccc(-c4ccccc4)cc3)c3cc(-c4ccccc4-c4ccccc4)ccc3-c3ccc4c(c3)sc3ccccc34)cc21 ; CC1(C)c2ccccc2-c2ccc(N(c3ccccc3-c3ccccc3)c3cc(-c4ccccc4-c4ccccc4)ccc3-c3ccc4c(c3)sc3ccccc34)cc21 ; CC1(C)c2ccccc2-c2ccc(N(c3ccc(-c4ccccc4)cc3-c3ccccc3)c3cc(-c4ccccc4-c4ccccc4)ccc3-c3ccc4c(c3)sc3ccccc34)cc21]

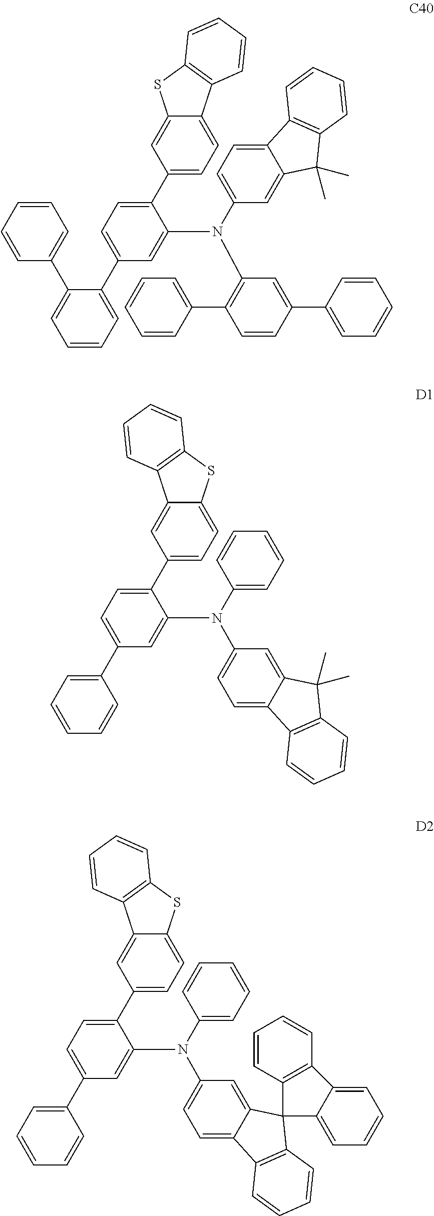 [SMILES: CC1(C)c2ccccc2-c2ccc(N(c3cc(-c4ccccc4)ccc3-c3ccccc3)c3cc(-c4ccccc4-c4ccccc4)ccc3-c3ccc4c(c3)sc3ccccc34)cc21 ; CC1(C)c2ccccc2-c2ccc(N(c3ccccc3)c3cc(-c4ccccc4)ccc3-c3ccc4sc5ccccc5c4c3)cc21 ; c1ccc(-c2ccc(-c3ccc4sc5ccccc5c4c3)c(N(c3ccccc3)c3ccc4c(c3)C3(c5ccccc5-c5ccccc53)c3ccccc3-4)c2)cc1]

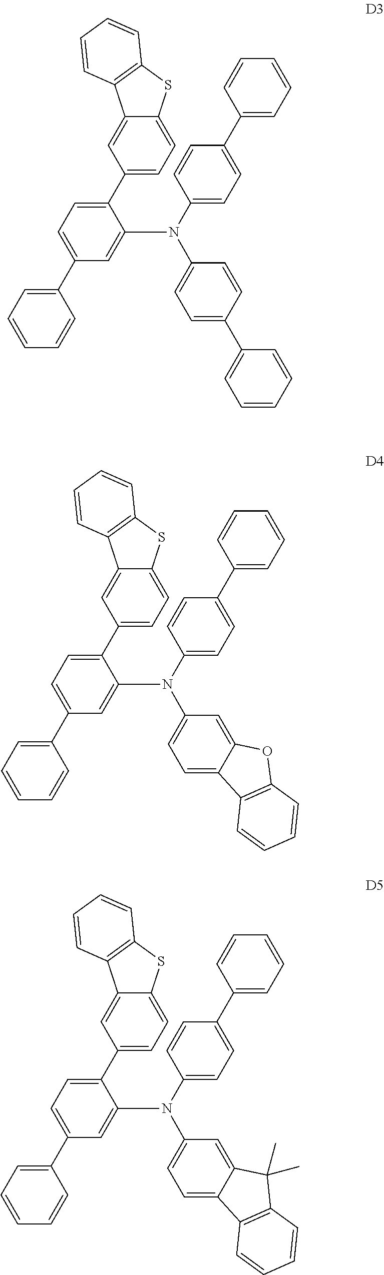 [SMILES: c1ccc(-c2ccc(N(c3ccc(-c4ccccc4)cc3)c3cc(-c4ccccc4)ccc3-c3ccc4sc5ccccc5c4c3)cc2)cc1 ; c1ccc(-c2ccc(N(c3ccc4c(c3)oc3ccccc34)c3cc(-c4ccccc4)ccc3-c3ccc4sc5ccccc5c4c3)cc2)cc1 ; CC1(C)c2ccccc2-c2ccc(N(c3ccc(-c4ccccc4)cc3)c3cc(-c4ccccc4)ccc3-c3ccc4sc5ccccc5c4c3)cc21]

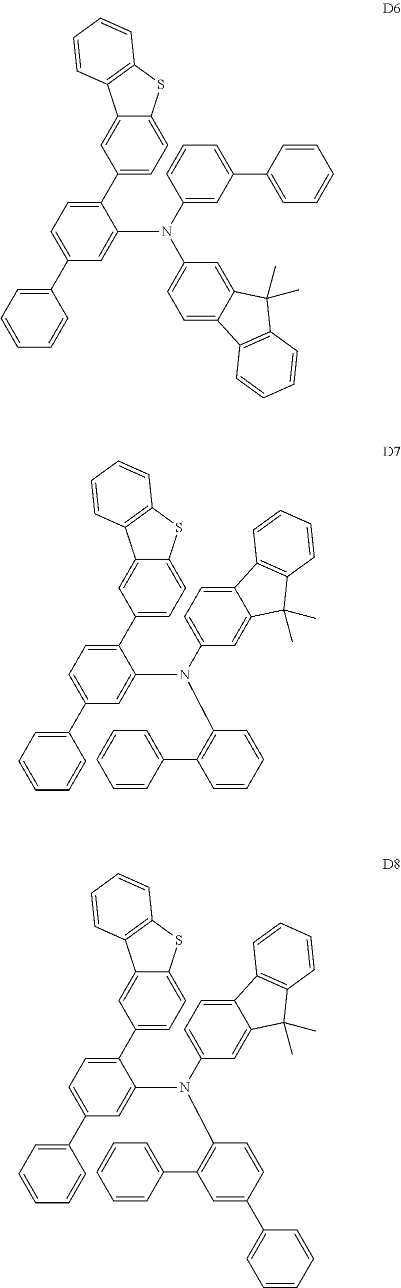 [SMILES: CC1(C)c2ccccc2-c2ccc(N(c3cccc(-c4ccccc4)c3)c3cc(-c4ccccc4)ccc3-c3ccc4sc5ccccc5c4c3)cc21 ; CC1(C)c2ccccc2-c2ccc(N(c3ccccc3-c3ccccc3)c3cc(-c4ccccc4)ccc3-c3ccc4sc5ccccc5c4c3)cc21 ; CC1(C)c2ccccc2-c2ccc(N(c3ccc(-c4ccccc4)cc3-c3ccccc3)c3cc(-c4ccccc4)ccc3-c3ccc4sc5ccccc5c4c3)cc21]

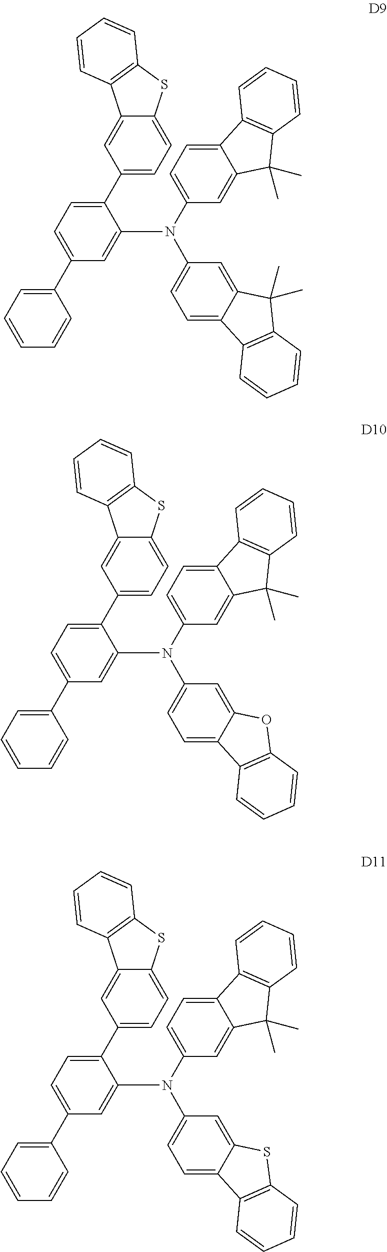 [SMILES: CC1(C)c2ccccc2-c2ccc(N(c3ccc4c(c3)C(C)(C)c3ccccc3-4)c3cc(-c4ccccc4)ccc3-c3ccc4sc5ccccc5c4c3)cc21 ; CC1(C)c2ccccc2-c2ccc(N(c3ccc4c(c3)oc3ccccc34)c3cc(-c4ccccc4)ccc3-c3ccc4sc5ccccc5c4c3)cc21 ; CC1(C)c2ccccc2-c2ccc(N(c3ccc4c(c3)sc3ccccc34)c3cc(-c4ccccc4)ccc3-c3ccc4sc5ccccc5c4c3)cc21]

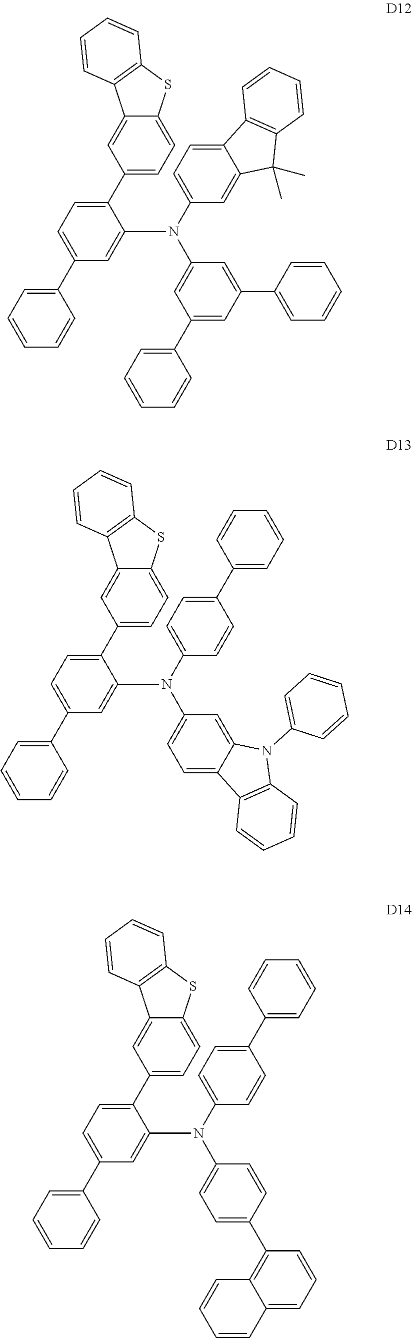 [SMILES: CC1(C)c2ccccc2-c2ccc(N(c3cc(-c4ccccc4)cc(-c4ccccc4)c3)c3cc(-c4ccccc4)ccc3-c3ccc4sc5ccccc5c4c3)cc21 ; c1ccc(-c2ccc(N(c3ccc4c5ccccc5n(-c5ccccc5)c4c3)c3cc(-c4ccccc4)ccc3-c3ccc4sc5ccccc5c4c3)cc2)cc1 ; c1ccc(-c2ccc(N(c3ccc(-c4cccc5ccccc45)cc3)c3cc(-c4ccccc4)ccc3-c3ccc4sc5ccccc5c4c3)cc2)cc1]

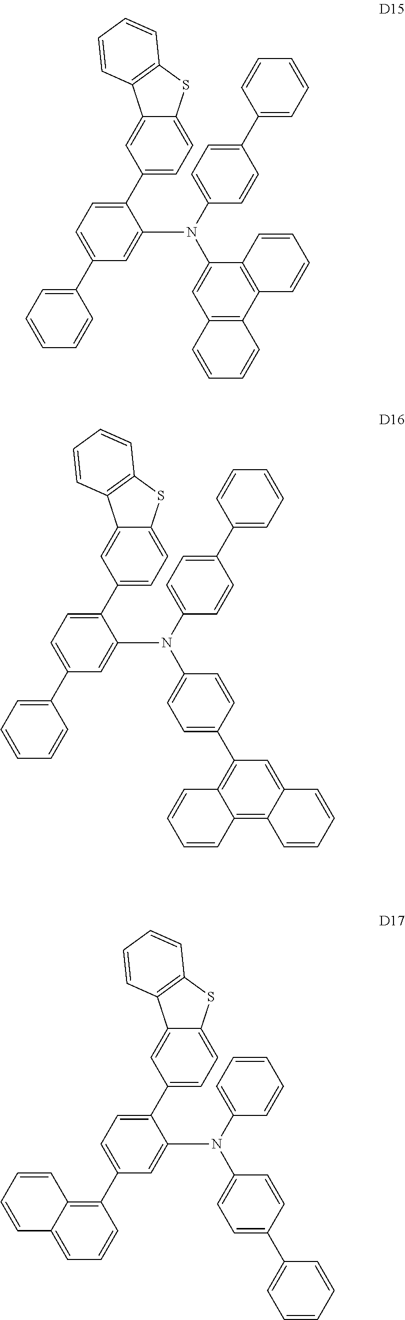 [SMILES: c1ccc(-c2ccc(N(c3cc(-c4ccccc4)ccc3-c3ccc4sc5ccccc5c4c3)c3cc4ccccc4c4ccccc34)cc2)cc1 ; c1ccc(-c2ccc(N(c3ccc(-c4cc5ccccc5c5ccccc45)cc3)c3cc(-c4ccccc4)ccc3-c3ccc4sc5ccccc5c4c3)cc2)cc1 ; c1ccc(-c2ccc(N(c3ccccc3)c3cc(-c4cccc5ccccc45)ccc3-c3ccc4sc5ccccc5c4c3)cc2)cc1]

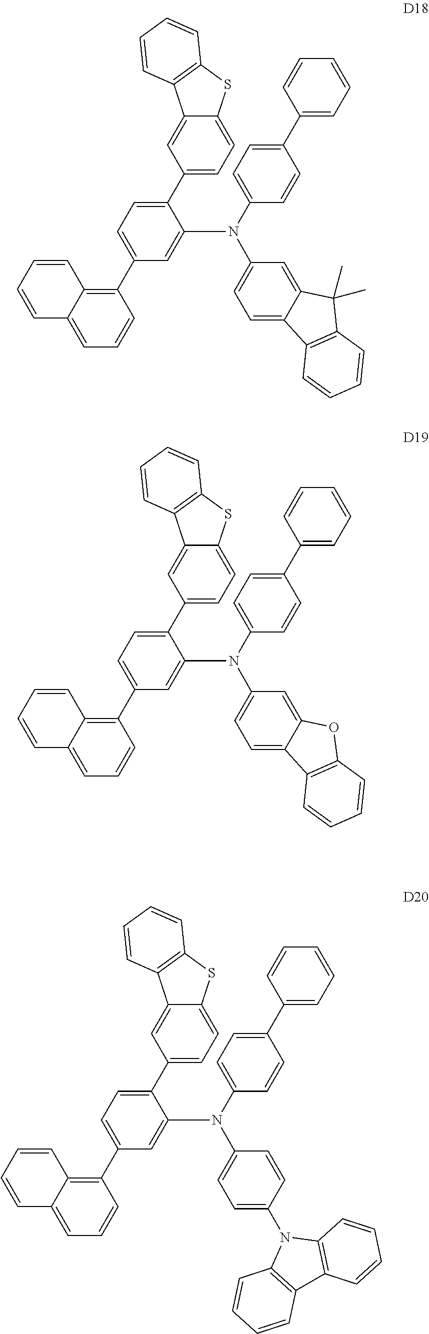 [SMILES: CC1(C)c2ccccc2-c2ccc(N(c3ccc(-c4ccccc4)cc3)c3cc(-c4cccc5ccccc45)ccc3-c3ccc4sc5ccccc5c4c3)cc21 ; c1ccc(-c2ccc(N(c3ccc4c(c3)oc3ccccc34)c3cc(-c4cccc5ccccc45)ccc3-c3ccc4sc5ccccc5c4c3)cc2)cc1 ; c1ccc(-c2ccc(N(c3ccc(-n4c5ccccc5c5ccccc54)cc3)c3cc(-c4cccc5ccccc45)ccc3-c3ccc4sc5ccccc5c4c3)cc2)cc1]

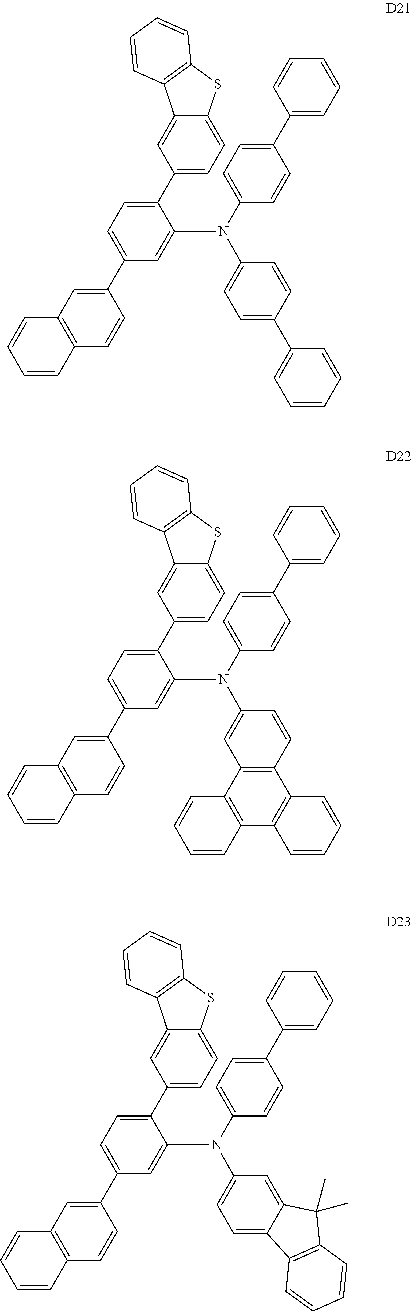 [SMILES: c1ccc(-c2ccc(N(c3ccc(-c4ccccc4)cc3)c3cc(-c4ccc5ccccc5c4)ccc3-c3ccc4sc5ccccc5c4c3)cc2)cc1 ; c1ccc(-c2ccc(N(c3ccc4c5ccccc5c5ccccc5c4c3)c3cc(-c4ccc5ccccc5c4)ccc3-c3ccc4sc5ccccc5c4c3)cc2)cc1 ; CC1(C)c2ccccc2-c2ccc(N(c3ccc(-c4ccccc4)cc3)c3cc(-c4ccc5ccccc5c4)ccc3-c3ccc4sc5ccccc5c4c3)cc21]

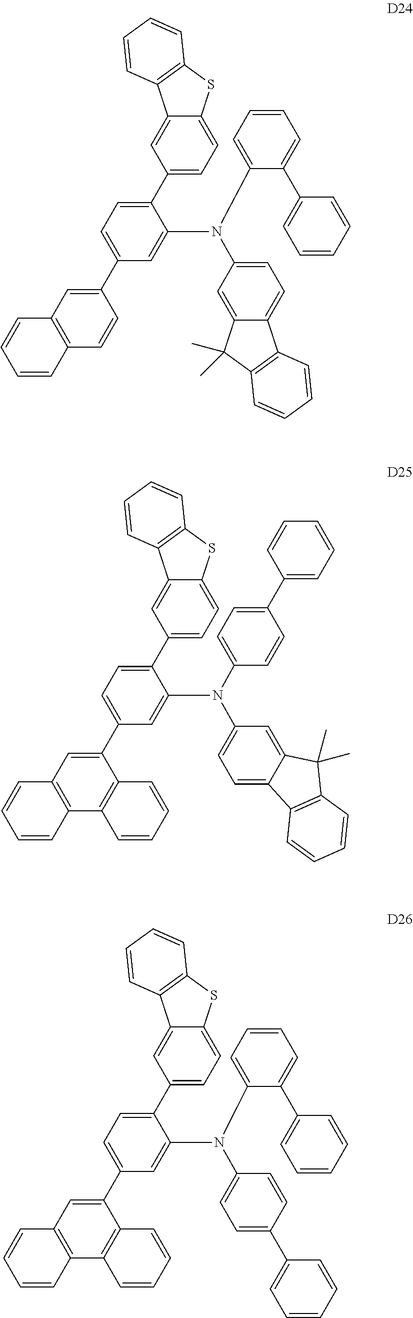 [SMILES: CC1(C)c2ccccc2-c2ccc(N(c3ccccc3-c3ccccc3)c3cc(-c4ccc5ccccc5c4)ccc3-c3ccc4sc5ccccc5c4c3)cc21 ; CC1(C)c2ccccc2-c2ccc(N(c3ccc(-c4ccccc4)cc3)c3cc(-c4cc5ccccc5c5ccccc45)ccc3-c3ccc4sc5ccccc5c4c3)cc21 ; c1ccc(-c2ccc(N(c3ccccc3-c3ccccc3)c3cc(-c4cc5ccccc5c5ccccc45)ccc3-c3ccc4sc5ccccc5c4c3)cc2)cc1]

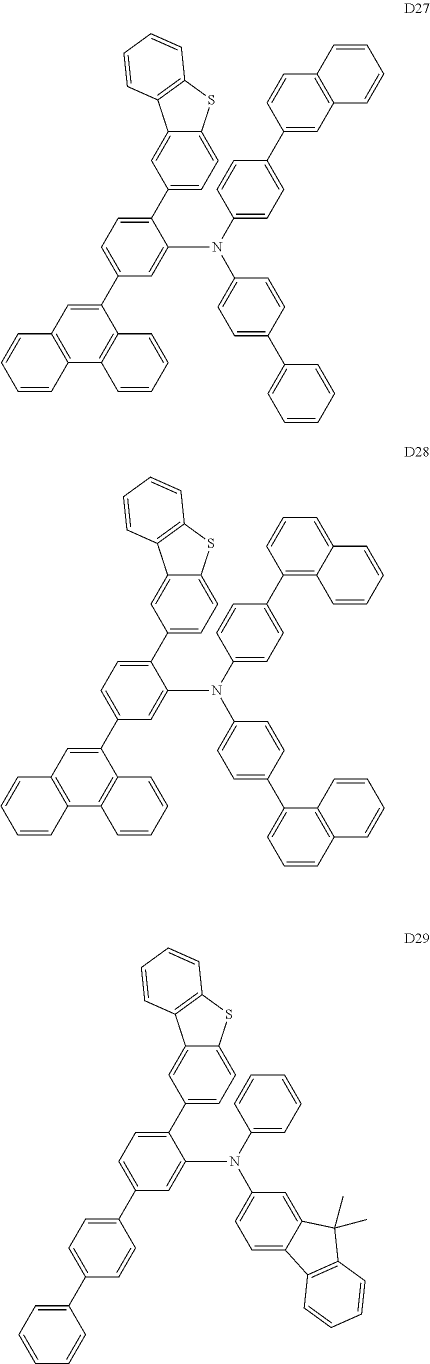 [SMILES: c1ccc(-c2ccc(N(c3ccc(-c4ccc5ccccc5c4)cc3)c3cc(-c4cc5ccccc5c5ccccc45)ccc3-c3ccc4sc5ccccc5c4c3)cc2)cc1 ; c1ccc2c(-c3ccc(N(c4ccc(-c5cccc6ccccc56)cc4)c4cc(-c5cc6ccccc6c6ccccc56)ccc4-c4ccc5sc6ccccc6c5c4)cc3)cccc2c1 ; CC1(C)c2ccccc2-c2ccc(N(c3ccccc3)c3cc(-c4ccc(-c5ccccc5)cc4)ccc3-c3ccc4sc5ccccc5c4c3)cc21]

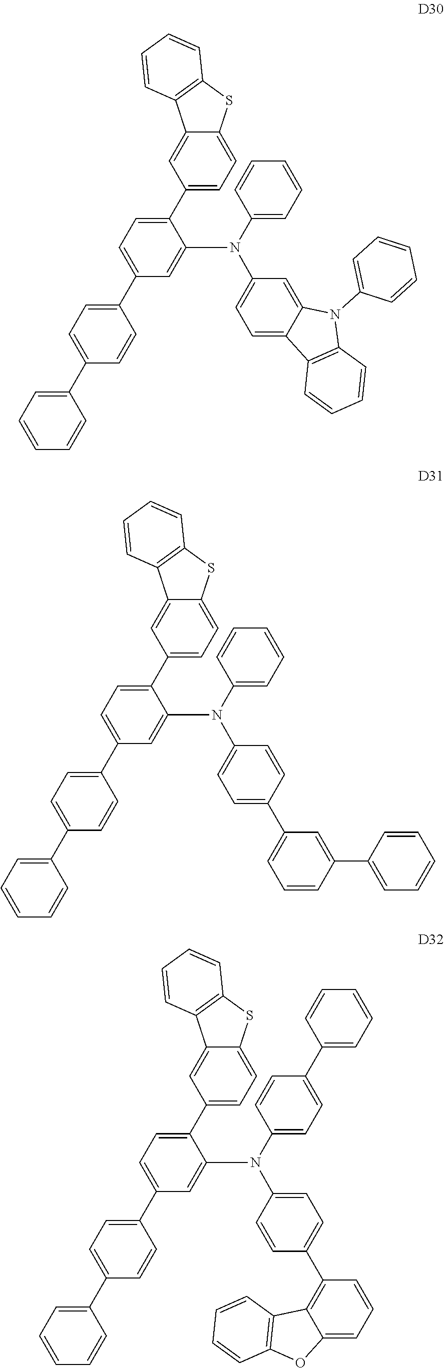 [SMILES: c1ccc(-c2ccc(-c3ccc(-c4ccc5sc6ccccc6c5c4)c(N(c4ccccc4)c4ccc5c6ccccc6n(-c6ccccc6)c5c4)c3)cc2)cc1 ; c1ccc(-c2ccc(-c3ccc(-c4ccc5sc6ccccc6c5c4)c(N(c4ccccc4)c4ccc(-c5cccc(-c6ccccc6)c5)cc4)c3)cc2)cc1 ; c1ccc(-c2ccc(-c3ccc(-c4ccc5sc6ccccc6c5c4)c(N(c4ccc(-c5ccccc5)cc4)c4ccc(-c5cccc6oc7ccccc7c56)cc4)c3)cc2)cc1]

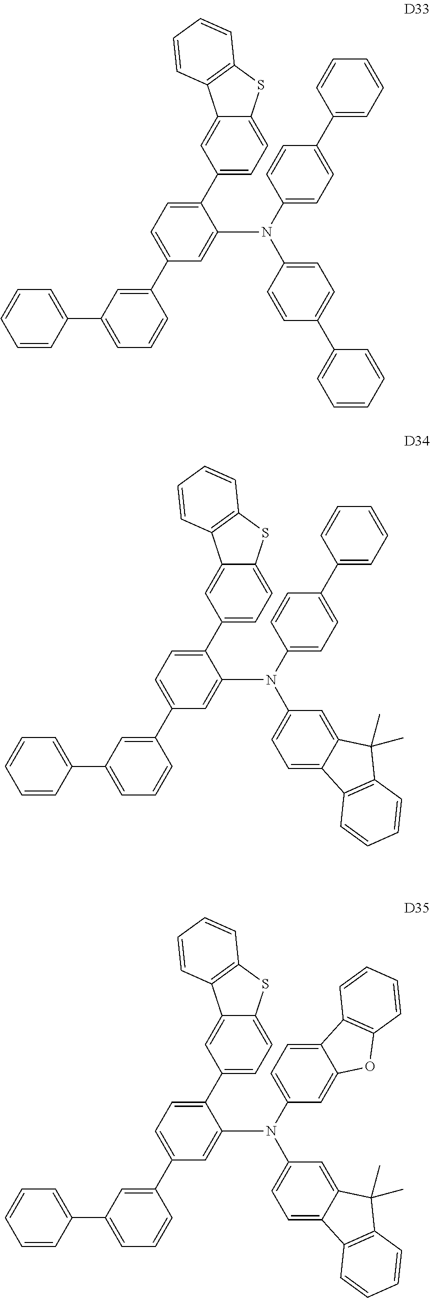 [SMILES: c1ccc(-c2ccc(N(c3ccc(-c4ccccc4)cc3)c3cc(-c4cccc(-c5ccccc5)c4)ccc3-c3ccc4sc5ccccc5c4c3)cc2)cc1 ; CC1(C)c2ccccc2-c2ccc(N(c3ccc(-c4ccccc4)cc3)c3cc(-c4cccc(-c5ccccc5)c4)ccc3-c3ccc4sc5ccccc5c4c3)cc21 ; CC1(C)c2ccccc2-c2ccc(N(c3ccc4c(c3)oc3ccccc34)c3cc(-c4cccc(-c5ccccc5)c4)ccc3-c3ccc4sc5ccccc5c4c3)cc21]

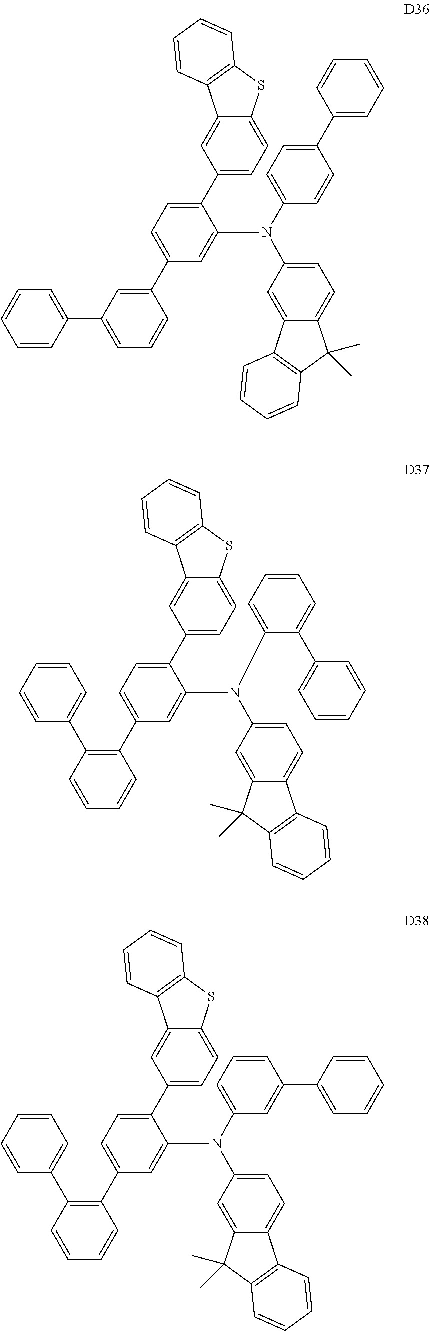 [SMILES: CC1(C)c2ccccc2-c2cc(N(c3ccc(-c4ccccc4)cc3)c3cc(-c4cccc(-c5ccccc5)c4)ccc3-c3ccc4sc5ccccc5c4c3)ccc21 ; CC1(C)c2ccccc2-c2ccc(N(c3ccccc3-c3ccccc3)c3cc(-c4ccccc4-c4ccccc4)ccc3-c3ccc4sc5ccccc5c4c3)cc21 ; CC1(C)c2ccccc2-c2ccc(N(c3cccc(-c4ccccc4)c3)c3cc(-c4ccccc4-c4ccccc4)ccc3-c3ccc4sc5ccccc5c4c3)cc21]

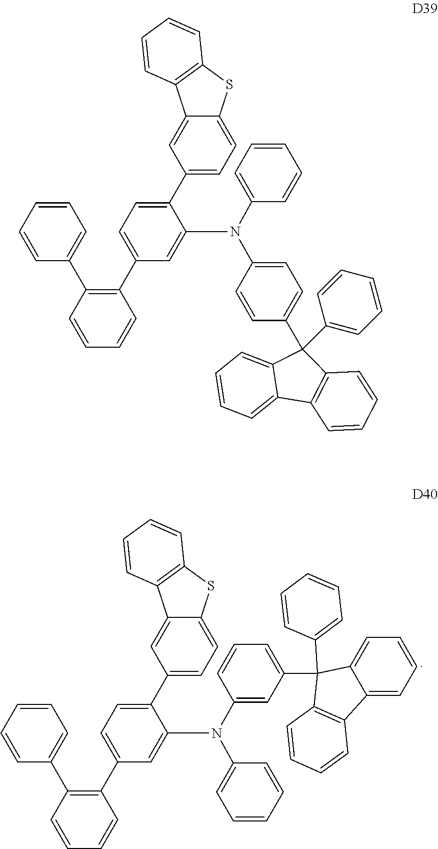 [SMILES: c1ccc(-c2ccccc2-c2ccc(-c3ccc4sc5ccccc5c4c3)c(N(c3ccccc3)c3ccc(C4(c5ccccc5)c5ccccc5-c5ccccc54)cc3)c2)cc1 ; c1ccc(-c2ccccc2-c2ccc(-c3ccc4sc5ccccc5c4c3)c(N(c3ccccc3)c3cccc(C4(c5ccccc5)c5ccccc5-c5ccccc54)c3)c2)cc1]

12. An organic electroluminescent device, comprising an anode and a cathode which are oppositely disposed, and a functional layer disposed between the anode and the cathode, wherein
the functional layer comprises the organic compound according to claim 1;
optionally, the functional layer comprises a hole auxiliary layer; and the hole auxiliary layer comprises the organic compound;
optionally, the organic electroluminescent device is a red organic electroluminescent device.

13. An electronic apparatus, comprising the organic electroluminescent device according to claim 12.
